(19) United States
(12) Patent Application Publication
(10) Pub. No.: US 2025/0376483 A1
(43) Pub. Date: Dec. 11, 2025

(54) PRODRUGS OF PAN-KRAS INHIBITORS

(71) Applicant: Mirati Therapeutics, Inc., Princeton, NJ (US)

(72) Inventors: Matthew Arnold MARX, San Diego, CA (US); Rongliang CHEN, San Diego, CA (US); Xiaolun WANG, San Diego, CA (US)

(21) Appl. No.: 18/875,584

(22) PCT Filed: Jun. 13, 2023
(86) PCT No.: PCT/US2023/025193

§ 371 Date: Dec. 16, 2024

Related U.S. Application Data
(60) Provisional application No. 63/352,524, filed on Jun. 15, 2022.
(60) Provisional application No. 63/459,547, filed on Apr. 14, 2023.

(51) Int. Cl. C07D 519/00; A61K 31/519; A61K 31/5377; A61K 31/55

(52) U.S. Cl. CPC C07D 519/00; A61K 31/519; A61K 31/5377; A61K 31/55

(57) ABSTRACT

The present invention relates to prodrugs of specific compounds that inhibits multiple mutated forms of KRas, i.e., the pan-KRas inhibitors. In particular, the present invention relates to prodrugs of these pan-KRas compound, pharmaceutical compositions comprising the prodrugs and methods of use therefor.

### FIELD OF THE INVENTION

The present invention relates to prodrugs of specific compounds that inhibit multiple mutated forms of KRas, i.e., the pan-KRas inhibitors. In particular, the present invention relates to prodrugs of pan-KRas compounds, pharmaceutical compositions comprising these prodrugs and methods of use therefor.

### BACKGROUND OF THE INVENTION

Kirsten Rat Sarcoma 2 Viral Oncogene Homolog (“KRas”) is a small GTPase and a member of the Ras family of oncogenes. KRas serves as a molecular switch cycling between inactive (GDP-bound) and active (GTP-bound) states to transduce upstream cellular signals received from multiple tyrosine kinases to downstream effectors to regulate a wide variety of processes, including cellular proliferation (e.g., see Alamgeer et al., (2013) Current Opin Pharmcol. 13:394-401).

The role of activated KRas in malignancy was observed over thirty years ago (e.g., see Santos et al., (1984) Science 223:661-664). Aberrant expression of KRas accounts for up to 20% of all cancers and oncogenic KRas mutations that stabilize GTP binding and lead to constitutive activation of KRas. KRas mutations at codons 12, 13, 61 and other positions of the KRas primary amino acid sequence are present in 88% of all pancreatic adenocarcinoma patients, 50% of all colon/rectal adenocarcinoma patients, and 32% lung adenocarcinoma patients (e.g., see Prior et all., (2020) Cancer Res 80:2969-74). A recent publication also suggested wild type Kras inhibition could be a viable therapeutic strategy to treat KRas^{WT} dependent cancers (e.g., see Bery et al., (2020) Nat. Commun. 11: 3233).

The well-known role of KRas in malignancy and the discovery of these frequent mutations in KRas in various tumor types made KRas a highly attractive target of the pharmaceutical industry for cancer therapy. Notwithstanding thirty years of large-scale discovery efforts to develop inhibitors of KRas for treating cancer, no KRas inhibitor has yet demonstrated sufficient safety and/or efficacy to obtain regulatory approval (e.g., see McCormick (2015) Clin Cancer Res. 21 (8):1797-1801).

Compounds that inhibit KRas activity are still highly desirable and under investigation, including those that disrupt effectors such as guanine nucleotide exchange factors (e.g., see Sun et al., (2012) Agnew Chem Int Ed Engl. 51(25):6140-6143 doi: 10.1002/anie201201358) as well recent advances in the covalent targeting of an allosteric pocket of KRas G12C (e.g., see Ostrem et al., (2013) Nature 503:548-551 and Fell et al., (2018) ACS Med. Chem. Lett. 9:1230-1234). Clearly there remains a continued interest and effort to develop inhibitors of KRas, particularly inhibitors of activating KRas mutants.

A compound having the following formula:

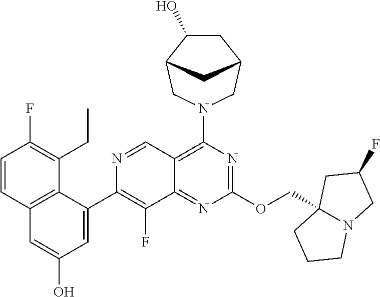 [SMILES: CCc1c(F)ccc2cc(O)cc(-c3ncc4c(N5C[C@@H]6C[C@H](C5)[C@H](O)C6)nc(OC[C@@]56CCCN5C[C@H](F)C6)nc4c3F)c12]

3-(7-(8-ethyl-7-fluoro-3-hydroxynaphthalen-1-yl)-8-fluoro-2-(((2R,7aS)-2-fluorohexahydro-1H-pyrrolizin-7α-yl)methoxy)pyrido[4,3-d]pyrimidin-4-yl)-3-azabicyclo[3.2.1]octan-6-ol
   is described in PCT International Application No. PCT/US2021/010065 (WO 2022/132200), as the species of Example 140.

Similarly, a compound having the formula:

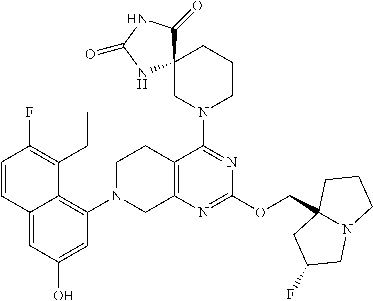

(R)-7-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-1,3,7-triazaspiro[4.5]decane-2,4-dione
   is described in PCT International Application No. PCT/US2021/063722 (WO 2022/133038), as the species of Example 196.

There is a need in the art to develop prodrugs of these and similar compounds which convert to these compounds in vivo and therefore, would be efficient for treating KRas-mediated cancers.

### SUMMARY OF THE INVENTION

In one aspect, the invention provides prodrugs of a compound of the following structure:

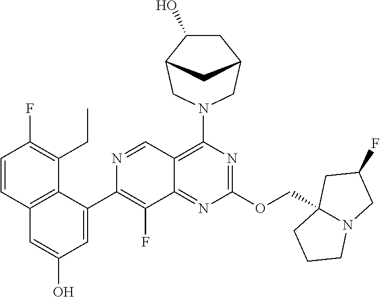 [SMILES: CCc1c(F)ccc2cc(O)cc(-c3ncc4c(N5C[C@@H]6C[C@H](C5)[C@H](O)C6)nc(OC[C@@]56CCCN5C[C@H](F)C6)nc4c3F)c12]

3-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-3-azabicyclo[3.2.1]octan-6-ol

This compound is described in PCT International Application No. PCT/US2021/010065 (WO 2022/132200), as the species of Example 140.

In another aspect, the invention provides prodrugs of a compound of the following structure:

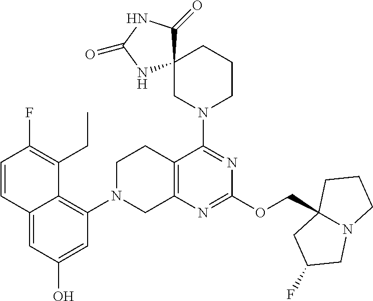 [SMILES: CCc1c(F)ccc2cc(O)cc(N3CCc4c(nc(OC[C@@]56CCCN5C[C@H](F)C6)nc4N4CCC[C@]5(C4)NC(=O)NC5=O)C3)c12]

(R)-7-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-1,3,7-triazaspiro[4.5]decane-2,4-dione

This compound is described in PCT International Application No. PCT/US2021/063722 (WO 2022/133038), as the species of Example 196.

In one aspect, the provided prodrugs have the generic structure of Formula (I):

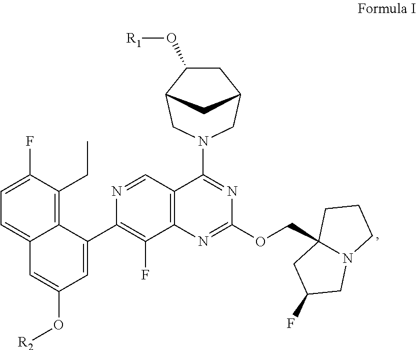

or a pharmaceutically acceptable salt thereof,
wherein
R₁ and R₂ are independently selected from the group consisting of:
H, R₃—*, R₃—O—CH₂—*,

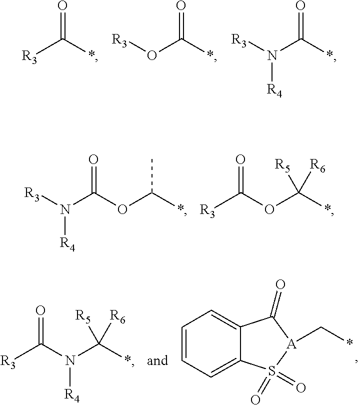

with the proviso that only one of R₁ and R₂ can be H;
A is CH or N;
R₃ is C₁-C₂₀ alkyl, (C₁-C₄ alkyl)₀₋₁-cycloalkyl, (C₁-C₄ alkyl)₀₋₁-heterocyclyl or (C₁-C₄ alkyl)₀₋₁-aryl, where R₃ is optionally substituted with C₁-C₄ alkyl or —O— C₁-C₄ alkyl;
R₄ is H or R₃;
or R₃ and R₄ on a nitrogen join to form a heterocycle; and
R₅ and R₆ are independently selected from the group consisting of H and C₁-C₆ alkyl.

In one aspect, R₁ is H and R₂ is R₃—*, R₃—O—CH₂—*,

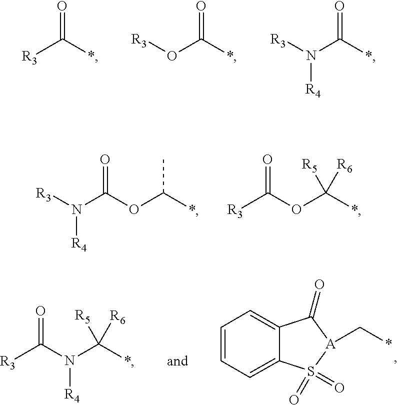

In another aspect, R₂ is H and R₁ is R₃—*, R₃—O—CH₂—*,

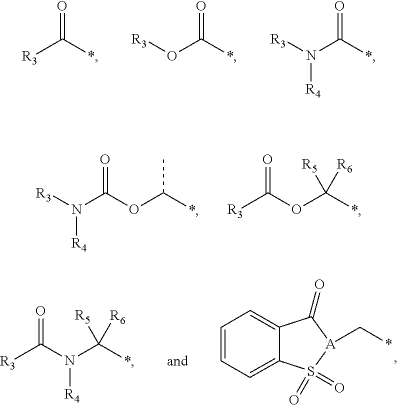

In one aspect, the provided prodrugs have the following structures:

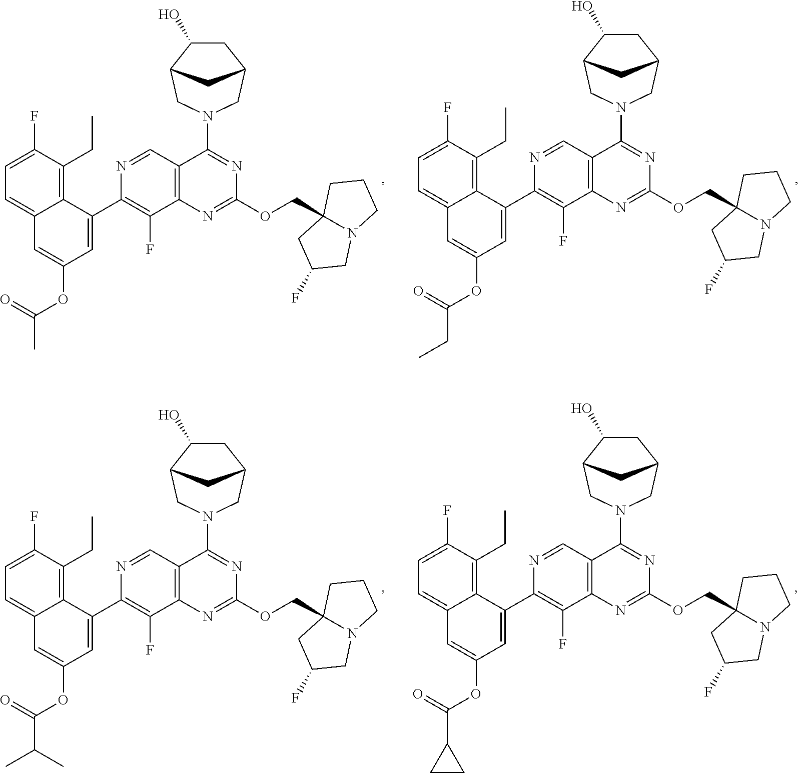

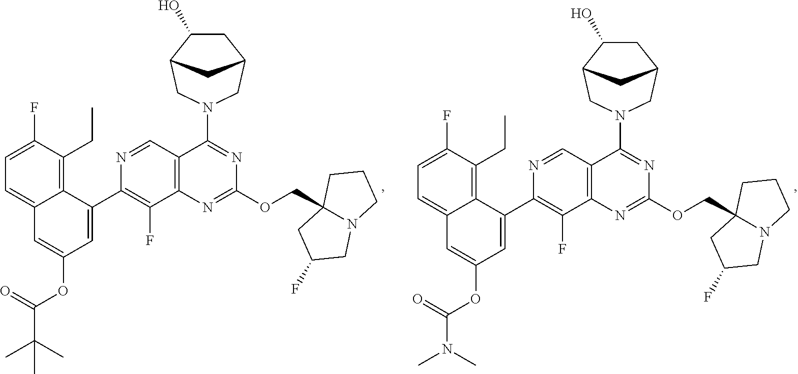

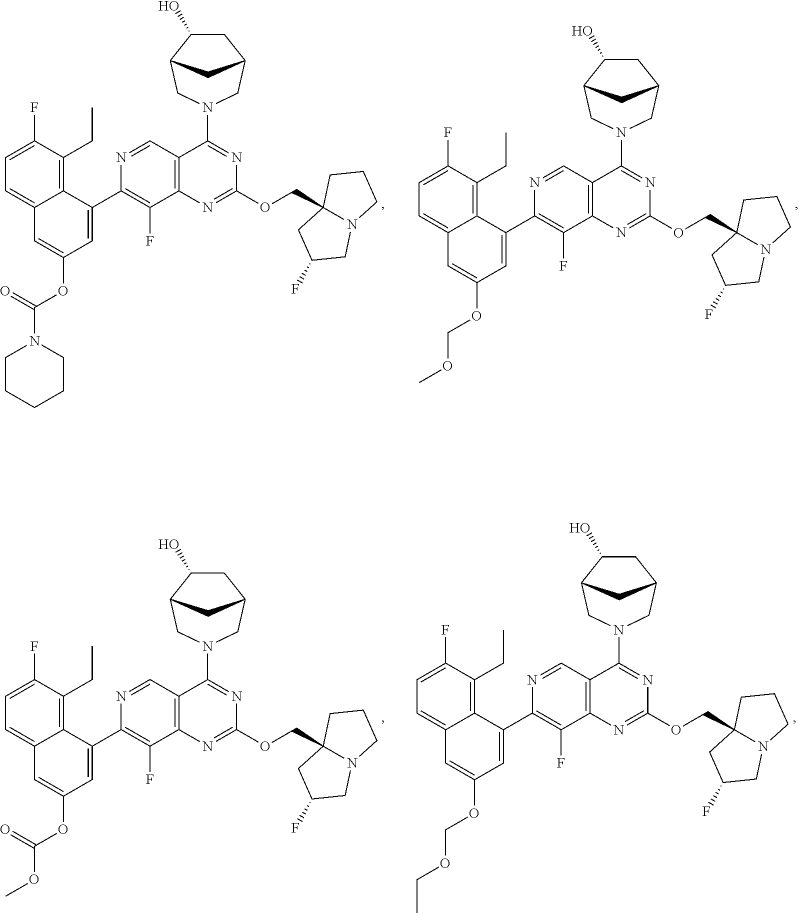

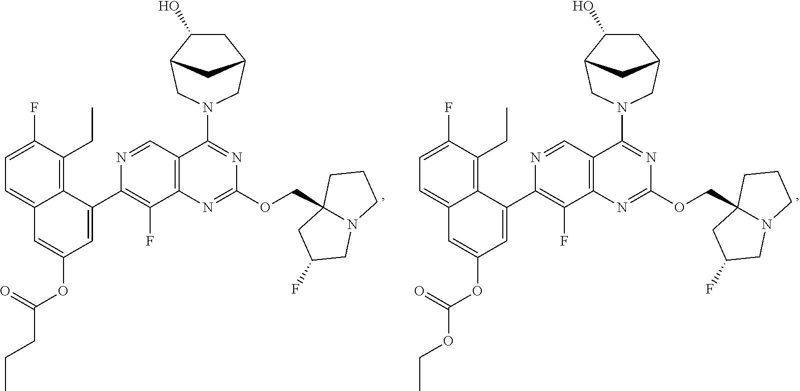

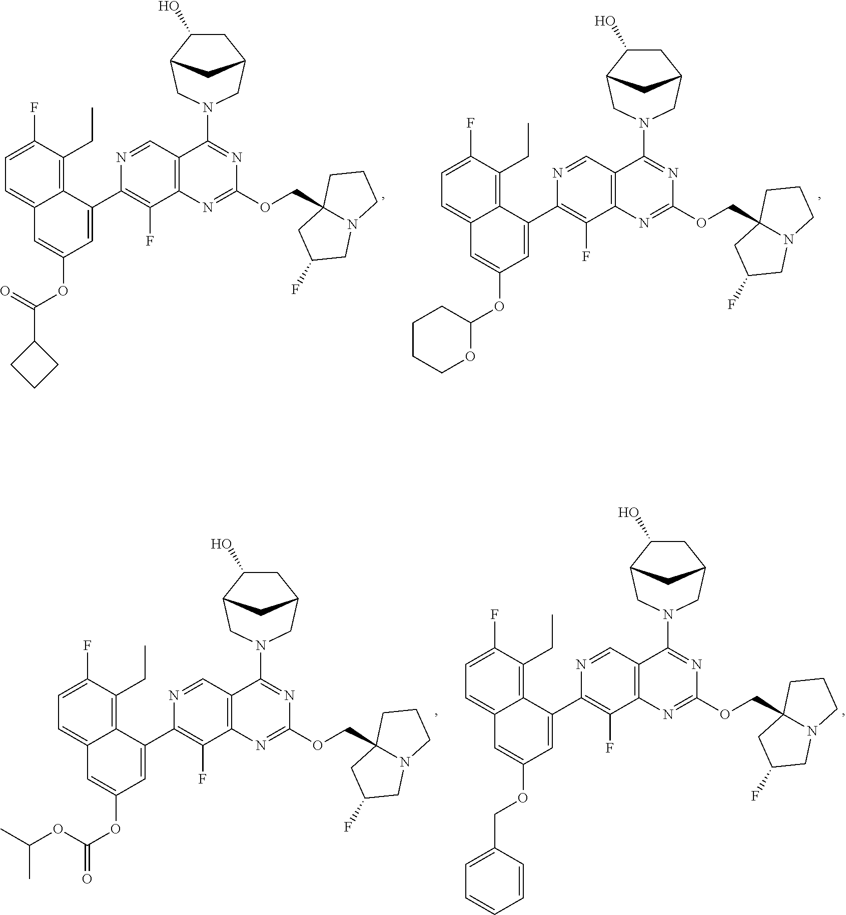 [SMILES: CCc1c(F)ccc2cc(OC(=O)C3CCC3)cc(-c3ncc4c(N5C[C@@H]6C[C@H](C5)[C@H](O)C6)nc(OC[C@@]56CCCN5C[C@H](F)C6)nc4c3F)c12 ; CCc1c(F)ccc2cc(OC3CCCCO3)cc(-c3ncc4c(N5C[C@@H]6C[C@H](C5)[C@H](O)C6)nc(OC[C@@]56CCCN5C[C@H](F)C6)nc4c3F)c12 ; CCc1c(F)ccc2cc(OC(=O)OC(C)C)cc(-c3ncc4c(N5C[C@@H]6C[C@H](C5)[C@H](O)C6)nc(OC[C@@]56CCCN5C[C@H](F)C6)nc4c3F)c12 ; CCc1c(F)ccc2cc(OCc3ccccc3)cc(-c3ncc4c(N5C[C@@H]6C[C@H](C5)[C@H](O)C6)nc(OC[C@@]56CCCN5C[C@H](F)C6)nc4c3F)c12]

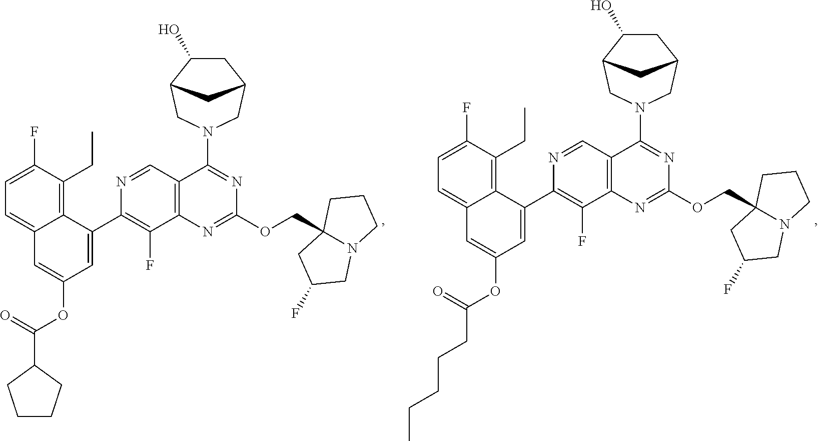 [SMILES: CCc1c(F)ccc2cc(OC(=O)C3CCCC3)cc(-c3ncc4c(N5C[C@@H]6C[C@H](C5)[C@H](O)C6)nc(OC[C@@]56CCCN5C[C@H](F)C6)nc4c3F)c12 ; CCCCCC(=O)Oc1cc(-c2ncc3c(N4C[C@@H]5C[C@H](C4)[C@H](O)C5)nc(OC[C@@]45CCCN4C[C@H](F)C5)nc3c2F)c2c(CC)c(F)ccc2c1]

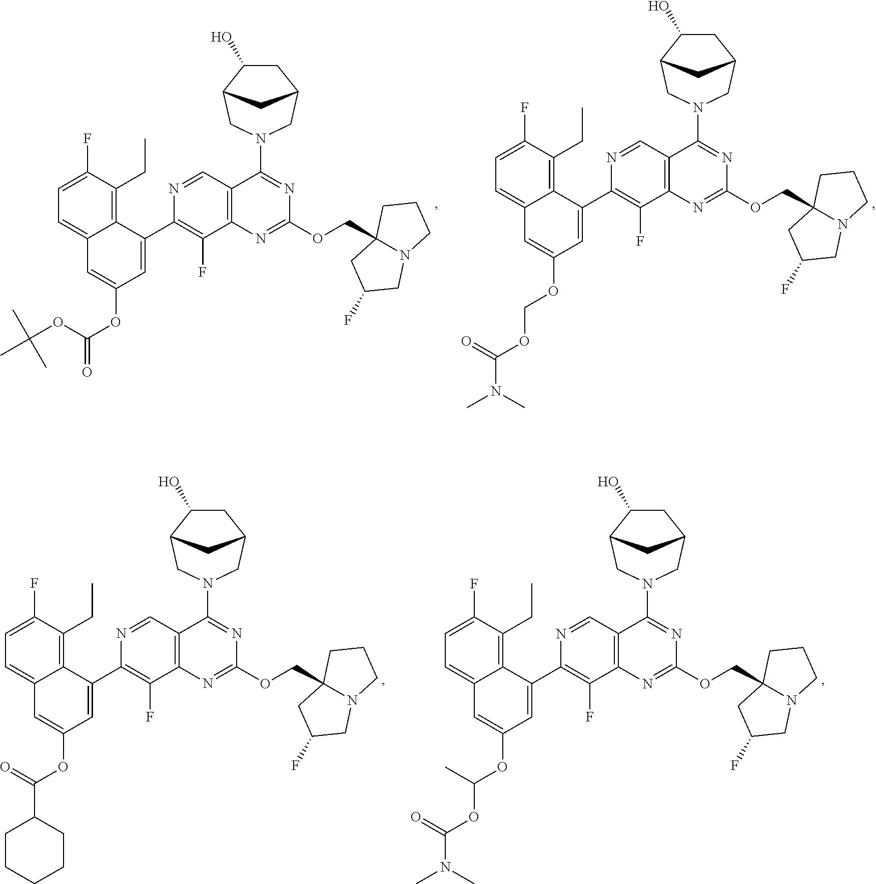 [SMILES: CCc1c(F)ccc2cc(OC(=O)OC(C)(C)C)cc(-c3ncc4c(N5C[C@@H]6C[C@H](C5)[C@H](O)C6)nc(OC[C@@]56CCCN5C[C@H](F)C6)nc4c3F)c12 ; CCc1c(F)ccc2cc(OCOC(=O)N(C)C)cc(-c3ncc4c(N5C[C@@H]6C[C@H](C5)[C@H](O)C6)nc(OC[C@@]56CCCN5C[C@H](F)C6)nc4c3F)c12 ; CCc1c(F)ccc2cc(OC(=O)C3CCCCC3)cc(-c3ncc4c(N5C[C@@H]6C[C@H](C5)[C@H](O)C6)nc(OC[C@@]56CCCN5C[C@H](F)C6)nc4c3F)c12 ; CCc1c(F)ccc2cc(OC(C)OC(=O)N(C)C)cc(-c3ncc4c(N5C[C@@H]6C[C@H](C5)[C@H](O)C6)nc(OC[C@@]56CCCN5C[C@H](F)C6)nc4c3F)c12]

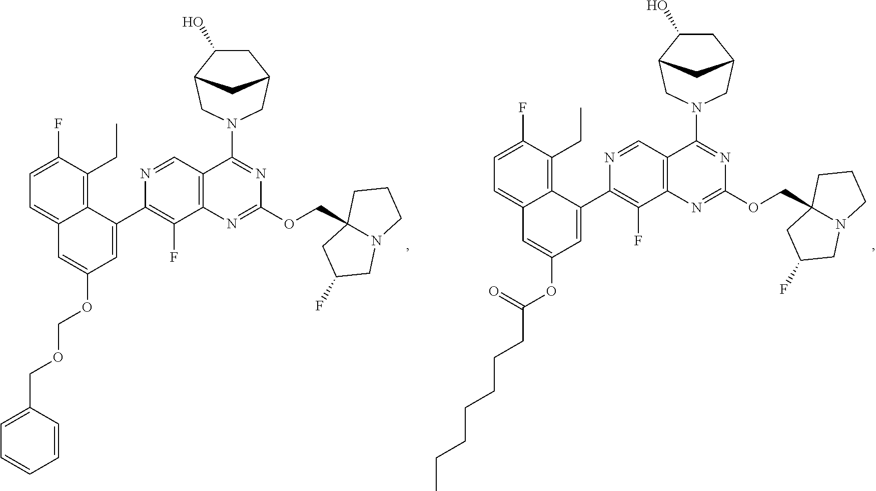 [SMILES: CCc1c(F)ccc2cc(OCOCc3ccccc3)cc(-c3ncc4c(N5C[C@@H]6C[C@H](C5)[C@H](O)C6)nc(OC[C@@]56CCCN5C[C@H](F)C6)nc4c3F)c12 ; CCCCCCCC(=O)Oc1cc(-c2ncc3c(N4C[C@@H]5C[C@H](C4)[C@H](O)C5)nc(OC[C@@]45CCCN4C[C@H](F)C5)nc3c2F)c2c(CC)c(F)ccc2c1]

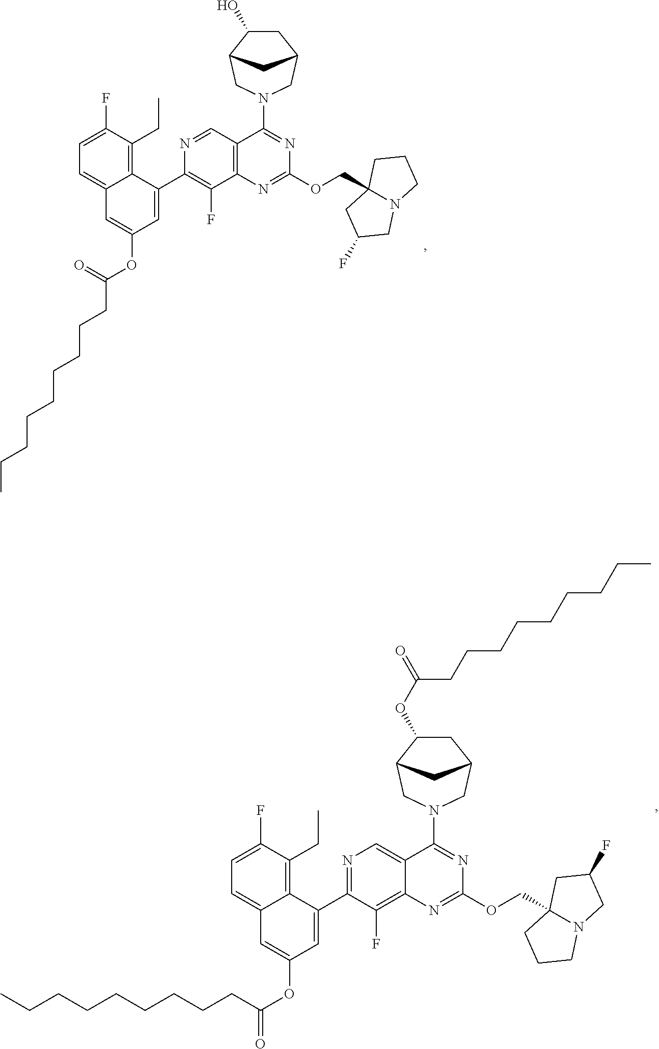 [SMILES: CCCCCCCCCC(=O)Oc1cc(-c2ncc3c(N4C[C@@H]5C[C@H](C4)[C@H](O)C5)nc(OC[C@@]45CCCN4C[C@H](F)C5)nc3c2F)c2c(CC)c(F)ccc2c1 ; CCCCCCCCCC(=O)Oc1cc(-c2ncc3c(N4C[C@@H]5C[C@H](C4)[C@H](OC(=O)CCCCCCCCC)C5)nc(OC[C@@]45CCCN4C[C@H](F)C5)nc3c2F)c2c(CC)c(F)ccc2c1]

and pharmaceutically acceptable salts thereof.

In another aspect of the invention, the provided prodrugs have the following generic structure:

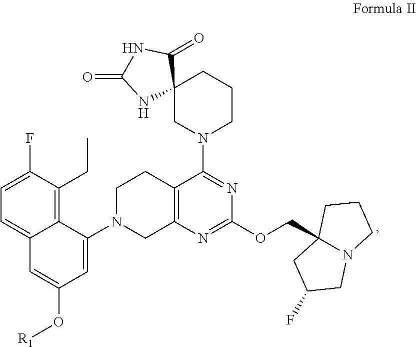

or a pharmaceutically acceptable salt thereof,
wherein R₁ is H R₃—*, R₃—O—CH₂—*,

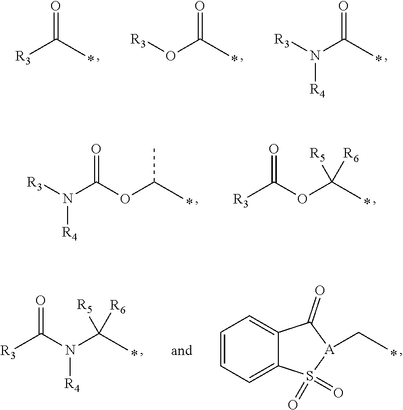

A is CH or N;
R₃ is C₁-C₂₀ alkyl, (C₁-C₄ alkyl)₀₋₁-cycloalkyl, (C₁-C₄ alkyl)₀₋₁-heterocyclyl or (C₁-C₄ alkyl)₀₋₁-aryl, where R₃ is optionally substituted with C₁-C₄ alkyl or —O— C₁-C₄ alkyl;
R₄ is H or R₃;
R₅ and R₆ are independently selected from the group consisting of H and C₁-C₆ alkyl.

In one aspect, the compounds of Formula II are selected from the group consisting

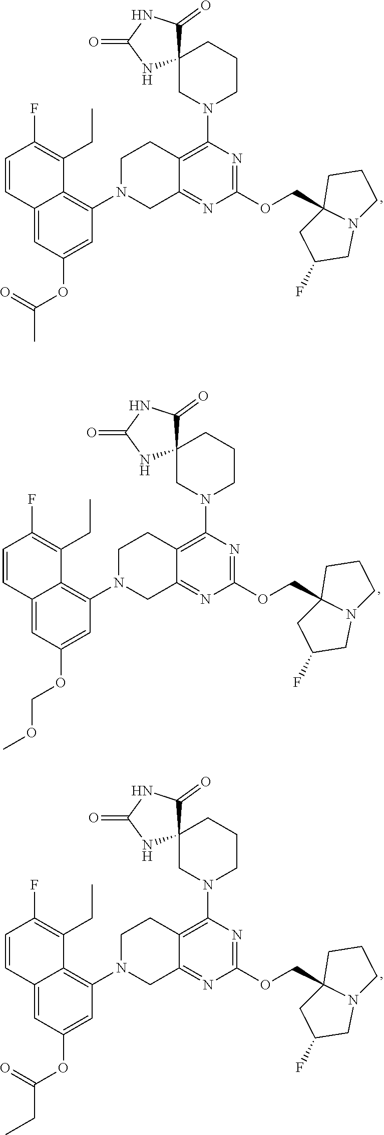 [SMILES: CCc1c(F)ccc2cc(OC(C)=O)cc(N3CCc4c(nc(OC[C@@]56CCCN5C[C@H](F)C6)nc4N4CCC[C@]5(C4)NC(=O)NC5=O)C3)c12 ; CCc1c(F)ccc2cc(OCOC)cc(N3CCc4c(nc(OC[C@@]56CCCN5C[C@H](F)C6)nc4N4CCC[C@]5(C4)NC(=O)NC5=O)C3)c12 ; CCC(=O)Oc1cc(N2CCc3c(nc(OC[C@@]45CCCN4C[C@H](F)C5)nc3N3CCC[C@]4(C3)NC(=O)NC4=O)C2)c2c(CC)c(F)ccc2c1]

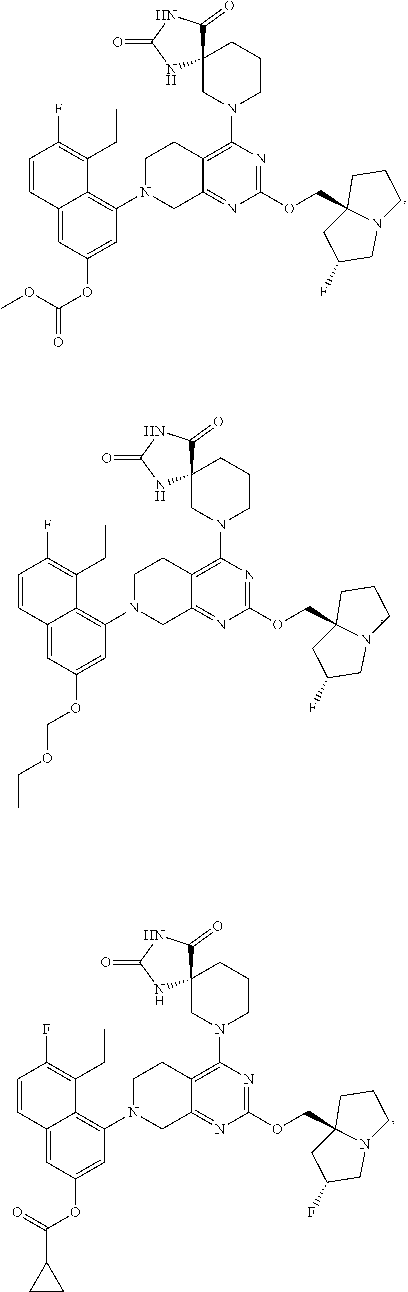 [SMILES: CCc1c(F)ccc2cc(OC(=O)OC)cc(N3CCc4c(nc(OC[C@@]56CCCN5C[C@H](F)C6)nc4N4CCC[C@]5(C4)NC(=O)NC5=O)C3)c12 ; CCOCOc1cc(N2CCc3c(nc(OC[C@@]45CCCN4C[C@H](F)C5)nc3N3CCC[C@]4(C3)NC(=O)NC4=O)C2)c2c(CC)c(F)ccc2c1 ; CCc1c(F)ccc2cc(OC(=O)C3CC3)cc(N3CCc4c(nc(OC[C@@]56CCCN5C[C@H](F)C6)nc4N4CCC[C@]5(C4)NC(=O)NC5=O)C3)c12]

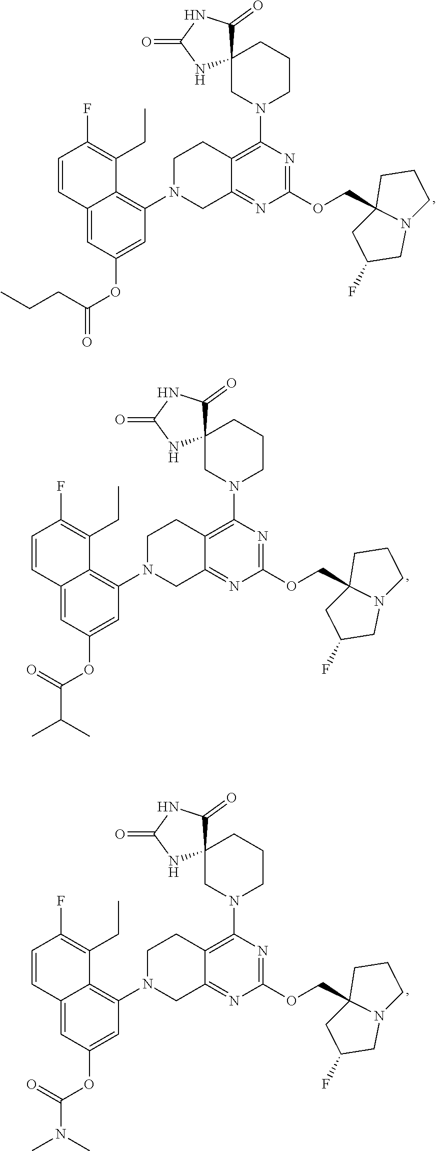 [SMILES: CCCC(=O)Oc1cc(N2CCc3c(nc(OC[C@@]45CCCN4C[C@H](F)C5)nc3N3CCC[C@]4(C3)NC(=O)NC4=O)C2)c2c(CC)c(F)ccc2c1 ; CCc1c(F)ccc2cc(OC(=O)C(C)C)cc(N3CCc4c(nc(OC[C@@]56CCCN5C[C@H](F)C6)nc4N4CCC[C@]5(C4)NC(=O)NC5=O)C3)c12 ; CCc1c(F)ccc2cc(OC(=O)N(C)C)cc(N3CCc4c(nc(OC[C@@]56CCCN5C[C@H](F)C6)nc4N4CCC[C@]5(C4)NC(=O)NC5=O)C3)c12]

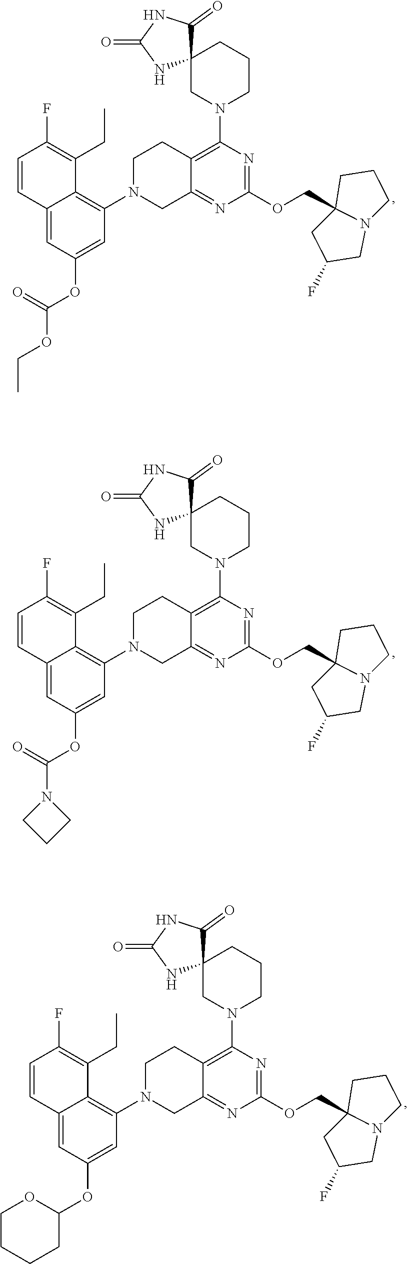 [SMILES: CCOC(=O)Oc1cc(N2CCc3c(nc(OC[C@@]45CCCN4C[C@H](F)C5)nc3N3CCC[C@]4(C3)NC(=O)NC4=O)C2)c2c(CC)c(F)ccc2c1 ; CCc1c(F)ccc2cc(OC(=O)N3CCC3)cc(N3CCc4c(nc(OC[C@@]56CCCN5C[C@H](F)C6)nc4N4CCC[C@]5(C4)NC(=O)NC5=O)C3)c12 ; CCc1c(F)ccc2cc(OC3CCCCO3)cc(N3CCc4c(nc(OC[C@@]56CCCN5C[C@H](F)C6)nc4N4CCC[C@]5(C4)NC(=O)NC5=O)C3)c12]

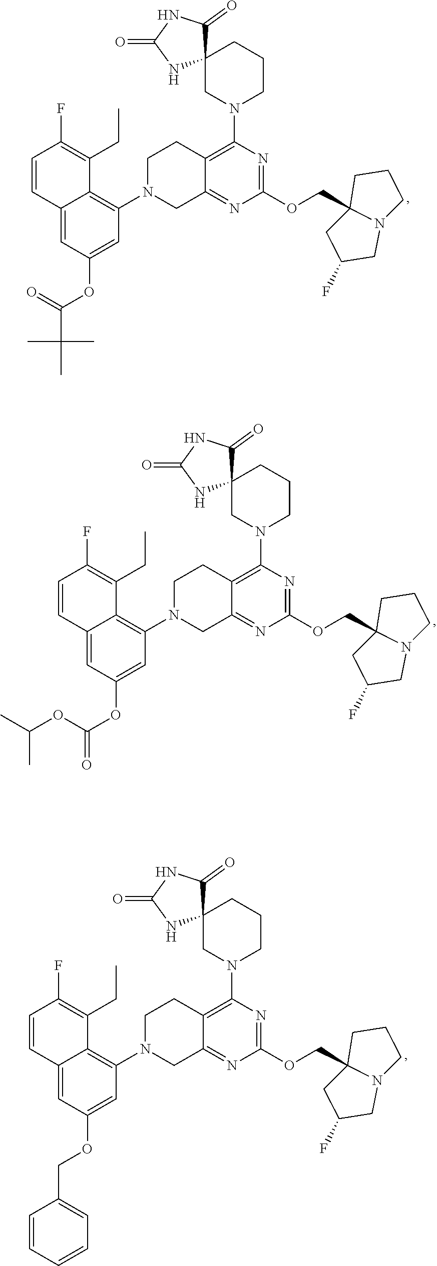 [SMILES: CCc1c(F)ccc2cc(OC(=O)C(C)(C)C)cc(N3CCc4c(nc(OC[C@@]56CCCN5C[C@H](F)C6)nc4N4CCC[C@]5(C4)NC(=O)NC5=O)C3)c12 ; CCc1c(F)ccc2cc(OC(=O)OC(C)C)cc(N3CCc4c(nc(OC[C@@]56CCCN5C[C@H](F)C6)nc4N4CCC[C@]5(C4)NC(=O)NC5=O)C3)c12 ; CCc1c(F)ccc2cc(OCc3ccccc3)cc(N3CCc4c(nc(OC[C@@]56CCCN5C[C@H](F)C6)nc4N4CCC[C@]5(C4)NC(=O)NC5=O)C3)c12]

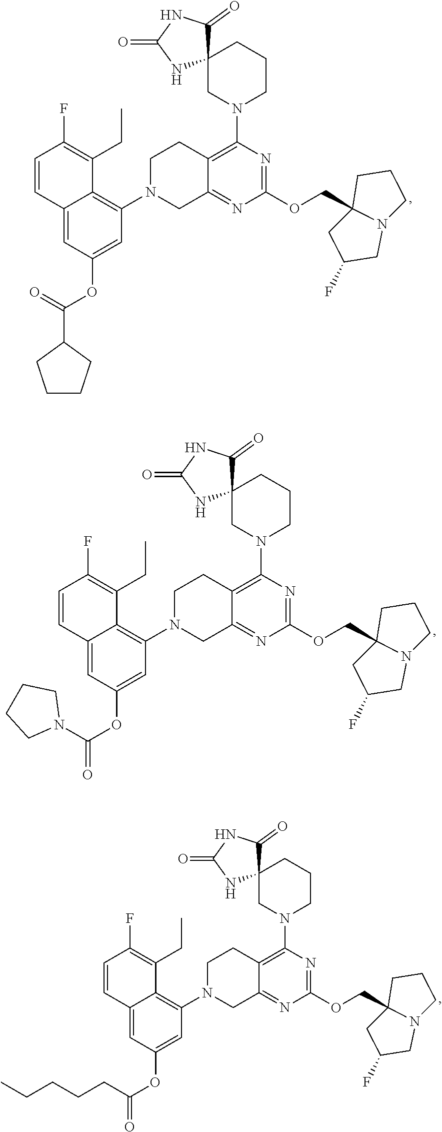 [SMILES: CCc1c(F)ccc2cc(OC(=O)C3CCCC3)cc(N3CCc4c(nc(OC[C@@]56CCCN5C[C@H](F)C6)nc4N4CCC[C@]5(C4)NC(=O)NC5=O)C3)c12 ; CCc1c(F)ccc2cc(OC(=O)N3CCCC3)cc(N3CCc4c(nc(OC[C@@]56CCCN5C[C@H](F)C6)nc4N4CCC[C@]5(C4)NC(=O)NC5=O)C3)c12 ; CCCCCC(=O)Oc1cc(N2CCc3c(nc(OC[C@@]45CCCN4C[C@H](F)C5)nc3N3CCC[C@]4(C3)NC(=O)NC4=O)C2)c2c(CC)c(F)ccc2c1]

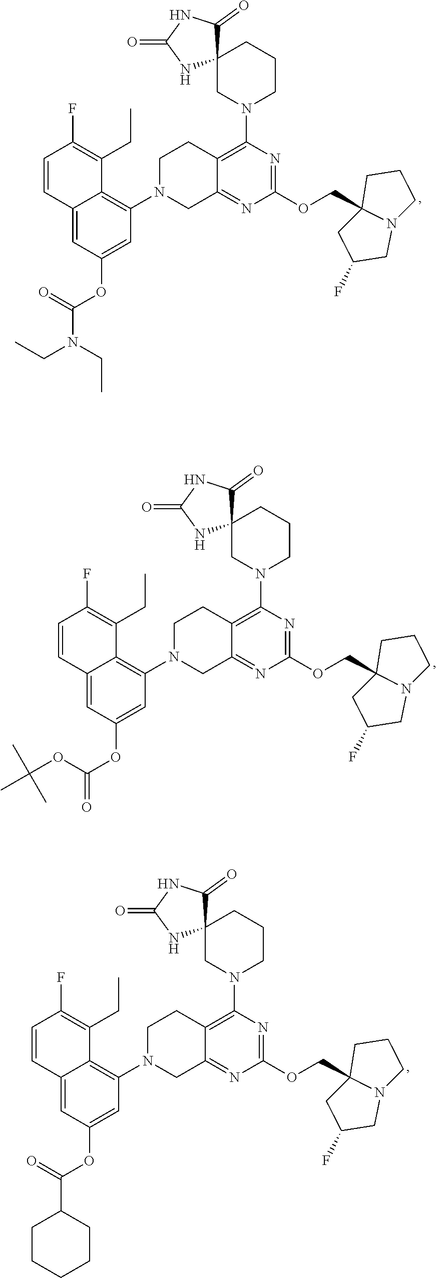 [SMILES: CCc1c(F)ccc2cc(OC(=O)N(CC)CC)cc(N3CCc4c(nc(OC[C@@]56CCCN5C[C@H](F)C6)nc4N4CCC[C@]5(C4)NC(=O)NC5=O)C3)c12 ; CCc1c(F)ccc2cc(OC(=O)OC(C)(C)C)cc(N3CCc4c(nc(OC[C@@]56CCCN5C[C@H](F)C6)nc4N4CCC[C@]5(C4)NC(=O)NC5=O)C3)c12 ; CCc1c(F)ccc2cc(OC(=O)C3CCCCC3)cc(N3CCc4c(nc(OC[C@@]56CCCN5C[C@H](F)C6)nc4N4CCC[C@]5(C4)NC(=O)NC5=O)C3)c12]

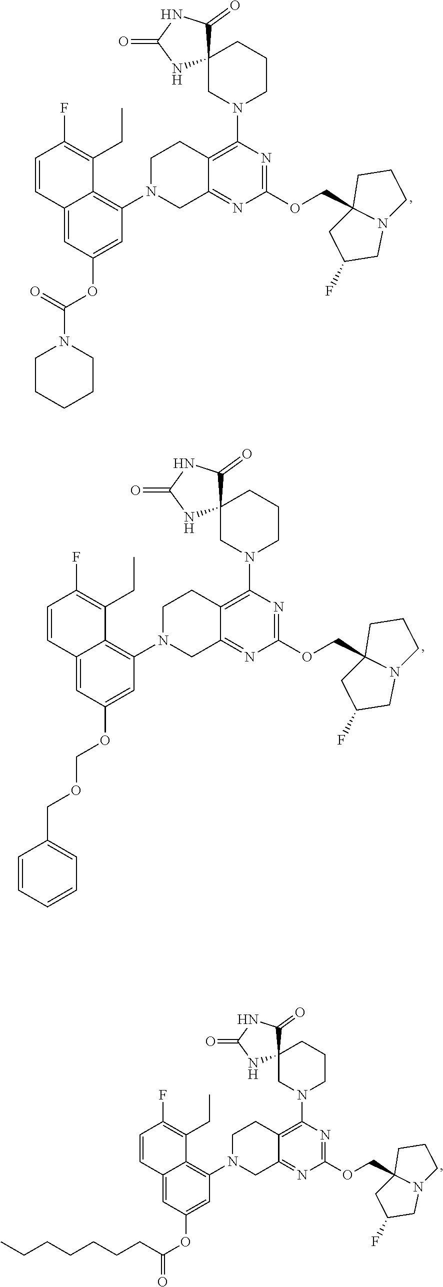 [SMILES: CCc1c(F)ccc2cc(OC(=O)N3CCCCC3)cc(N3CCc4c(nc(OC[C@@]56CCCN5C[C@H](F)C6)nc4N4CCC[C@]5(C4)NC(=O)NC5=O)C3)c12 ; CCc1c(F)ccc2cc(OCOCc3ccccc3)cc(N3CCc4c(nc(OC[C@@]56CCCN5C[C@H](F)C6)nc4N4CCC[C@]5(C4)NC(=O)NC5=O)C3)c12 ; CCCCCCCC(=O)Oc1cc(N2CCc3c(nc(OC[C@@]45CCCN4C[C@H](F)C5)nc3N3CCC[C@]4(C3)NC(=O)NC4=O)C2)c2c(CC)c(F)ccc2c1]

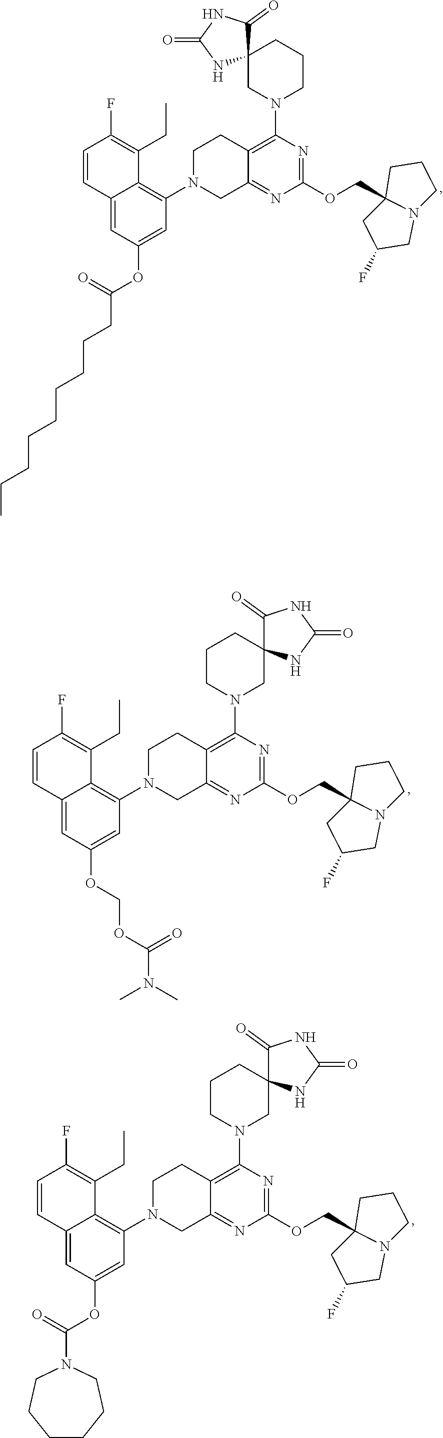 [SMILES: CCCCCCCCCC(=O)Oc1cc(N2CCc3c(nc(OC[C@@]45CCCN4C[C@H](F)C5)nc3N3CCC[C@]4(C3)NC(=O)NC4=O)C2)c2c(CC)c(F)ccc2c1 ; CCc1c(F)ccc2cc(OCOC(=O)N(C)C)cc(N3CCc4c(nc(OC[C@@]56CCCN5C[C@H](F)C6)nc4N4CCC[C@]5(C4)NC(=O)NC5=O)C3)c12 ; CCc1c(F)ccc2cc(OC(=O)N3CCCCCC3)cc(N3CCc4c(nc(OC[C@@]56CCCN5C[C@H](F)C6)nc4N4CCC[C@]5(C4)NC(=O)NC5=O)C3)c12]

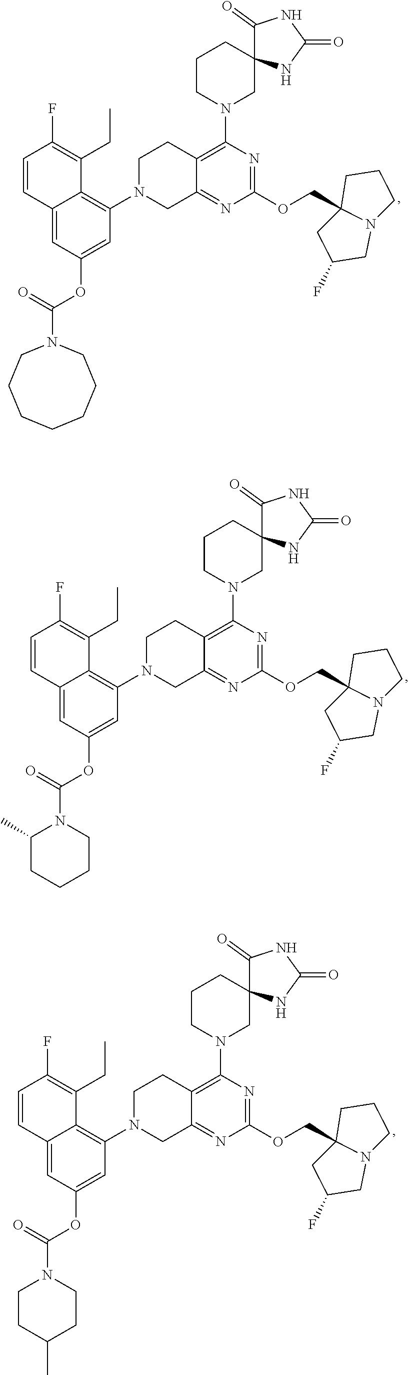 [SMILES: CCc1c(F)ccc2cc(OC(=O)N3CCCCCCC3)cc(N3CCc4c(nc(OC[C@@]56CCCN5C[C@H](F)C6)nc4N4CCC[C@]5(C4)NC(=O)NC5=O)C3)c12 ; CCc1c(F)ccc2cc(OC(=O)N3CCCC[C@@H]3C)cc(N3CCc4c(nc(OC[C@@]56CCCN5C[C@H](F)C6)nc4N4CCC[C@]5(C4)NC(=O)NC5=O)C3)c12 ; CCc1c(F)ccc2cc(OC(=O)N3CCC(C)CC3)cc(N3CCc4c(nc(OC[C@@]56CCCN5C[C@H](F)C6)nc4N4CCC[C@]5(C4)NC(=O)NC5=O)C3)c12]

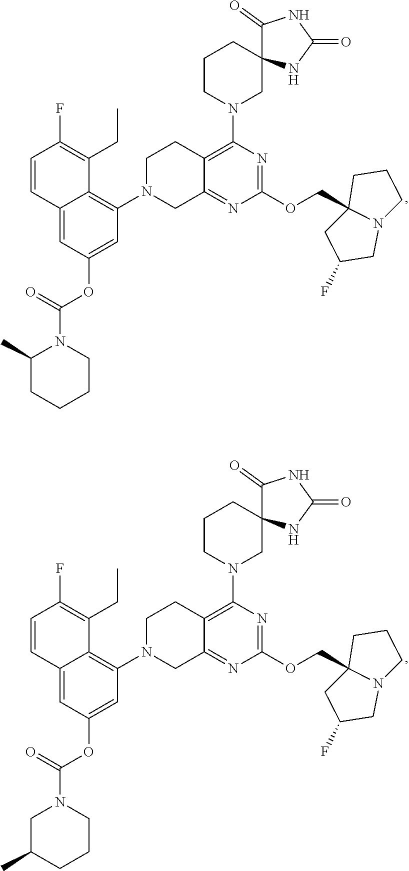 [SMILES: CCc1c(F)ccc2cc(OC(=O)N3CCCC[C@H]3C)cc(N3CCc4c(nc(OC[C@@]56CCCN5C[C@H](F)C6)nc4N4CCC[C@]5(C4)NC(=O)NC5=O)C3)c12 ; CCc1c(F)ccc2cc(OC(=O)N3CCC[C@@H](C)C3)cc(N3CCc4c(nc(OC[C@@]56CCCN5C[C@H](F)C6)nc4N4CCC[C@]5(C4)NC(=O)NC5=O)C3)c12]

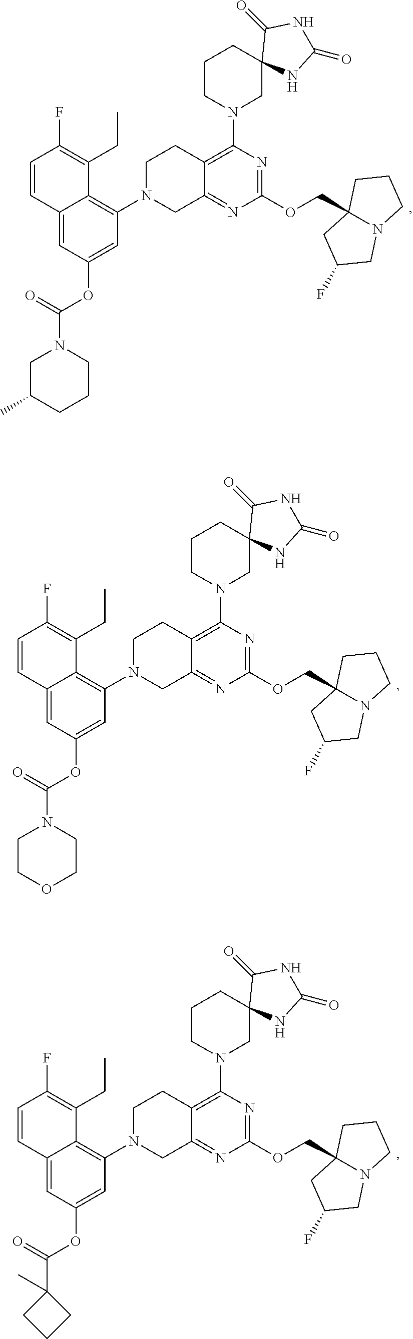 [SMILES: CCc1c(F)ccc2cc(OC(=O)N3CCC[C@H](C)C3)cc(N3CCc4c(nc(OC[C@@]56CCCN5C[C@H](F)C6)nc4N4CCC[C@]5(C4)NC(=O)NC5=O)C3)c12 ; CCc1c(F)ccc2cc(OC(=O)N3CCOCC3)cc(N3CCc4c(nc(OC[C@@]56CCCN5C[C@H](F)C6)nc4N4CCC[C@]5(C4)NC(=O)NC5=O)C3)c12 ; CCc1c(F)ccc2cc(OC(=O)C3(C)CCC3)cc(N3CCc4c(nc(OC[C@@]56CCCN5C[C@H](F)C6)nc4N4CCC[C@]5(C4)NC(=O)NC5=O)C3)c12]

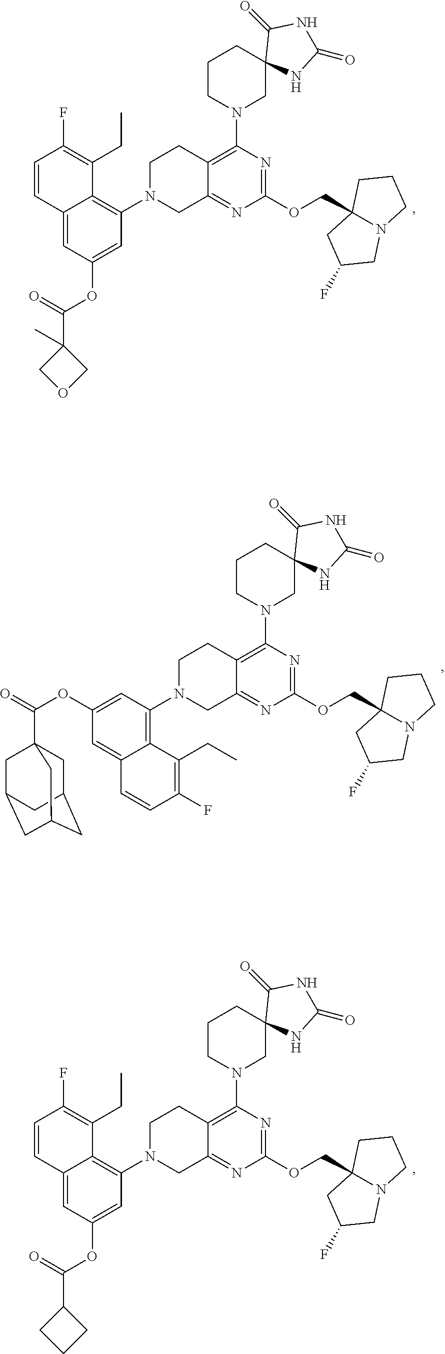 [SMILES: CCc1c(F)ccc2cc(OC(=O)C3(C)COC3)cc(N3CCc4c(nc(OC[C@@]56CCCN5C[C@H](F)C6)nc4N4CCC[C@]5(C4)NC(=O)NC5=O)C3)c12 ; CCc1c(F)ccc2cc(OC(=O)C34CC5CC(CC(C5)C3)C4)cc(N3CCc4c(nc(OC[C@@]56CCCN5C[C@H](F)C6)nc4N4CCC[C@]5(C4)NC(=O)NC5=O)C3)c12 ; CCc1c(F)ccc2cc(OC(=O)C3CCC3)cc(N3CCc4c(nc(OC[C@@]56CCCN5C[C@H](F)C6)nc4N4CCC[C@]5(C4)NC(=O)NC5=O)C3)c12]

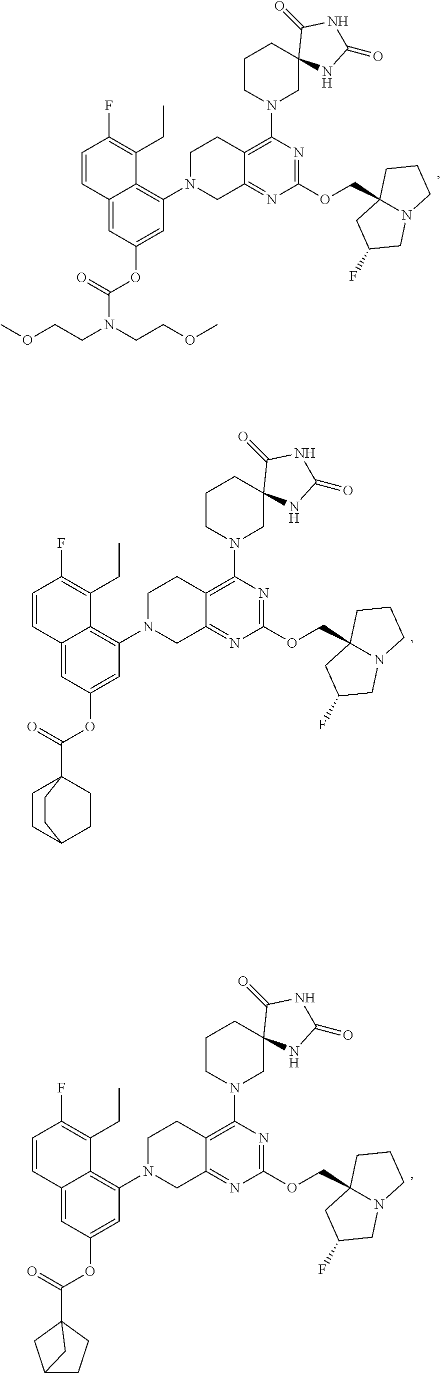 [SMILES: CCc1c(F)ccc2cc(OC(=O)N(CCOC)CCOC)cc(N3CCc4c(nc(OC[C@@]56CCCN5C[C@H](F)C6)nc4N4CCC[C@]5(C4)NC(=O)NC5=O)C3)c12 ; CCc1c(F)ccc2cc(OC(=O)C34CCC(CC3)CC4)cc(N3CCc4c(nc(OC[C@@]56CCCN5C[C@H](F)C6)nc4N4CCC[C@]5(C4)NC(=O)NC5=O)C3)c12 ; CCc1c(F)ccc2cc(OC(=O)C34CCC(C3)C4)cc(N3CCc4c(nc(OC[C@@]56CCCN5C[C@H](F)C6)nc4N4CCC[C@]5(C4)NC(=O)NC5=O)C3)c12]

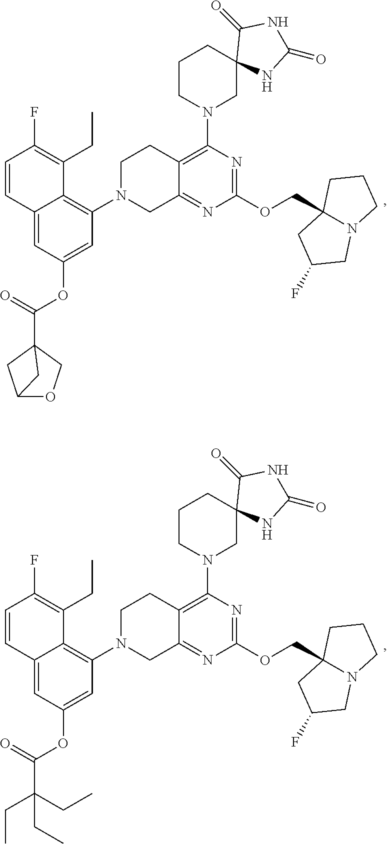 [SMILES: CCc1c(F)ccc2cc(OC(=O)C34COC(C3)C4)cc(N3CCc4c(nc(OC[C@@]56CCCN5C[C@H](F)C6)nc4N4CCC[C@]5(C4)NC(=O)NC5=O)C3)c12 ; CCc1c(F)ccc2cc(OC(=O)C(CC)(CC)CC)cc(N3CCc4c(nc(OC[C@@]56CCCN5C[C@H](F)C6)nc4N4CCC[C@]5(C4)NC(=O)NC5=O)C3)c12]

and pharmaceutically acceptable salts thereof.

In another aspect of the invention, the provided prodrugs have the following structure:

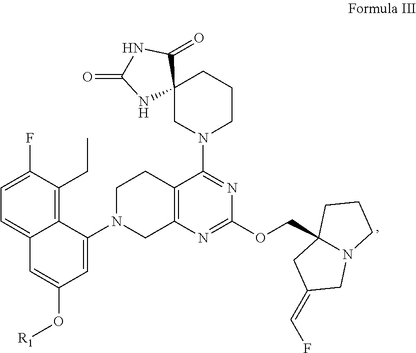

or a pharmaceutically acceptable salt thereof, wherein
R₁ is selected from the group consisting of:
R₃—*, R₃—O—CH₂—*,

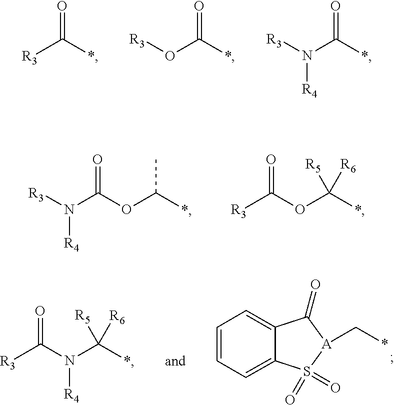

A is CH or N;
R₃ is C₁-C₂₀ alkyl, (C₁-C₄ alkyl)₀₋₁-cycloalkyl, (C₁-C₄ alkyl)₀₋₁-heterocyclyl or (C₁-C₄ alkyl)₀₋₁-aryl, where R₃ is optionally substituted with C₁-C₄ alkyl or —O— C₁-C₄ alkyl;
R₄ is H or R₃; and
R₅ and R₆ are independently selected from the group consisting of H and C₁-C₆ alkyl.

In one aspect, the compounds of Formula III are selected from the group consisting of:

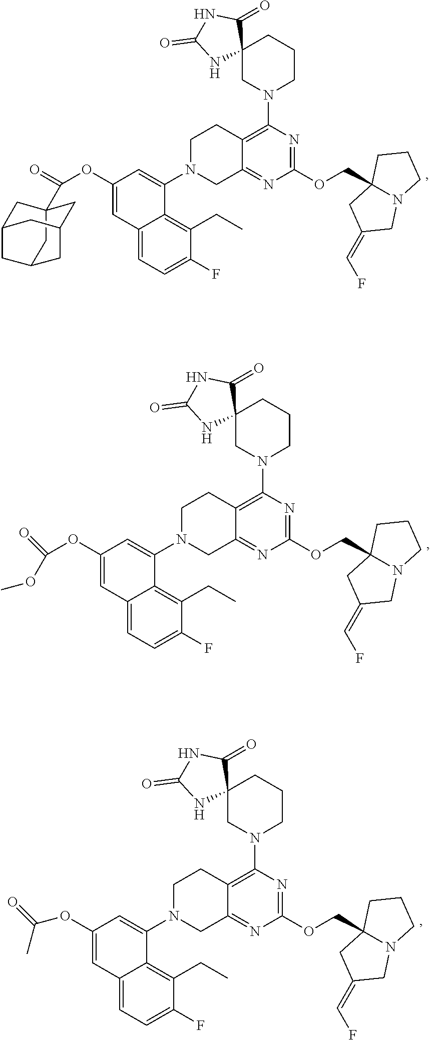 [SMILES: CCc1c(F)ccc2cc(OC(=O)C34CC5CC(CC(C5)C3)C4)cc(N3CCc4c(nc(OC[C@@]56CCCN5C/C(=C\F)C6)nc4N4CCC[C@]5(C4)NC(=O)NC5=O)C3)c12 ; CCc1c(F)ccc2cc(OC(=O)OC)cc(N3CCc4c(nc(OC[C@@]56CCCN5C/C(=C\F)C6)nc4N4CCC[C@]5(C4)NC(=O)NC5=O)C3)c12 ; CCc1c(F)ccc2cc(OC(C)=O)cc(N3CCc4c(nc(OC[C@@]56CCCN5C/C(=C\F)C6)nc4N4CCC[C@]5(C4)NC(=O)NC5=O)C3)c12]

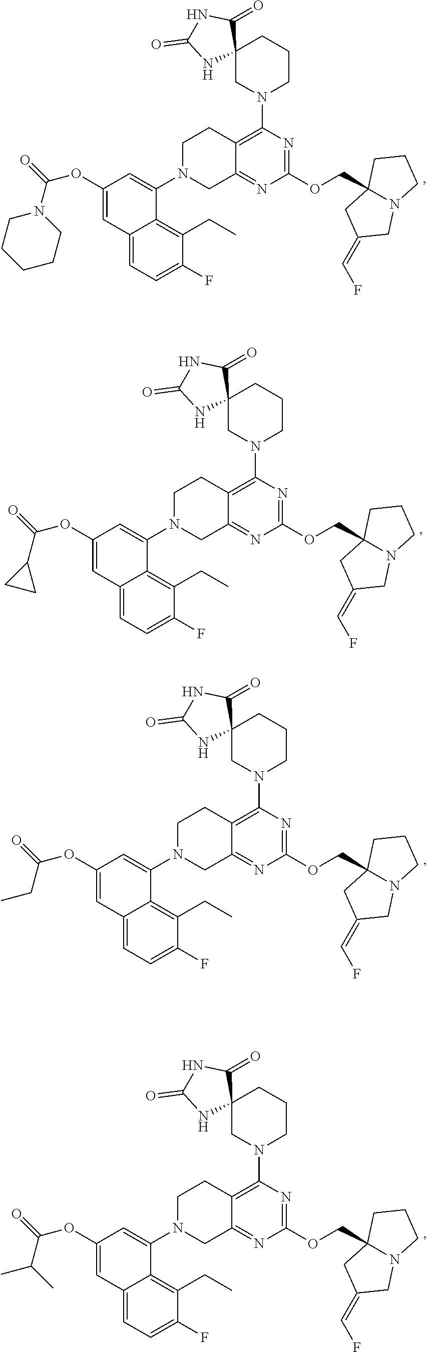 [SMILES: CCc1c(F)ccc2cc(OC(=O)N3CCCCC3)cc(N3CCc4c(nc(OC[C@@]56CCCN5C/C(=C\F)C6)nc4N4CCC[C@]5(C4)NC(=O)NC5=O)C3)c12 ; CCc1c(F)ccc2cc(OC(=O)C3CC3)cc(N3CCc4c(nc(OC[C@@]56CCCN5C/C(=C\F)C6)nc4N4CCC[C@]5(C4)NC(=O)NC5=O)C3)c12 ; CCC(=O)Oc1cc(N2CCc3c(nc(OC[C@@]45CCCN4C/C(=C\F)C5)nc3N3CCC[C@]4(C3)NC(=O)NC4=O)C2)c2c(CC)c(F)ccc2c1 ; CCc1c(F)ccc2cc(OC(=O)C(C)C)cc(N3CCc4c(nc(OC[C@@]56CCCN5C/C(=C\F)C6)nc4N4CCC[C@]5(C4)NC(=O)NC5=O)C3)c12]

and pharmaceutically acceptable salts thereof.

In another aspect of the invention, the provided prodrugs have the following generic structure:

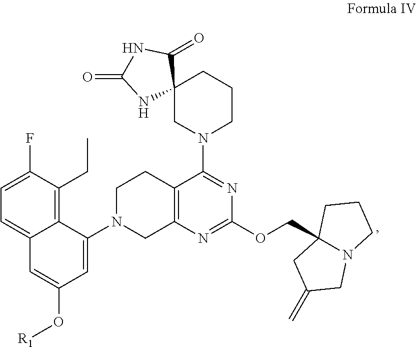

or a pharmaceutically acceptable salt thereof, wherein
R₁ is selected from the group consisting of:
R₃—*, R₃—O—CH₂—*,

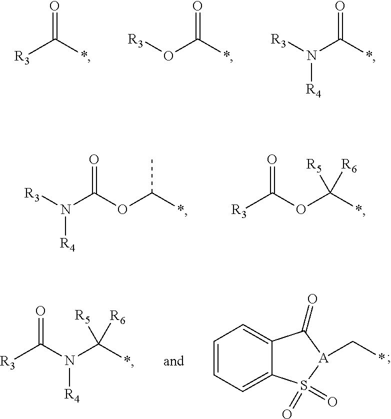

A is CH or N;
R₃ is C₁-C₂₀ alkyl, (C₁-C₄ alkyl)₀₋₁-cycloalkyl, (C₁-C₄ alkyl)₀₋₁-heterocyclyl or (C₁-C₄ alkyl)₀₋₁-aryl, where R₃ is optionally substituted with C₁-C₄ alkyl or —O— C₁-C₄ alkyl;
R₄ is H or R₃; and
R₅ and R₆ are independently selected from the group consisting of H and C₁-C₆ alkyl.

In one aspect, the compounds of Formula IV are selected from the group consisting of:

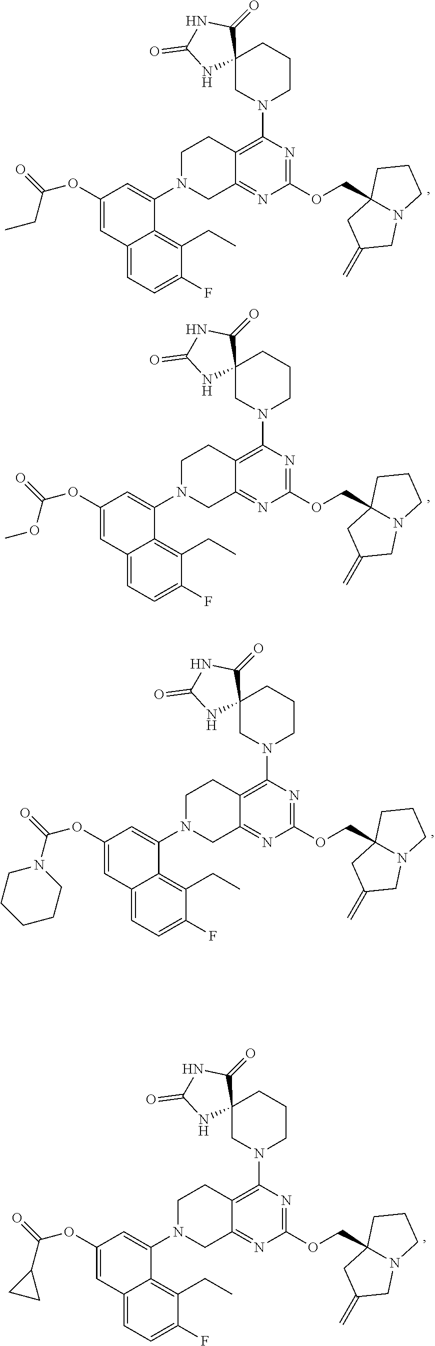

and pharmaceutically acceptable salts thereof.

In another aspect of the invention, pharmaceutical compositions are provided comprising a therapeutically effective amount of a prodrug compound of the present invention or a pharmaceutically acceptable salt thereof and a pharmaceutically acceptable excipient.

In yet another aspect of the invention, methods for inhibiting the activity of cells containing wild type KRas or one or more KRas mutations, for instance the KRas mutations G12A, G12C, G12D, G12R, G12S, G12V, G13D and/or Q61H, in a in a cell, comprising contacting the cell with prodrugs of the present invention, or a pharmaceutically acceptable salt thereof, or a pharmaceutical composition thereof as defined herein. In one embodiment, the contacting is in vitro. In one embodiment, the contacting is in vivo.

Also provided herein is a method of inhibiting cell proliferation, in vitro or in vivo, the method comprising contacting a cell with an effective amount of a prodrug compound of the present invention, or a pharmaceutically acceptable salt thereof, or a pharmaceutical composition thereof as defined herein.

Also provided are methods for treating cancer in a patient comprising administering a therapeutically effective amount of a prodrug compound or pharmaceutical composition of the present invention or a pharmaceutically acceptable salt thereof to a patient in need thereof.

Also provided herein is a method of treating a KRas wild type, KRas G12A, G12C, G12D, G12R, G12S, G12V, G13D and/or Q61H-associated disease or disorder in a patient in need of such treatment, the method comprising administering to the patient a therapeutically effective amount of a prodrug compound of the present invention, or a pharmaceutically acceptable salt thereof, or a pharmaceutical composition thereof as defined herein.

Also provided herein are prodrug compounds, or a pharmaceutically acceptable salt thereof, or a pharmaceutical composition thereof as defined herein for use in therapy.

Also provided herein are prodrug compounds, or a pharmaceutically acceptable salt thereof or a pharmaceutical composition thereof as defined herein for use in the treatment of cancer.

Also provided herein are prodrug compounds, or a pharmaceutically acceptable salt thereof for use in the inhibition of KRas wild type or multiple types of KRas mutations, for instance KRas G12A, G12C, G12D, G12R, G12S, G12V, G13D and/or Q61H mutations.

Also provided herein are prodrug compounds, or a pharmaceutically acceptable salt thereof or a pharmaceutical composition thereof as defined herein, for use in the treatment of a KRas wild type associated disease or disorder or a KRas mutation G12A, G12C, G12D, G12R, G12S, G12V, G13D and/or Q61H-associated disease or disorder.

Also provided herein is the use of a prodrug compound of the invention, or a pharmaceutically acceptable salt thereof, as defined herein in the manufacture of a medicament for the treatment of cancer.

Also provided herein is a use of a prodrug compound of the invention, or a pharmaceutically acceptable salt thereof, as defined herein in the manufacture of a medicament for the inhibition of activity of the wild type form of KRas or mutated forms of KRas, including the mutations: G12A, G12C, G12D, G12R, G12S, G12V, G13D and/or Q61H.

Also provided herein is the use of a prodrug compound of the invention, or a pharmaceutically acceptable salt thereof, as defined herein, in the manufacture of a medicament for the treatment of a KRas wild type associated disease or disorder or a KRas G12A, G12C, G12D, G12R, G12S, G12V, G13D and/or Q61H-associated disease or disorder.

Also provided herein is a method for treating cancer in a patient in need thereof, the method comprising (a) determining that the cancer is associated with KRas wild type or a KRas G12A, G12C, G12D, G12R, G12S, G12V, G13D and/or Q61H mutation (i.e., a KRas G12A, G12C, G12D, G12R, G12S, G12V, G13D and/or Q61H-associated cancer); and (b) administering to the patient a therapeutically effective amount of a prodrug compound of the invention, or a pharmaceutically acceptable salt thereof, or a pharmaceutical composition thereof.

One potential utility of the herein-described prodrugs of the pan-KRas inhibitors is for the treatment of cancers that develop resistance following long-term treatment with KRas G12C inhibitors. Thus, embodiments of the invention include those wherein a patient suffering from cancer is treated with a herein-described prodrug compound after treatment with a G12C inhibitor becomes ineffective or less effective due to the emergence of resistance-imparting mutations.

Treatment of KRas G12C mutant cancers with covalent KRas G12C inhibitors such as adagrasib (MRTX849) or sotorasib (AMG510) may result in the incorporation of additional mutations that confer resistance to adagrasib. These mutations could confer resistance through numerous mechanisms.

Mutations that change the mutant cysteine at codon 12 to another amino acid would render the current covalent KRas G12C inhibitors ineffective since current inhibitors make a covalent bond with the mutant cysteine amino acid side chain. Likewise, in patients that have one wild type KRas allele in addition to the KRas G12C-mutant allele, mutations in the wild type codon 12 glycine to another codon would allow bypass signaling in these tumors through the novel mutant protein. The repertoire of codon 12 mutations that can occur with a single nucleotide substitution in the wild type gene (glycine codon) includes mutations commonly observed in cancer such as G12S, G12V, G12R, G12C. The repertoire of codon 12 mutations that can occur with single nucleotide base substitutions of the cysteine codon 12 include mutations not frequently observed in cancer, G12Y, G12F and G12W, in addition to G12S and G12R.

Second-site mutations may also occur in another location in the KRas G12C mutant gene that confers resistance to KRas G12C inhibitor treatment. These mutations may confer resistance through different mechanisms. RAS proteins are small GTPases that normally cycle between an active, GTP-bound state and an inactive, GDP-bound state. RAS proteins are loaded with GTP through guanine nucleotide exchange factors (GEFs; e.g., SOS1) which are activated by upstream receptor tyrosine kinases, triggering subsequent interaction with effector proteins that activate RAS-dependent signaling. RAS proteins hydrolyze GTP to GDP through their intrinsic GTPase activity which is dramatically enhanced by GTPase-activating proteins (GAPs). Mutations at codons 12 and 13 in RAS proteins impair GAP-stimulated GTP hydrolysis leaving RAS predominantly in the GTP-bound, active state. Covalent KRas G12C inhibitors in current clinical development only bind GDP-bound KRas G12C. Mutations such as Q61 codon mutations, which may or may not occur on the same allele as the G12C mutation, reduce the intrinsic GTPase activity of KRas and may represent a mechanism of resistance to KRas G12C inhibitor treatment by shifting KRas into the GTP-loaded state where it is not susceptible to covalent inhibition. Co-mutations such as R68, H95 and Y96 may be present along with the KRas G12C mutation and may diminish the binding affinity of KRas G12C inhibitors to the Switch II binding pocket.

The herein-described prodrug compounds of the invention may demonstrate activity against common as well as uncommon codon 12 mutations or mutations that occur in the KRas protein that diminish binding of KRas G12C inhibitors to the KRas protein.

### DETAILED DESCRIPTION OF THE INVENTION

The present invention relates to prodrugs of specific compounds that inhibits multiple mutated forms of KRas, i.e., the pan-KRas inhibitors which inhibits KRas wild type and/or multiple mutated forms of KRas, for instance KRas G12A, G12C, G12D, G12R, G12S, G12V, G13D and/or Q61H mutations. In particular, the present invention relates to compounds that are prodrugs of a compound having the following formula:

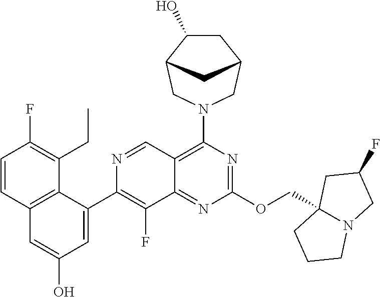 [SMILES: CCc1c(F)ccc2cc(O)cc(-c3ncc4c(N5C[C@@H]6C[C@H](C5)[C@H](O)C6)nc(OC[C@@]56CCCN5C[C@H](F)C6)nc4c3F)c12]

3-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-3-azabicyclo[3.2.1]octan-6-ol

This compound is described in PCT International Application No. PCT/US2021/010065 (WO 2022/132200), as the species of Example 140. The PCT application also describes methods of preparation of this compound.

Similarly, a compound having the formula:

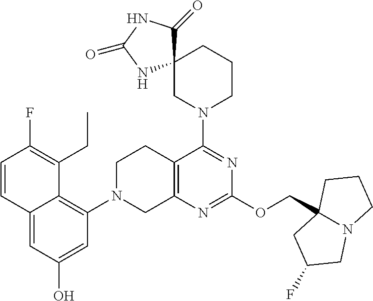

(R)-7-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-1,3,7-triazaspiro[4.5]decane-2,4-dione
   is described in PCT International Application No. PCT/US2021/063722 (WO 2022/133038), as the species of Example 196. The PCT application also describes methods of preparation of this compound.

The prodrugs of the invention, when administered in vivo, inhibit the activity of KRas wild type and/or KRas mutations such as G12A, G12C, G12D, G12R, G12S, G12V, G13D and/or Q61H, pharmaceutical compositions comprising a therapeutically effective amount of the compounds and methods of use therefor.

### Definitions

Unless defined otherwise, all technical and scientific terms used herein have the same meaning as is commonly understood by one of skill in the art to which this invention belongs. All patents, patent applications, and publications referred to herein are incorporated by reference.

As used herein, “wild type KRas” refers to a non-mutant form of a mammalian KRas protein. The assignment of amino acid codon and residue positions for human KRas is based on the amino acid sequence identified by UniProtKB/Swiss-Prot P01116: Variantp.Gly12Asp. As used herein, a “wild type KRas inhibitor” refers to compounds of the present invention that are represented by Formulas (I)-(IV) or their metabolites, as described herein. These compounds are capable of negatively modulating or inhibiting all or a portion of the enzymatic activity of wild type KRas G12A. A “wild type KRas-associated disease or disorder” as used herein refers to diseases or disorders associated with or mediated by or having wild type KRas. A non-limiting example of a wild type KRas-associated disease or disorder is a wild type KRas-associated cancer.

As used herein, “KRas G12A” refers to a mutant form of a mammalian KRas protein that contains an amino acid substitution of an alanine for a glycine at amino acid position 12. The assignment of amino acid codon and residue positions for human KRas is based on the amino acid sequence identified by UniProtKB/Swiss-Prot P01116: Variantp.Gly12Asp. As used herein, a “KRas G12A inhibitor” refers to compounds of the present invention that are represented by Formulas (I)-(IV) or their metabolites, as described herein. These compounds are capable of negatively modulating or inhibiting all or a portion of the enzymatic activity of KRas G12A. A “KRas G12A-associated disease or disorder” as used herein refers to diseases or disorders associated with or mediated by or having a KRas G12A mutation. A non-limiting example of a KRas G12A-associated disease or disorder is a KRas G12A-associated cancer.

As used herein, “KRas G12C” refers to a mutant form of a mammalian KRas protein that contains an amino acid substitution of a cysteine for a glycine at amino acid position 12. The assignment of amino acid codon and residue positions for human KRas is based on the amino acid sequence identified by UniProtKB/Swiss-Prot P01116: Variantp.Gly12Asp. As used herein, a “KRas G12C inhibitor” refers to compounds of the present invention that are represented by Formulas (I)-(IV) or their metabolites, as described herein. These compounds are capable of negatively modulating or inhibiting all or a portion of the enzymatic activity of KRas G12C. A “KRas G12C-associated disease or disorder” as used herein refers to diseases or disorders associated with or mediated by or having a KRas G12C mutation. A non-limiting example of a KRas G12C-associated disease or disorder is a KRas G12CD-associated cancer.

As used herein, “KRas G12D” refers to a mutant form of a mammalian KRas protein that contains an amino acid substitution of an aspartic acid for a glycine at amino acid position 12. The assignment of amino acid codon and residue positions for human KRas is based on the amino acid sequence identified by UniProtKB/Swiss-Prot P01116: Variantp.Gly12Asp. As used herein, a “KRas G12D inhibitor” refers to compounds of the present invention that are represented by Formulas (I)-(IV) or their metabolites, as described herein. These compounds are capable of negatively modulating or inhibiting all or a portion of the enzymatic activity of KRas G12D. A “KRas G12D-associated disease or disorder” as used herein refers to diseases or disorders associated with or mediated by or having a KRas G12D mutation. A non-limiting example of a KRas G12D-associated disease or disorder is a KRas G12D-associated cancer.

As used herein, “KRas G12R” refers to a mutant form of a mammalian KRas protein that contains an amino acid substitution of an arginine for a glycine at amino acid position 12. The assignment of amino acid codon and residue positions for human KRas is based on the amino acid sequence identified by UniProtKB/Swiss-Prot P01116: Variantp.Gly12Asp. As used herein, a “KRas G12R inhibitor” refers to compounds of the present invention that are represented by Formulas (I)-(IV) or their metabolites, as described herein. These compounds are capable of negatively modulating or inhibiting all or a portion of the enzymatic activity of KRas G12R. A “KRas G12R-associated disease or disorder” as used herein refers to diseases or disorders associated with or mediated by or having a KRas G12R mutation. A non-limiting example of a KRas G12R-associated disease or disorder is a KRas G12R-associated cancer.

As used herein, “KRas G12S” refers to a mutant form of a mammalian KRas protein that contains an amino acid substitution of a serine for a glycine at amino acid position 12. The assignment of amino acid codon and residue positions for human KRas is based on the amino acid sequence identified by UniProtKB/Swiss-Prot P01116: Variantp.Gly12Asp. As used herein, a “KRas G12S inhibitor” refers to compounds of the present invention that are represented by Formulas (I)-(IV) or their metabolites, as described herein. These compounds are capable of negatively modulating or inhibiting all or a portion of the enzymatic activity of KRas G12S. A “KRas G12S-associated disease or disorder” as used herein refers to diseases or disorders associated with or mediated by or having a KRas G12S mutation. A non-limiting example of a KRas G12S-associated disease or disorder is a KRas G12S-associated cancer.

As used herein, “KRas G12V” refers to a mutant form of a mammalian KRas protein that contains an amino acid substitution of a valine for a glycine at amino acid position 12. The assignment of amino acid codon and residue positions for human KRas is based on the amino acid sequence identified by UniProtKB/Swiss-Prot P01116: Variantp.Gly12Asp. As used herein, a “KRas G12V inhibitor” refers to compounds of the present invention that are represented by Formulas (I)-(IV) or their metabolites, as described herein. These compounds are capable of negatively modulating or inhibiting all or a portion of the enzymatic activity of KRas G12V. A “KRas G12V-associated disease or disorder” as used herein refers to diseases or disorders associated with or mediated by or having a KRas G12V mutation. A non-limiting example of a KRas G12V-associated disease or disorder is a KRas G12V-associated cancer.

As used herein, “KRas G13D” refers to a mutant form of a mammalian KRas protein that contains an amino acid substitution of an aspartic acid for a glycine at amino acid position 13. The assignment of amino acid codon and residue positions for human KRas is based on the amino acid sequence identified by UniProtKB/Swiss-Prot P01116: Variantp.Gly12Asp. As used herein, a “KRas G13D inhibitor” refers to compounds of the present invention that are represented by Formulas (I)-(IV) or their metabolites, as described herein. These compounds are capable of negatively modulating or inhibiting all or a portion of the enzymatic activity of KRas G13D. A “KRas G13D-associated disease or disorder” as used herein refers to diseases or disorders associated with or mediated by or having a KRas G13D mutation. A non-limiting example of a KRas G13D-associated disease or disorder is a KRas G13D-associated cancer.

As used herein, “KRas Q61H” refers to a mutant form of a mammalian KRas protein that contains an amino acid substitution of a histidine for a glutamine at amino acid position 61. The assignment of amino acid codon and residue positions for human KRas is based on the amino acid sequence identified by UniProtKB/Swiss-Prot P01116: Variantp.Gly12Asp. As used herein, a “KRas Q61H inhibitor” refers to compounds of the present invention that are represented by Formulas (I)-(IV) or their metabolites, as described herein. These compounds are capable of negatively modulating or inhibiting all or a portion of the enzymatic activity of KRas Q61H. A “KRas Q61H-associated disease or disorder” as used herein refers to diseases or disorders associated with or mediated by or having a KRas Q61H mutation. A non-limiting example of a KRas Q61H-associated disease or disorder is a KRas Q61H-associated cancer.

As used herein, the term “subject,” “individual,” or “patient,” used interchangeably, refers to any animal, including mammals such as mice, rats, other rodents, rabbits, dogs, cats, swine, cattle, sheep, horses, primates, and humans. In some embodiments, the patient is a human. In some embodiments, the subject has experienced and/or exhibited at least one symptom of the disease or disorder to be treated and/or prevented. In some embodiments, the subject has been identified or diagnosed as having a cancer having wild type KRas or a KRas G12A, G12C, G12D, G12R, G12S, G12V, G13D and/or Q61H mutation (e.g., as determined using a regulatory agency-approved, e.g., FDA-approved, assay or kit). In some embodiments, the subject has a tumor that is positive for wild type KRas or a KRas G12A, G12C, G12D, G12R, G12S, G12V, G13D and/or Q61H mutation (e.g., as determined using a regulatory agency-approved assay or kit). The subject can be a subject with a tumor(s) that is positive for wild type KRas or a KRas G12A, G12C, G12D, G12R, G12S, G12V, G13D and/or Q61H mutation (e.g., identified as positive using a regulatory agency-approved, e.g., FDA-approved, assay or kit). The subject can be a subject whose tumors have wild type KRas or a KRas G12A, G12C, G12D, G12R, G12S, G12V, G13D and/or Q61H mutation (e.g., where the tumor is identified as such using a regulatory agency-approved, e.g., FDA-approved, kit or assay). In some embodiments, the subject is suspected of having wild type KRas or a KRas G12A, KRas G12C, KRas G12D, KRas G12R, KRas G12S, KRas G12V, KRas G13D or KRas Q61H gene-associated cancer. In some embodiments, the subject has a clinical record indicating that the subject has a tumor that has wild type KRas or a KRas G12A, G12C, G12D, G12R, G12S, G12V, G13D and/or Q61H mutation (and optionally the clinical record indicates that the subject should be treated with any of the compositions provided herein).

As used herein, the term “prodrug” refers to a drug precursor compound, that undergoes transformation in vivo to yield a parent compound or a pharmaceutically acceptable salt, hydrate or solvate of the parent compound. The transformation may occur by various mechanisms by metabolic or chemical processes. A discussion of the use of prodrugs is provided by. “Prodrugs: Challenges and Rewards, Parts 1 and 2,” Vol. V of the Biotechnology: Pharmaceutical Aspects (Ronald T. Borchardt and C. Russel Middaugh, series editors), ed. Valentino J. Stella, Ronald T. Borchardt, Michael J. Hageman, Reza Oliyai, Hans Maag, Jefferson W. Tilley, American Association of Pharmaceutical Scientists and Springer, 2007. Particularly favored prodrugs are those that increase the bioavailability of the parent compounds when such compounds are administered to a patient (e.g., by allowing orally administered compound to be more readily absorbed into blood) or which enhance delivery of the parent compound to a biological compartment (e.g., the brain or lymphatic system).

The compounds of the invention, which are prodrugs, may themselves have prodrugs. In that case, the term “prodrug” refers to any pharmaceutically acceptable salt, ester, salt of an ester or other derivative of a compound of this invention, which, upon administration to a recipient, is capable of providing, either directly or indirectly, a compound of this invention or a pharmaceutically active metabolite or residue thereof.

As used herein, the term “metabolite” refers to any substance produced during metabolism, i.e., digestion or other bodily chemical processes.

In some embodiments of any of the methods or uses described herein, an assay is used to determine whether the patient has wild type KRas or a KRas G12A, G12C, G12D, G12R, G12S, G12V, G13D and/or Q61H mutation using a sample (e.g., a biological sample or a biopsy sample (e.g., a paraffin-embedded biopsy sample) from a patient (e.g., a patient suspected of having wild type KRas-associated or a KRas G12A, G12C, G12D, G12R, G12S, G12V, G13D and/or Q61H-associated cancer, a patient having one or more symptoms of wild type KRas-associated or a KRas G12A, G12C, G12D, G12R, G12S, G12V, G13D and/or Q61H-associated cancer, and/or a patient that has an increased risk of developing wild type KRas-associated or a KRas G12A, G12C, G12D, G12R, G12S, G12V, G13D and/or Q61H-associated cancer) can include, for example, next generation sequencing, immunohistochemistry, fluorescence microscopy, break apart FISH analysis, Southern blotting, Western blotting, FACS analysis, Northern blotting, and PCR-based amplification (e.g., RT-PCR and quantitative real-time RT-PCR). As is well-known in the art, the assays are typically performed, e.g., with at least one labelled nucleic acid probe or at least one labelled antibody or antigen-binding fragment thereof.

The term “regulatory agency” is a country's agency for the approval of the medical use of pharmaceutical agents with the country. For example, a non-limiting example of a regulatory agency is the U.S. Food and Drug Administration (FDA).

The term “acyl” refers to —C(O)CH₃.

The term “alkyl” as employed herein refers to straight and branched chain aliphatic groups. For instance, the terms “C1-C6 alkyl”, “C1-C4 alkyl” and “C1-C3 alkyl” as employed herein refers to straight and branched chain aliphatic groups having from 1-6 carbon atoms, or 1-4 carbon atoms, or 1-3 carbon atoms, respectively. Examples of alkyl groups include, without limitation, methyl, ethyl, propyl, isopropyl, butyl, isobutyl, sec-butyl, tert-butyl, pentyl, and hexyl.

The terms “C1-C3 haloalkyl” and “C1-C4 haloalkyl” refer to a C1-C3 alkyl chain or C1-C4 alkyl chain, respectively, as defined herein in which one or more hydrogen has been replaced by a halogen. Examples include trifluoromethyl, difluoromethyl and fluoromethyl.

An “C1-C4 alkylene,” group is a C1-C4 alkyl group, as defined hereinabove, that is positioned between and serves to connect two other chemical groups. Exemplary alkylene groups include, without limitation, methylene, ethylene, propylene, and butylene.

The terms “C1-C3 alkoxy” and “C1-C4 alkoxy” refer to —OC1-C3 alkyl and —OC1-C4 alkyl, respectively, wherein the alkyl portion is as defined herein above.

The term “cycloalkyl” as employed herein includes saturated and partially unsaturated cyclic hydrocarbon groups having 3 to 12 carbons, for example 3 to 8 carbons, and as a further example 3 to 6 carbons, wherein the cycloalkyl group additionally is optionally substituted with C₁-C₆ alkyl or other groups as defined herein. Examples of cycloalkyl groups include, without limitation, cyclopropyl, cyclobutyl, cyclopentyl, cyclopentenyl, cyclohexyl, cyclohexenyl, cycloheptyl, and cyclooctyl. The term “cycloalkyl” also includes bridged cycloalkyls, such as bicyclo[1.1.1]pentanyl.

As used herein, the terms “C1-C3 hydroxyalkyl” and “C1-C4 hydroxyalkyl” refer to —C1-C3 alkylene-OH and —C1-C4 alkylene-OH, respectively.

As used herein, the term “C2-C4 hydroxyalkynyl” refers to —C2-C4 alkynylene-OH.

An “aryl” group is a C₆-C₁₄ aromatic moiety comprising one to three aromatic rings, which is optionally substituted with one or more R⁸ or R⁹ groups as defined herein. As one embodiment, the aryl group is a C₆-C₁₀ aryl group. Examples of aryl groups include, without limitation, phenyl, naphthyl, anthracenyl, fluorenyl, and dihydrobenzofuranyl. “Aryl” also refers to bicyclic or tricyclic ring systems in which one or two rings, respectively, of said aryl ring system may be saturated or partially saturated, and wherein if said ring system includes two saturated rings, said saturated rings may be fused or spirocyclic. An example of an aryl ring system comprising two saturated rings wherein the rings are spirocyclic includes the following ring system:

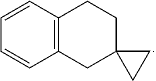 [SMILES: c1ccc2c(c1)CCC1(CC1)C2]

An “araC1-C6 alkyl” or “arylalkyl” group comprises an aryl group covalently linked to an alkyl group, either of which may independently be optionally substituted or unsubstituted. An example of an aralkyl group is (C₆-C₁₀)aryl(C₁-C₆)alkyl-, including, without limitation, benzyl, phenethyl, and naphthylmethyl. An example of a substituted araC1-C6 alkyl is wherein the alkyl group is substituted with hydroxyalkyl.

A “heterocyclyl” or “heterocyclic” group is a ring structure having from 3 to 12 atoms, for example 4 to 8 atoms, wherein one or more atoms are selected from the group consisting of N, O, and S wherein the ring N atom may be oxidized to N—O, and the ring S atom may be oxidized to SO or SO₂, the remainder of the ring atoms being carbon. The heterocyclyl may be a monocyclic, a bicyclic, a spirocyclic or a bridged ring system. The heterocyclic group may optionally be substituted on a ring nitrogen atom with alkyl, aralkyl, alkylcarbonyl, on sulfur with lower alkyl, and on carbon with lower alkyl. Examples of heterocyclic groups include, without limitation, epoxy, azetidinyl, aziridinyl, tetrahydrofuranyl, tetrahydropyranyl, pyrrolidinyl, pyrrolidinonyl, piperidinyl, piperazinyl, imidazolidinyl, imidazopyridinyl, thiazolidinyl, dithianyl, trithianyl, dioxolanyl, oxazolidinyl, oxazolidinonyl, decahydroquinolinyl, piperidonyl, 4-piperidinonyl, quinuclidinyl, thiomorpholinyl, thiomorpholinyl 1,1 dioxide, morpholinyl, azepanyl, oxazepanyl, azabicyclohexanyls, azabicycloheptanyl, azabicyclooctanyls, azabicyclononanyls (e.g., octahydroindolizinyl), azaspiroheptanyls, dihydro-1H,3H,5H-oxazolo[3,4-c]oxazolyl, tetrahydro-1′H,3′H-spiro[cyclopropane-1,2′-pyrrolizine], hexahydro-1H-pyrrolizinyl, hexahydro-1H-pyrrolo[2,1-c][1,4]oxazinyl, octahydroindolizinyl, oxaazaspirononanyls, oxaazaspirooctanyls, diazaspirononanyls, oxaazabiocycloheptanyls, hexahydropyrrolizinyl 4(1H)-oxide, tetrahydro-2H-thiopyranyl 1-oxide and tetrahydro-2H-thiopyranyl 1,1-dioxide. Specifically excluded from the scope of this term are compounds having adjacent annular O and/or S atoms.

As used herein, the term “heteroaryl” refers to groups having 5 to 14 ring atoms, preferably 5, 6, 9, or 10 ring atoms; having 6, 10, or 14 π electrons shared in a cyclic array; and having, in addition to carbon atoms, from one to three heteroatoms per ring, or from one to three heteroatoms in at least one ring, selected from the group consisting of N, O, and S. Examples of heteroaryl groups include acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, 6,7-dihydro-5H-pyrrolo[1,2-a]imidazole, furanyl, furazanyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, methylenedioxyphenyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolinyl, 2H-pyrrolyl, pyrrolyl, quinazolinyl, quinolinyl, 4H-quinolizinyl, quinoxalinyl, quinuclidinyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl. “Heteroaryl” also refers to bicyclic ring systems having, in addition to carbon atoms, from one to three heteroatoms per ring selected from the group consisting of N, O, and S in which one ring system may be saturated or partially saturated.

As used herein, “an effective amount” of a compound is an amount that is sufficient to negatively modulate or inhibit the activity of one or more of wild type KRas, KRas G12A, KRas G12C, KRas G12D, KRas G12R, KRas G12S, KRas G12V, KRas G13D or KRas Q61H. Such amount may be administered as a single dosage or may be administered according to a regimen, whereby it is effective.

As used herein, a “therapeutically effective amount” of a compound is an amount that is sufficient to ameliorate, or in some manner reduce a symptom or stop or reverse progression of a condition, or negatively modulate or inhibit the activity of one or more of wild type KRas, KRas G12A, KRas G12C, KRas G12D, KRas G12R, KRas G12S, KRas G12V, KRas G13D or KRas Q61H. Such amount may be administered as a single dosage or may be administered according to a regimen, whereby it is effective.

As used herein, treatment means any manner in which the symptoms or pathology of a condition, disorder or disease are ameliorated or otherwise beneficially altered. Treatment also encompasses any pharmaceutical use of the compositions herein.

As used herein, amelioration of the symptoms of a particular disorder by administration of a particular pharmaceutical composition refers to any lessening, whether permanent or temporary, lasting or transient that can be attributed to or associated with administration of the composition.

### Compounds

In one embodiment of the invention there are provided prodrug compounds of a compound of the following structure:

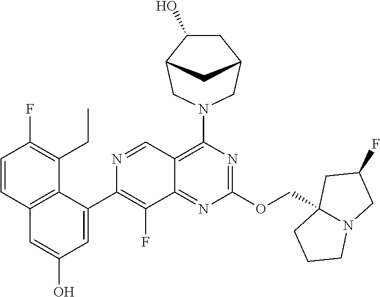 [SMILES: CCc1c(F)ccc2cc(O)cc(-c3ncc4c(N5C[C@@H]6C[C@H](C5)[C@H](O)C6)nc(OC[C@@]56CCCN5C[C@H](F)C6)nc4c3F)c12]

3-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-3-azabicyclo[3.2.1]octan-6-ol

This compound is described in PCT International Application No. PCT/US2021/010065 (WO 2022/132200), as the species of Example 140.

Similarly, a compound having the formula:

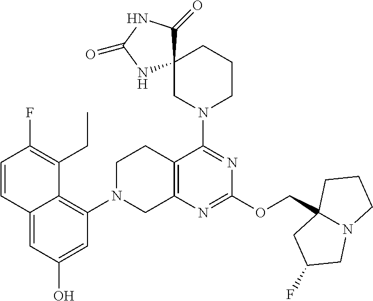

(R)-7-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-1,3,7-triazaspiro[4.5]decane-2,4-dione
   is described in PCT International Application No. PCT/US2021/063722 (WO 2022/133038), as the species of Example 196.

In one aspect, the provided prodrugs have the generic structure of Formula (I):

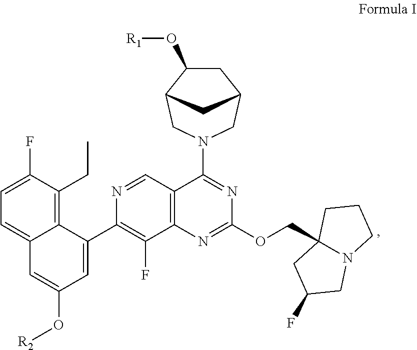

or a pharmaceutically acceptable salt thereof,
wherein
R₁ and R₂ are independently selected from the group consisting of:
H, R₃—*, R₃—O—CH₂—*,

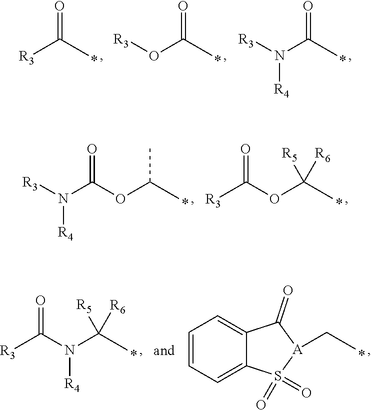

with the proviso that only one of R₁ and R₂ can be H;
A is CH or N;
R₃ is C₁-C₂₀ alkyl, (C₁-C₄ alkyl)₀₋₁-cycloalkyl, (C₁-C₄ alkyl)₀₋₁-heterocyclyl or (C₁-C₄ alkyl)₀₋₁-aryl, where R₃ is optionally substituted with C₁-C₄ alkyl or —O— C₁-C₄ alkyl; R₄ is H or R₃; and
R₅ and R₆ are independently selected from the group consisting of H and C₁-C₆ alkyl.

In one aspect, R₁ is H and R₂ is R₃—*, R₃—O—CH₂—*,

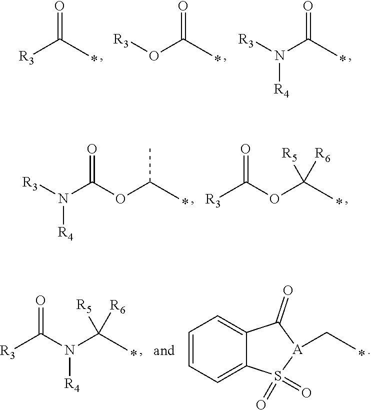

In another aspect, R₂ is H and R₁ is R₃—*, R₃—O—CH₂—*,

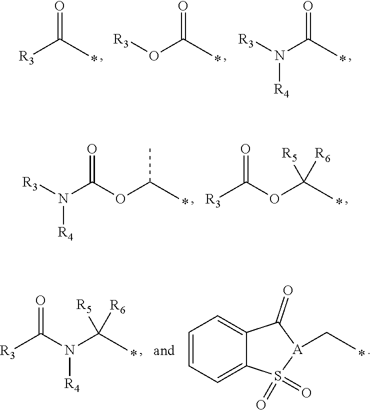

In one aspect, the provided prodrugs have the following structures:

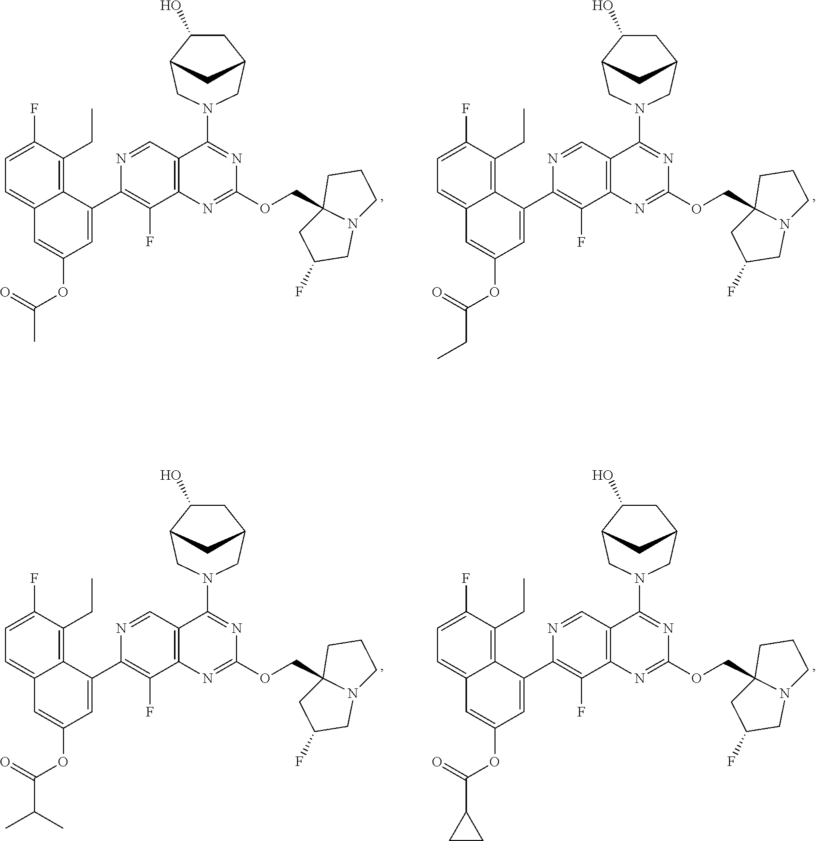 [SMILES: CCc1c(F)ccc2cc(OC(C)=O)cc(-c3ncc4c(N5C[C@@H]6C[C@H](C5)[C@H](O)C6)nc(OC[C@@]56CCCN5C[C@H](F)C6)nc4c3F)c12 ; CCC(=O)Oc1cc(-c2ncc3c(N4C[C@@H]5C[C@H](C4)[C@H](O)C5)nc(OC[C@@]45CCCN4C[C@H](F)C5)nc3c2F)c2c(CC)c(F)ccc2c1 ; CCc1c(F)ccc2cc(OC(=O)C(C)C)cc(-c3ncc4c(N5C[C@@H]6C[C@H](C5)[C@H](O)C6)nc(OC[C@@]56CCCN5C[C@H](F)C6)nc4c3F)c12 ; CCc1c(F)ccc2cc(OC(=O)C3CC3)cc(-c3ncc4c(N5C[C@@H]6C[C@H](C5)[C@H](O)C6)nc(OC[C@@]56CCCN5C[C@H](F)C6)nc4c3F)c12]

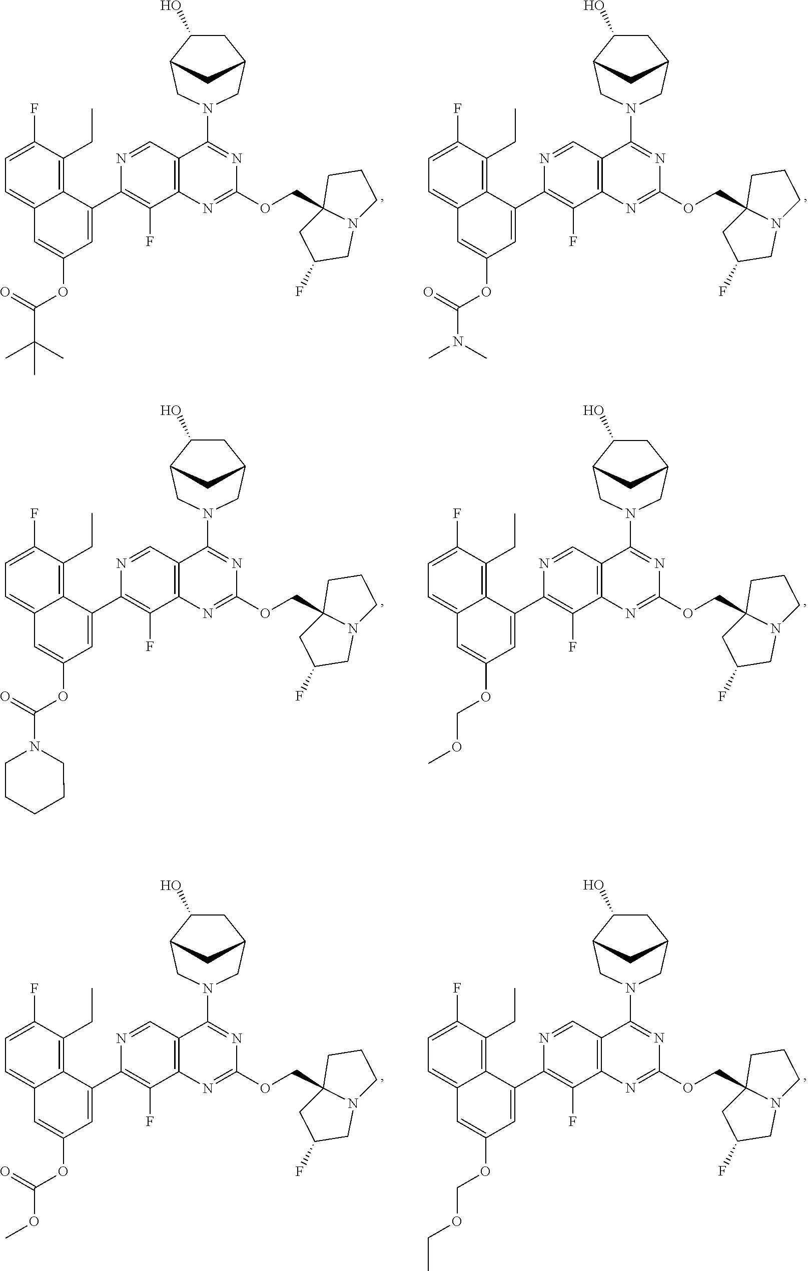

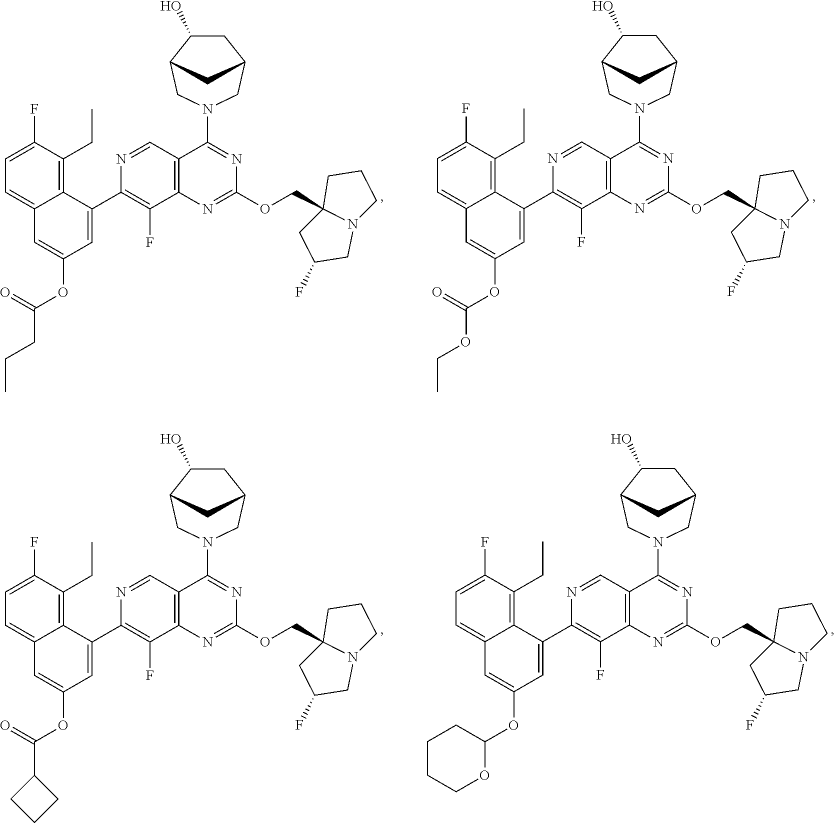 [SMILES: CCCC(=O)Oc1cc(-c2ncc3c(N4C[C@@H]5C[C@H](C4)[C@H](O)C5)nc(OC[C@@]45CCCN4C[C@H](F)C5)nc3c2F)c2c(CC)c(F)ccc2c1 ; CCOC(=O)Oc1cc(-c2ncc3c(N4C[C@@H]5C[C@H](C4)[C@H](O)C5)nc(OC[C@@]45CCCN4C[C@H](F)C5)nc3c2F)c2c(CC)c(F)ccc2c1 ; CCc1c(F)ccc2cc(OC(=O)C3CCC3)cc(-c3ncc4c(N5C[C@@H]6C[C@H](C5)[C@H](O)C6)nc(OC[C@@]56CCCN5C[C@H](F)C6)nc4c3F)c12 ; CCc1c(F)ccc2cc(OC3CCCCO3)cc(-c3ncc4c(N5C[C@@H]6C[C@H](C5)[C@H](O)C6)nc(OC[C@@]56CCCN5C[C@H](F)C6)nc4c3F)c12]

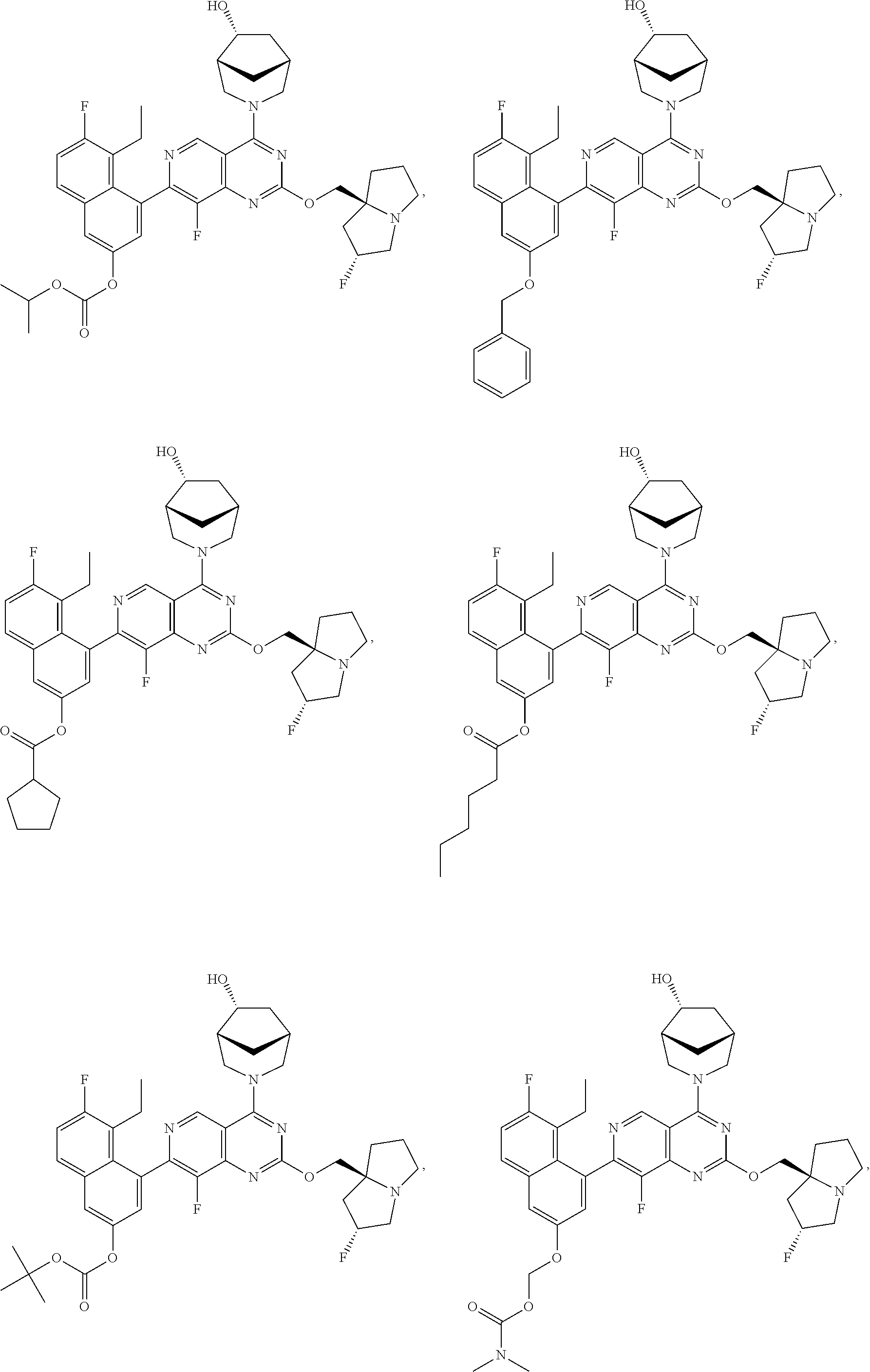

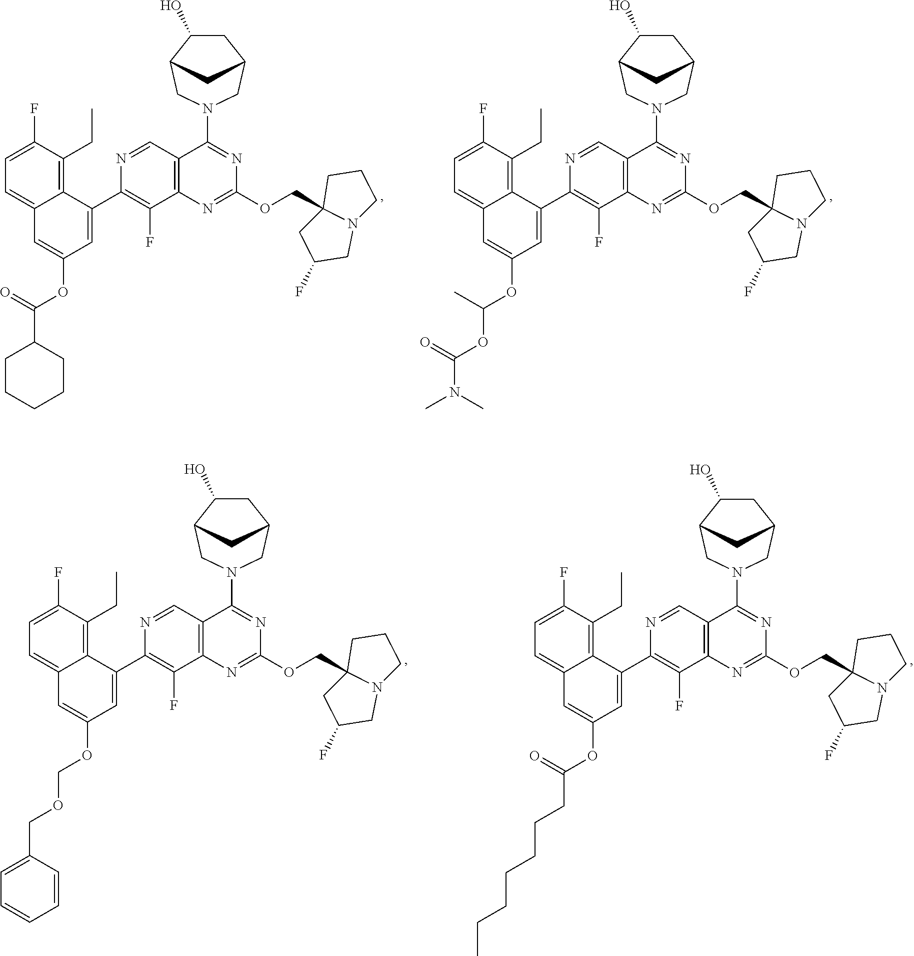 [SMILES: CCc1c(F)ccc2cc(OC(=O)C3CCCCC3)cc(-c3ncc4c(N5C[C@@H]6C[C@H](C5)[C@H](O)C6)nc(OC[C@@]56CCCN5C[C@H](F)C6)nc4c3F)c12 ; CCc1c(F)ccc2cc(OC(C)OC(=O)N(C)C)cc(-c3ncc4c(N5C[C@@H]6C[C@H](C5)[C@H](O)C6)nc(OC[C@@]56CCCN5C[C@H](F)C6)nc4c3F)c12 ; CCc1c(F)ccc2cc(OCOCc3ccccc3)cc(-c3ncc4c(N5C[C@@H]6C[C@H](C5)[C@H](O)C6)nc(OC[C@@]56CCCN5C[C@H](F)C6)nc4c3F)c12 ; CCCCCCCC(=O)Oc1cc(-c2ncc3c(N4C[C@@H]5C[C@H](C4)[C@H](O)C5)nc(OC[C@@]45CCCN4C[C@H](F)C5)nc3c2F)c2c(CC)c(F)ccc2c1]

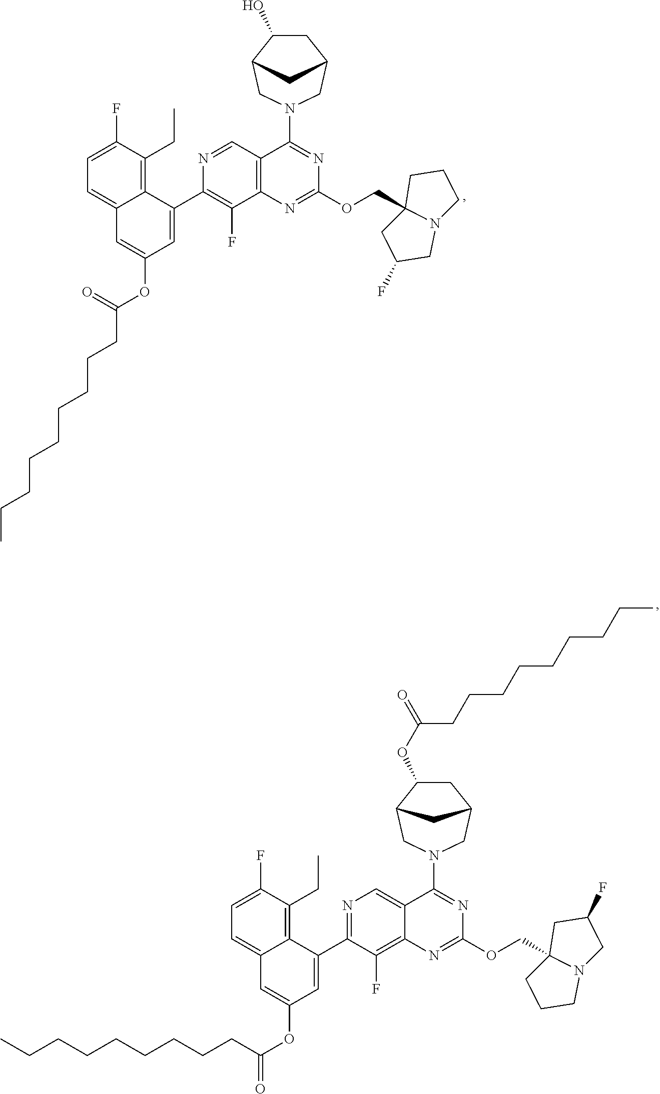 [SMILES: CCCCCCCCCC(=O)Oc1cc(-c2ncc3c(N4C[C@@H]5C[C@H](C4)[C@H](O)C5)nc(OC[C@@]45CCCN4C[C@H](F)C5)nc3c2F)c2c(CC)c(F)ccc2c1 ; CCCCCCCCCC(=O)Oc1cc(-c2ncc3c(N4C[C@@H]5C[C@H](C4)[C@H](OC(=O)CCCCCCCCC)C5)nc(OC[C@@]45CCCN4C[C@H](F)C5)nc3c2F)c2c(CC)c(F)ccc2c1]

and pharmaceutically acceptable salts thereof.

In another aspect of the invention, the provided prodrugs have the following generic structure:

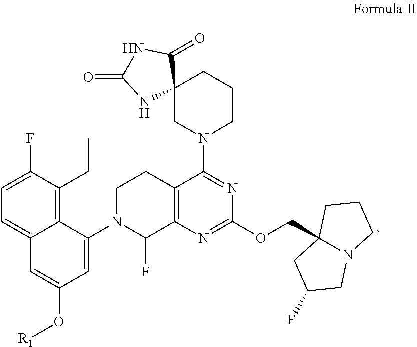

or a pharmaceutically acceptable salt thereof,
wherein R₁ is H, R₃—*, R₃—O—CH₂—*,

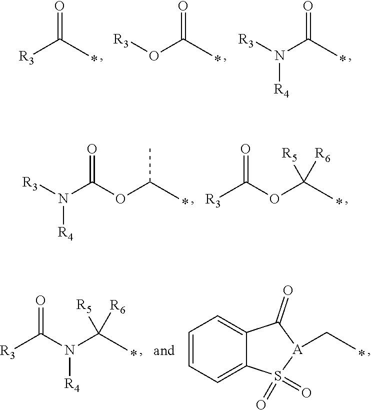

A is CH or N;
R₃ is C₁-C₂₀ alkyl, (C₁-C₄ alkyl)₀₋₁-cycloalkyl, (C₁-C₄ alkyl)₀₋₁-heterocyclyl or (C₁-C₄ alkyl)₀₋₁-aryl, where R₃ is optionally substituted with C₁-C₄ alkyl or —O— C₁-C₄ alkyl;
R₄ is H or R₃;
R₅ and R₆ are independently selected from the group consisting of H and C₁-C₆ alkyl.

In one aspect, the compounds of Formula II are selected from the group consisting of:

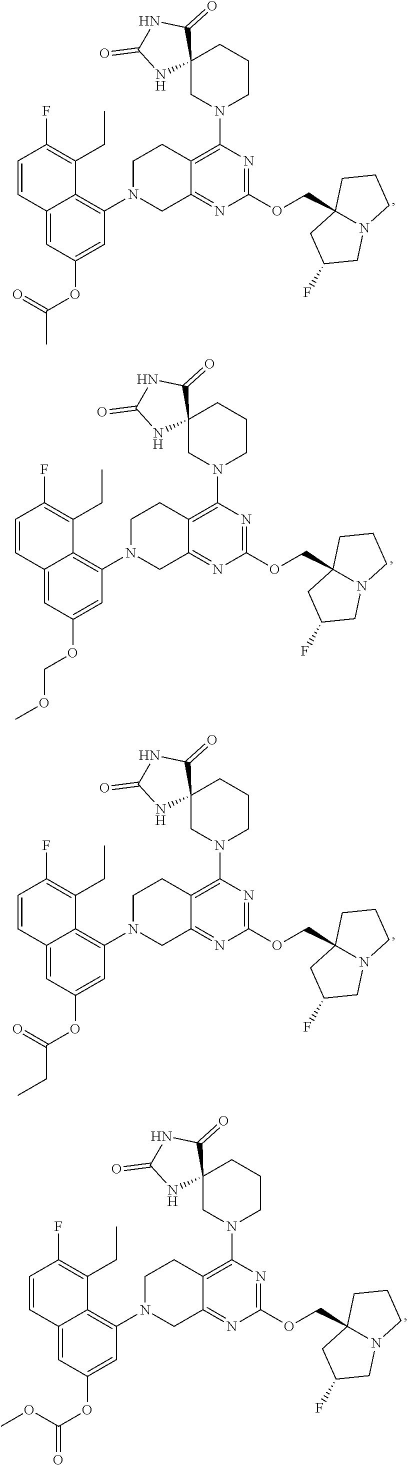 [SMILES: CCc1c(F)ccc2cc(OC(C)=O)cc(N3CCc4c(nc(OC[C@@]56CCCN5C[C@H](F)C6)nc4N4CCC[C@]5(C4)NC(=O)NC5=O)C3)c12 ; CCc1c(F)ccc2cc(OCOC)cc(N3CCc4c(nc(OC[C@@]56CCCN5C[C@H](F)C6)nc4N4CCC[C@]5(C4)NC(=O)NC5=O)C3)c12 ; CCC(=O)Oc1cc(N2CCc3c(nc(OC[C@@]45CCCN4C[C@H](F)C5)nc3N3CCC[C@]4(C3)NC(=O)NC4=O)C2)c2c(CC)c(F)ccc2c1 ; CCc1c(F)ccc2cc(OC(=O)OC)cc(N3CCc4c(nc(OC[C@@]56CCCN5C[C@H](F)C6)nc4N4CCC[C@]5(C4)NC(=O)NC5=O)C3)c12]

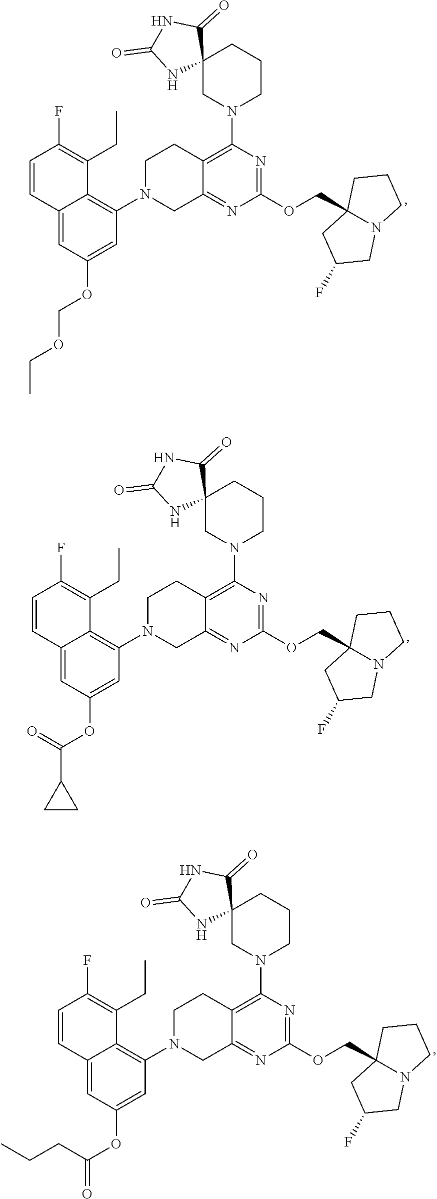 [SMILES: CCOCOc1cc(N2CCc3c(nc(OC[C@@]45CCCN4C[C@H](F)C5)nc3N3CCC[C@]4(C3)NC(=O)NC4=O)C2)c2c(CC)c(F)ccc2c1 ; CCc1c(F)ccc2cc(OC(=O)C3CC3)cc(N3CCc4c(nc(OC[C@@]56CCCN5C[C@H](F)C6)nc4N4CCC[C@]5(C4)NC(=O)NC5=O)C3)c12 ; CCCC(=O)Oc1cc(N2CCc3c(nc(OC[C@@]45CCCN4C[C@H](F)C5)nc3N3CCC[C@]4(C3)NC(=O)NC4=O)C2)c2c(CC)c(F)ccc2c1]

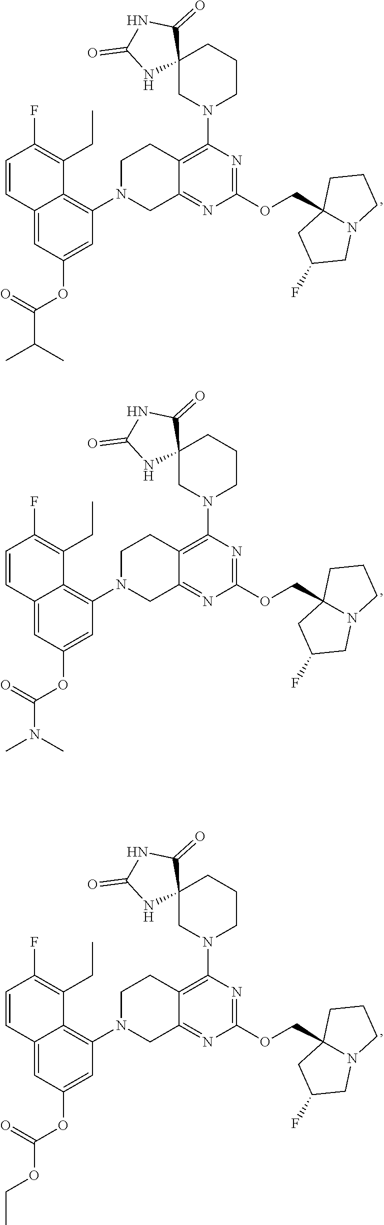 [SMILES: CCc1c(F)ccc2cc(OC(=O)C(C)C)cc(N3CCc4c(nc(OC[C@@]56CCCN5C[C@H](F)C6)nc4N4CCC[C@]5(C4)NC(=O)NC5=O)C3)c12 ; CCc1c(F)ccc2cc(OC(=O)N(C)C)cc(N3CCc4c(nc(OC[C@@]56CCCN5C[C@H](F)C6)nc4N4CCC[C@]5(C4)NC(=O)NC5=O)C3)c12 ; CCOC(=O)Oc1cc(N2CCc3c(nc(OC[C@@]45CCCN4C[C@H](F)C5)nc3N3CCC[C@]4(C3)NC(=O)NC4=O)C2)c2c(CC)c(F)ccc2c1]

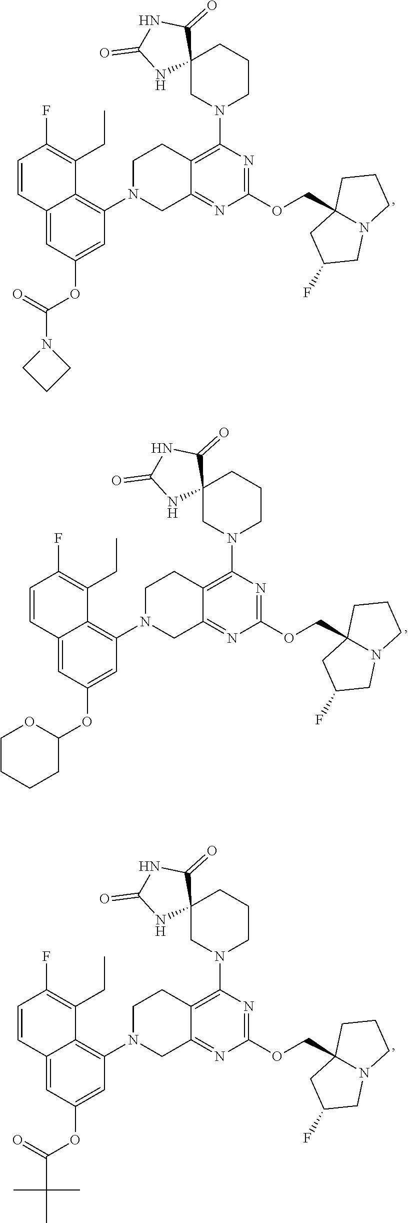 [SMILES: CCc1c(F)ccc2cc(OC(=O)N3CCC3)cc(N3CCc4c(nc(OC[C@@]56CCCN5C[C@H](F)C6)nc4N4CCC[C@]5(C4)NC(=O)NC5=O)C3)c12 ; CCc1c(F)ccc2cc(OC3CCCCO3)cc(N3CCc4c(nc(OC[C@@]56CCCN5C[C@H](F)C6)nc4N4CCC[C@]5(C4)NC(=O)NC5=O)C3)c12 ; CCc1c(F)ccc2cc(OC(=O)C(C)(C)C)cc(N3CCc4c(nc(OC[C@@]56CCCN5C[C@H](F)C6)nc4N4CCC[C@]5(C4)NC(=O)NC5=O)C3)c12]

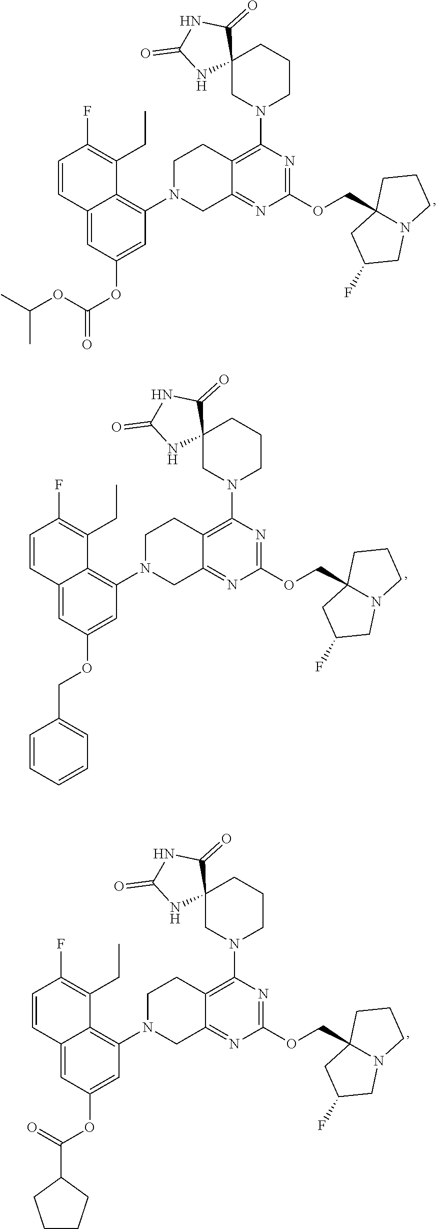 [SMILES: CCc1c(F)ccc2cc(OC(=O)OC(C)C)cc(N3CCc4c(nc(OC[C@@]56CCCN5C[C@H](F)C6)nc4N4CCC[C@]5(C4)NC(=O)NC5=O)C3)c12 ; CCc1c(F)ccc2cc(OCc3ccccc3)cc(N3CCc4c(nc(OC[C@@]56CCCN5C[C@H](F)C6)nc4N4CCC[C@]5(C4)NC(=O)NC5=O)C3)c12 ; CCc1c(F)ccc2cc(OC(=O)C3CCCC3)cc(N3CCc4c(nc(OC[C@@]56CCCN5C[C@H](F)C6)nc4N4CCC[C@]5(C4)NC(=O)NC5=O)C3)c12]

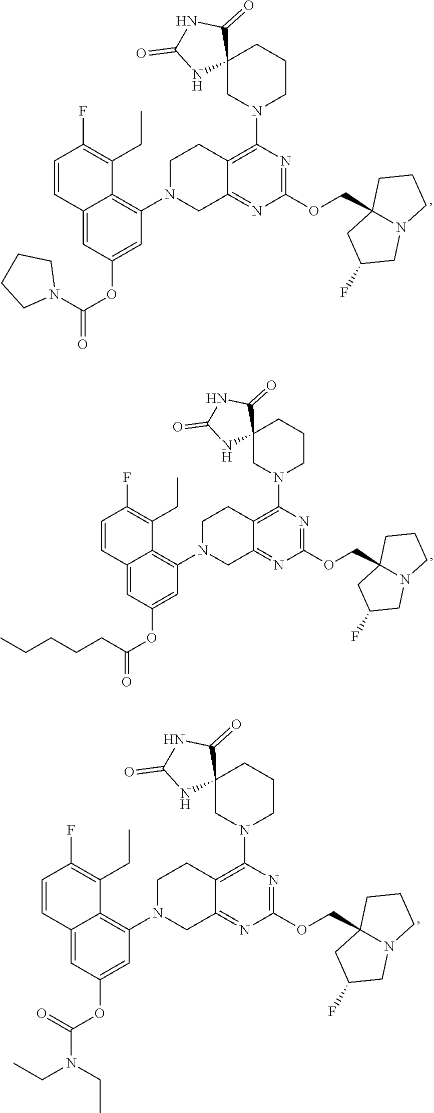 [SMILES: CCc1c(F)ccc2cc(OC(=O)N3CCCC3)cc(N3CCc4c(nc(OC[C@@]56CCCN5C[C@H](F)C6)nc4N4CCC[C@]5(C4)NC(=O)NC5=O)C3)c12 ; CCCCCC(=O)Oc1cc(N2CCc3c(nc(OC[C@@]45CCCN4C[C@H](F)C5)nc3N3CCC[C@]4(C3)NC(=O)NC4=O)C2)c2c(CC)c(F)ccc2c1 ; CCc1c(F)ccc2cc(OC(=O)N(CC)CC)cc(N3CCc4c(nc(OC[C@@]56CCCN5C[C@H](F)C6)nc4N4CCC[C@]5(C4)NC(=O)NC5=O)C3)c12]

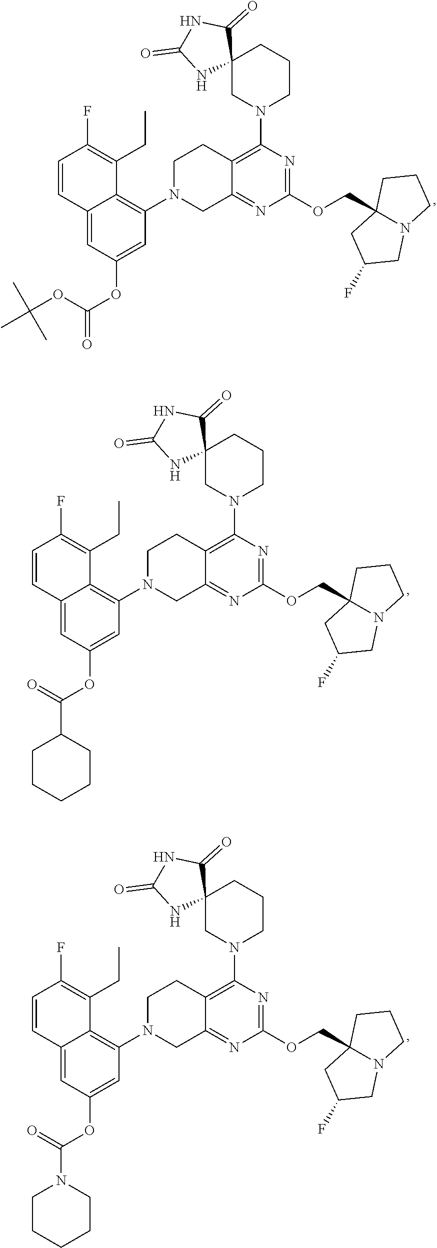 [SMILES: CCc1c(F)ccc2cc(OC(=O)OC(C)(C)C)cc(N3CCc4c(nc(OC[C@@]56CCCN5C[C@H](F)C6)nc4N4CCC[C@]5(C4)NC(=O)NC5=O)C3)c12 ; CCc1c(F)ccc2cc(OC(=O)C3CCCCC3)cc(N3CCc4c(nc(OC[C@@]56CCCN5C[C@H](F)C6)nc4N4CCC[C@]5(C4)NC(=O)NC5=O)C3)c12 ; CCc1c(F)ccc2cc(OC(=O)N3CCCCC3)cc(N3CCc4c(nc(OC[C@@]56CCCN5C[C@H](F)C6)nc4N4CCC[C@]5(C4)NC(=O)NC5=O)C3)c12]

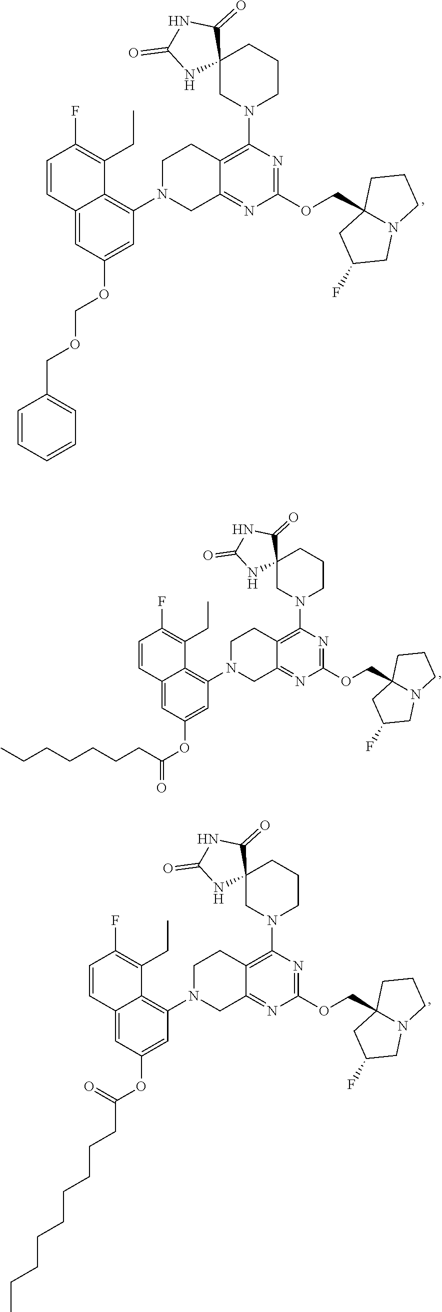 [SMILES: CCc1c(F)ccc2cc(OCOCc3ccccc3)cc(N3CCc4c(nc(OC[C@@]56CCCN5C[C@H](F)C6)nc4N4CCC[C@]5(C4)NC(=O)NC5=O)C3)c12 ; CCCCCCCC(=O)Oc1cc(N2CCc3c(nc(OC[C@@]45CCCN4C[C@H](F)C5)nc3N3CCC[C@]4(C3)NC(=O)NC4=O)C2)c2c(CC)c(F)ccc2c1 ; CCCCCCCCCC(=O)Oc1cc(N2CCc3c(nc(OC[C@@]45CCCN4C[C@H](F)C5)nc3N3CCC[C@]4(C3)NC(=O)NC4=O)C2)c2c(CC)c(F)ccc2c1]

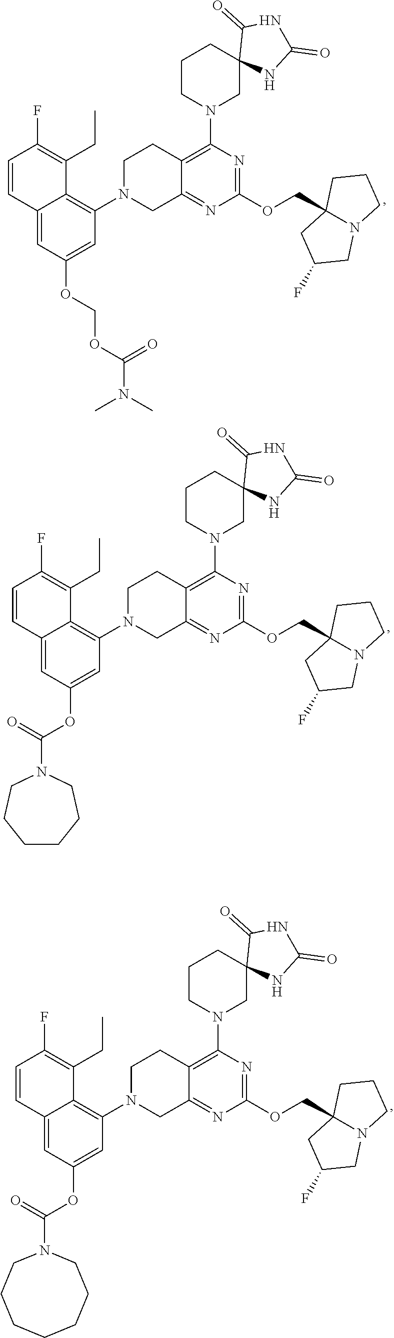 [SMILES: CCc1c(F)ccc2cc(OCOC(=O)N(C)C)cc(N3CCc4c(nc(OC[C@@]56CCCN5C[C@H](F)C6)nc4N4CCC[C@]5(C4)NC(=O)NC5=O)C3)c12 ; CCc1c(F)ccc2cc(OC(=O)N3CCCCCC3)cc(N3CCc4c(nc(OC[C@@]56CCCN5C[C@H](F)C6)nc4N4CCC[C@]5(C4)NC(=O)NC5=O)C3)c12 ; CCc1c(F)ccc2cc(OC(=O)N3CCCCCCC3)cc(N3CCc4c(nc(OC[C@@]56CCCN5C[C@H](F)C6)nc4N4CCC[C@]5(C4)NC(=O)NC5=O)C3)c12]

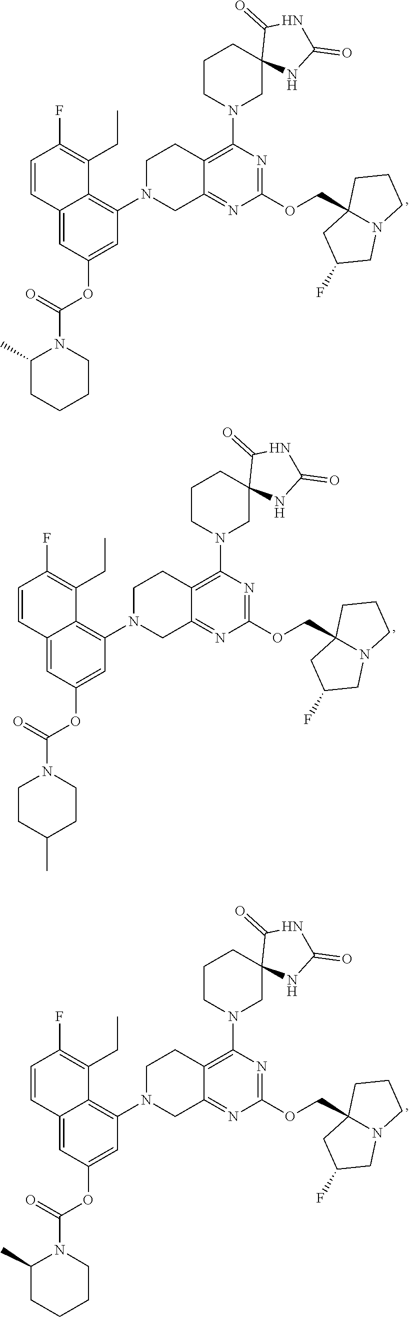 [SMILES: CCc1c(F)ccc2cc(OC(=O)N3CCCC[C@@H]3C)cc(N3CCc4c(nc(OC[C@@]56CCCN5C[C@H](F)C6)nc4N4CCC[C@]5(C4)NC(=O)NC5=O)C3)c12 ; CCc1c(F)ccc2cc(OC(=O)N3CCC(C)CC3)cc(N3CCc4c(nc(OC[C@@]56CCCN5C[C@H](F)C6)nc4N4CCC[C@]5(C4)NC(=O)NC5=O)C3)c12 ; CCc1c(F)ccc2cc(OC(=O)N3CCCC[C@H]3C)cc(N3CCc4c(nc(OC[C@@]56CCCN5C[C@H](F)C6)nc4N4CCC[C@]5(C4)NC(=O)NC5=O)C3)c12]

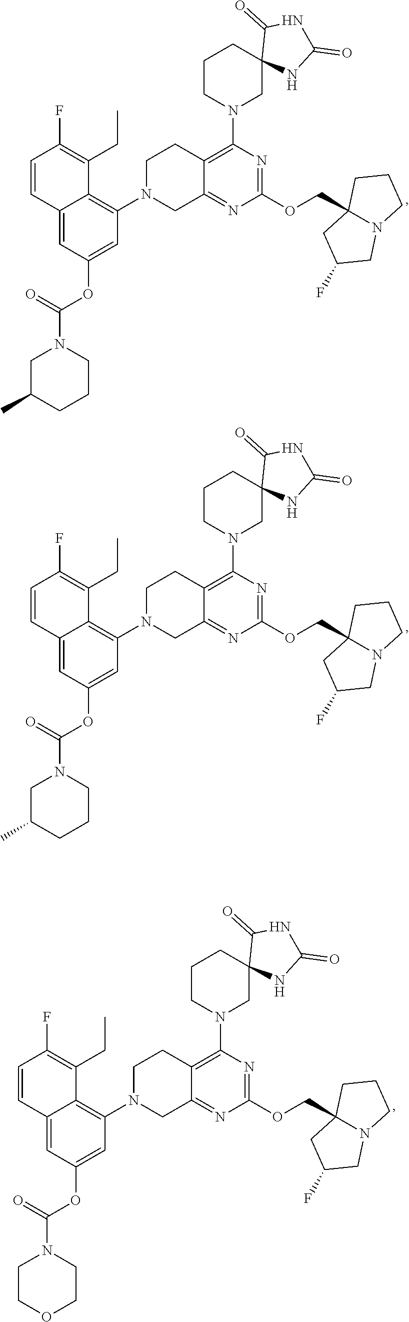 [SMILES: CCc1c(F)ccc2cc(OC(=O)N3CCC[C@@H](C)C3)cc(N3CCc4c(nc(OC[C@@]56CCCN5C[C@H](F)C6)nc4N4CCC[C@]5(C4)NC(=O)NC5=O)C3)c12 ; CCc1c(F)ccc2cc(OC(=O)N3CCC[C@H](C)C3)cc(N3CCc4c(nc(OC[C@@]56CCCN5C[C@H](F)C6)nc4N4CCC[C@]5(C4)NC(=O)NC5=O)C3)c12 ; CCc1c(F)ccc2cc(OC(=O)N3CCOCC3)cc(N3CCc4c(nc(OC[C@@]56CCCN5C[C@H](F)C6)nc4N4CCC[C@]5(C4)NC(=O)NC5=O)C3)c12]

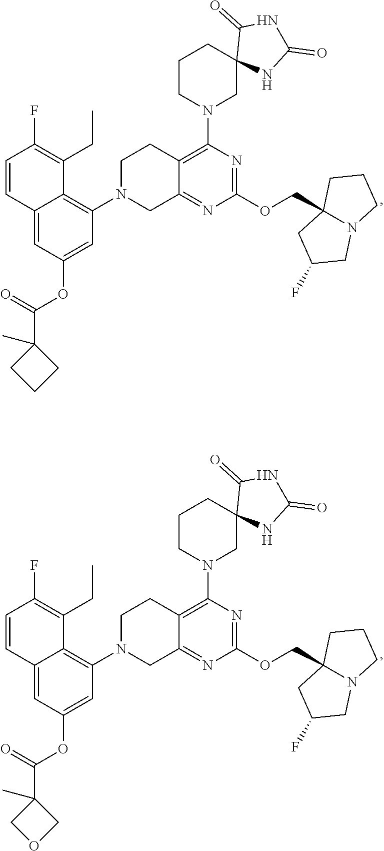 [SMILES: CCc1c(F)ccc2cc(OC(=O)C3(C)CCC3)cc(N3CCc4c(nc(OC[C@@]56CCCN5C[C@H](F)C6)nc4N4CCC[C@]5(C4)NC(=O)NC5=O)C3)c12 ; CCc1c(F)ccc2cc(OC(=O)C3(C)COC3)cc(N3CCc4c(nc(OC[C@@]56CCCN5C[C@H](F)C6)nc4N4CCC[C@]5(C4)NC(=O)NC5=O)C3)c12]

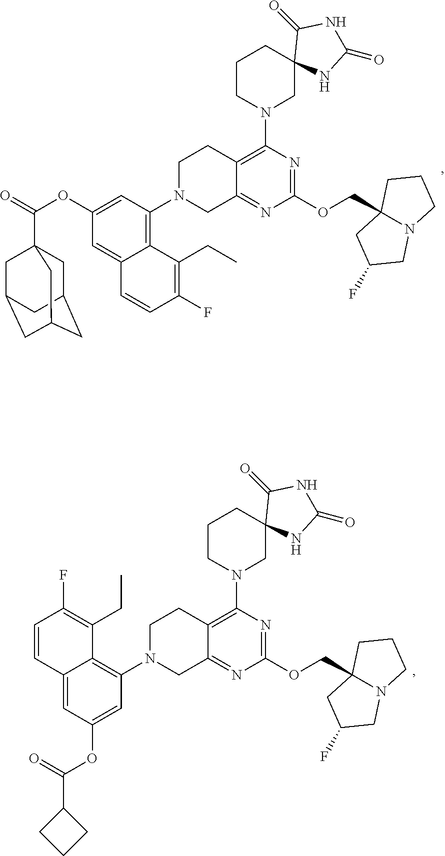 [SMILES: CCc1c(F)ccc2cc(OC(=O)C34CC5CC(CC(C5)C3)C4)cc(N3CCc4c(nc(OC[C@@]56CCCN5C[C@H](F)C6)nc4N4CCC[C@]5(C4)NC(=O)NC5=O)C3)c12 ; CCc1c(F)ccc2cc(OC(=O)C3CCC3)cc(N3CCc4c(nc(OC[C@@]56CCCN5C[C@H](F)C6)nc4N4CCC[C@]5(C4)NC(=O)NC5=O)C3)c12]

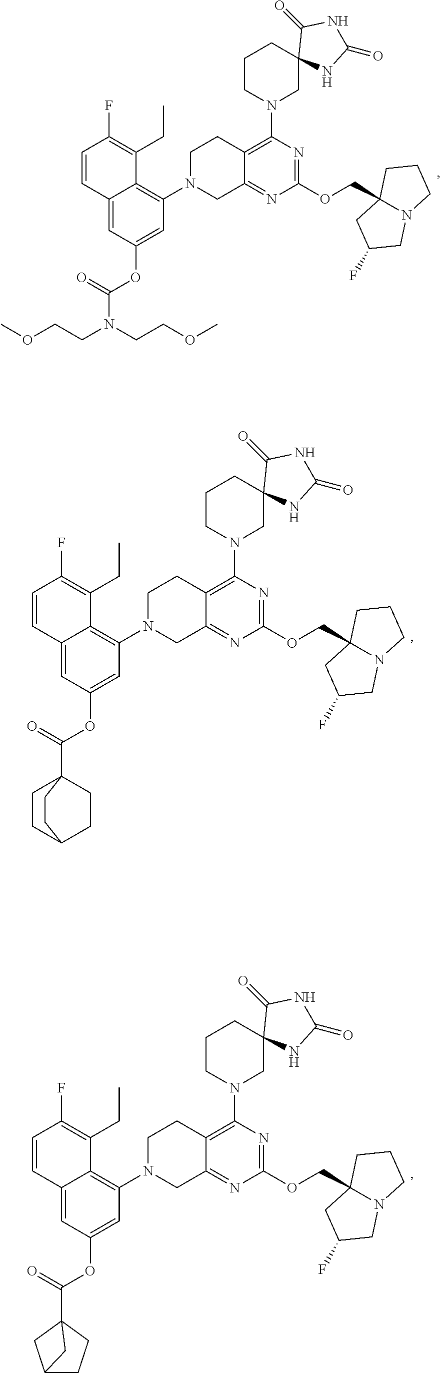 [SMILES: CCc1c(F)ccc2cc(OC(=O)N(CCOC)CCOC)cc(N3CCc4c(nc(OC[C@@]56CCCN5C[C@H](F)C6)nc4N4CCC[C@]5(C4)NC(=O)NC5=O)C3)c12 ; CCc1c(F)ccc2cc(OC(=O)C34CCC(CC3)CC4)cc(N3CCc4c(nc(OC[C@@]56CCCN5C[C@H](F)C6)nc4N4CCC[C@]5(C4)NC(=O)NC5=O)C3)c12 ; CCc1c(F)ccc2cc(OC(=O)C34CCC(C3)C4)cc(N3CCc4c(nc(OC[C@@]56CCCN5C[C@H](F)C6)nc4N4CCC[C@]5(C4)NC(=O)NC5=O)C3)c12]

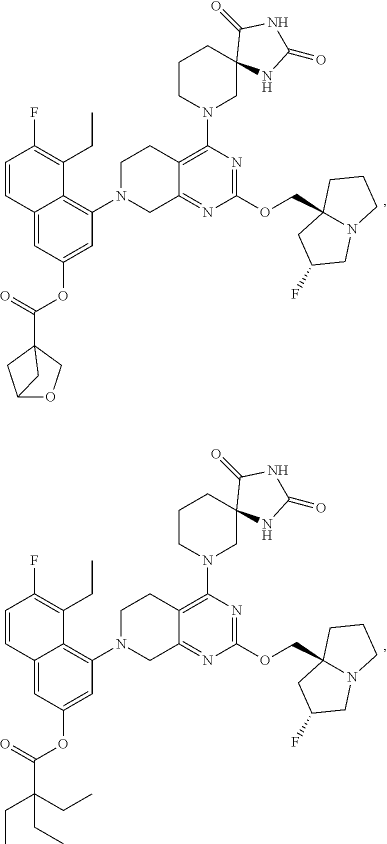 [SMILES: CCc1c(F)ccc2cc(OC(=O)C34COC(C3)C4)cc(N3CCc4c(nc(OC[C@@]56CCCN5C[C@H](F)C6)nc4N4CCC[C@]5(C4)NC(=O)NC5=O)C3)c12 ; CCc1c(F)ccc2cc(OC(=O)C(CC)(CC)CC)cc(N3CCc4c(nc(OC[C@@]56CCCN5C[C@H](F)C6)nc4N4CCC[C@]5(C4)NC(=O)NC5=O)C3)c12]

and pharmaceutically acceptable salts thereof.

In another aspect of the invention, the provided prodrugs have the following structure:

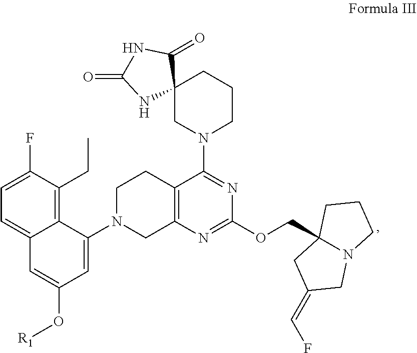

or a pharmaceutically acceptable salt thereof, wherein
R₁ is selected from the group consisting of:
R₃—*, R₃—O—CH₂—*,

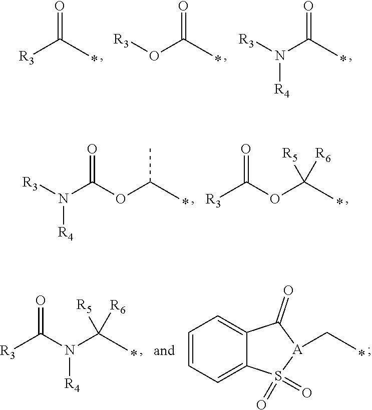

A is CH or N;
R₃ is C₁-C₂₀ alkyl, (C₁-C₄ alkyl)₀₋₁-cycloalkyl, (C₁-C₄ alkyl)₀₋₁-heterocyclyl or (C₁-C₄ alkyl)₀₋₁-aryl, where R₃ is optionally substituted with C₁-C₄ alkyl or —O— C₁-C₄ alkyl;
R₄ is H or R₃; and
R₅ and R₆ are independently selected from the group consisting of H and C₁-C₆ alkyl.

In one aspect, the compounds of Formula III are selected from the group consisting of:

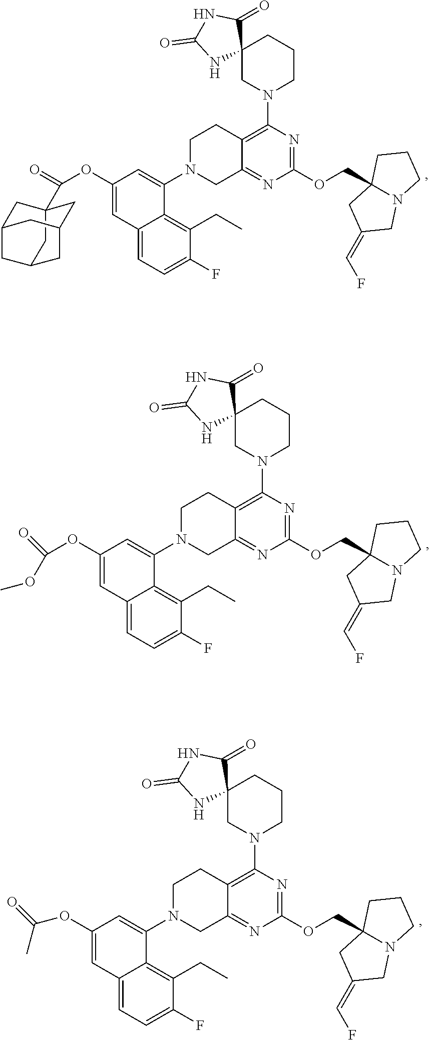 [SMILES: CCc1c(F)ccc2cc(OC(=O)C34CC5CC(CC(C5)C3)C4)cc(N3CCc4c(nc(OC[C@@]56CCCN5C/C(=C\F)C6)nc4N4CCC[C@]5(C4)NC(=O)NC5=O)C3)c12 ; CCc1c(F)ccc2cc(OC(=O)OC)cc(N3CCc4c(nc(OC[C@@]56CCCN5C/C(=C\F)C6)nc4N4CCC[C@]5(C4)NC(=O)NC5=O)C3)c12 ; CCc1c(F)ccc2cc(OC(C)=O)cc(N3CCc4c(nc(OC[C@@]56CCCN5C/C(=C\F)C6)nc4N4CCC[C@]5(C4)NC(=O)NC5=O)C3)c12]

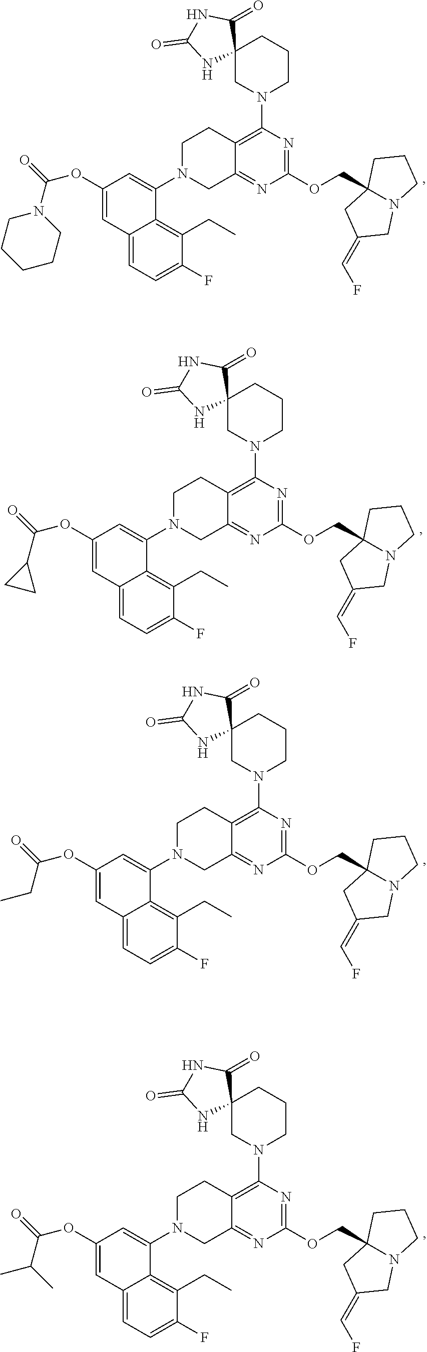 [SMILES: CCc1c(F)ccc2cc(OC(=O)N3CCCCC3)cc(N3CCc4c(nc(OC[C@@]56CCCN5C/C(=C\F)C6)nc4N4CCC[C@]5(C4)NC(=O)NC5=O)C3)c12 ; CCc1c(F)ccc2cc(OC(=O)C3CC3)cc(N3CCc4c(nc(OC[C@@]56CCCN5C/C(=C\F)C6)nc4N4CCC[C@]5(C4)NC(=O)NC5=O)C3)c12 ; CCC(=O)Oc1cc(N2CCc3c(nc(OC[C@@]45CCCN4C/C(=C\F)C5)nc3N3CCC[C@]4(C3)NC(=O)NC4=O)C2)c2c(CC)c(F)ccc2c1 ; CCc1c(F)ccc2cc(OC(=O)C(C)C)cc(N3CCc4c(nc(OC[C@@]56CCCN5C/C(=C\F)C6)nc4N4CCC[C@]5(C4)NC(=O)NC5=O)C3)c12]

and pharmaceutically acceptable salts thereof.

In another aspect of the invention, the provided prodrugs have the following generic structure:

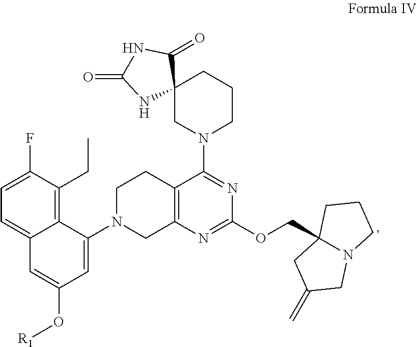

or a pharmaceutically acceptable salt thereof, wherein
R₁ is selected from the group consisting of:
R₃—*, R₃—O—CH₂—*,

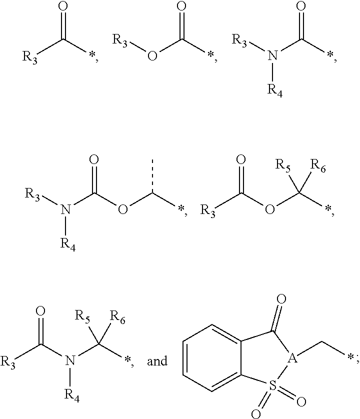

A is CH or N;
R₃ is C₁-C₂₀ alkyl, (C₁-C₄ alkyl)₀₋₁-cycloalkyl, (C₁-C₄ alkyl)₀₋₁-heterocyclyl or (C₁-C₄ alkyl)₀₋₁-aryl, where R₃ is optionally substituted with C₁-C₄ alkyl or —O— C₁-C₄ alkyl;
R₄ is H or R₃; and
R₅ and R₆ are independently selected from the group consisting of H and C₁-C₆ alkyl.

In one aspect, the compounds of Formula IV are selected from the group consisting of:

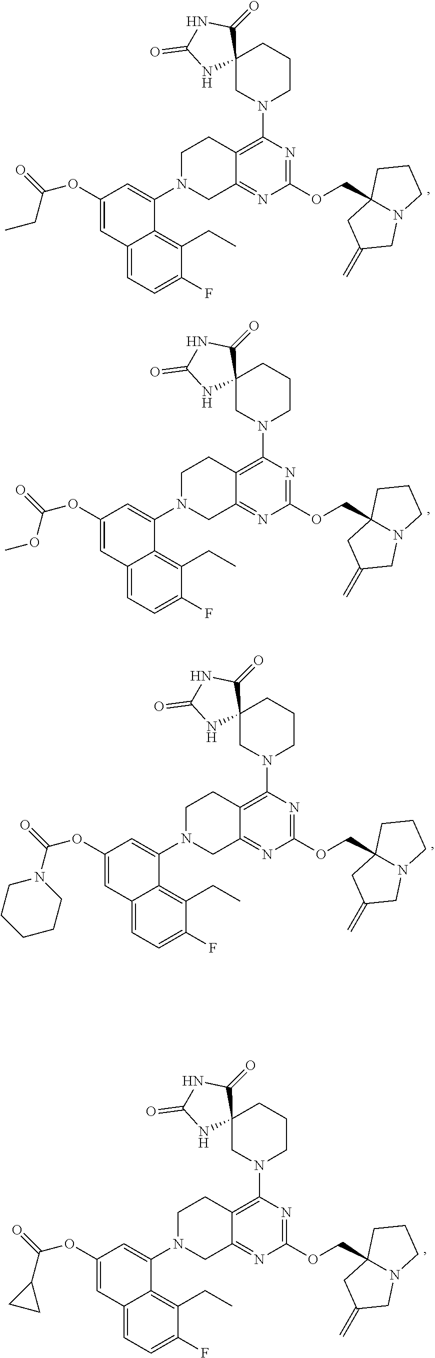 [SMILES: C=C1CN2CCC[C@@]2(COc2nc3c(c(N4CCC[C@]5(C4)NC(=O)NC5=O)n2)CCN(c2cc(OC(=O)CC)cc4ccc(F)c(CC)c24)C3)C1 ; C=C1CN2CCC[C@@]2(COc2nc3c(c(N4CCC[C@]5(C4)NC(=O)NC5=O)n2)CCN(c2cc(OC(=O)OC)cc4ccc(F)c(CC)c24)C3)C1 ; C=C1CN2CCC[C@@]2(COc2nc3c(c(N4CCC[C@]5(C4)NC(=O)NC5=O)n2)CCN(c2cc(OC(=O)N4CCCCC4)cc4ccc(F)c(CC)c24)C3)C1 ; C=C1CN2CCC[C@@]2(COc2nc3c(c(N4CCC[C@]5(C4)NC(=O)NC5=O)n2)CCN(c2cc(OC(=O)C4CC4)cc4ccc(F)c(CC)c24)C3)C1]

and pharmaceutically acceptable salts thereof.

In one embodiment, the compounds of Formulas (I)-(IV) include bis-hydrochloride, tris-hydrochloride, trifluoroacetic acid, bis-trifluoroacetic acid, and tris-trifluoracetic acid salts of the above compounds. The provided prodrug compounds or pharmaceutically acceptable salt thereof may be formulated into pharmaceutical compositions.

### Pharmaceutical Compositions

In another aspect, the invention provides pharmaceutical compositions comprising a prodrug of the invention and a pharmaceutically acceptable carrier, excipient, or diluent. Prodrug compounds of the invention may be formulated by any method well known in the art and may be prepared for administration by any route, including, without limitation, parenteral, intraperitoneal, intradermal, intracardiac, intraventricular, intracranial, intracerebrospinal, intrasynovial, intrathecal administration, intramuscular injection, intravitreous injection, intravenous injection, intra-arterial injection, oral, buccal, sublingual, transdermal, topical, intranasal, intratracheal, intrarectal, subcutaneous, and topical administration. In certain embodiments, compounds of the invention are administered intravenously in a hospital setting. In one embodiment, administration may be by the oral route. In some embodiments, the provided pharmaceutical compositions may be administered to a subject in need of treatment by injection systemically, such as by intravenous injection; or by injection or application to the relevant site, such as by direct injection via syringe, or direct application to the site when the site is exposed in surgery; or by topical administration.

Parenteral administration can be by bolus injection or continuous infusion. Pharmaceutical compositions for injection may be presented in unit dosage form, e.g., in ampoules or in multi-dose containers, with an added preservative.

The provided pharmaceutical compositions can also be formulated as a depot preparation. Such long acting formulations may be administered by implantation (for example subcutaneously or intramuscularly) or by intramuscular injection. Thus, for example, the formulations may be modified with suitable polymeric or hydrophobic materials (for example as an emulsion in an acceptable oil) or ion exchange resins, or as sparingly soluble derivatives, for example, as a sparingly soluble salt.

The pharmaceutical compositions may, if desired, be presented in a vial, pack or a medical device, including but not limited to a dispenser device which may contain one or more unit dosage forms containing the active ingredient. In one embodiment the dispenser device can comprise a syringe having a single dose of the liquid formulation ready for injection. The syringe can be accompanied by instructions for administration.

The characteristics of the carrier will depend on the route of administration. As used herein, the term “pharmaceutically acceptable” means a non-toxic material that is compatible with a biological system such as a cell, cell culture, tissue, or organism, and that does not interfere with the effectiveness of the biological activity of the active ingredient(s). Thus, compositions according to the invention may contain, in addition to the inhibitor, diluents, fillers, salts, buffers, stabilizers, solubilizers, and other materials well known in the art. The preparation of pharmaceutically acceptable formulations is described in, e.g., Remington's Pharmaceutical Sciences, 18th Edition, ed. A. Gennaro, Mack Publishing Co., Easton, Pa., 1990.

As used herein, the term pharmaceutically acceptable salt refers to salts that retain the desired biological activity of the above-identified compounds and exhibit minimal or no undesired toxicological effects. Examples of such salts include, but are not limited to acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalacturonic acid. The compounds can also be administered as pharmaceutically acceptable quaternary salts known by those skilled in the art, which specifically include the quaternary ammonium salt of the formula —NR+Z—, wherein R is hydrogen, alkyl, or benzyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

The active compound is included in the pharmaceutically acceptable carrier or diluent in an amount sufficient to deliver to a patient a therapeutically effective amount without causing serious toxic effects in the patient treated. In one embodiment, a dose of the active compound for all of the above-mentioned conditions is in the range from about 0.01 to 300 mg/kg, for example 0.1 to 100 mg/kg per day, and as a further example 0.5 to about 25 mg per kilogram body weight of the recipient per day. A typical topical dosage will range from 0.01-3% wt/wt in a suitable carrier. The effective dosage range of the pharmaceutically acceptable derivatives can be calculated based on the weight of the parent compound to be delivered. If the derivative exhibits activity in itself, the effective dosage can be estimated as above using the weight of the derivative, or by other means known to those skilled in the art.

The pharmaceutical compositions comprising compounds of the present invention may be used in the methods of use described herein.

### Methods of Use

In yet another aspect, the invention provides for methods for inhibiting wild type KRas, KRas G12A, KRas G12C, KRas G12D, KRas G12R, KRas G12S, KRas G12V and/or KRas Q61H activity in a cell, comprising contacting the cell in which inhibition of wild type KRas, KRas G12A, KRas G12C, KRas G12D, KRas G12R, KRas G12S, KRas G12V and/or Q61H activity is desired with an effective amount of a prodrug compound of the invention, pharmaceutically acceptable salts thereof, or pharmaceutical compositions containing the compound or pharmaceutically acceptable salt thereof. In one embodiment, the contacting is in vitro. In one embodiment, the contacting is in vivo.

As used herein, the term “contacting” refers to the bringing together of indicated moieties in an in vitro system or an in vivo system. For example, “contacting” wild type KRas, KRas G12A, KRas G12C, KRas G12D, KRas G12R, KRas G12S, KRas G12V, KRas G13D and/or KRas Q61H with a compound provided herein includes the administration of a prodrug compound provided herein to an individual or patient, such as a human, having wild type KRas or a KRas G12A, KRas G12C, KRas G12D, KRas G12R, KRas G12S, KRas G12V, KRas G13D and/or KRas Q61H mutation, as well as, for example, introducing a prodrug compound provided herein into a sample containing a cellular or purified preparation containing wild type KRas or a KRas G12A, KRas G12C, KRas G12D, KRas G12R, KRas G12S, KRas G12V, KRas G13D or KRas Q61H mutation.

In one embodiment, a cell in which inhibition of wild type KRas or KRas G12A, KRas G12C, KRas G12D, KRas G12R, KRas G12S, KRas G12V, KRas G13D and/or KRas Q61H activity is desired is contacted with an effective amount of a prodrug compound or pharmaceutically acceptable salt thereof to negatively modulate the activity of one or more of wild type KRas or KRas G12A, KRas G12C, KRas G12D, KRas G12R, KRas G12S, KRas G12V, KRas G13D and KRas Q61H.

By negatively modulating the activity of one or more of wild type KRas or KRas G12A, KRas G12C, KRas G12D, KRas G12R, KRas G12S, KRas G12V, KRas G13D and KRas Q61H, the methods described herein are designed to inhibit undesired cellular proliferation resulting from enhanced wild type KRas or KRas G12A, KRasG12C, KRas G12D, KRas G12R, KRas G12S, KRas G12V, KRas G13D and/or KRas Q61H activity within the cell. The cells may be contacted in a single dose or multiple doses in accordance with a particular treatment regimen to affect the desired negative modulation of wild type KRas or KRas G12A, KRas G12C, KRas G12D, KRas G12R, KRas G12S, KRas G12V, KRas G13D and/or KRas Q61H. The ability of compounds to bind one or more of wild type KRas or KRas G12A, KRas G12C, KRas G12D, KRas G12R, KRas G12S, KRas G12V, KRas G13D and KRas Q61H may be monitored in vitro using well known methods. In addition, the inhibitory activity of exemplary compounds in cells may be monitored, for example, by measuring the inhibition of one or more of wild type KRas or KRas G12A, KRas G12C, KRas G12D, KRas G12R, KRas G12S, KRas G12V, KRas G13D and/or KRas Q61H activity of the amount of phosphorylated ERK.

In another aspect, methods of treating cancer in a patient in need thereof, comprising administering to said patient a therapeutically effective amount of a prodrug compound of the invention or a pharmaceutically acceptable salt thereof, or a pharmaceutical composition comprising the compound or pharmaceutically acceptable salt thereof are provided.

The compositions and methods provided herein may be used for the treatment of a wild type KRas-associated or KRas G12A, KRas G12C, KRas G12D, KRas G12R, KRas G12S, KRas G12V, KRas G13D and/or KRas Q61H-associated cancer in a patient in need thereof, comprising administering to said patient a therapeutically effective amount of a prodrug compound of Formulas (I)-(IV) or their metabolites, a pharmaceutically acceptable salt thereof, or a pharmaceutical composition comprising the compound or pharmaceutically acceptable salt thereof are provided. In one embodiment, the wild type KRas-associated or KRas G12A, KRas G12C, KRas G12D, KRas G12R, KRas G12S, KRas G12V, KRas G13D and/or KRas Q61H-associated cancer is lung cancer.

The compositions and methods provided herein may be used for the treatment of a wide variety of cancers including tumors such as lung, prostate, breast, brain, skin, cervical carcinomas, testicular carcinomas, etc. More particularly, cancers that may be treated by the compositions and methods of the invention include, but are not limited to tumor types such as astrocytic, breast, cervical, colorectal, endometrial, esophageal, gastric, head and neck, hepatocellular, laryngeal, lung, oral, ovarian, prostate and thyroid carcinomas and sarcomas. More specifically, these compounds can be used to treat: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; and Adrenal glands: neuroblastoma. In certain embodiments, the cancer is non-small cell lung cancer, small cell lung cancer, colorectal cancer, rectal cancer or pancreatic cancer. In certain embodiments, the cancer is non-small cell lung cancer.

The concentration and route of administration to the patient will vary depending on the cancer to be treated. The prodrug compounds, pharmaceutically acceptable salts thereof and pharmaceutical compositions comprising such compounds and salts also may be co-administered with other anti-neoplastic compounds, e.g., chemotherapy, or used in combination with other treatments, such as radiation or surgical intervention, either as an adjuvant prior to surgery or post-operatively.

Also provided herein are prodrug compounds, or a pharmaceutically acceptable salt thereof, or a pharmaceutical composition thereof as defined herein for use in therapy.

Also provided herein are prodrug compounds, or a pharmaceutically acceptable salt thereof or a pharmaceutical composition thereof as defined herein for use in the treatment of cancer.

Also provided herein are prodrug compounds, or a pharmaceutically acceptable salt thereof for use in the inhibition of KRas wild type or multiple types of KRas mutations, for instance KRas G12A, G12C, G12D, G12R, G12S, G12V, G13D and/or Q61H mutations.

Also provided herein are prodrug compounds, or a pharmaceutically acceptable salt thereof or a pharmaceutical composition thereof as defined herein, for use in the treatment of a KRas wild type associated disease or disorder or a KRas mutation G12A, G12C, G12D, G12R, G12S, G12V, G13D and/or Q61H-associated disease or disorder.

Also provided herein is the use of a prodrug compound of the invention, or a pharmaceutically acceptable salt thereof, as defined herein in the manufacture of a medicament for the treatment of cancer.

Also provided herein is a use of a prodrug compound of the invention, or a pharmaceutically acceptable salt thereof, as defined herein in the manufacture of a medicament for the inhibition of activity of the wild type form of KRas or mutated forms of KRas, including the mutations: G12A, G12C, G12D, G12R, G12S, G12V, G13D and/or Q61H.

Also provided herein is the use of a prodrug compound of the invention, or a pharmaceutically acceptable salt thereof, as defined herein, in the manufacture of a medicament for the treatment of a KRas wild type associated disease or disorder or a KRas G12A, G12C, G12D, G12R, G12S, G12V, G13D and/or Q61H-associated disease or disorder.

Also provided herein is a method for treating cancer in a patient in need thereof, the method comprising (a) determining that the cancer is associated with KRas wild type or a KRas G12A, G12C, G12D, G12R, G12S, G12V, G13D and/or Q61H mutation (i.e., a KRas G12A, G12C, G12D, G12R, G12S, G12V, G13D and/or Q61H-associated cancer); and (b) administering to the patient a therapeutically effective amount of a prodrug compound of the invention, or a pharmaceutically acceptable salt thereof, or a pharmaceutical composition thereof

One skilled in the art will recognize that, both in vivo and in vitro trials using suitable, known and generally accepted cell and/or animal models are predictive of the ability of a test compound to treat or prevent a given disorder.

One skilled in the art will further recognize that human clinical trials including first-in-human, dose ranging and efficacy trials, in healthy patients and/or those suffering from a given disorder, may be completed according to methods well known in the clinical and medical arts.

### Reaction Schemes and Examples

The prodrug compounds of the present invention may be prepared from commercially available reagents using well-known synthetic methods and reaction schemes described herein, and using other well-known methods, for example, similar to those described in PCT/US2021/010065 (WO 2022/132200), or using other reagents and conventional methods well known to those skilled in the art.

The compounds of the present invention may have one or more chiral center and may be synthesized as stereoisomeric mixtures, isomers of identical constitution that differ in the arrangement of their atoms in space. The compounds may be used as mixtures or the individual components/isomers may be separated using commercially available reagents and conventional methods for isolation of stereoisomers and enantiomers well-known to those skilled in the art, e.g., using CHIRALPAK® (Sigma-Aldrich) or CHIRALCEL® (Diacel Corp) chiral chromatographic HPLC columns according to the manufacturer's instructions. Alternatively, compounds of the present invention may be synthesized using optically pure, chiral reagents and intermediates to prepare individual isomers or enantiomers. Unless otherwise indicated, all chiral (enantiomeric and diastereomeric) and racemic forms are within the scope of the invention. Unless otherwise indicated, whenever the specification, including the claims, refers to compounds of the invention, the term “compound” is to be understood to encompass all chiral (enantiomeric and diastereomeric) and racemic forms.

The compounds of the present invention may be in anhydrous, solvated or hydrated forms, and all such forms are included within the scope of the invention.

The following Examples are intended to illustrate further certain embodiments of the invention and are not intended to limit the scope of the invention.

### Example 1

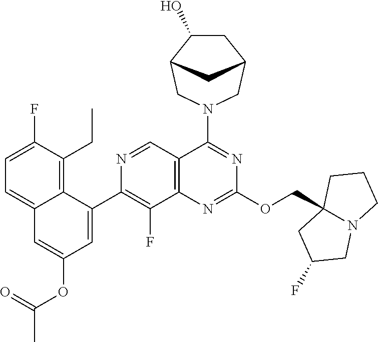 [SMILES: CCc1c(F)ccc2cc(OC(C)=O)cc(-c3ncc4c(N5C[C@@H]6C[C@H](C5)[C@H](O)C6)nc(OC[C@@]56CCCN5C[C@H](F)C6)nc4c3F)c12]

### 5-ethyl-6-fluoro-4-(8-fluoro-2-(((2R,7aS)-2-fluorotetrahydro-1H-pyrrolizin-7a(5H)-yl)methoxy)-4-((1R,5R,6R)-6-hydroxy-3-azabicyclo[3.2.1]octan-3-yl)pyrido[4,3-d]pyrimidin-7-yl)naphthalen-2-yl acetate

Step A. 5-ethyl-6-fluoro-4-(8-fluoro-2-(((2R,7aS)-2-fluorohexahydro-1H-pyrrolizin-7a-yl)methoxy)-4-((1R,5R,6R)-6-hydroxy-3-azabicyclo[3.2.1]octan-3-yl)pyrido[4,3-d]pyrimidin-7-yl)naphthalen-2-yl acetate: To a solution of (1R,5R,6R)-3-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-3-azabicyclo[3.2.1]octan-6-ol (200 mg, 1.0 equiv) and TEA (98.0 mg, 3.0 equiv) in dichloromethane (2 mL) was added acetyl chloride (25.3 mg, 1.0 equiv) at −40° C. The reaction was stirred at −40° C. for 0.5 hours. The mixture was diluted with water (3 mL) and extracted with dichloromethane (3×2 mL). The combined organic layers were dried over anhydrous sodium sulfate, concentrated and purified with prep-HPLC [Waters Xbridge 150×25 mm×5 μm; A: water (NH₄HCO₃), B: ACN, B %: 53%-83% over 8 min] to afford the title compound (101 mg, 46% yield) as white solid; ¹H NMR (400 MHz, DMSO-d6) δ=9.39-9.22 (m, 1H), 8.01 (dd, J=6.0, 9.2 Hz, 1H), 7.90 (d, J=2.4 Hz, 1H), 7.52 (dt, J=2.4, 9.2 Hz, 1H), 7.37 (dd, J=2.4, 6.4 Hz, 1H), 5.39-5.16 (m, 1H), 4.87-4.67 (m, 2H), 4.57 (br d, J=12.0 Hz, 1H), 4.25-4.06 (m, 2H), 4.01 (dd, J=1.6, 10.4 Hz, 1H), 3.74 (br t, J=10.4 Hz, 1H), 3.40-3.32 (m, 1H), 3.17-2.93 (m, 3H), 2.90-2.73 (m, 1H), 2.44-2.37 (m, 1H), 2.36-2.29 (m, 4H), 2.24-1.97 (m, 6H), 1.88-1.71 (m, 4H), 1.70-1.62 (m, 1H), 1.32-1.19 (m, 1H), 0.83-0.65 (m, 3H); LCMS (ESI, M+1): m/z=662.4.

### Example 2

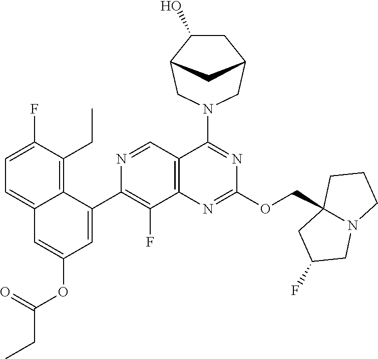 [SMILES: CCC(=O)Oc1cc(-c2ncc3c(N4C[C@@H]5C[C@H](C4)[C@H](O)C5)nc(OC[C@@]45CCCN4C[C@H](F)C5)nc3c2F)c2c(CC)c(F)ccc2c1]

### 5-ethyl-6-fluoro-4-(8-fluoro-2-(((2R,7aS)-2-fluorotetrahydro-1H-pyrrolizin-7a(5H)-yl)methoxy)-4-((1R,5R,6R)-6-hydroxy-3-azabicyclo[3.2.1]octan-3-yl)pyrido[4,3-d]pyrimidin-7-yl)naphthalen-2-yl propionate

Synthesized according to EXAMPLE 1. The title compound was obtained as white solid. 1H NMR (400 MHz, DMSO-d6) δ=9.38-9.20 (m, 1H), 8.01 (dd, J=6.0, 9.2 Hz, 1H), 7.90 (d, J=2.4 Hz, 1H), 7.52 (dt, J=2.0, 9.2 Hz, 1H), 7.37 (dd, J=2.4, 6.0 Hz, 1H), 5.41-5.16 (m, 1H), 4.89-4.68 (m, 2H), 4.57 (br d, J=11.6 Hz, 1H), 4.26-4.08 (m, 2H), 4.01 (d, J=10.4 Hz, 1H), 3.74 (br t, J=9.6 Hz, 1H), 3.42-3.32 (m, 1H), 3.16-2.95 (m, 3H), 2.89-2.76 (m, 1H), 2.69-2.61 (m, 2H), 2.44-2.37 (m, 1H), 2.36-2.31 (m, 1H), 2.26-1.97 (m, 6H), 1.88-1.70 (m, 4H), 1.69-1.61 (m, 1H), 1.30-1.20 (m, 1H), 1.16 (dt, J=1.2, 7.6 Hz, 3H), 0.75 (dt, J=2.4, 7.2 Hz, 3H); LCMS (ESI, M+1): m/z=676.3.

### Example 3

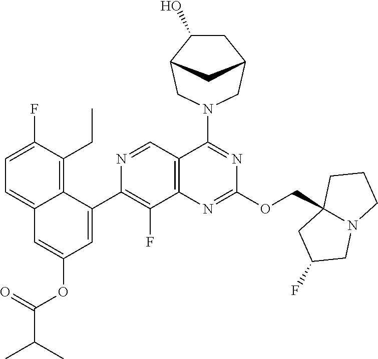 [SMILES: CCc1c(F)ccc2cc(OC(=O)C(C)C)cc(-c3ncc4c(N5C[C@@H]6C[C@H](C5)[C@H](O)C6)nc(OC[C@@]56CCCN5C[C@H](F)C6)nc4c3F)c12]

### 5-ethyl-6-fluoro-4-(8-fluoro-2-(((2R,7aS)-2-fluorotetrahydro-1H-pyrrolizin-7a(5H)-yl)methoxy)-4-((1R,5R,6R)-6-hydroxy-3-azabicyclo[3.2.1]octan-3-yl)pyrido[4,3-d]pyrimidin-7-yl)naphthalen-2-yl isobutyrate

Synthesized according to EXAMPLE 1. The title compound was obtained as white solid. 1H NMR (400 MHz, DMSO-d6) δ=9.39-9.18 (m, 1H), 8.01 (dd, J=6.0, 9.2 Hz, 1H), 7.91 (d, J=2.4 Hz, 1H), 7.53 (dt, J=2.0, 9.2 Hz, 1H), 7.35 (dd, J=2.4, 5.2 Hz, 1H), 5.45-5.14 (m, 1H), 4.87-4.67 (m, 2H), 4.57 (br d, J=11.2 Hz, 1H), 4.21-4.08 (m, 2H), 4.01 (d, J=10.4 Hz, 1H), 3.83-3.67 (m, 1H), 3.40-3.32 (m, 1H), 3.18-2.95 (m, 3H), 2.95-2.72 (m, 2H), 2.42 (td, J=7.6, 15.2 Hz, 1H), 2.33 (br d, J=2.0 Hz, 1H), 2.26-1.95 (m, 6H), 1.88-1.70 (m, 4H), 1.69-1.61 (m, 1H), 1.26 (d, J=7.2 Hz, 7H), 0.75 (dt, J=2.8, 7.2 Hz, 3H); LCMS (ESI, M+1): m/z=690.3.

### Example 4

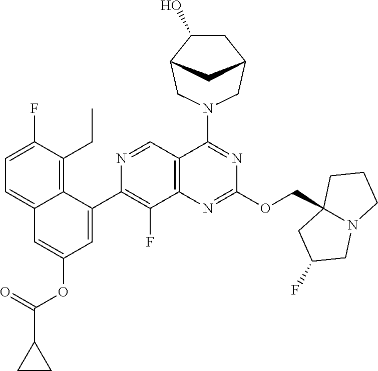 [SMILES: CCc1c(F)ccc2cc(OC(=O)C3CC3)cc(-c3ncc4c(N5C[C@@H]6C[C@H](C5)[C@H](O)C6)nc(OC[C@@]56CCCN5C[C@H](F)C6)nc4c3F)c12]

### 5-ethyl-6-fluoro-4-(8-fluoro-2-(((2R,7aS)-2-fluorotetrahydro-1H-pyrrolizin-7a(5H)-yl)methoxy)-4-((1R,5R,6R)-6-hydroxy-3-azabicyclo[3.2.1]octan-3-yl)pyrido[4,3-d]pyrimidin-7-yl)naphthalen-2-yl cyclopropanecarboxylate

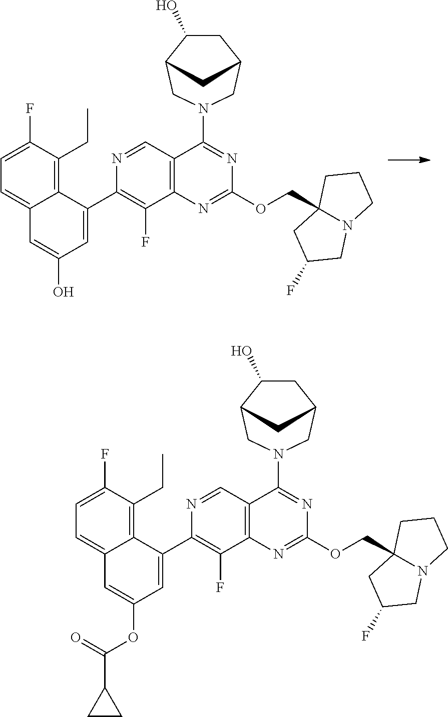 [SMILES: CCc1c(F)ccc2cc(O)cc(-c3ncc4c(N5C[C@@H]6C[C@H](C5)[C@H](O)C6)nc(OC[C@@]56CCCN5C[C@H](F)C6)nc4c3F)c12 ; CCc1c(F)ccc2cc(OC(=O)C3CC3)cc(-c3ncc4c(N5C[C@@H]6C[C@H](C5)[C@H](O)C6)nc(OC[C@@]56CCCN5C[C@H](F)C6)nc4c3F)c12]

Step A. 5-ethyl-6-fluoro-4-(8-fluoro-2-(((2R,7aS)-2-fluorohexahydro-1H-pyrrolizin-7a-yl)methoxy)-4-((1R,5R,6R)-6-hydroxy-3-azabicyclo[3.2.1]octan-3-yl)pyrido[4,3-d]pyrimidin-7-yl)naphthalen-2-yl cyclopropanecarboxylate: To a solution of (1R,5R,6R)-3-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-3-azabicyclo[3.2.1]octan-6-ol (100 mg, 1.0 equiv) and TEA (49.0 mg, 3.0 equiv) in DCM (2 mL) was added cyclopropanecarbonyl chloride (25.3 mg, 1.5 equiv) at −40° C. The reaction was stirred at −40° C. for 3 hours. The mixture was concentrated and purified with prep-HPLC [Waters Xbridge 150×25 mm×5 μm; A: water (NH₄HCO₃), B: ACN, B %: 58%-88% over 8 min] to afford the title compound (66.5 mg, 59% yield) as white solid. 1H NMR (400 MHz, DMSO-d6) δ=9.43-9.21 (m, 1H), 8.12-7.87 (m, 2H), 7.61-7.45 (m, 1H), 7.43-7.30 (m, 1H), 5.44-5.14 (m, 1H), 4.95-4.62 (m, 2H), 4.61-4.48 (m, 1H), 4.26-4.05 (m, 2H), 4.04-3.92 (m, 1H), 3.92-3.62 (m, 1H), 3.39-3.32 (m, 1H), 3.19-2.95 (m, 3H), 2.90-2.75 (m, 1H), 2.36-2.30 (m, 2H), 2.21-2.03 (m, 5H), 2.01-1.92 (m, 2H), 1.90-1.72 (m, 4H), 1.69-1.60 (m, 1H), 1.32-0.96 (m, 5H), 0.81-0.65 (m, 3H); LCMS (ESI, M+1): m/z=688.2.

### Example 5

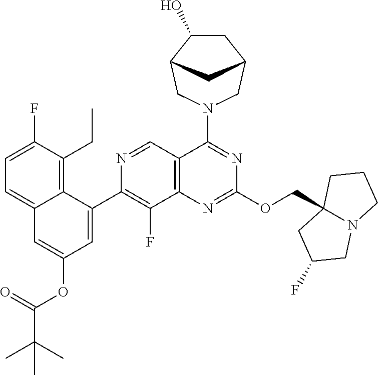 [SMILES: CCc1c(F)ccc2cc(OC(=O)C(C)(C)C)cc(-c3ncc4c(N5C[C@@H]6C[C@H](C5)[C@H](O)C6)nc(OC[C@@]56CCCN5C[C@H](F)C6)nc4c3F)c12]

### 5-ethyl-6-fluoro-4-(8-fluoro-2-(((2R,7aS)-2-fluorotetrahydro-1H-pyrrolizin-7a(5H)-yl)methoxy)-4-((1R,5R,6R)-6-hydroxy-3-azabicyclo[3.2.1]octan-3-yl)pyrido[4,3-d]pyrimidin-7-yl)naphthalen-2-yl pivalate

Synthesized according to EXAMPLE 4. The title compound was obtained as white solid. 1H NMR (400 MHz, DMSO-d6) δ=9.38-9.22 (m, 1H), 8.07-7.96 (m, 1H), 7.91 (d, J=2.4 Hz, 1H), 7.61-7.47 (m, 1H), 7.38-7.25 (m, 1H), 5.43-5.17 (m, 1H), 4.92-4.67 (m, 2H), 4.57 (br d, J=12.0 Hz, 1H), 4.23-4.07 (m, 2H), 4.05-3.96 (m, 1H), 3.80-3.70 (m, 1H), 3.51-3.35 (m, 1H), 3.15-2.96 (m, 3H), 2.89-2.76 (m, 1H), 2.44-2.31 (m, 2H), 2.25-1.93 (m, 6H), 1.88-1.70 (m, 4H), 1.69-1.59 (m, 1H), 1.33 (s, 9H), 1.28-1.18 (m, 1H), 0.84-0.67 (m, 3H); LCMS (ESI, M+1): m/z=704.1.

### Example 6

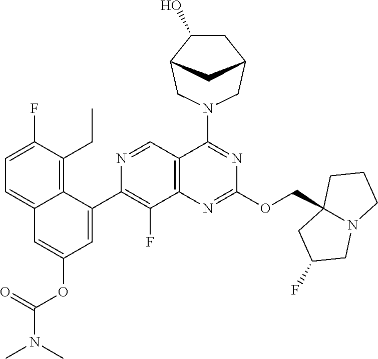 [SMILES: CCc1c(F)ccc2cc(OC(=O)N(C)C)cc(-c3ncc4c(N5C[C@@H]6C[C@H](C5)[C@H](O)C6)nc(OC[C@@]56CCCN5C[C@H](F)C6)nc4c3F)c12]

### 5-ethyl-6-fluoro-4-(8-fluoro-2-(((2R,7aS)-2-fluorotetrahydro-1H-pyrrolizin-7a(5H)-yl)methoxy)-4-((1R,5R,6R)-6-hydroxy-3-azabicyclo[3.2.1]octan-3-yl)pyrido[4,3-d]pyrimidin-7-yl)naphthalen-2-yl dimethylcarbamate

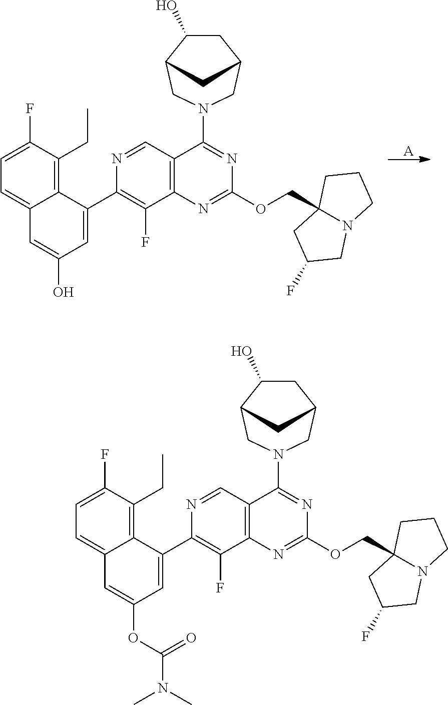

Step A. 5-ethyl-6-fluoro-4-(8-fluoro-2-(((2R,7aS)-2-fluorotetrahydro-1H-pyrrolizin-7a(5H)-yl)methoxy)-4-((1R,5R,6R)-6-hydroxy-3-azabicyclo[3.2.1]octan-3-yl)pyrido[4,3-d]pyrimidin-7-yl)naphthalen-2-yl dimethylcarbamate: To a solution of (1R,5R,6R)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-6-ol (100 mg, 1.0 equiv) in DMF (1 mL) were added K₂CO₃ (111 mg, 5.0 equiv) and N,N-dimethylcarbamoyl chloride (26.0 mg, 1.50 equiv) at 0° C. The reaction mixture was stirred at 20° C. for 12 hours. The mixture was filtered and purified with prep-HPLC [Waters Xbridge 150×25 mm×5 μm; A: water (NH₄HCO₃), B: ACN, B %: 51%-81% over 8 min] to afford the title compound (33.8 mg, 30% yield) as white solid; 1H NMR (400 MHz, DMSO-d6) δ=9.22-9.39 (m, 1H), 8.05-7.94 (m, 1H), 7.89 (d, J=2.4 Hz, 1H), 7.46-7.59 (m, 1H), 7.33 (dd, J=9.2, 2.4 Hz, 1H), 5.15-5.38 (m, 1H), 4.47-4.92 (m, 3H), 3.94-4.25 (m, 3H), 3.63-3.84 (m, 1H), 3.39-3.33 (m, 1H), 2.99-3.16 (m, 6H), 2.93 (s, 3H), 2.82 (m, 1H), 2.31-2.39 (m, 2H), 2.10-2.25 (m, 3H), 1.95-2.10 (m, 3H), 1.59-1.88 (m, 5H), 1.17-1.35 (m, 1H), 0.65-0.84 (in, 3H); L CMS (ESI, M+1): m/z=691.2.

### Example 7

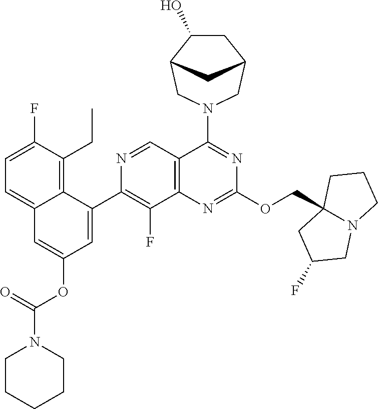 [SMILES: CCc1c(F)ccc2cc(OC(=O)N3CCCCC3)cc(-c3ncc4c(N5C[C@@H]6C[C@H](C5)[C@H](O)C6)nc(OC[C@@]56CCCN5C[C@H](F)C6)nc4c3F)c12]

### 5-ethyl-6-fluoro-4-(8-fluoro-2-(((2R,7aS)-2-fluorotetrahydro-1H-pyrrolizin-7a(5H)-yl)methoxy)-4-((1R,5R,6R)-6-hydroxy-3-azabicyclo[3.2.1]octan-3-yl)pyrido[4,3-d]pyrimidin-7-yl)naphthalen-2-yl piperidine-1-carboxylate

Synthesized according to EXAMPLE 6. The title compound was obtained as white solid. 1H NMR (400 MHz, DMSO-d6) δ=9.23-9.38 (m, 1H), 7.96-8.06 (m, 1H), 7.86-7.95 (m, 1H), 7.44-7.57 (m, 1H), 7.29-7.38 (m, 1H), 5.18-5.39 (m, 1H), 4.67-4.91 (m, 2H), 4.51-4.65 (m, 1H), 4.09-4.22 (m, 2H), 3.97-4.07 (m, 1H), 3.70-3.81 (m, 1H), 3.53-3.66 (m, 2H), 3.50-3.36 (m, 3H), 2.99-3.15 (m, 3H), 2.76-2.89 (m, 1H), 1.95-2.29 (m, 7H), 1.47-1.91 (m, 12H), 1.22-1.32 (m, 1H), 0.82-0.70 (m, 3H); LCMS (ESI, M+1): m/z=731.2.

### Example 8

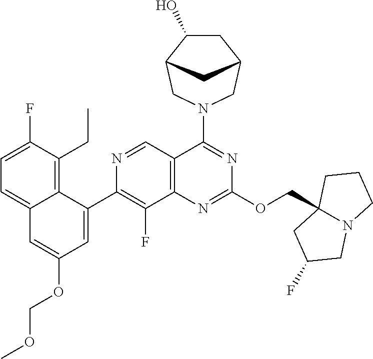 [SMILES: CCc1c(F)ccc2cc(OCOC)cc(-c3ncc4c(N5C[C@@H]6C[C@H](C5)[C@H](O)C6)nc(OC[C@@]56CCCN5C[C@H](F)C6)nc4c3F)c12]

### (1R,5R,6R)-3-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-6-ol

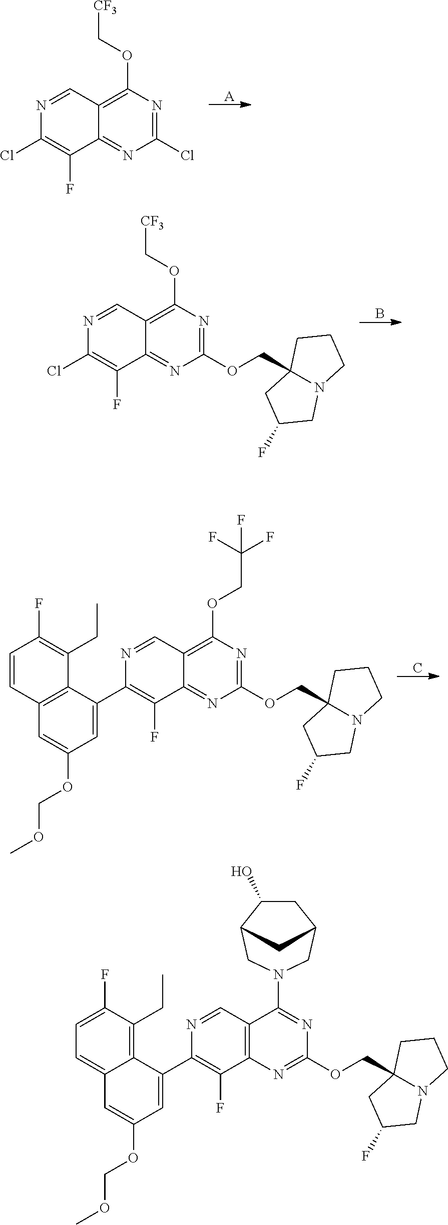

Step A. 7-chloro-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine: To a mixture of 2,7-dichloro-8-fluoro-4-(2,2,2-trifluoroethoxy)pyrido[4,3-d]pyrimidine (33.3 g, 1.0 equiv), DIEA (54.5 g, 4.0 equiv) and 4 Å molecular sieve (4.00 g) in THF (340 mL) was added ((2R,7aS)-2-fluorotetrahydro-1H-pyrrolizin-7a(5H)-yl)methanol (20.1 g, 1.2 equiv). The reaction was stirred at 40° C. for 14 hours. The mixture was quenched by adding water and concentrated to remove the organic solvent. The mixture was extracted with EA (2×200 mL), dried over Na₂SO₄, concentrated and purified with reversed phase flash chromatography [water (FA, 0.1%)/acetonitrile] to afford the title compound (28.8 g, 62% yield) as yellow solid. 1H NMR (400 MHz, chloroform-d) δ=8.98 (s, 1H), 5.40-5.19 (m, 1H), 5.02 (q, J=8.0 Hz, 1H, 2H), 4.40-4.27 (m, 2H), 3.34-3.12 (m, 3H), 3.05-2.94 (m, 1H), 2.32-2.06 (m, 3H), 2.03-1.84 (m, 3H); LCMS (ESI, M+1): m/z=439.1.

Step B. 7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine: To the solution of 7-chloro-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine (1.00 g, 1.0 equiv), 2-(8-ethyl-7-fluoro-3-(methoxymethoxy)naphthalen-1-yl)-4,4,5,5-tetramethyl-1,3,2-dioxaborolane (1.64 g, 2.0 equiv) and Cs₂CO₃ (1.5 M, 3.0 equiv) in methoxycyclopentane (15 mL) was added CataCXium A Pd G3 (332 mg, 0.20 equiv). The reaction was stirred at 100° C. for 2 hours. The mixture was quenched by adding water and concentrated to remove the organic solvent. The mixture was extracted with EtOAc (2×10 mL), dried over Na₂SO₄, concentrated and purified with reversed phase flash chromatography [C18, 0.1% formic acid condition] to afford the title compound (820 mg, 56% yield) as yellow solid; LCMS (ESI, M+1): m/z=637.3.

Step C. (1R,5R,6R)-3-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-6-ol: To a solution of 7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine (100 mg, 1.0 equiv) in DMF (0.5 mL) were added 4 Å MS (20.0 mg), (1R,5R,6R)-3-azabicyclo[3.2.1]octan-6-ol (31.9 mg, 1.6 equiv) and DIPEA (40.6 mg, 2.0 equiv). The reaction was stirred at 40° C. for 12 hours. The mixture was filtered and purified with prep-HPLC (column: UniSil 3-100 C18 Ultra (150*25 mm*3 μm); mobile phase: [water (FA)-ACN]; B %: 26%-46%, 7 min) to afford the title compound (59.9 mg, FA salt) as white solid; 1H NMR (400 MHz, METHANOL-d4) δ=9.25 (d, J=18.0 Hz, 1H), 7.86-7.73 (m, 1H), 7.65-7.55 (m, 1H), 7.39-7.16 (m, 2H), 5.48-5.26 (m, 3H), 4.59 (s, 3H), 4.45-4.37 (m, 1H), 4.35-4.25 (m, 2H), 3.89-3.72 (m, 1H), 3.54-3.35 (m, 6H), 3.20-3.07 (m, 1H), 2.55-2.14 (m, 8H), 2.12-2.01 (m, 2H), 2.00-1.88 (m, 2H), 1.87-1.75 (m, 1H), 1.43-1.26 (m, 2H), 0.86-0.71 (m, 3H); LCMS (ESI, M+1): m/z=664.5.

### Example 9

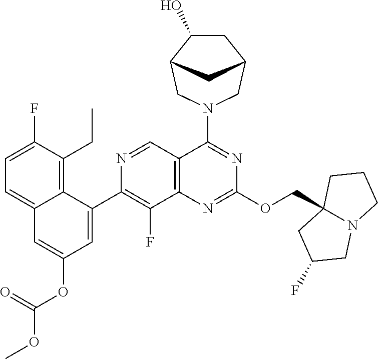 [SMILES: CCc1c(F)ccc2cc(OC(=O)OC)cc(-c3ncc4c(N5C[C@@H]6C[C@H](C5)[C@H](O)C6)nc(OC[C@@]56CCCN5C[C@H](F)C6)nc4c3F)c12]

### 5-ethyl-6-fluoro-4-(8-fluoro-2-(((2R,7aS)-2-fluorotetrahydro-1H-pyrrolizin-7a(5H)-yl)methoxy)-4-((1R,5R,6R)-6-hydroxy-3-azabicyclo[3.2.1]octan-3-yl)pyrido[4,3-d]pyrimidin-7-yl)naphthalen-2-yl methyl carbonate

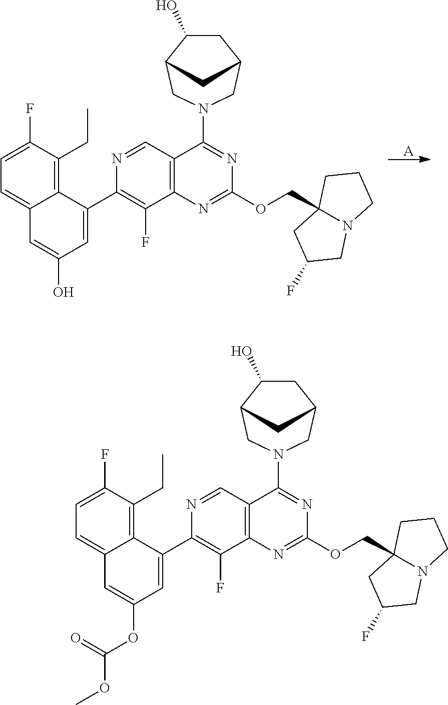

Step A. 5-ethyl-6-fluoro-4-(8-fluoro-2-(((2R,7aS)-2-fluorotetrahydro-1H-pyrrolizin-7a(5H)-yl)methoxy)-4-((1R,5R,6R)-6-hydroxy-3-azabicyclo[3.2.1]octan-3-yl)pyrido[4,3-d]pyrimidin-7-yl)naphthalen-2-yl methyl carbonate: To a solution of (1R,5R,6R)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-6-ol (500 mg, 1.00 equiv) and TEA (408 mg, 5.00 equiv) in THE (10.0 mL) was added dimethyl dicarbonate (216 mg, 2 equiv) at 0° C. The reaction was stirred at 0° C. for 0.5 hours. The mixture was diluted with water (15.0 mL) and extracted with ethyl acetate (3×10.0 mL). The combined organic layers were washed with brine (2×15.0 mL), dried over Na₂SO₄, filtered, concentrated under reduced pressure and triturated with methyl tertiary butyl ether at 25° C. for 30 minutes to afford the title compound (1.05 g, 82% yield) as a white solid; 1H NMR (400 MHz, METHANOL-d4) δ=0.82 (br d, J=7.72 Hz, 3H) 1.40 (br d, J=14.92 Hz, 1H) 1.78-2.04 (m, 5H) 2.13-2.58 (m, 8H) 3.00 (br s, 1H) 3.21 (br d, J=18.56 Hz, 2H) 3.35-3.52 (m, 2H) 3.75-3.85 (m, 1H) 3.90 (br s, 3H) 4.17-4.35 (m, 3H) 4.79 (br d, J=10.64 Hz, 1H) 5.00 (br s, 1H) 5.20-5.40 (m, 1H) 7.29-7.49 (m, 2H) 7.89 (br s, 2H) 9.24 (br d, J=18.12 Hz, 1H); LCMS (ESI, M+1): m/z=678.3.

### Example 10

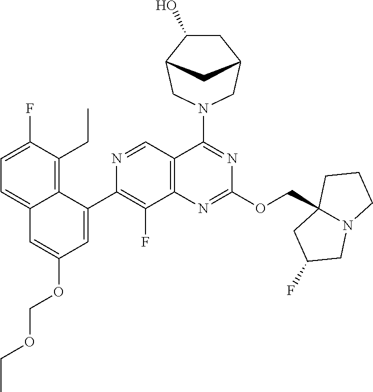 [SMILES: CCOCOc1cc(-c2ncc3c(N4C[C@@H]5C[C@H](C4)[C@H](O)C5)nc(OC[C@@]45CCCN4C[C@H](F)C5)nc3c2F)c2c(CC)c(F)ccc2c1]

### (1R,5R,6R)-3-(7-(3-(ethoxymethoxy)-8-ethyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-6-ol

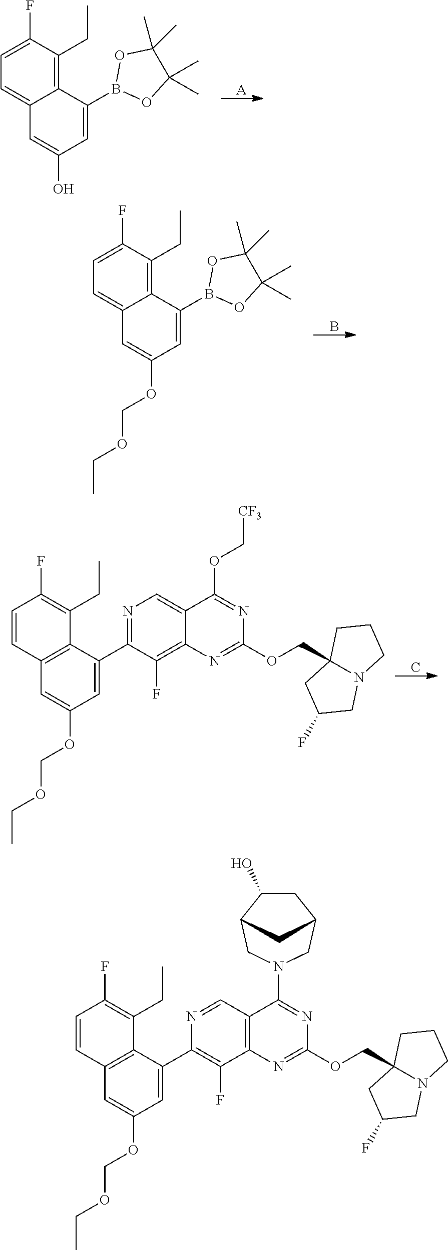

Step A. 2-(3-(ethoxymethoxy)-8-ethyl-7-fluoronaphthalen-1-yl)-4,4,5,5-tetramethyl-1,3,2-dioxaborolane: To a solution of 5-ethyl-6-fluoro-4-(4,4,5,5-tetramethyl-1,3,2-dioxaborolan-2-yl)naphthalen-2-ol (0.500 g, 1.0 equiv) and chloromethoxyethane (224 mg, 1.5 equiv) in DCM (25 mL) were added chloromethoxyethane (224 mg, 1.5 equiv) and DIEA (818 mg, 1.10 mL, 4.0 equiv) at 0° C. The reaction was stirred at 25° C. for 16 hours. The mixture was diluted with water (25 mL) and extracted with DCM (3×25 mL). The combined organic layer was dried over Na₂SO₄, filtered and concentrated under vacuum. The residue was purified by flash silica gel chromatography (ISCO®; 20.0 g SepaFlash® Silica Flash Column, Eluent of 0-100% Ethyl acetate/Petroleum ethergradient @10 mL/min) to afford the title compound (204 mg, 86% yield) as yellow solid; LCMS (ESI, M+1): m/z=375.1.

Step B. 7-(3-(ethoxymethoxy)-8-ethyl-7-fluoronaphthalen-1-yl)-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine: To a solution of 7-chloro-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine (117 mg, 1.0 equiv), 2-[3-(ethoxymethoxy)-8-ethyl-7-fluoro-1-naphthyl]-4,4,5,5-tetramethyl-1,3,2-dioxaborolane (0.10 g, 1.0 equiv) and K₃PO₄ (1.5 M, 534 μL, 3.0 equiv) in CPME (5 mL) was added [4-(di-tert-butylphosphino)-N,N-dimethylaniline-2-(2′-aminobiphenyl)]palladium(II) methanesulfonate (17.0 mg, 0.10 equiv). The reaction was degassed and purged with N2 for 3 times, and stirred at 70° C. for 1 hour under N2 atmosphere. The mixture was diluted with H₂O (5 mL) and extracted with DCM (3×5 mL). The combined organic layers were dried over Na₂SO₄, filtered, concentrated under vacuum and purified with flash silica gel chromatography (ISCO®; 20.0 g SepaFlash® Silica Flash Column, Eluent of 0˜100% Ethyl acetate/Petroleum ethergradient @50 mL/min) to afford the title compound (158 mg, 64% yield) as yellow solid; LCMS (ESI, M+1): m/z=651.2.

Step C. (1R,5R,6R)-3-(7-(3-(ethoxymethoxy)-8-ethyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-6-ol: To a solution of 7-[3-(ethoxymethoxy)-8-ethyl-7-fluoro-1-naphthyl]-8-fluoro-2-[[(2R,8S)-2-fluoro-1,2,3,5,6,7-hexahydropyrrolizin-8-yl]methoxy]-4-(2,2,2-trifluoroethoxy)pyrido[4,3-d]pyrimidine (0.20 g, 1.0 equiv) and (1R,5R,6R)-3-azabicyclo[3.2.1]octan-6-ol (93.8 mg, 2.4 equiv) in DMF (14 mL) were added 4A molecular sieve (0.20 g) and DIEA (119 mg, 3.0 equiv). The reaction was stirred at 40° C. for 12 hours. The mixture was diluted with H₂O (14 mL) and extracted with EtOAc (3×14 mL). The combined organic layers were dried over Na₂SO₄, filtered, concentrated under vacuum and purified with prep-HPLC (FA condition; column: Phenomenex luna C18 150*25 mm*10 um; mobile phase: [water(FA)-ACN]; B %: 21%-51%, min) to afford the title compound (0.13 g, 32% yield) as white solid; 1H NMR (400 MHz, DMSO-d6) δ=9.40-9.24 (m, 1H), 7.90 (dd, J=6.0, 9.0 Hz, 1H), 7.67 (d, J=2.2 Hz, 1H), 7.43 (br d, J=2.1 Hz, 1H), 7.23 (dd, J=2.6, 9.6 Hz, 1H), 5.42-5.32 (m, 3H), 5.22 (br s, 1H), 4.93-4.65 (m, 2H), 4.63-4.53 (m, 1H), 4.14 (br dd, J=10.3, 13.7 Hz, 2H), 4.01 (d, J=10.6 Hz, 1H), 3.72 (q, J=7.1 Hz, 3H), 3.11-3.01 (m, 3H), 2.83 (br d, J=6.6 Hz, 1H), 2.38-2.30 (m, 2H), 2.20-1.99 (m, 6H), 1.88-1.65 (m, 5H), 1.30-1.20 (m, 1H), 1.16 (t, J=7.1 Hz, 3H), 0.78-0.68 (m, 3H); LCMS (ESI, M+1): m/z=678.2.

### Example 11

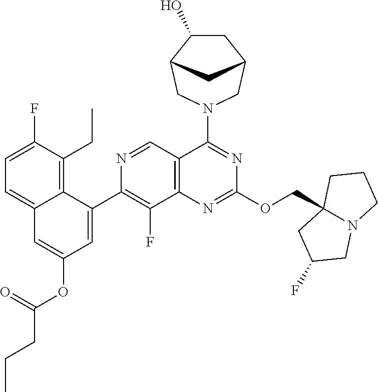 [SMILES: CCCC(=O)Oc1cc(-c2ncc3c(N4C[C@@H]5C[C@H](C4)[C@H](O)C5)nc(OC[C@@]45CCCN4C[C@H](F)C5)nc3c2F)c2c(CC)c(F)ccc2c1]

### 5-ethyl-6-fluoro-4-(8-fluoro-2-(((2R,7aS)-2-fluorotetrahydro-1H-pyrrolizin-7a(5H)-yl)methoxy)-4-((1R,5R,6R)-6-hydroxy-3-azabicyclo[3.2.1]octan-3-yl)pyrido[4,3-d]pyrimidin-7-yl)naphthalen-2-yl butyrate

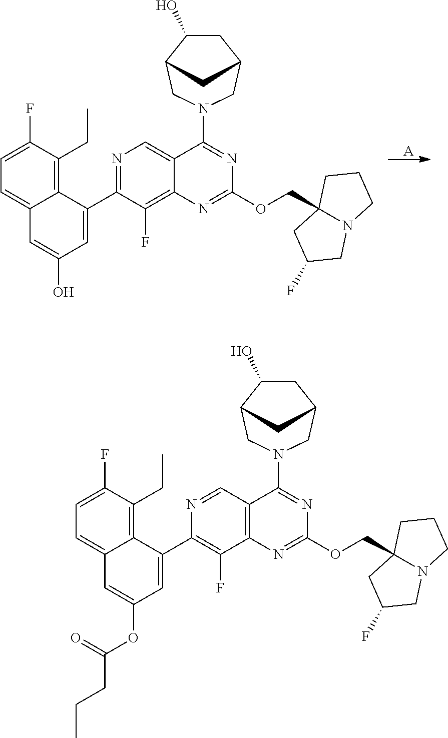

Step A. 5-ethyl-6-fluoro-4-(8-fluoro-2-(((2R,7aS)-2-fluorohexahydro-1H-pyrrolizin-7a-yl)methoxy)-4-((1R,5R,6R)-6-hydroxy-3-azabicyclo[3.2.1]octan-3-yl)pyrido[4,3-d]pyrimidin-7-yl)naphthalen-2-yl butyrate: To a solution of (1R,5R,6R)-3-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-3-azabicyclo[3.2.1]octan-6-ol (100 mg, 1.0 equiv) in DCM (1 mL) were added TEA (49.0 mg, 3.0 equiv) and butyryl chloride (17.2 mg, 1.0 equiv). The reaction was stirred at −40° C. for 0.5 hours. The mixture was diluted with water (10 mL) and extracted with DCM (3×5 mL). The combined organic layers were washed with brine (5 mL), dried over sodium sulfate, concentrated and purified with prep-HPLC (column: Waters Xbridge C18 150×50 mm×10 m; mobile phase: [water(NH₄HCO₃)-ACN]; B %: 58%-88%, 10 min) to afford the title compound (27.6 mg, 24% yield) as white solid; 1H NMR (400 MHz, CHLOROFORM-d) δ=9.05 (d, J=5.2 Hz, 1H), 7.77-7.69 (m, 2H), 7.31 (t, J=9.2 Hz, 1H), 7.23 (s, 1H), 5.40-5.21 (m, 1H), 5.03 (br t, J=13.2 Hz, 1H), 4.32 (br dd, J=5.2, 10.4 Hz, 2H), 4.28-4.17 (m, 2H), 3.83 (br d, J=12.8 Hz, 1H), 3.47 (br d, J=13.6 Hz, 1H), 3.34-3.17 (m, 3H), 3.04-2.93 (m, 1H), 2.61-2.49 (m, 3H), 2.43-2.19 (m, 6H), 2.14 (br d, J=10.0 Hz, 1H), 2.01-1.90 (m, 3H), 1.88-1.83 (m, 3H), 1.82-1.77 (m, 2H), 1.05 (t, J=7.2 Hz, 3H), 0.90-0.80 (m, 3H); LCMS (ESI, M+1): m/z=690.4.

### Example 12

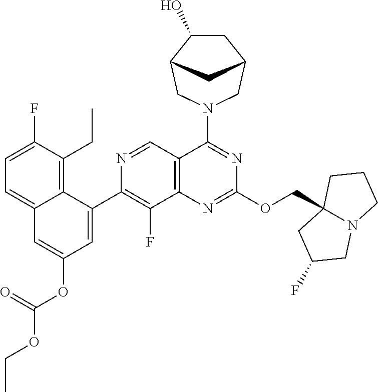 [SMILES: CCOC(=O)Oc1cc(-c2ncc3c(N4C[C@@H]5C[C@H](C4)[C@H](O)C5)nc(OC[C@@]45CCCN4C[C@H](F)C5)nc3c2F)c2c(CC)c(F)ccc2c1]

### ethyl (5-ethyl-6-fluoro-4-(8-fluoro-2-(((2R,7aS)-2-fluorotetrahydro-1H-pyrrolizin-7a(5H)-yl)methoxy)-4-((1R,5R,6R)-6-hydroxy-3-azabicyclo[3.2.1]octan-3-yl)pyrido[4,3-d]pyrimidin-7-yl)naphthalen-2-yl) carbonate

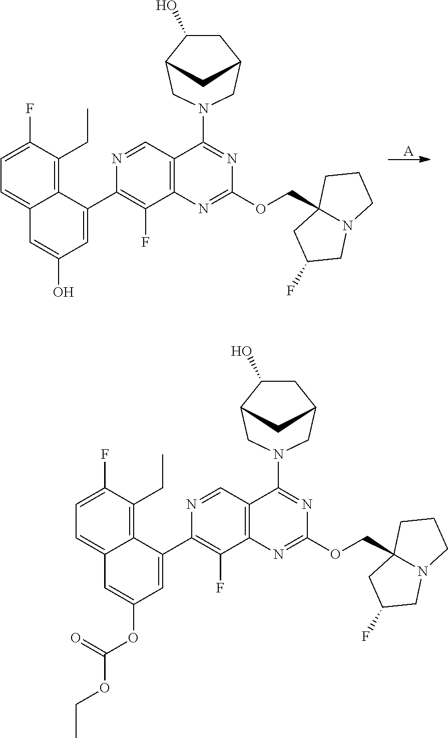

Step A. ethyl (5-ethyl-6-fluoro-4-(8-fluoro-2-(((2R,7aS)-2-fluorotetrahydro-1H-pyrrolizin-7a(5H)-yl)methoxy)-4-((1R,5R,6R)-6-hydroxy-3-azabicyclo[3.2.1]octan-3-yl)pyrido[4,3-d]pyrimidin-7-yl)naphthalen-2-yl) carbonate: To a solution of (1R,5R,6R)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-6-ol (500 mg, 1.00 equiv) and TEA (408 mg, 5.00 equiv) in THE (10.0 mL) was added diethyl dicarbonate (131 mg, 1.00 equiv) at 0° C. The reaction was stirred at 0° C. for 0.5 hours. The mixture was diluted with water (15 mL) and extracted with ethyl acetate (3×10 mL). The combined organic layers were washed with brine (3×15 mL), dried over Na₂SO₄, filtered, concentrated under reduced pressure and triturated with methyl tertiary butyl ether at 25° C. for 30 minutes to afford the title compound (1.06 g, 82% yield) as a white solid; 1H NMR (400 MHz, METHANOL-d4) δ=0.82 (br d, J=7.72 Hz, 3H) 1.32-1.42 (m, 4H) 1.82 (br s, 1H) 1.87-2.05 (m, 4H) 2.14-2.43 (m, 7H) 2.55 (br s, 1H) 3.01 (br s, 1H) 3.18 (br s, 1H) 3.23 (br s, 2H) 3.40-3.51 (m, 1H) 3.80 (br t, J=14.48 Hz, 1H) 4.17-4.37 (m, 5H) 4.77 (br d, J=10.52 Hz, 1H) 5.02 (br d, J=11.00 Hz, 1H) 5.17-5.44 (m, 1H) 7.27-7.50 (m, 2H) 7.84-7.98 (m, 2H) 9.24 (br d, J=18.12 Hz, 1H); LCMS (ESI, M+1): m/z=692.3.

### Example 13

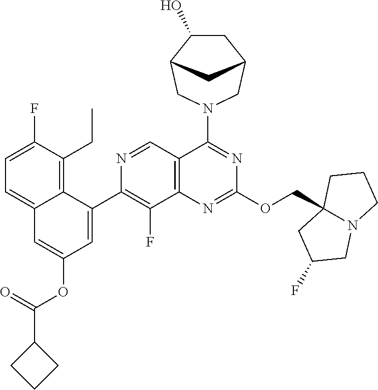 [SMILES: CCc1c(F)ccc2cc(OC(=O)C3CCC3)cc(-c3ncc4c(N5C[C@@H]6C[C@H](C5)[C@H](O)C6)nc(OC[C@@]56CCCN5C[C@H](F)C6)nc4c3F)c12]

### 5-ethyl-6-fluoro-4-(8-fluoro-2-(((2R,7aS)-2-fluorotetrahydro-1H-pyrrolizin-7a(5H)-yl)methoxy)-4-((1R,5R,6R)-6-hydroxy-3-azabicyclo[3.2.1]octan-3-yl)pyrido[4,3-d]pyrimidin-7-yl)naphthalen-2-yl cyclobutanecarboxylate

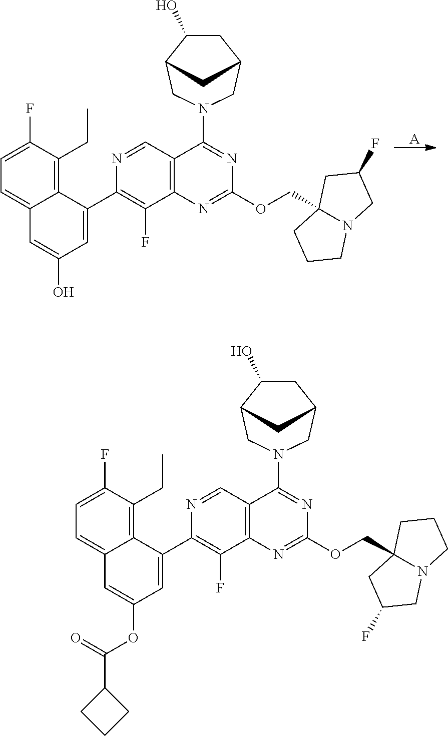

Step A: 5-ethyl-6-fluoro-4-(8-fluoro-2-(((2R,7aS)-2-fluorotetrahydro-1H-pyrrolizin-7a(5H)-yl)methoxy)-4-((1R,5R,6R)-6-hydroxy-3-azabicyclo[3.2.1]octan-3-yl)pyrido[4,3-d]pyrimidin-7-yl)naphthalen-2-yl cyclobutanecarboxylate: To a mixture of (1R,5R,6R)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-6-ol (100 mg, 1.0 equiv) and TEA (49.0 mg, 3.0 equiv) in DCM (1.0 mL) was added cyclobutanecarbonyl chloride (19.1 mg, 1 equiv) in one portion at −40° C. under N2. The reaction mixture was stirred at −40° C. for 4 hours. The reaction mixture was diluted with water (3 mL) and extracted with dichloromethane (3×2 mL). The combined organic layers were washed with brine (30 mL), dried over Na₂SO₄, concentrated and purified with prep-HPLC (column: Phenomenex Luna C18 150×25 mm×10 um; mobile phase: [water (FA)-ACN]; B %: 29%-49%, 58 min) to afford the title compound (20.1 mg, 16.5% yield, HCOOH salt) as a white solid; SFC: ee>95%, Column: Chiralpak IC-3 50×4.6 mm I.D., 3 μm, Mobile phase: Phase A for CO₂, and Phase B for MeOH+ACN(0.05% DEA); Isocratic elution: 40% B in A, Flow rate: 3 mL/min; Detector: PDA; Column Temp: 35 C; Back Pressure: 100 Bar, tR=1.505; 1H NMR (400 MHz, METHANOL-d4) δ=9.25 (d, J=17.2 Hz, 1H), 7.96-7.87 (m, 1H), 7.81 (d, J=2.4 Hz, 1H), 7.45-7.36 (m, 1H), 7.32-7.24 (m, 1H), 5.48-5.26 (m, 1H), 5.04-4.93 (m, 1H), 4.84-4.75 (m, 1H), 4.38 (br d, J=4.0 Hz, 1H), 4.35-4.26 (m, 2H), 3.90-3.74 (m, 1H), 3.56-3.41 (m, 3H), 3.39-3.36 (m, 1H), 3.16-3.04 (m, 1H), 2.61-2.52 (m, 1H), 2.49-2.39 (m, 4H), 2.39-2.31 (m, 3H), 2.30-2.21 (m, 4H), 2.21-2.16 (m, 1H), 2.15-1.99 (m, 4H), 1.98-1.91 (m, 2H), 1.85-1.77 (m, 1H), 1.40 (br d, J=13.6 Hz, 1H), 0.89-0.80 (m, 3H); F NMR (400 MHz, METHANOL-d4) δ=−116.77, −139.11, −173.75; LCMS (ESI, M+1): m/z=702.2.

### Example 14

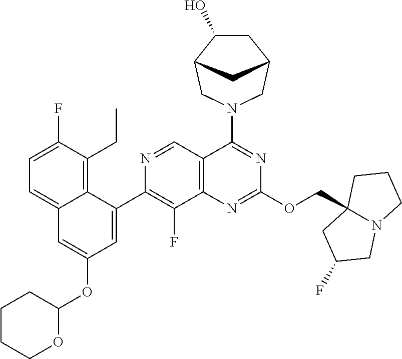 [SMILES: CCc1c(F)ccc2cc(OC3CCCCO3)cc(-c3ncc4c(N5C[C@@H]6C[C@H](C5)[C@H](O)C6)nc(OC[C@@]56CCCN5C[C@H](F)C6)nc4c3F)c12]

### (1R,5R,6R)-3-(7-(8-ethyl-7-fluoro-3-((tetrahydro-2H-pyran-2-yl)oxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-6-ol

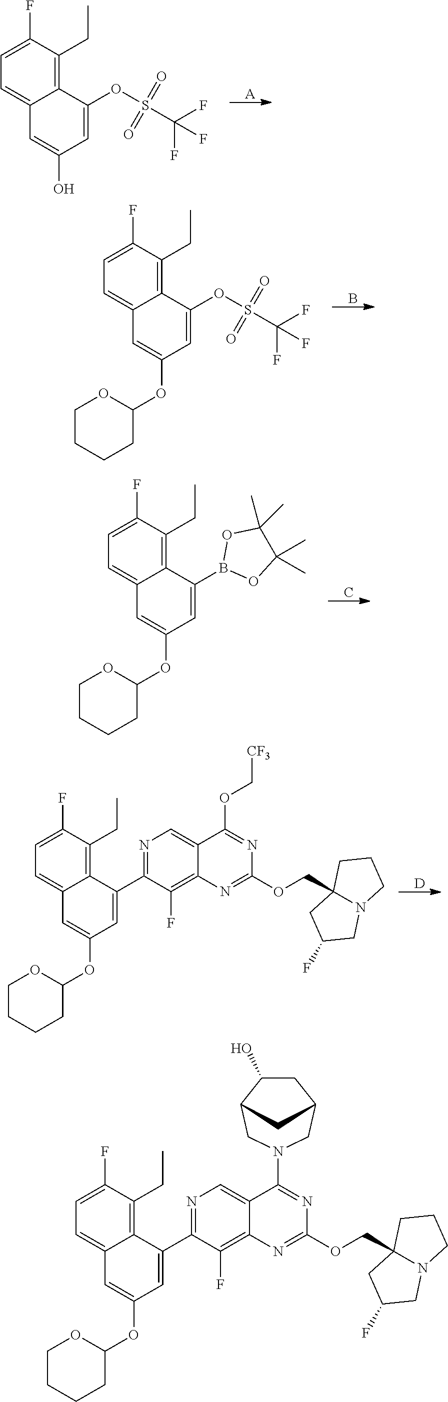

Step A. 8-ethyl-7-fluoro-3-((tetrahydro-2H-pyran-2-yl)oxy)naphthalen-1-yl trifluoromethanesulfonate: To a solution of 8-ethyl-7-fluoro-3-hydroxynaphthalen-1-yl trifluoromethanesulfonate (300 mg, 1.0 equiv) and TsOH (7.64 mg, 0.05 equiv) in DCM (3 mL) was added 3,4-dihydro-2H-pyran (74.6 mg, 1.0 equiv). The reaction was stirred at 0° C. for 1 hour. The mixture was diluted with water (3 mL) and extracted with ethyl acetate (4 mL). The organic layer was dried over sodium sulfate, filtered and concentrated to afford the title compound (500 mg, crude) as yellow oil.

Step B. 2-(8-ethyl-7-fluoro-3-((tetrahydro-2H-pyran-2-yl)oxy)naphthalen-1-yl)-4,4,5,5-tetramethyl-1,3,2-dioxaborolane: To a mixture of 8-ethyl-7-fluoro-3-((tetrahydro-2H-pyran-2-yl)oxy)naphthalen-1-yl trifluoromethanesulfonate (50.0 mg, 1.0 equiv), 4,4,4′,4′,5,5,5′,5′-octamethyl-2,2′-bi(1,3,2-dioxaborolane) (60.1 mg, 2.0 equiv), KOAc (34.9 mg, 3.0 equiv) and cyclopentyl(diphenyl)phosphane;dichloropalladium;iron (17.3 mg, 0.2 equiv) in dioxane (1 mL) was degassed and purged with N2 for 3 times. The mixture was stirred at 100° C. for 3 hours under N2 atmosphere. The mixture was diluted with water (1 mL) and extracted with ethyl acetate (2 mL). The organic layer was dried over sodium sulfate, concentrated and purified with column chromatography (SiO₂, Petroleum ether/Ethyl acetate=100/1 to 30/1) to afford the title compound (190 mg, 85% yield) as a black solid; 1H NMR (400 MHz, CD3OD) δ=7.61 (dd, J=6.0, 9.2 Hz, 1H), 7.45 (d, J=2.4 Hz, 1H), 7.32 (d, J=2.8 Hz, 1H), 7.21 (t, J=9.6 Hz, 1H), 5.59-5.48 (m, 1H), 3.96-3.82 (m, 1H), 3.71-3.56 (m, 1H), 3.11 (dq, J=2.4, 7.6 Hz, 2H), 2.10-1.97 (m, 1H), 1.96-1.80 (m, 2H), 1.77-1.65 (m, 2H), 1.64-1.54 (m, 1H), 1.26-1.19 (m, 15H).

Step C. 7-(8-ethyl-7-fluoro-3-((tetrahydro-2H-pyran-2-yl)oxy)naphthalen-1-yl)-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine: To a solution of 7-chloro-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine (105 mg, 1.0 equiv) and 2-(8-ethyl-7-fluoro-3-((tetrahydro-2H-pyran-2-yl)oxy)naphthalen-1-yl)-4,4,5,5-tetramethyl-1,3,2-dioxaborolane (144 mg, 1.5 equiv) in methoxycyclopentane (1 mL) were added cataCXium® A Pd G3 (34.9 mg, 0.2 equiv) and Cs₂CO₃ (1.5 M, 3.0 equiv) in aqueous solution was degassed and purged with N2 for 3 times. The mixture was stirred at 80° C. for 3 hours under N2 atmosphere. The mixture was diluted with water (2 mL) and extracted with ethyl acetate (3 mL). The organic layer was dried over sodium sulfate, filtered and concentrated and purified by reversed-phase HPLC (0.1% FA condition) to afford the title compound (76.0 mg, 34% yield) as a black solid; LCMS (ESI, M+1): m/z=677.2.

Step D. (1R,5R,6R)-3-(7-(8-ethyl-7-fluoro-3-((tetrahydro-2H-pyran-2-yl)oxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-6-ol: To a solution of 7-(8-ethyl-7-fluoro-3-((tetrahydro-2H-pyran-2-yl)oxy)naphthalen-1-yl)-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine (60.0 mg, 1.0 equiv) and (1R,5R,6R)-3-azabicyclo[3.2.1]octan-6-ol (16.9 mg, 1.5 equiv) in DMF (0.5 ml) was added DIEA (34.4 mg, 3.0 equiv). The reaction was stirred at 50° C. for 14 hours. The mixture was filtered and purified by reversed-phase HPLC (column: Waters Xbridge 150×25 mm×5 um; mobile phase: [water(NH₄HCO₃)-ACN]; B %: 62%-92%, 9 min) to afford the title compound (8.80 mg, 14% yield) as a white solid; 1H NMR (400 MHz, CD3OD) δ=9.29-9.18 (m, 1H), 7.84-7.72 (m, 1H), 7.62 (d, J=2.6 Hz, 1H), 7.37-7.18 (m, 2H), 5.69-5.59 (m, 1H), 5.43-5.20 (m, 1H), 4.83-4.70 (m, 2H), 4.39-4.27 (m, 2H), 4.26-4.18 (m, 1H), 3.99-3.88 (m, 1H), 3.85-3.74 (m, 1H), 3.70-3.61 (m, 1H), 3.47 (br dd, J=12.8, 19.1 Hz, 1H), 3.28-3.11 (m, 3H), 3.07-2.95 (m, 1H), 2.58-2.44 (m, 1H), 2.44-2.36 (m, 1H), 2.36-2.10 (m, 6H), 2.06-1.96 (m, 3H), 1.94-1.85 (m, 4H), 1.85-1.77 (m, 1H), 1.76-1.67 (m, 2H), 1.62 (dt, J=3.6, 7.6 Hz, 1H), 1.40 (br d, J=14.4 Hz, 1H), 0.86-0.75 (m, 3H); LCMS (ESI, M+1): m/z=704.5.

### Example 15

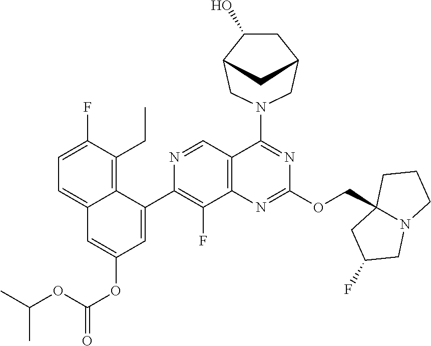 [SMILES: CCc1c(F)ccc2cc(OC(=O)OC(C)C)cc(-c3ncc4c(N5C[C@@H]6C[C@H](C5)[C@H](O)C6)nc(OC[C@@]56CCCN5C[C@H](F)C6)nc4c3F)c12]

### 5-ethyl-6-fluoro-4-(8-fluoro-2-(((2R,7aS)-2-fluorotetrahydro-1H-pyrrolizin-7a(5H)-yl)methoxy)-4-((1R,5R,6R)-6-hydroxy-3-azabicyclo[3.2.1]octan-3-yl)pyrido[4,3-d]pyrimidin-7-yl)naphthalen-2-yl isopropyl carbonate

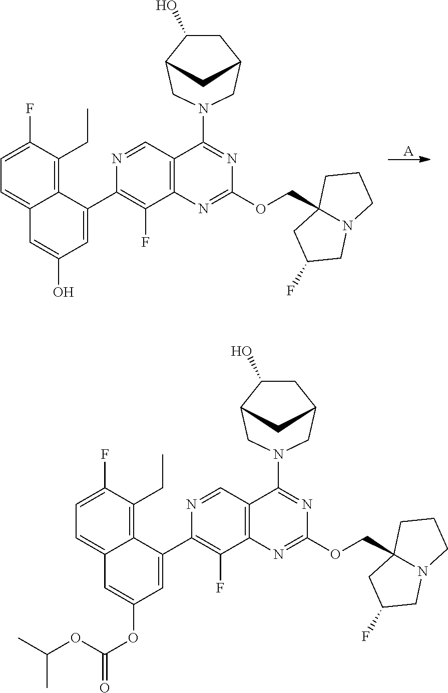

Step A. 5-ethyl-6-fluoro-4-(8-fluoro-2-(((2R,7aS)-2-fluorotetrahydro-1H-pyrrolizin-7a(5H)-yl)methoxy)-4-((1R,5R,6R)-6-hydroxy-3-azabicyclo[3.2.1]octan-3-yl)pyrido[4,3-d]pyrimidin-7-yl)naphthalen-2-yl isopropyl carbonate: To a solution of (1R,5R,6R)-3-[7-(8-ethyl-7-fluoro-3-hydroxy-1-naphthyl)-8-fluoro-2-[[(2R,8S)-2-fluoro-1,2,3,5,6,7-hexahydropyrrolizin-8-yl]methoxy]pyrido[4,3-d]pyrimidin-4-yl]-3-azabicyclo[3.2.1]octan-6-ol (50.0 mg, 1.0 equiv) and TEA (24.5 mg, 3.0 equiv) in DCM (2 mL) was added isopropyl carbonochloridate (9.89 mg, 1.0 equiv). The reaction was stirred at 0° C. for 3.5 hours. The mixture was quenched by adding H₂O (5 mL) at 0° C. and extracted with DCM (3×5 mL). The combined organic layers were dried over Na₂SO₄, concentrated and purified with prep-HPLC [Waters Xbridge 150×25 mm×5 μm; A: water (NH₄HCO₃), B: ACN; B %: 65%-95%, over 8 min] to afford the title compound (14.5 mg, 25% yield) as white solid; 1H NMR (400 MHz, DMSO-d6) δ=9.38-9.25 (m, 1H), 8.08-7.97 (m, 2H), 7.62-7.42 (m, 2H), 5.22 (br s, 1H), 4.99-4.68 (m, 3H), 4.64-4.27 (m, 1H), 4.22-4.09 (m, 2H), 4.15-3.81 (m, 1H), 3.75 (br s, 1H), 3.40-3.35 (m, 1H), 3.16-2.97 (m, 3H), 2.90-2.44 (m, 1H), 2.38-2.29 (m, 2H), 2.21-1.95 (m, 6H), 1.90-1.73 (m, 4H), 1.68 (br s, 1H), 1.41-1.34 (m, 6H), 1.28-1.20 (m, 1H), 0.75 (m, 3H); LCMS (ESI, M+1): m/z=706.4.

### Example 16

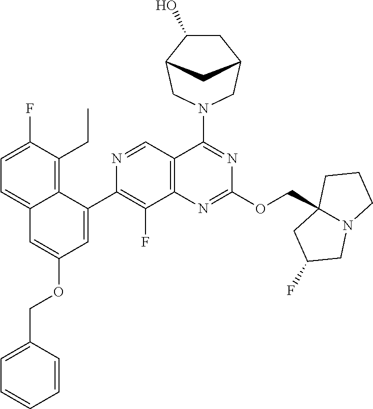 [SMILES: CCc1c(F)ccc2cc(OCc3ccccc3)cc(-c3ncc4c(N5C[C@@H]6C[C@H](C5)[C@H](O)C6)nc(OC[C@@]56CCCN5C[C@H](F)C6)nc4c3F)c12]

### (1R,5R,6R)-3-(7-(3-(benzyloxy)-8-ethyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-6-ol

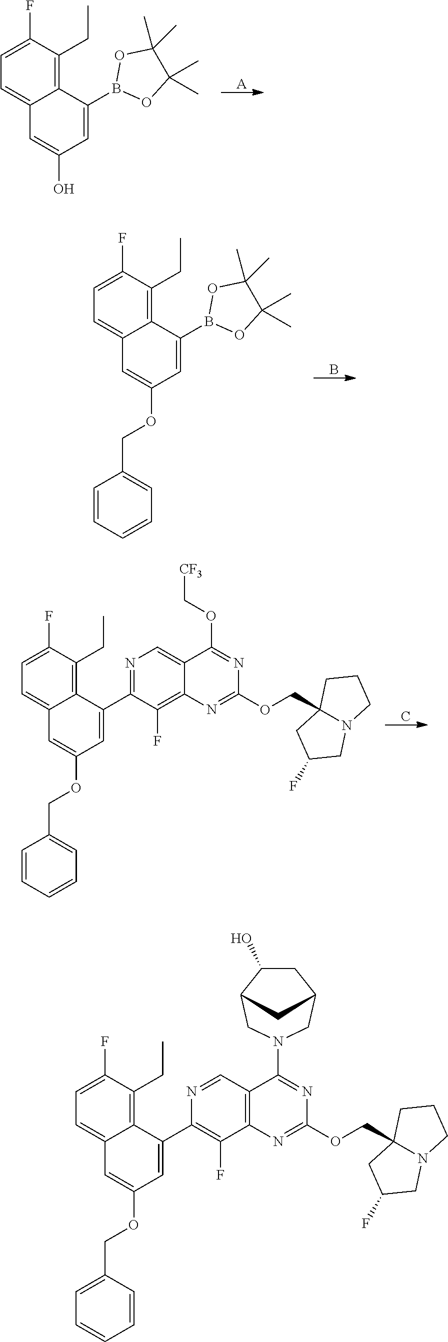

Step A. 2-(3-(benzyloxy)-8-ethyl-7-fluoronaphthalen-1-yl)-4,4,5,5-tetramethyl-1,3,2-dioxaborolane: To a solution of 5-ethyl-6-fluoro-4-(4,4,5,5-tetramethyl-1,3,2-dioxaborolan-2-yl)naphthalen-2-ol (500 mg, 1.0 equiv) in CH₃CN (10 mL) were added DIEA (408 mg, 2.0 equiv) and (bromomethyl)benzene (540 mg, 2.0 equiv). The reaction was stirred at 25° C. for 12 hours. The mixture was quenched by adding water (10 mL) and concentrated to remove the organic solvent. The mixture was extracted with EtOAc (3×10 mL), dried over Na₂SO₄, concentrated and purified with column chromatography to afford the title compound (600 mg) as a white solid; LCMS (ESI, M+1): m/z=407.3.

Step B. 7-(3-(benzyloxy)-8-ethyl-7-fluoronaphthalen-1-yl)-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine: To a solution of 2-(3-(benzyloxy)-8-ethyl-7-fluoronaphthalen-1-yl)-4,4,5,5-tetramethyl-1,3,2-dioxaborolane (200 mg, 1.0 equiv) and 7-chloro-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine (194 mg, 0.90 equiv) in CMPE (5 mL) were added K₃PO₄ (1.5 M, 0.3 mL, 1.0 equiv) and CataCXium A Pd G3 (3.58 mg, 0.01 equiv). The reaction was stirred at 80° C. for 12 hours under N2 atmosphere. The mixture was quenched by adding water (10 mL) and concentrated to remove the organic solvent. The mixture was extracted with EtOAc (3×10 mL), dried over Na₂SO₄, concentrated to afford the crude product as brown oil and used into the next step without further purification; LCMS (ESI, M+1): m/z=683.4.

Step C. (1R,5R,6R)-3-(7-(3-(benzyloxy)-8-ethyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-6-ol: To a solution of 7-(3-(benzyloxy)-8-ethyl-7-fluoronaphthalen-1-yl)-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine (100 mg, 1.0 equiv) in DMF (1 mL) were added 4 Å MS (20.0 mg), (1R,5R,6R)-3-azabicyclo[3.2.1]octan-6-ol (29.8 mg, 1.6 equiv) and DIPEA (36.2 mg, 2.0 equiv). The reaction was stirred at 40° C. for 12 hours. The mixture was filtered and purified with prep-HPLC (column: Unisil 3-100 C18 Ultra 150×50 mm×3 m; mobile phase: [water(FA)-ACN]; B %: 31%-61%, 7 min) to afford the title compound (45.1 mg, 41% yield) as yellow solid; 1H NMR (400 MHz, DMSO-d6) δ=9.38-9.16 (m, 1H), 7.91-7.81 (m, 1H), 7.67 (d, J=2.8 Hz, 1H), 7.52 (d, J=7.2 Hz, 2H), 7.47-7.38 (m, 3H), 7.37-7.30 (m, 1H), 7.23 (dd, J=2.6, 8.2 Hz, 1H), 5.41-5.19 (m, 3H), 4.88-4.76 (m, 1H), 4.74-4.48 (m, 2H), 4.21-3.97 (m, 3H), 3.80-3.67 (m, 1H), 3.14-3.02 (m, 3H), 2.89-2.78 (m, 1H), 2.71-2.59 (m, 1H), 2.33 (dd, J=1.8, 3.4 Hz, 2H), 2.18-2.00 (m, 6H), 1.88-1.72 (m, 4H), 1.70-1.61 (m, 1H), 1.27-1.23 (m, 1H), 0.72 (dt, J=3.8, 7.2 Hz, 3H); LCMS (ESI, M+1): m/z=710.3.

### Example 17

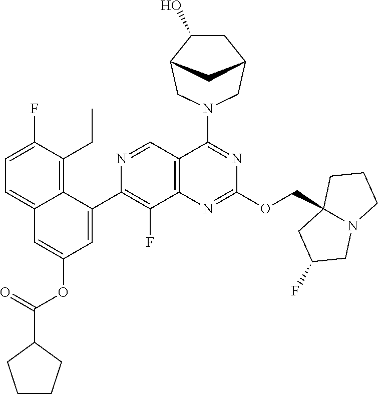 [SMILES: CCc1c(F)ccc2cc(OC(=O)C3CCCC3)cc(-c3ncc4c(N5C[C@@H]6C[C@H](C5)[C@H](O)C6)nc(OC[C@@]56CCCN5C[C@H](F)C6)nc4c3F)c12]

### 5-ethyl-6-fluoro-4-(8-fluoro-2-(((2R,7aS)-2-fluorotetrahydro-1H-pyrrolizin-7a(5H)-yl)methoxy)-4-((1R,5R,6R)-6-hydroxy-3-azabicyclo[3.2.1]octan-3-yl)pyrido[4,3-d]pyrimidin-7-yl)naphthalen-2-yl cyclopentanecarboxylate

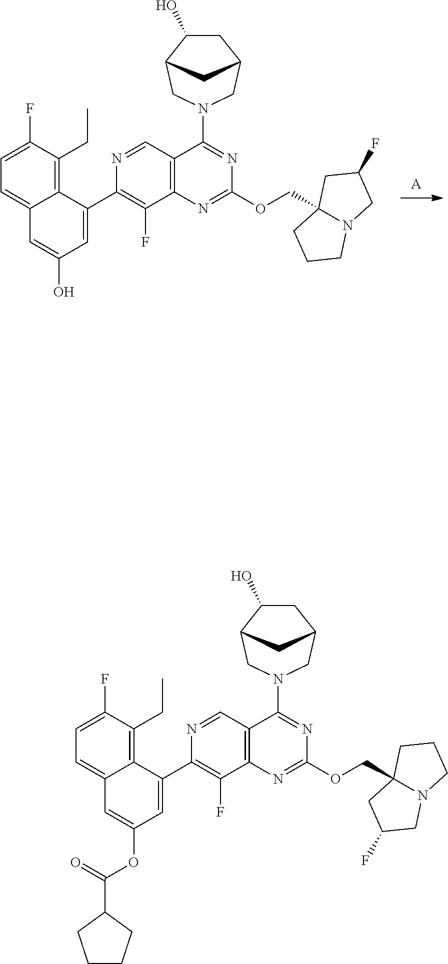

Step A. 5-ethyl-6-fluoro-4-(8-fluoro-2-(((2R,7aS)-2-fluorohexahydro-1H-pyrrolizin-7a-yl)methoxy)-4-((1R,5R,6R)-6-hydroxy-3-azabicyclo[3.2.1]octan-3-yl)pyrido[4,3-d]pyrimidin-7-yl)naphthalen-2-yl cyclopentanecarboxylate: To a mixture of (1R,5R,6R)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-6-ol (100 mg, 1.0 equiv) and TEA (49.0 mg, 3.0 equiv) in DCM (1.0 mL) was added cyclopentanecarbonyl chloride (21.4 mg, 1.0 equiv) in one portion at −40° C. under N2. The reaction mixture was stirred at −40° C. for 0.5 hours. The mixture was diluted with water (3 mL) and extracted with dichloromethane (3×2 mL). The combined organic layers were dried over Na₂SO₄, concentrated and purified by prep-HPLC (column: Phenomenex luna C18 150×25 mm×10 um; mobile phase: [water (FA)-ACN]; B %: 28%-58%, 10 min) to afford the title compound (27.0 mg, 22.8% yield) as a white solid; SFC: ee>95%, Column: Chiralpak AS-3 50×4.6 mm I.D., 3 μm Mobile phase: Phase A for CO₂, and Phase B for MeOH (0.05% DEA); Gradient elution: B in A from 5% to 40%; Flow rate: 3 mL/min; Detector: PDA; Column Temp: 35 C; Back Pressure: 100 Bar, tR=1.417; 1H NMR (400 MHz, METHANOL-d4) δ=9.24 (d, J=16.3 Hz, 1H), 7.94-7.87 (m, 1H), 7.79 (d, J=2.6 Hz, 1H), 7.43-7.35 (m, 1H), 7.30-7.23 (m, 1H), 5.40-5.23 (m, 1H), 5.04-4.95 (m, 1H), 4.77 (br d, J=12.4 Hz, 1H), 4.37-4.29 (m, 2H), 4.27-4.20 (m, 1H), 3.85-3.76 (m, 1H), 3.51-3.42 (m, 1H), 3.28-3.22 (m, 2H), 3.14-2.99 (m, 2H), 2.60-2.49 (m, 1H), 2.44-2.33 (m, 2H), 2.30-2.21 (m, 4H), 2.20-2.12 (m, 2H), 2.08-1.98 (m, 5H), 1.97-1.87 (m, 3H), 1.81-1.75 (m, 2H), 1.74-1.64 (m, 2H), 1.43-1.28 (m, 2H), 0.86-0.79 (m, 3H); H NMR (400 MHz, METHANOL-d4) δ=−116.78, −139.04, −173.73; LCMS (ESI, M+1): m/z=716.2.

### Example 18

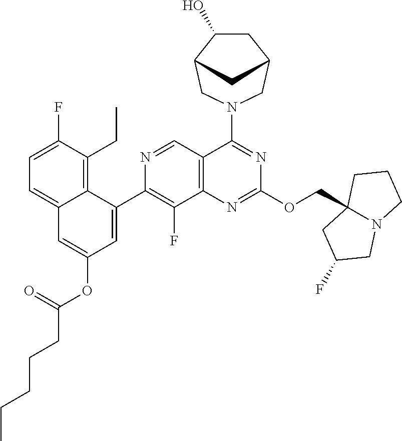 [SMILES: CCCCCC(=O)Oc1cc(-c2ncc3c(N4C[C@@H]5C[C@H](C4)[C@H](O)C5)nc(OC[C@@]45CCCN4C[C@H](F)C5)nc3c2F)c2c(CC)c(F)ccc2c1]

### 5-ethyl-6-fluoro-4-(8-fluoro-2-(((2R,7aS)-2-fluorotetrahydro-1H-pyrrolizin-7a(5H)-yl)methoxy)-4-((1R,5R,6R)-6-hydroxy-3-azabicyclo[3.2.1]octan-3-yl)pyrido[4,3-d]pyrimidin-7-yl)naphthalen-2-yl hexanoate

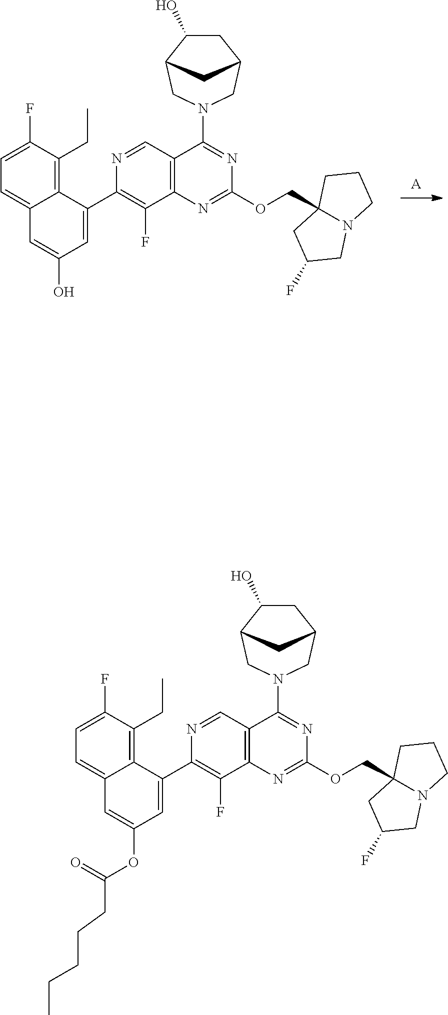

Step A. 5-ethyl-6-fluoro-4-(8-fluoro-2-(((2R,7aS)-2-fluorotetrahydro-1H-pyrrolizin-7a(5H)-yl)methoxy)-4-((1R,5R,6R)-6-hydroxy-3-azabicyclo[3.2.1]octan-3-yl)pyrido[4,3-d]pyrimidin-7-yl)naphthalen-2-yl hexanoate: To a solution of (1R,5R,6R)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-6-ol (100 mg, 1.0 equiv) and TEA (49.0 mg 3.0 equiv) in DCM (2 mL) was added hexanoyl chloride (32.6 mg, 1.5 equiv) at 0° C. The reaction was stirred at 20° C. for 6 hours. The mixture was quenched by adding H₂O (5 mL) at 0° C. and extracted with DCM (3×5 mL). The combined organic layers were dried over Na₂SO₄, concentrated and purified with prep-HPLC [Phenomenex luna C18 150×25 mm×10 μm; A: water (FA), B: ACN; B %: 33%-63%, over 10 min] to afford the title compound (37.5 mg, 31% yield) as white solid; 1H NMR (400 MHz, DMSO-d6) δ=9.40-9.26 (m, 1H), 8.19-8.16 (m, 1H), 8.08-7.97 (m, 1H), 7.97-7.86 (m, 1H), 7.41-7.33 (m, 1H), 5.40-5.16 (m, 1H), 4.93-4.64 (m, 2H), 4.63-4.51 (m, 1H), 4.25-4.10 (m, 2H), 4.08-3.97 (m, 1H), 3.80-3.71 (m, 1H), 3.31-3.27 (m, 1H), 3.05 (br s, 2H), 3.04-2.99 (m, 1H), 2.88-2.78 (m, 1H), 2.66-2.58 (m, 2H), 2.46-2.31 (m, 2H), 2.26-2.12 (m, 3H), 2.11-1.97 (m, 3H), 1.89-1.72 (m, 4H), 1.72-1.61 (m, 3H), 1.42-1.30 (m, 4H), 1.29-1.22 (m, 1H), 0.94-0.87 (m, 3H), 0.81-0.71 (m, 3H); LCMS (ESI, M+1): m/z=718.5.

### Example 19

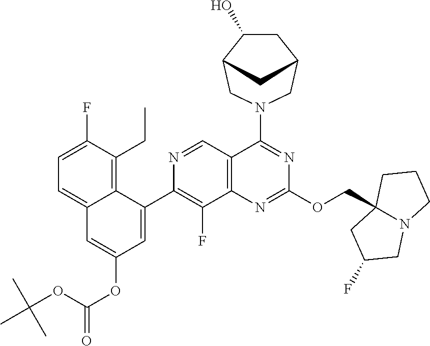 [SMILES: CCc1c(F)ccc2cc(OC(=O)OC(C)(C)C)cc(-c3ncc4c(N5C[C@@H]6C[C@H](C5)[C@H](O)C6)nc(OC[C@@]56CCCN5C[C@H](F)C6)nc4c3F)c12]

### tert-butyl (5-ethyl-6-fluoro-4-(8-fluoro-2-(((2R,7aS)-2-fluorotetrahydro-1H-pyrrolizin-7a(5H)-yl)methoxy)-4-((1R,5R,6R)-6-hydroxy-3-azabicyclo[3.2.1]octan-3-yl)pyrido[4,3-d]pyrimidin-7-yl)naphthalen-2-yl) carbonate

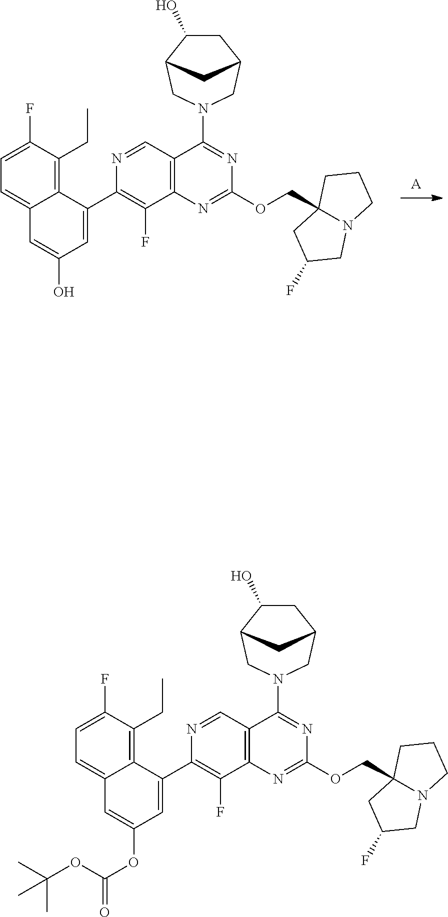

Step A. tert-butyl (5-ethyl-6-fluoro-4-(8-fluoro-2-(((2R,7aS)-2-fluorotetrahydro-1H-pyrrolizin-7a(5H)-yl)methoxy)-4-((1R,5R,6R)-6-hydroxy-3-azabicyclo[3.2.1]octan-3-yl)pyrido[4,3-d]pyrimidin-7-yl)naphthalen-2-yl) carbonate: To a solution of (1R,5R,6R)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-6-ol (50.0 mg, 1.0 equiv), Boc₂O (38.7 mg, 2.2 equiv), TEA (24.5 mg, 3.0 equiv) and K₂CO₃ (11.1 mg, 1.0 equiv) in DMF (0.5 mL) was added 1,4,7,10,13,16-hexaoxacyclooctadecane (21.3 mg, 1.0 equiv). The reaction was stirred at 25° C. for 17.5 hours. The mixture was filtered and purified with prep-HPLC [Waters Xbridge 150×25 mm×5 μm; A: water (NH₄HCO₃), B: ACN; B %: 62%-92% over 8 min] to afford the title compound (39.3 mg, 67% yield) as white solid; 1H NMR (400 MHz, DMSO-d6) δ=9.40-9.24 (m, 1H), 8.11-7.91 (m, 2H), 7.61-7.48 (m, 1H), 7.47-7.38 (m, 1H), 5.39-5.17 (m, 1H), 4.91-4.68 (m, 2H), 4.64-4.52 (m, 1H), 4.23-4.07 (m, 2H), 4.06-3.97 (m, 1H), 3.80-3.66 (m, 1H), 3.39-3.33 (m, 1H), 3.15-2.96 (m, 3H), 2.89-2.75 (m, 1H), 2.41-2.31 (m, 2H), 2.24-1.93 (m, 6H), 1.89-1.60 (m, 5H), 1.55-1.42 (m, 9H), 1.29-1.21 (m, 1H), 0.83-0.67 (m, 3H); LCMS (ESI, M+1): m/z=720.4.

### Example 20

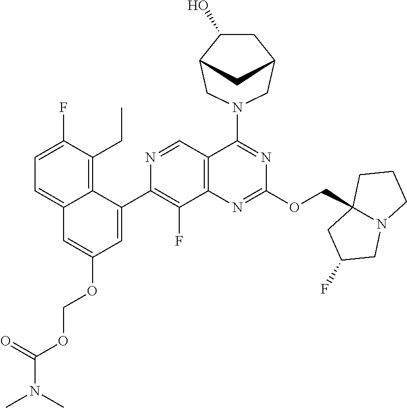 [SMILES: CCc1c(F)ccc2cc(OCOC(=O)N(C)C)cc(-c3ncc4c(N5C[C@@H]6C[C@H](C5)[C@H](O)C6)nc(OC[C@@]56CCCN5C[C@H](F)C6)nc4c3F)c12]

### ((5-ethyl-6-fluoro-4-(8-fluoro-2-(((2R,7aS)-2-fluorotetrahydro-1H-pyrrolizin-7a(5H)-yl)methoxy)-4-((1R,5R,6R)-6-hydroxy-3-azabicyclo[3.2.1]octan-3-yl)pyrido[4,3-d]pyrimidin-7-yl)naphthalen-2-yl)oxy)methyl dimethylcarbamate

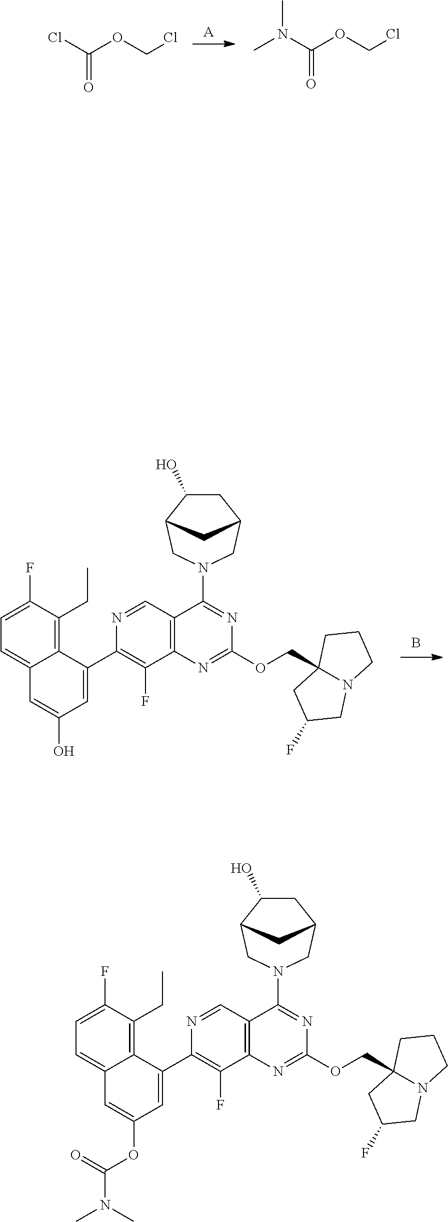

Step A. chloromethyl dimethylcarbamate: To a solution of chloromethyl carbonochloridate (2.79 g, 1.0 equiv) and DIEA (8.39 g, 3.0 equiv) in THF (10 mL) was slowly added dimethylamine (2 M, 6.49 mL, 0.6 equiv) at −60° C. The reaction was stirred at −60° C. for 0.5 hours. The mixture was quenched by adding water (30 mL) at 0° C. and extracted with ethyl acetate (4×30 mL). The combined organic layers were washed with brine (30 mL), dried over anhydrous sodium sulfate, concentrated and purified with column chromatography [SiO₂, Petroleum ether/Ethyl acetate=50/1 to 10/1] to afford the title compound (600 mg, 20% yield) as yellow oil; 1H NMR (400 MHz, CHLOROFORM-d) δ=6.05-5.52 (m, 2H), 3.11-2.83 (m, 6H).

Step B. ((5-ethyl-6-fluoro-4-(8-fluoro-2-(((2R,7aS)-2-fluorotetrahydro-1H-pyrrolizin-7a(5H)-yl)methoxy)-4-((1R,5R,6R)-6-hydroxy-3-azabicyclo[3.2.1]octan-3-yl)pyrido[4,3-d]pyrimidin-7-yl)naphthalen-2-yl)oxy)methyl dimethylcarbamate: To a mixture of (1R,5R,6R)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-6-ol (60.0 mg, 1.0 equiv) and K₂CO₃ (40.2 mg, 3.0 equiv) in DMF (1.0 mL) was added chloromethyl dimethylcarbamate (14.7 mg, 1.10 equiv). The reaction was stirred at 20° C. for 12 hours. The mixture was filtered and purified with prep-HPLC [Waters Xbridge C18 150×50 mm×10 μm; A: water (NH₄HCO₃), B: ACN; B %: 55%-58%, over 9 min] to afford the title compound (38.0 mg, 53% yield) as white solid; 1H NMR (400 MHz, DMSO-d6) δ=9.38-9.22 (m, 1H), 7.96-7.92 (m, 1H), 7.75 (d, J=2.8 Hz, 1H), 7.48-7.43 (m, 1H), 7.26-7.23 (m, 1H), 5.89 (s, 2H), 5.39-5.15 (m, 1H), 4.88-4.75 (m, 1H), 4.75-4.66 (m, 1H), 4.64-4.51 (m, 1H), 4.23-4.07 (m, 2H), 4.03-3.99 (m, 1H), 3.81-3.66 (m, 1H), 3.40-3.35 (m, 1H), 3.12-2.98 (m, 3H), 2.85 (d, J=7.6 Hz, 6H), 2.83-2.74 (m, 1H), 2.40-2.30 (m, 2H), 2.19-1.97 (m, 6H), 1.89-1.60 (m, 5H), 1.31-1.20 (m, 1H), 0.79-0.67 (m, 3H); LCMS (ESI, M+1): m/z=721.6.

### Example 21

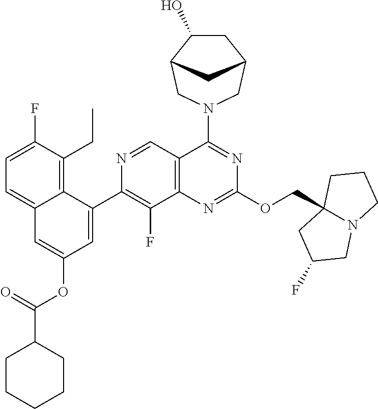 [SMILES: CCc1c(F)ccc2cc(OC(=O)C3CCCCC3)cc(-c3ncc4c(N5C[C@@H]6C[C@H](C5)[C@H](O)C6)nc(OC[C@@]56CCCN5C[C@H](F)C6)nc4c3F)c12]

### 5-ethyl-6-fluoro-4-(8-fluoro-2-(((2R,7aS)-2-fluorotetrahydro-1H-pyrrolizin-7a(5H)-yl)methoxy)-4-((1R,5R,6R)-6-hydroxy-3-azabicyclo[3.2.1]octan-3-yl)pyrido[4,3-d]pyrimidin-7-yl)naphthalen-2-yl cyclohexanecarboxylate

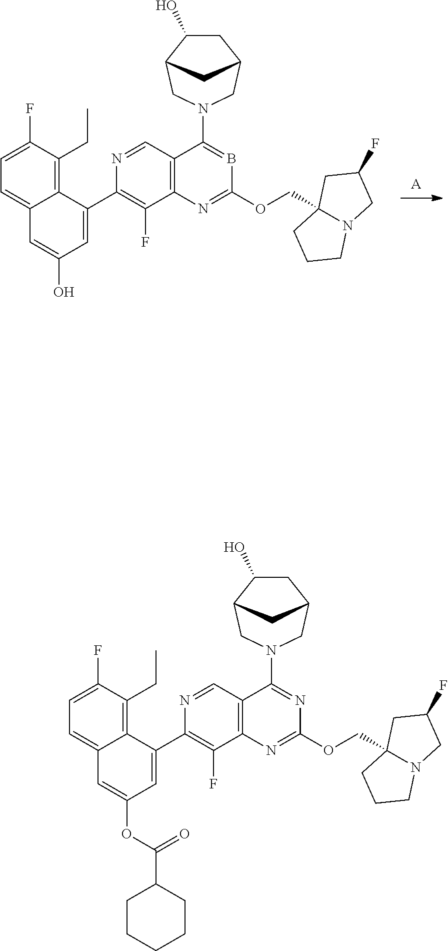

Step A. 5-ethyl-6-fluoro-4-(8-fluoro-2-(((2R,7aS)-2-fluorohexahydro-1H-pyrrolizin-7a-yl)methoxy)-4-((1R,5R,6R)-6-hydroxy-3-azabicyclo[3.2.1]octan-3-yl)pyrido[4,3-d]pyrimidin-7-yl)naphthalen-2-yl cyclohexanecarboxylate: To a mixture of (1R,5R,6R)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-6-ol (100 mg, 1.0 equiv) and TEA (49.0 mg, 3.0 equiv) in DCM (1.0 mL) was added cyclohexanecarbonyl chloride (23.7 mg, 1.0 equiv) in one portion at −40° C. under N2. The reaction mixture was stirred at −40° C. for 0.5 hours. The mixture was diluted with water (3 mL) and extracted with dichloromethane (3×2 mL). The combined organic layers were dried over Na₂SO₄, concentrated and purified with prep-HPLC {column: Phenomenex luna C18 150×25 mm×10 um; mobile phase [water (FA)-ACN]; B %: 28%-58%, 10 min} to afford the title compound (14.5 mg, 11.9% yield) as white solid; SFC: ee>95%, Column: Chiralpak AS-3 50×4.6 mm I.D., 3 μm; Mobile phase: Phase A for CO₂, and Phase B for MeOH (0.05% DEA); Gradient elution: B in A from 5% to 40%; Flow rate: 3 mL/min; Detector: PDA; Column Temp: 35 C; Back Pressure: 100 Bar, tR=1.453; 1H NMR (400 MHz, METHANOL-d4) δ=9.25 (d, J=16.5 Hz, 1H), 7.93-7.87 (m, 1H), 7.79 (d, J=2.5 Hz, 1H), 7.43-7.36 (m, 1H), 7.29-7.22 (m, 1H), 5.49-5.31 (m, 1H), 5.05-4.94 (m, 1H), 4.76 (br s, 1H), 4.49-4.42 (m, 1H), 4.39-4.28 (m, 2H), 3.87-3.75 (m, 1H), 3.61-3.51 (m, 1H), 3.50-3.43 (m, 3H), 3.21-3.13 (m, 1H), 2.71-2.62 (m, 1H), 2.60-2.46 (m, 2H), 2.42-2.34 (m, 2H), 2.28-2.20 (m, 4H), 2.14-2.06 (m, 4H), 2.01-1.91 (m, 2H), 1.86-1.79 (m, 3H), 1.74-1.67 (m, 1H), 1.64-1.56 (m, 2H), 1.45-1.37 (m, 3H), 1.35-1.29 (m, 1H), 0.86-0.79 (m, 3H); F NMR (400 MHz, METHANOL-d4) δ=−116.74, −139.20, −173.84; LCMS (ESI, M+1): m/z=730.4.

### Example 22

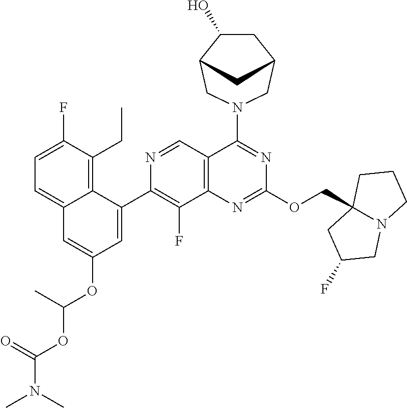 [SMILES: CCc1c(F)ccc2cc(OC(C)OC(=O)N(C)C)cc(-c3ncc4c(N5C[C@@H]6C[C@H](C5)[C@H](O)C6)nc(OC[C@@]56CCCN5C[C@H](F)C6)nc4c3F)c12]

### 1-((5-ethyl-6-fluoro-4-(8-fluoro-2-(((2R,7aS)-2-fluorotetrahydro-1H-pyrrolizin-7a(5H)-yl)methoxy)-4-((1R,5R,6R)-6-hydroxy-3-azabicyclo[3.2.1]octan-3-yl)pyrido[4,3-d]pyrimidin-7-yl)naphthalen-2-yl)oxy)ethyl dimethylcarbamate

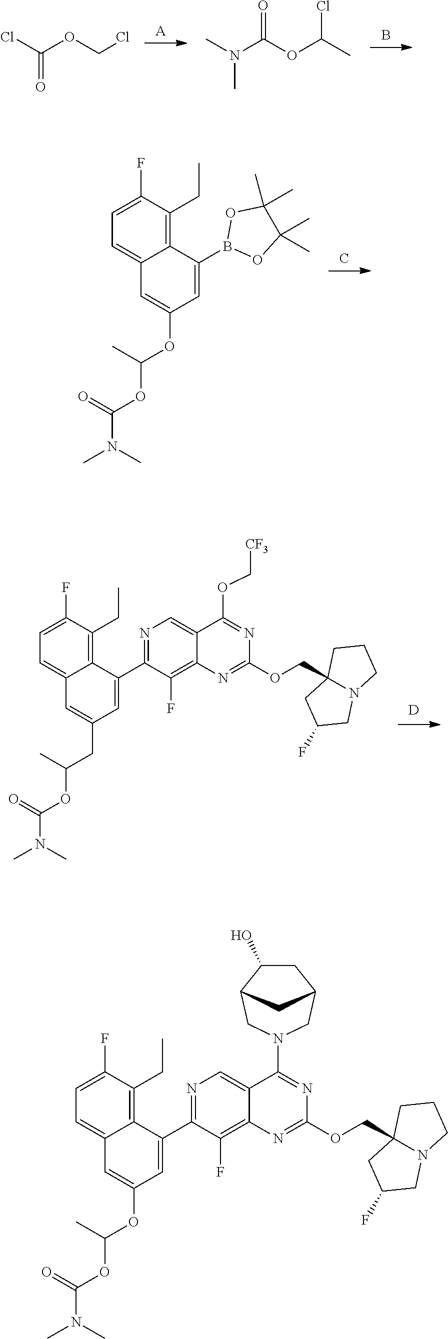

Step A. 1-chloroethyl dimethylcarbamate: To a solution of 1-chloroethyl carbonochloridate (6.00 g, 1.0 equiv) and TEA (4.25 g, 1.0 equiv) in DCM (20 mL) was added N-methylmethanamine (2 M, 0.7 equiv) at 0° C. The mixture was stirred at 0° C. for 1 hour. The mixture was quenched by adding water (10 mL) and extracted with DCM (3×20 mL), dried over Na₂SO₄, concentrated and purified with column chromatography (SiO₂, petroleum ether/ethyl acetate 50/1 to 20/1) to afford title compound (210 mg, 3.3% yield) as a yellow oil; 1H NMR (400 MHz, CHLOROFORM-d) δ=6.57 (q, J=6.0 Hz, 1H), 2.95 (d, J=5.6 Hz, 6H), 1.81 (d, J=6.0 Hz, 3H).

Step B. 1-((5-ethyl-6-fluoro-4-(4,4,5,5-tetramethyl-1,3,2-dioxaborolan-2-yl)naphthalen-2-yl)oxy)ethyl dimethylcarbamate: To a solution of 5-ethyl-6-fluoro-4-(4,4,5,5-tetramethyl-1,3,2-dioxaborolan-2-yl)naphthalen-2-ol (430 mg, 1.0 equiv) and 1-chloroethyl dimethylcarbamate (206 mg, 1.0 equiv) in acetonitrile (5 mL) was added K₂CO₃ (563 mg, 3.0 equiv). The reaction was stirred at 25° C. for 16 hours. The mixture was quenched by adding water (5 mL) and extracted with EA (3×5 mL), dried over Na₂SO₄, concentrated and purified with column chromatography (SiO₂, petroleum ether/ethyl acetate 15/1 to 10/1) to afford title compound (290 mg, 44% yield) as yellow oil; 1H NMR (400 MHz, CHLOROFORM-d) δ=7.58 (dd, J=6.0, 9.2 Hz, 1H), 7.39-7.31 (m, 2H), 7.20 (t, J=9.2 Hz, 1H), 6.64 (q, J=5.2 Hz, 1H), 3.12 (m, 2H), 2.97-2.82 (m, 6H), 1.66 (d, J=5.2 Hz, 3H), 1.44 (s, 12H), 1.27 (t, J=7.6 Hz, 3H).

Step C. 1-((5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-yl)oxy)ethyl dimethylcarbamate: A mixture of 7-chloro-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine (230 mg, 1.0 equiv), 1-((5-ethyl-6-fluoro-4-(4,4,5,5-tetramethyl-1,3,2-dioxaborolan-2-yl)naphthalen-2-yl)oxy)ethyl dimethylcarbamate (282 mg, 1.2 equiv), CataCXium A Pd G3 (38.0 mg, 0.1 equiv) and Cs₂CO₃ (1.5 M in water, 0.6 mL) in methoxycyclopentane (3 mL) was degassed and purged with N2 for 3 times. The reaction was stirred at 80° C. for 4.5 hours under N2 atmosphere. The mixture was quenched by adding water and extracted with EtOAc, dried over Na₂SO₄, concentrated and purified with reversed phase flash [water (0.1% FA)/acetonitrile] to afford title compound (200 mg, 52% yield) as yellow oil; ¹H NMR (400 MHz, DMSO-d6) δ=9.21 (s, 1H), 7.90 (dd, J=6.0, 8.8 Hz, 1H), 7.69 (dd, J=2.8, 7.2 Hz, 1H), 7.47 (t, J=9.2 Hz, 1H), 7.23 (dd, J=2.4, 4.8 Hz, 1H), 6.64-6.54 (m, 1H), 5.35-5.19 (m, 1H), 4.30-4.15 (dd, J=2.8, 8.0 Hz, 2H), 3.31 (s, 2H), 3.13-3.00 (m, 3H), 2.83-2.75 (m, 7H), 2.37-2.30 (m, 1H), 2.19-2.07 (m, 3H), 1.91-1.73 (m, 4H), 1.59 (dd, J=2.0, 5.2 Hz, 3H), 0.71 (br t, J=7.2 Hz, 3H); LCMS (ESI, M+1): m/z=608.3.

Step D. 1-((5-ethyl-6-fluoro-4-(8-fluoro-2-(((2R,7aS)-2-fluorotetrahydro-1H-pyrrolizin-7a(5H)-yl)methoxy)-4-((1R,5R,6R)-6-hydroxy-3-azabicyclo[3.2.1]octan-3-yl)pyrido[4,3-d]pyrimidin-7-yl)naphthalen-2-yl)oxy)ethyl dimethylcarbamate: To a solution of (1R,5R,6R)-3-azabicyclo[3.2.1]octan-6-ol (18.0 mg, 2.0 equiv), DIEA (27.4 mg, 3.0 equiv) and 4 Å molecular sieve (10 mg) in DMF (0.5 mL) was added 1-((5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-yl)oxy)ethyl dimethylcarbamate (50 mg, 1.0 equiv). The reaction was stirred at 40° C. for 16 hours. The mixture was filtered and the filtrate was purified with prep-HPLC [column: Waters Xbridge 150×25 mm×5 μm; A: water (NH₄HCO₃), B: ACN; B %: 58%-88% over 8 min] to afford title compound (15.9 mg, 29% yield) as a white solid; SFC: >99% ee, column: (S,S)Whelk-01 50×4.6 mm I.D., 3.5 m, mobile phase: 40% EtOH (0.05% DEA) in CO₂, flow rate: 3 mL/min, detector: 220 nm, tR1: 1.552 min, tR2:1.988; 1H NMR (400 MHz, DMSO-d6) δ=9.37-9.24 (d, J=3.2, 1H), 7.88 (dd, J=6.4, 9.0 Hz, 1H), 7.70-7.61 (m, 1H), 7.45 (dt, J=2.4, 9.6 Hz, 1H), 7.26-7.17 (m, 1H), 6.63-6.54 (m, 1H), 5.41-5.13 (d, J=5.4, 1H), 4.90-4.76 (m, 1H), 4.75-4.67 (m, 1H), 4.62-4.53 (m, 1H), 4.21-4.10 (m, 2H), 4.08-3.97 (m, 1H), 3.74 (br dd, J=12.0, 19.6 Hz, 1H), 3.30 (s, 2H), 3.18-2.99 (m, 3H), 2.87-2.75 (m, 7H), 2.37-2.30 (m, 2H), 2.21-2.03 (m, 5H), 1.91-1.62 (m, 5H), 1.59 (dd, J=2.0, 5.2 Hz, 3H), 1.29-1.20 (m, 1H), 0.73 (dt, J=3.4, 7.3 Hz, 3H); LCMS (ESI, M+1): m/z=735.4.

### Example 23

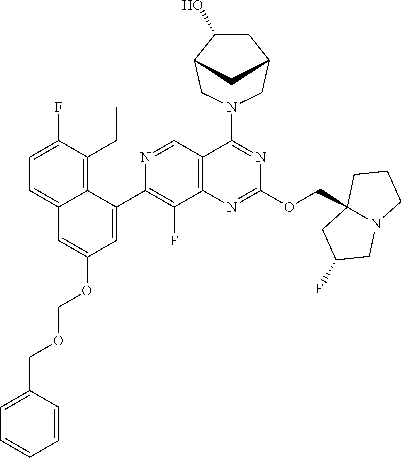 [SMILES: CCc1c(F)ccc2cc(OCOCc3ccccc3)cc(-c3ncc4c(N5C[C@@H]6C[C@H](C5)[C@H](O)C6)nc(OC[C@@]56CCCN5C[C@H](F)C6)nc4c3F)c12]

### (1R,5R,6R)-3-(7-(3-((benzyloxy)methoxy)-8-ethyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-6-ol

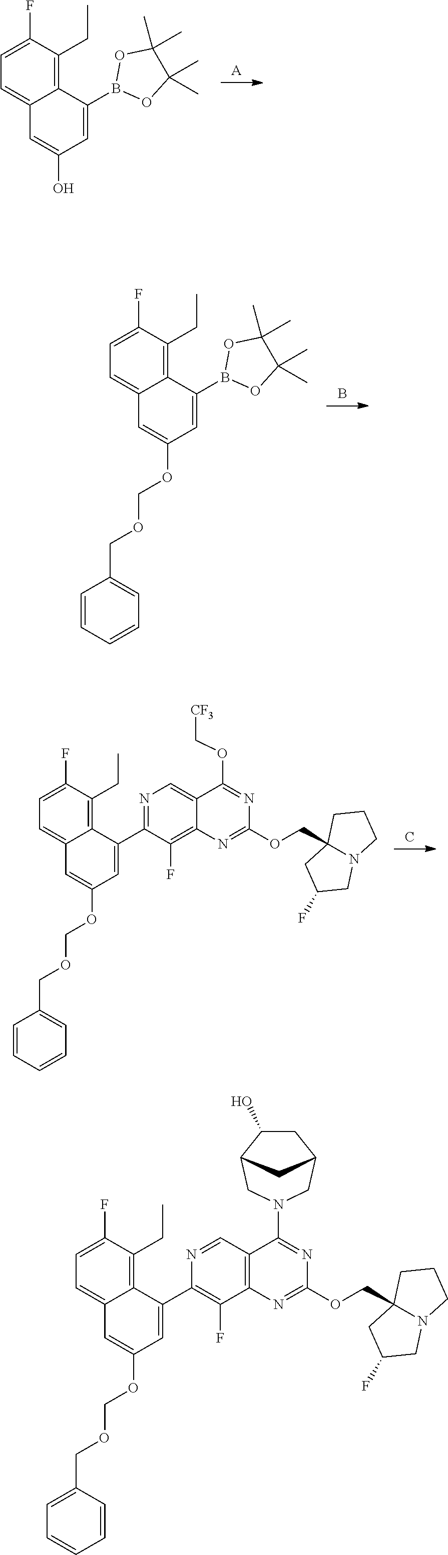

Step A. 2-(3-((benzyloxy)methoxy)-8-ethyl-7-fluoronaphthalen-1-yl)-4,4,5,5-tetramethyl-1,3,2-dioxaborolane: To a solution of 5-ethyl-6-fluoro-4-(4,4,5,5-tetramethyl-1,3,2-dioxaborolan-2-yl)naphthalen-2-ol (500 mg, 1.0 equiv) in CH₃CN (5.0 mL) were added DIEA (408 mg, 2.0 equiv) and ((chloromethoxy)methyl)benzene (495 mg, 2.0 equiv). The reaction was stirred at 25° C. for 12 hours. The mixture was quenched by adding water and concentrated to remove the organic solvent. The mixture was extracted with EtOAc (2×5 mL), dried over Na₂SO₄, concentrated and purified with column chromatography to afford the title compound (560 mg, 81% yield) as colorless liquid.

Step B. 7-(3-((benzyloxy)methoxy)-8-ethyl-7-fluoronaphthalen-1-yl)-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine: To a solution of 2-(3-((benzyloxy)methoxy)-8-ethyl-7-fluoronaphthalen-1-yl)-4,4,5,5-tetramethyl-1,3,2-dioxaborolane (200 mg, 1.0 equiv) in CMPE (10 mL) were added K₃PO₄ (1.5 M, 0.3 mL, 1.0 equiv), 7-chloro-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine and APhos Pd G3 (2.59 mg, 0.01 equiv). The reaction was stirred at 70° C. for 12 hours under N2 atmosphere. The mixture was quenched by adding water and concentrated to remove the organic solvent. The mixture was extracted with EtOAc (3×3 mL), dried over Na₂SO₄, concentrated to afford the crude product as brown oil; LCMS (ESI, M+1): m/z=713.4.

Step C. (1R,5R,6R)-3-(7-(3-((benzyloxy)methoxy)-8-ethyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-6-ol: To a solution of 7-(3-((benzyloxy)methoxy)-8-ethyl-7-fluoronaphthalen-1-yl)-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine (100 mg, 1.0 equiv) in DMF (1.0 mL) were added 4 Å MS (20.0 mg), (1R,5R,6R)-3-azabicyclo[3.2.1]octan-6-ol (28.5 mg, 1.6 equiv) and DIPEA (36.2 mg, 2.0 equiv). The reaction was stirred at 40° C. for 12 hours. The mixture was filtered and purified with prep-HPLC (column: Phenomenex Luna C18 150*25 mm*10 um; mobile phase: [water(FA)-ACN]; B %: 27%-57%, 9 min and column: Waters Xbridge 150*25 mm*Sum; mobile phase: [water (ammonia hydroxide v/v)-ACN]; B %: 68%-98%, 9 min) to afford the title compound (26.8 mg, 25% yield) as white solid; 1H NMR (400 MHz, DMSO-d6) δ=9.43-9.16 (m, 1H), 7.96-7.83 (m, 1H), 7.71 (d, J=2.8 Hz, 1H), 7.50-7.19 (m, 7H), 5.52-5.44 (m, 2H), 5.38-5.19 (m, 1H), 4.87-4.68 (m, 4H), 4.63-4.53 (m, 1H), 4.28-3.95 (m, 3H), 3.82-3.69 (m, 1H), 3.19-2.98 (m, 3H), 2.91-2.79 (m, 1H), 2.39-2.31 (m, 2H), 2.21-1.98 (m, 6H), 1.94-1.71 (m, 4H), 1.69-1.59 (m, 1H), 1.31-1.17 (m, 2H), 0.78-0.60 (m, 3H); LCMS (ESI, M+1): m/z=740.3.

### Example 24

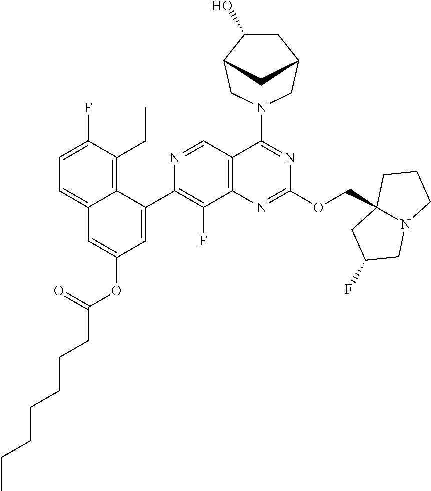 [SMILES: CCCCCCCC(=O)Oc1cc(-c2ncc3c(N4C[C@@H]5C[C@H](C4)[C@H](O)C5)nc(OC[C@@]45CCCN4C[C@H](F)C5)nc3c2F)c2c(CC)c(F)ccc2c1]

### 5-ethyl-6-fluoro-4-(8-fluoro-2-(((2R,7aS)-2-fluorotetrahydro-1H-pyrrolizin-7a(5H)-yl)methoxy)-4-((1R,5R,6R)-6-hydroxy-3-azabicyclo[3.2.1]octan-3-yl)pyrido[4,3-d]pyrimidin-7-yl)naphthalen-2-yl octanoate

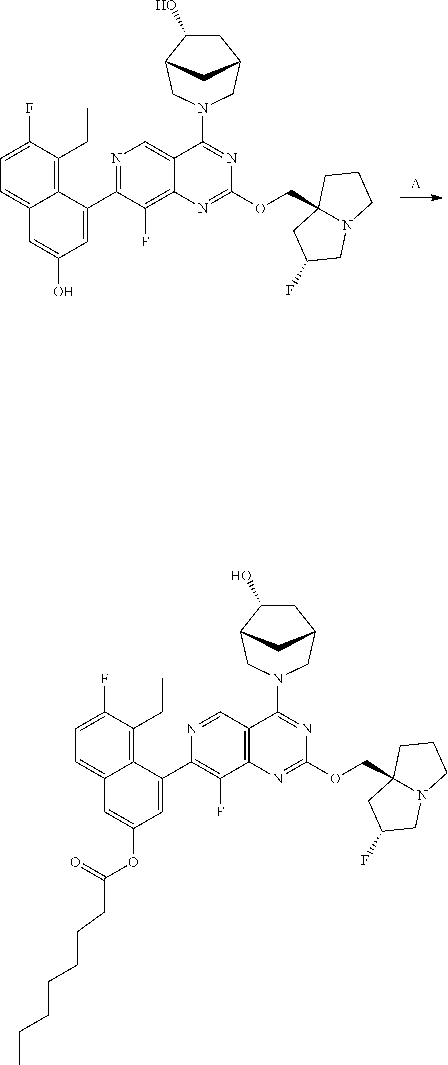

Step A. 5-ethyl-6-fluoro-4-(8-fluoro-2-(((2R,7aS)-2-fluorohexahydro-1H-pyrrolizin-7a-yl)methoxy)-4-((1R,5R,6R)-6-hydroxy-3-azabicyclo[3.2.1]octan-3-yl)pyrido[4,3-d]pyrimidin-7-yl)naphthalen-2-yl octanoate: To a solution of (1R,5R,6R)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-6-ol (100 mg, 1.0 equiv) in DCM (1 mL) were added TEA (49.0 mg, 3.0 equiv) and octanoyl chloride (26.2 mg, 1.0 equiv). The reaction was stirred at −40° C. for 0.5 hours. The mixture was diluted with water (2 mL) and extracted with DCM (3×1 mL). The combined organic layers were washed with brine (1 mL), dried over sodium sulfate, concentrated and purified with prep-HPLC (column: Phenomenex luna C18 150×25 mm×10 μm; mobile phase: [water(FA)-ACN]; B %: 32%-62%, 10 min) to afford the title compound (7.56 mg, 6% yield) as white solid; 1H NMR (400 MHz, CHLOROFORM-d) δ=9.05 (d, J=4.8 Hz, 1H), 7.76-7.70 (m, 2H), 7.31 (t, J=9.2 Hz, 1H), 7.23 (d, J=2.4 Hz, 1H), 5.43-5.24 (m, 1H), 5.09 (br t, J=14.8 Hz, 1H), 4.38-4.32 (m, 2H), 4.27 (br t, J=11.2 Hz, 1H), 3.87-3.80 (m, 1H), 3.50-3.27 (m, 3H), 3.27-3.19 (m, 1H), 3.08-2.97 (m, 1H), 2.62-2.50 (m, 3H), 2.37 (br d, J=17.6 Hz, 3H), 2.32-2.22 (m, 3H), 2.22-2.15 (m, 2H), 2.00 (br dd, J=6.4, 11.7 Hz, 3H), 1.85 (br s, 2H), 1.80-1.73 (m, 2H), 1.46-1.27 (m, 8H), 1.23-1.07 (m, 1H), 0.92-0.81 (m, 6H); LCMS (ESI, M+1): m/z=746.5.

### Example 25

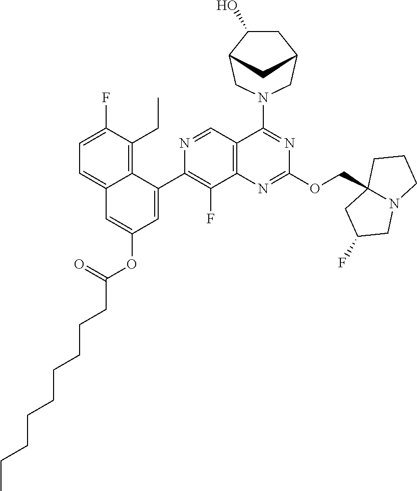 [SMILES: CCCCCCCCCC(=O)Oc1cc(-c2ncc3c(N4C[C@@H]5C[C@H](C4)[C@H](O)C5)nc(OC[C@@]45CCCN4C[C@H](F)C5)nc3c2F)c2c(CC)c(F)ccc2c1]

### 5-ethyl-6-fluoro-4-(8-fluoro-2-(((2R,7aS)-2-fluorotetrahydro-1H-pyrrolizin-7a(5H)-yl)methoxy)-4-((1R,5R,6R)-6-hydroxy-3-azabicyclo[3.2.1]octan-3-yl)pyrido[4,3-d]pyrimidin-7-yl)naphthalen-2-yl decanoate

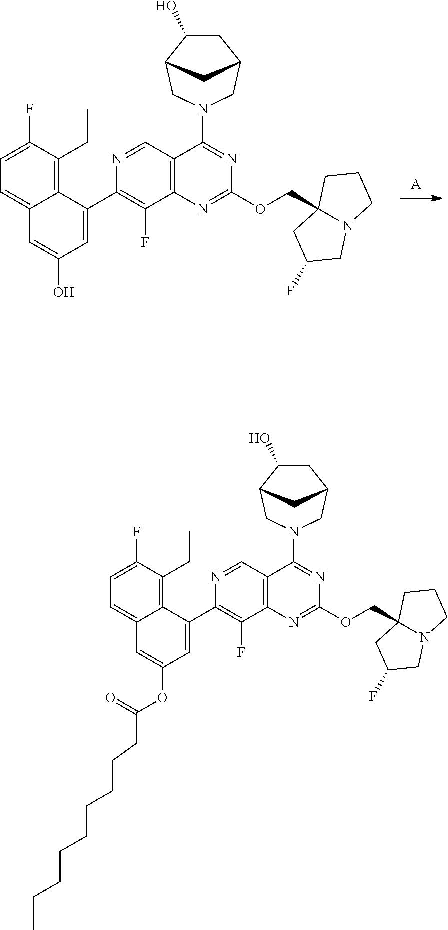

Step A. 5-ethyl-6-fluoro-4-(8-fluoro-2-(((2R,7aS)-2-fluorotetrahydro-1H-pyrrolizin-7a(5H)-yl)methoxy)-4-((1R,5R,6R)-6-hydroxy-3-azabicyclo[3.2.1]octan-3-yl)pyrido[4,3-d]pyrimidin-7-yl)naphthalen-2-yl decanoate: To a solution of (1R,5R,6R)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-6-ol (3.00 g, 1.0 equiv) and TEA (1.47 g, 3.0 equiv) in dichloromethane (30 mL) was added decanoyl chloride (1.11 g, 1.2 equiv). The reaction was stirred at 0° C. for 2 hours and 20° C. for 10 hours. The mixture was quenched by adding water (20 mL) and extracted with dichloromethane (4×30 mL). The combined organic layers were dried over anhydrous sodium sulfate, concentrated and purified with prep-HPLC [Phenomenex luna C18 150×40 mm×15 μm; A: water (FA), B: ACN; B %: 37%-67%, over 15 min] to afford the title compound (2.28 g, 61% yield) as white solid; 1H NMR (400 MHz, DMSO-d6) δ=9.39-9.22 (m, 1H), 8.02-7.98 (m, 1H), 7.89 (d, J=1.6 Hz, 1H), 7.53-7.49 (m, 1H), 7.34 (s, 1H), 5.37-5.16 (m, 1H), 4.87-4.67 (m, 2H), 4.59-4.56 (m, 1H), 4.21-4.09 (m, 2H), 4.01 (br d, J=10.0 Hz, 1H), 3.72 (br t, J=12.0 Hz, 1H), 3.33-3.27 (m, 1H), 3.15-2.97 (m, 3H), 2.87-2.76 (m, 1H), 2.60 (br t, J=7.2 Hz, 2H), 2.32 (br s, 1H), 2.27-2.10 (m, 3H), 2.09-1.95 (m, 3H), 1.85-1.70 (m, 4H), 1.64-1.61 (m, 3H), 1.42-1.15 (m, 14H), 0.87-0.71 (m, 6H); LCMS (ESI, M+1): m/z=774.6.

### Example 26

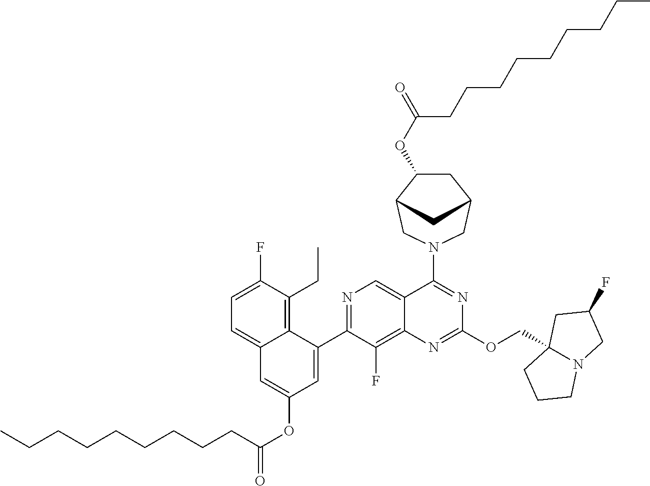 [SMILES: CCCCCCCCCC(=O)Oc1cc(-c2ncc3c(N4C[C@@H]5C[C@H](C4)[C@H](OC(=O)CCCCCCCCC)C5)nc(OC[C@@]45CCCN4C[C@H](F)C5)nc3c2F)c2c(CC)c(F)ccc2c1]

### 4-(4-((1R,5R,6R)-6-(decanoyloxy)-3-azabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-yl decanoate

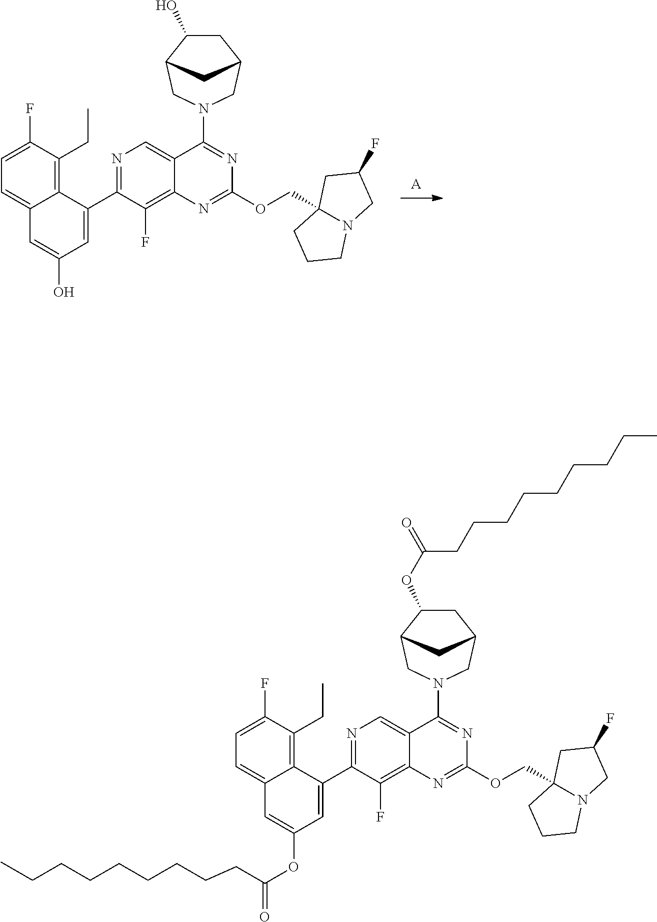

Step A: 4-(4-((1R,5R,6R)-6-(decanoyloxy)-3-azabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-yl decanoate: (1R,5R,6R)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-6-ol (0.3 g) was dissolved in DCM (6 mL). Then decanoic acid (4 eq), DMAP (0.4 eq), EDCI (4.4 eq) were added to the reaction sequentially. The reaction was stirred overnight. Upon completion, the reaction mixture was diluted with DCM (6 mL) and washed with water (10 mL×2). The organic layer was separated, dried over Na₂SO₄, and concentrated. The residue is purified by flash column chromatography using EtOAc/Heptane as eluent to provide the title compound as colorless oil (0.25 g, 56% yield). 1H NMR (400 MHz, CDCl3): δ 9.12 (d, 1H), 7.75-7.70 (m, 2H), 7.30 (t, 1H), 7.20 (dd, 1H), 5.31 (d, 1H), 5.10 (d, 1H), 4.95 (q, 1H), 4.64 (q, 1H), 4.28 (q, 1H), 4.14 (dd, 1H), 3.68 (d, 1H), 3.37 (d, 1H), 3.25-3.10 (m, 3H), 3.00-2.94 (m, 1H), 2.59-2.45 (m, 5H), 2.37-2.14 (m, 5H), 2.02-1.72 (m, 10H), 1.56-1.54 (m, 1H), 1.41-1.19 (m, 28H), 0.19-1.81 (m, 10H). MS: ESI-MS calcd for C54H72F3N505 [M+H]+ 928.56, found 928.51.

### Example 27

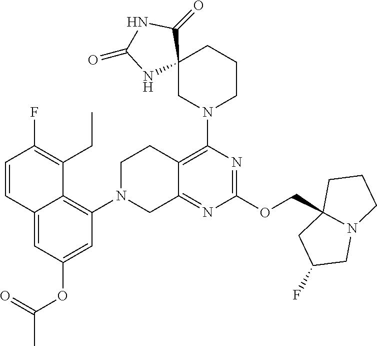 [SMILES: CCc1c(F)ccc2cc(OC(C)=O)cc(N3CCc4c(nc(OC[C@@]56CCCN5C[C@H](F)C6)nc4N4CCC[C@]5(C4)NC(=O)NC5=O)C3)c12]

### 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl acetate

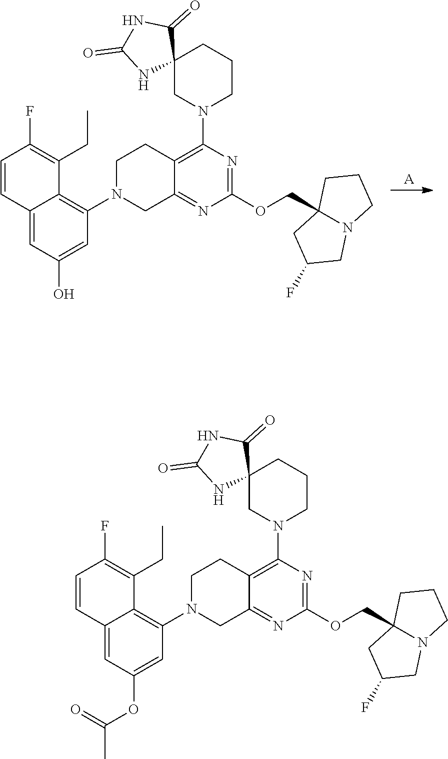

Step A. 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl acetate: To a solution of (R)-7-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione (100 mg, 1.0 equiv) and TEA (46.9 mg, 3.0 equiv) in THE (2 mL) was added Ac2O (48.9 mg, 3.1 equiv) at −40° C. The reaction was stirred at −40° C. for 3.5 hours. The mixture was quenched with water (5 mL) and extracted with ethyl acetate (2×10 mL). The organic layer was concentrate and purified with prep-HPLC[column: Phenomenex luna CIS 150×25 mm×10 μm; A: water (FA), B: ACN, B %: 19%-49% over 10 minutes] to afford the title compound (50.1 mg, 44% yield) as white solid; 1H NMR (400 MHz, CD3OD) δ=7.73 (dd, J=5.8, 8.8 Hz, 1H), 7.43 (t, J=2.8 Hz, 1H), 7.29 (t, J=9.4 Hz, 1H), 7.18 (dd, J=2.2, 9.8 Hz, 1H), 5.49-5.23 (m, 1H), 4.72-4.51 (m, 1H), 4.31-3.91 (m, 5H), 3.70 (d, J=17.8 Hz, 1H), 3.56-3.33 (m, 7H), 3.24-3.11 (m, 2H), 3.10-2.98 (m, 1H), 2.85-2.67 (m, 1H), 2.47-2.32 (m, 1H), 2.32-2.29 (m, 3H), 2.28-1.75 (m, 9H), 1.12 (dt, J=2.0, 7.2 Hz, 3H); LCMS (ESI, M+1): m/z=690.3.

### Example 28

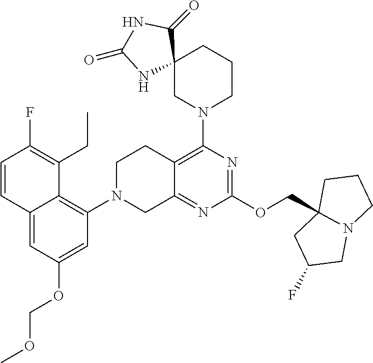 [SMILES: CCc1c(F)ccc2cc(OCOC)cc(N3CCc4c(nc(OC[C@@]56CCCN5C[C@H](F)C6)nc4N4CCC[C@]5(C4)NC(=O)NC5=O)C3)c12]

### (R)-7-(7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione

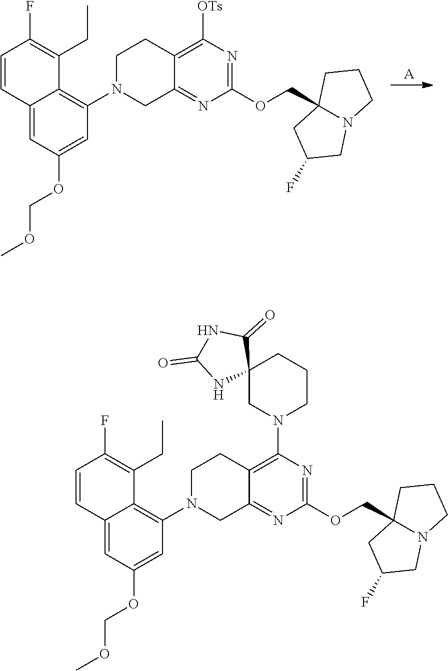

Step A. (R)-7-(7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione: To a solution of 7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl 4-methylbenzenesulfonate (140 mg, 1.0 equiv) and (R)-1,3,7-triazaspiro[4.5]decane-2,4-dione (56.0 mg, 1.6 equiv) in DMF (0.6 mL) were added DIPEA (52.1 mg, 2.0 equiv) and 4 Å molecular sieve (20 mg). The reaction mixture was stirred at 40° C. for 16 hours. The mixture was filtered and purified with prep-HPLC [column: Phenomenex C18 75×30 mm×3 m; mobile phase: water(FA)-ACN; B %: 20%-50% over 5 minutes] and [column: Waters Xbridge 150×25 mm×5 μm; mobile phase: water (NH₄HCO₃)-ACN; B %: 50%-80% over 8 minutes] to afford the title compound (68.3 mg, 48% yield) as white solid; ¹H NMR (400 MHz, CD3OD) δ=7.65-7.59 (m, 1H), 7.28-7.24 (m, 1H), 7.20 (t, J=9.2 Hz, 1H), 7.11 (dd, J=2.4, 10.8 Hz, 1H), 5.35-5.30 (m, 1H), 5.28 (d, J=3.2 Hz, 2H), 4.27-3.91 (m, 5H), 3.65 (d, J=17.6 Hz, 1H), 3.49 (d, J=2.4 Hz, 4H), 3.44-3.33 (m, 3H), 3.30-2.90 (m, 7H), 2.83-2.67 (m, 1H), 2.33-2.04 (m, 4H), 2.01-1.76 (m, 6H), 1.15-1.05 (m, 3H); LCMS (ESI, M+1): m/z=692.5.

### Example 29

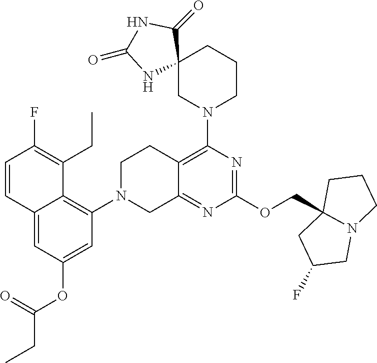 [SMILES: CCC(=O)Oc1cc(N2CCc3c(nc(OC[C@@]45CCCN4C[C@H](F)C5)nc3N3CCC[C@]4(C3)NC(=O)NC4=O)C2)c2c(CC)c(F)ccc2c1]

### 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl propionate

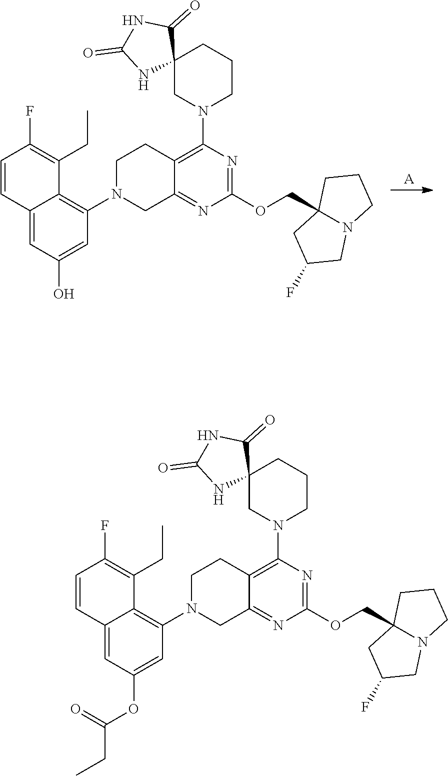

Step A. 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-ylpropionate: To a solution of (R)-7-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione (110 mg, 1.0 equiv) and TEA (51.5 mg, 3.0 equiv) in THF (1.5 mL) was added propionic anhydride (22.1 mg, 1.0 equiv) in THF (0.5 mL) dropwise at −40° C. The reaction was stirred at −40° C. for 1 hour and 25° C. for 15 hours. The mixture was diluted with H2O (5 mL) and extracted with ethyl acetate (4×3 mL). The organic layers were concentrated and purified with prep-HPLC [column: Phenomenex luna C18 150×25 mm×10 m; mobile phase: water(FA)-ACN; B %: 25%-55% over 10 minutes] to afford the title compound (44.0 mg, 35% yield) as white solid; 1H NMR (400 MHz, CD3OD) δ=7.73 (dd, J=6.0, 8.8 Hz, 1H), 7.43 (t, J=2.4 Hz, 1H), 7.29 (t, J=9.2 Hz, 1H), 7.17 (dd, J=2.4, 9.2 Hz, 1H), 5.43-5.22 (m, 1H), 4.29-3.88 (m, 5H), 3.70 (br d, J=17.6 Hz, 1H), 3.61-3.33 (m, 7H), 3.28-2.97 (m, 4H), 2.65 (dq, J=2.0, 7.6 Hz, 3H), 2.46-1.80 (m, 10H), 1.25 (dt, J=2.0, 7.6 Hz, 3H), 1.12 (dt, J=2.4, 7.2 Hz, 3H); LCMS (ESI, M+1): m/z=704.5.

### Example 30

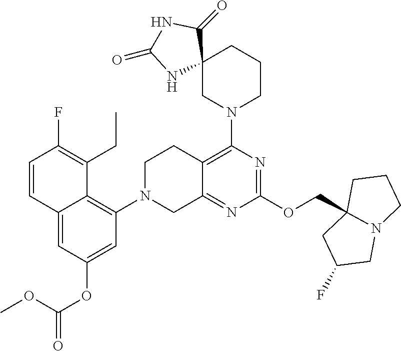 [SMILES: CCc1c(F)ccc2cc(OC(=O)OC)cc(N3CCc4c(nc(OC[C@@]56CCCN5C[C@H](F)C6)nc4N4CCC[C@]5(C4)NC(=O)NC5=O)C3)c12]

### 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl methyl carbonate

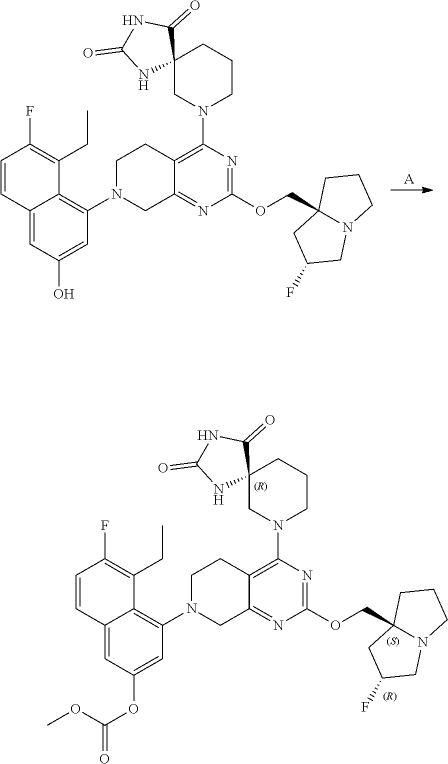

Step A. 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl methyl carbonate: To a solution of (R)-7-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione (50.0 mg, 1.00 equiv) and TEA (15.6 mg, 2.0 equiv) in THF (2.00 mL) was added dimethyl dicarbonate (20.7 mg, 2.0 equiv). The reaction was stirred at 0° C. for 1 hour. The mixture was quenched with NH₄Cl (10.0 mL) at 25° C., diluted with water (5.00 mL) and extracted with ethyl acetate (2×20 mL). The combined organic layers were washed with brine (10 mL), dried over Na₂SO₄, filtered, concentrated under reduced pressure and purified with prep-HPLC (Xtimate C18 150*40 mm*10 um; mobile phase: [water (TFA)-ACN]; B %: 30%-60%, 10 min). The collected fractions were neutralized with NaHCO₃ (2.00 M, 2.00 mL), extracted with ethyl acetate (2×10 mL), dried over Na₂SO₄, filtered and concentrated under reduced pressure to afford tittle compound (4.36 mg, 7.6% yield) as a white solid; 1H NMR (400 MHz, DMSO-d6) δ=10.78 (br s, 1H), 8.68-8.43 (m, 1H), 7.86 (br s, 1H), 7.73-7.58 (m, 1H), 7.54-7.22 (m, 2H), 5.32-5.15 (m, 1H), 4.07-3.77 (m, 9H), 3.76-3.53 (m, 2H), 3.27-2.64 (m, 10H), 2.06-1.89 (m, 5H), 1.83-1.69 (m, 5H), 1.07 (br s, 3H); LCMS (ESI, M+1): m/z=706.2.

### Example 31

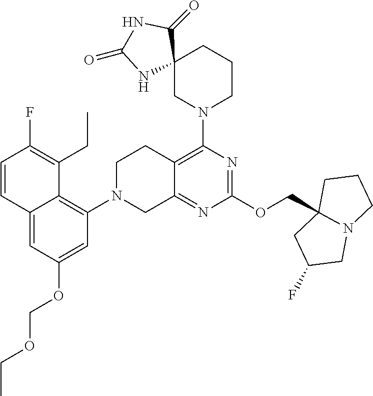 [SMILES: CCOCOc1cc(N2CCc3c(nc(OC[C@@]45CCCN4C[C@H](F)C5)nc3N3CCC[C@]4(C3)NC(=O)NC4=O)C2)c2c(CC)c(F)ccc2c1]

### (R)-7-(7-(3-(ethoxymethoxy)-8-ethyl-7-fluoronaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione

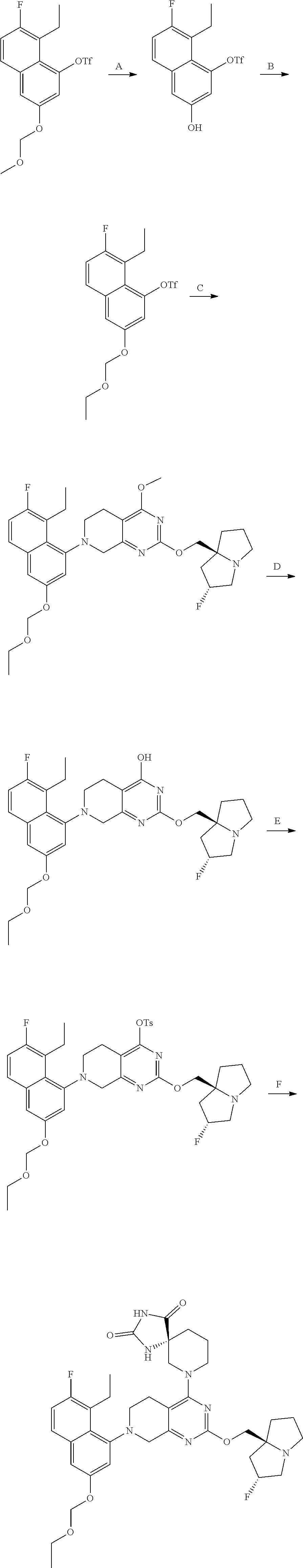

Step A. 8-ethyl-7-fluoro-3-hydroxy-8,8a-dihydronaphthalen-1-yl trifluoromethanesulfonate: 8-ethyl-7-fluoro-3-(methoxymethoxy)naphthalen-1-yl trifluoromethanesulfonate (5.0 g, 1.0 equiv) was added into HCl·MeOH (4 M, 50.0 mL) in portions at 0° C. The reaction mixture was stirred at 0° C. for 2 hours. The mixture was concentrated at room temperature. The residue was dissolved in ethyl acetate (100 mL) and saturated NaHCO₃ aqueous (100 mL). The mixture was diluted with H₂O (100 mL) and extracted with ethyl acetate (4×50 mL). The organic layers were dried over anhydrous Na₂SO₄ and concentrated to afford the title compound (4.4 g, 99% yield, crude) as red oil.

Step B. 3-(ethoxymethoxy)-8-ethyl-7-fluoronaphthalen-1-yl trifluoromethanesulfonate: To a solution of (8-ethyl-7-fluoro-3-hydroxy-8,8a-dihydronaphthalen-1-yl) trifluoromethanesulfonate (2 g, 1.0 equiv) and DIPEA (2.28 g, 3.0 equiv) in dichloromethane (20 mL) was added chloromethoxyethane (833 mg, 1.5 equiv) dropwise at 0° C. The reaction mixture was stirred at 20° C. for 1 hour. The mixture was diluted with H₂O (60 mL) and extracted with dichloromethane (3×20 mL). The organic layer was dried over anhydrous Na₂SO₄, concentrated and purified with column chromatography [SiO₂, Petroleum ether/Ethyl acetate=20/1] to afford the title compound (2.2 g, 94% yield) as light yellow oil; ¹H NMR (400 MHz, CDCl3) β=7.59 (dd, J=5.6, 9.2 Hz, 1H), 7.40 (d, J=2.4 Hz, 1H), 7.33 (d, J=2.4 Hz, 1H), 7.26-7.21 (m, 1H), 5.29 (s, 2H), 3.74 (q, J=7.2 Hz, 2H), 3.20 (dq, J=2.8, 7.2 Hz, 2H), 1.21 (dt, J=5.2, 7.2 Hz, 6H).

Step C. 7-(3-(ethoxymethoxy)-8-ethyl-7-fluoronaphthalen-1-yl)-2-(((2R,7aS)-2-fluorotetrahydro-1H-pyrrolizin-7a(5H)-yl)methoxy)-4-methoxy-5,6,7,8-tetrahydropyrido[3,4-d]pyrimidine: To a mixture of 2-(((2R,7aS)-2-fluorotetrahydro-1H-pyrrolizin-7a(5H)-yl)methoxy)-4-methoxy-5,6,7,8-tetrahydropyrido[3,4-d]pyrimidine (1.0 g, 1.0 equiv), 3-(ethoxymethoxy)-8-ethyl-7-fluoronaphthalen-1-yl trifluoromethanesulfonate (1.48 g, 1.2 eq) in dioxane (10 mL) was added Cs₂CO₃ (2.53 g, 2.5 equiv) and 4 Å molecular sieve (50 mg). Xantphos Pd G4 (298 mg, 0.1 equiv) was added under N2 atmosphere, and the reaction mixture was stirred at 90° C. for 14 hours. The mixture was diluted with H₂O (30 mL) and extracted with ethyl acetate (4×15 mL). The combined organic layers were dried over anhydrous Na₂SO₄, concentrated and purified with reversed phase flash [water (FA, 0.1%)/acetonitrile=1/1] to afford the title compound (346 mg, 19% yield) as brown gum; LCMS (ESI, M+1): m/z=569.4.

Step D. 7-(3-(ethoxymethoxy)-8-ethyl-7-fluoronaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-ol: To a solution of 7-(3-(ethoxymethoxy)-8-ethyl-7-fluoronaphthalen-1-yl)-2-(((2R,7aS)-2-fluorotetrahydro-1H-pyrrolizin-7a(5H)-yl)methoxy)-4-methoxy-5,6,7,8-tetrahydropyrido[3,4-d]pyrimidine (340 mg, 1.0 equiv) in DMF (5 mL) was added ethylsulfanylsodium (176 mg, 3.5 equiv). The mixture was stirred at 60° C. for 2 hours. The mixture was diluted with H₂O (40 mL) and extracted with ethyl acetate (3×30 mL). The organic layer was dried over anhydrous Na₂SO₄ and concentrated to afford the title compound (330 mg, crude) as brown oil; LCMS (ESI, M+1): m/z=555.2.

Step E. 7-(3-(ethoxymethoxy)-8-ethyl-7-fluoronaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl 4-methylbenzenesulfonate: To a solution of 7-(3-(ethoxymethoxy)-8-ethyl-7-fluoronaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-ol (300 mg, 1.0 equiv), DIEA (209 mg, 3.0 equiv) in THE (7 mL) was added DMAP (6.61 mg, 0.1 equiv) and 4-methylbenzenesulfonyl chloride (155 mg, 1.5 equiv) at 0° C. The reaction was stirred at 20° C. for 1.5 hours. The mixture was diluted with H₂O (20 mL) and extracted with ethyl acetate (4×20 mL). The organic layer was dried over anhydrous Na₂SO₄, concentrated and purified with column chromatography [Al₂O₃, ethyl acetate] to afford the title compound (270 mg, 67% yield) as brown gum; LCMS (ESI, M+1): m/z=709.2.

Step F. (R)-7-(7-(3-(ethoxymethoxy)-8-ethyl-7-fluoronaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione: To a solution of 7-(3-(ethoxymethoxy)-8-ethyl-7-fluoronaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl 4-methylbenzenesulfonate (150 mg, 1.0 equiv) and (R)-1,3,7-triazaspiro[4.5]decane-2,4-dione (64.4 mg, 1.8 equiv) in DMF (2 mL) was added DIEA (54.7 mg, 2.0 equiv) and 4 Å molecular sieve (20 mg). The reaction mixture was stirred at 40° C. for 14 hours. The mixture was filtered and purified with reversed phase flash [water (FA, 0.1%)/acetonitrile=2/3] and prep-HPLC [column: Waters Xbridge 150×25 mm×5 m; mobile phase: water (NH₄HCO₃)-ACN; B %: 54%-84% over 8 minutes] to afford the title compound (34.3 mg, 22% yield) as white solid; ¹H NMR (400 MHz, DMSO-d6) δ=10.87-10.63 (m, 1H), 8.60-8.48 (m, 1H), 7.83-7.64 (m, 1H), 7.38-7.27 (m, 2H), 7.20-7.06 (m, 1H), 5.37-5.31 (m, 2H), 5.31-5.14 (m, 1H), 4.07-3.76 (m, 5H), 3.74-3.59 (m, 3H), 3.50-3.34 (m, 2H), 3.27-3.11 (m, 3H), 3.09-2.93 (m, 4H), 2.93-2.58 (m, 3H), 2.10-1.67 (m, 10H), 1.19-1.11 (m, 3H), 1.06 (dt, J=2.4, 7.2 Hz, 3H); LCMS (ESI, M+1): m/z=706.5.

### Example 32

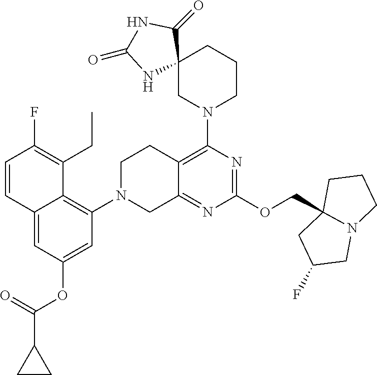 [SMILES: CCc1c(F)ccc2cc(OC(=O)C3CC3)cc(N3CCc4c(nc(OC[C@@]56CCCN5C[C@H](F)C6)nc4N4CCC[C@]5(C4)NC(=O)NC5=O)C3)c12]

### 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl cyclopropanecarboxylate

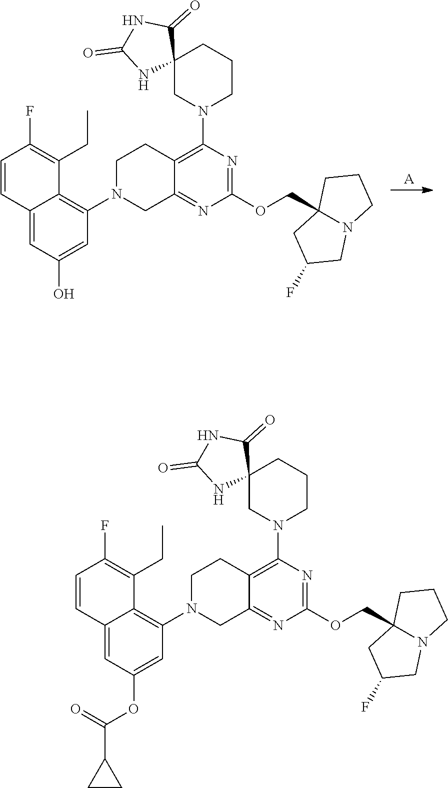

Step A. 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl cyclopropanecarboxylate: To a solution of (R)-7-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione (200 mg, 1.0 equiv) and TEA (93.7 mg, 3.0 equiv) in DCM (2 mL) was added cyclopropanecarbonyl chloride (38.7 mg, 1.2 equiv) at −40° C. The reaction was stirred at 0° C. for 5 hours. The reaction mixture was quenched by adding H₂O (5 mL) at 0° C. and extracted with DCM (3×5 mL). The combined organic layers were dried over Na₂SO₄, concentrated and purified with prep-HPLC [Phenomenex Luna C18 150×25 mm×10 μm; A: water(FA), B: ACN; B %: 20%-50% over 9 min] to afford the title compound (73.2 mg, 3200 yield) as white solid, 1H NMR (400 MHz, METHANOL-d4) δ=7.78-7.69 (m, 1H), 7.48-7.36 (m, 1H), 7.32-7.25 (m, 1H), 7.19-7.11 (m, 1H), 5.39-5.18 (m, 1H), 4.22-3.93 (in, 5H), 3.75-3.63 (m, 1H), 3.54-3.37 (m, 4H), 3.29-3.14 (m, 4H), 3.14-2.95 (m, 2H), 2.91-2.68 (m, 2H), 2.39-2.07 (m, 4H), 2.04-1.73 (m, 6H), 1.39-1.27 (m, 5H), 1.19-1.07 (in, 3H); LCMS (ESI, M+1): m/z=716.5.

### Example 33

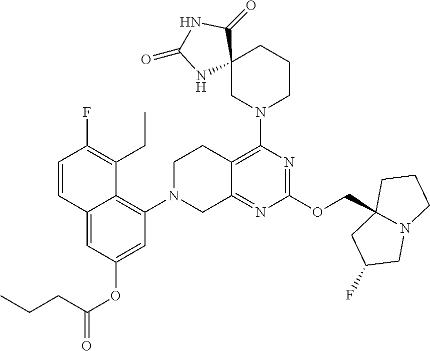 [SMILES: CCCC(=O)Oc1cc(N2CCc3c(nc(OC[C@@]45CCCN4C[C@H](F)C5)nc3N3CCC[C@]4(C3)NC(=O)NC4=O)C2)c2c(CC)c(F)ccc2c1]

### 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl butyrate

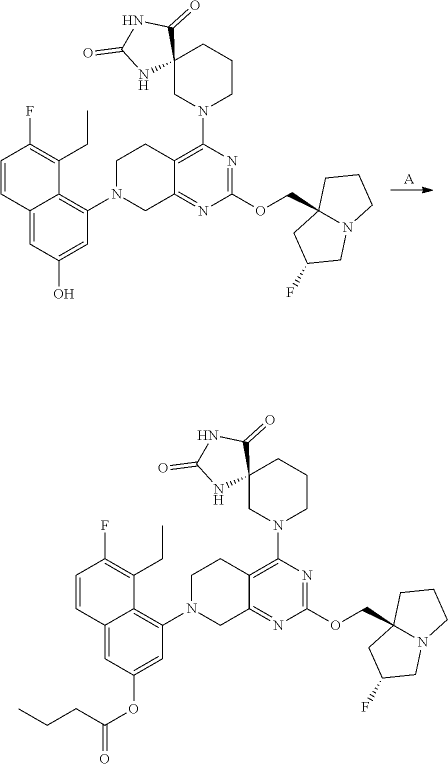

Step A. 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl butyrate: To a solution of (5R)-9-[7-(8-ethyl-7-fluoro-3-hydroxy-1-naphthyl)-2-[[(2R,8S)-2-fluoro-1,2,3,5,6,7-hexahydropyrrolizin-8-yl]methoxy]-6,8-dihydro-5H-pyrido[3,4-d]pyrimidin-4-yl]-1,3,9-triazaspiro[4.5]decane-2,4-dione (100 mg, 1.0 equiv) and TEA (46.9 mg, 3.0 equiv) in DCM (2 mL) added butanoyl chloride (24.7 mg, 1.5 equiv) at 0° C. The reaction was stirred at 0° C. for 8 hours. The mixture was quenched by adding H₂O (5 mL) at 0° C. and extracted with DCM (3×5 mL). The combined organic layers were dried over Na₂SO₄, concentrated and purified with prep-HPLC [Waters Xbridge 150×25 mm×5 μm; A: water (NH₄HCO₃), B: ACN; B %: 60%-90%, 8 min] and [Waters Xbridge 150×25 mm×5 μm; A: water (NH₄HCO₃), B: ACN; B %: 53%-83%, 8 min] to afford the title compound (6.70 mg, 6.0% yield) as pink solid; 1H NMR (400 MHz, METHANOL-d4) δ=7.78-7.72 (m, 1H), 7.52-7.46 (m, 1H), 7.35-7.26 (m, 1H), 7.23-7.16 (m, 1H), 5.37-5.20 (m, 1H), 4.23-3.95 (m, 5H), 3.76-3.67 (m, 1H), 3.59-3.36 (m, 5H), 3.27-3.18 (m, 3H), 3.16-2.94 (m, 3H), 2.84-2.71 (m, 1H), 266-2.59 (m, 2H), 2.37-2.20 (m, 1H), 2.20-2.07 (m, 3H), 2.04-1.77 (m, 8H), 1.18-1.11 (m, 3H), 1.10-1.01 (m, 3H); LCMS (ESI, M+1): m/z=718.2.

### Example 34

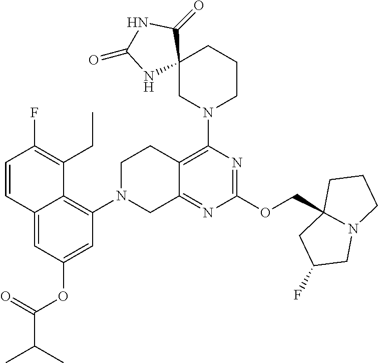 [SMILES: CCc1c(F)ccc2cc(OC(=O)C(C)C)cc(N3CCc4c(nc(OC[C@@]56CCCN5C[C@H](F)C6)nc4N4CCC[C@]5(C4)NC(=O)NC5=O)C3)c12]

### 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl isobutyrate

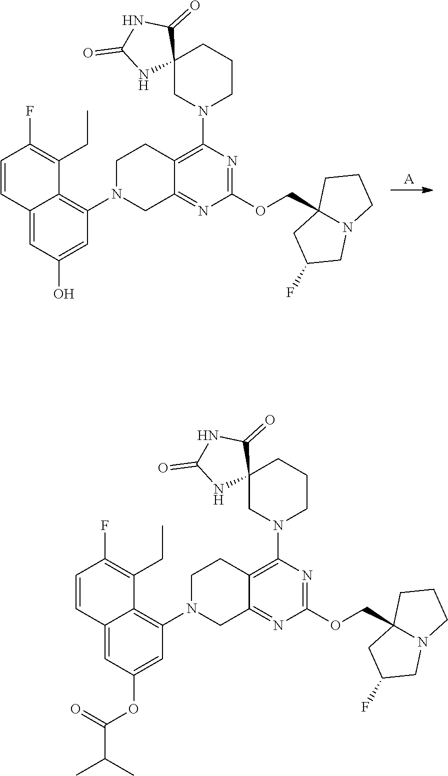

Step A. 4-(4-((R)-2,4-dioxo-1,3,7-triazaspiro[4.5]decan-7-yl)-2-((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl isobutyrate: To a solution of (R)-7-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione (200 mg, 1.0 equiv) and TEA (93.7 mg, 3.0 equiv) in DCM (2 mL) was added 2-methylpropanoyl chloride (39.5 mg, 1.2 equiv) at −40° C. The reaction was stirred at 20° C. for 6.5 hours. The mixture was quenched by adding H₂O (5 mL) at 0° C. and extracted with DCM (3×5 mL). The combined organic layers were dried over Na₂SO₄, concentrated and purified with prep-HPLC [Phenomenex Luna C18 150×25 mm×10 μm; A: water(FA), B: ACN; B %: 23%-53% over 9 min] to afford the title compound (90.6 mg, 40% yield) as white solid; 1H NMR (400 MHz, METHANOL-d4) δ=7.83-7.67 (m, 1H), 7.47-7.38 (m, 1H), 7.34-7.23 (m, 1H), 7.19-7.11 (m, 1H), 5.41-5.16 (m, 1H), 4.22-3.92 (m, 5H), 3.80-3.65 (m, 1H), 3.57-3.33 (m, 5H), 3.29-2.97 (m, 6H), 2.92-2.67 (m, 2H), 2.37-2.07 (m, 4H), 2.04-1.97 (m, 6H), 1.42-1.31 (m, 6H), 1.20-1.10 (m, 3H); LCMS (ESI, M+1): m/z=718.5.

### Example 35

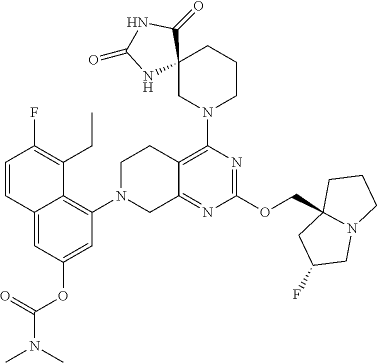 [SMILES: CCc1c(F)ccc2cc(OC(=O)N(C)C)cc(N3CCc4c(nc(OC[C@@]56CCCN5C[C@H](F)C6)nc4N4CCC[C@]5(C4)NC(=O)NC5=O)C3)c12]

### 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl dimethylcarbamate

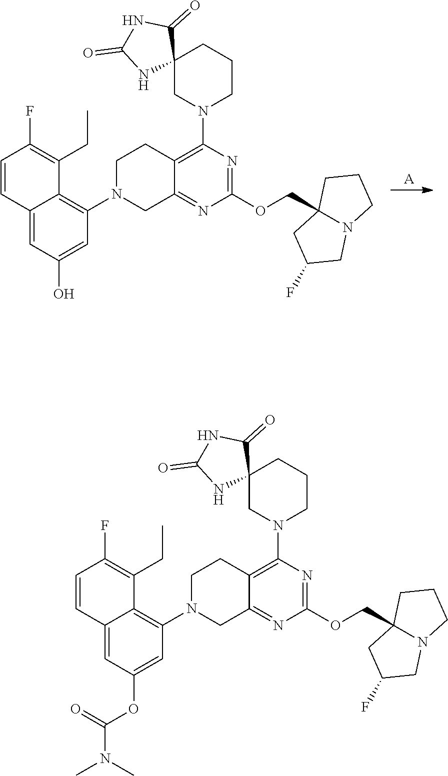

Step A. 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yldimethylcarbamate: To a solution of (R)-7-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione (110 mg, 1.0 equiv) and TEA (51.5 mg, 3.0 equiv) in THF (1.5 mL) was added dimethylcarbamic chloride (27.4 mg, 1.5 equiv) in THF (0.5 mL) dropwise at −10° C. The reaction was stirred at −40° C. for 1 hour and 25° C. for 14 hours. K₂CO₃ (47.0 mg, 2.0 equiv) was added at 0° C. and the mixture was stirred at 25° C. for 36 hours. The mixture was diluted with H₂O (5 mL) and extracted with ethyl acetate (4×3 mL). The combined organic layer was concentrated and purified with prep-HPLC [column: Phenomenex luna C18 150×25 mm×10 μm; mobile phase: water(FA)-ACN; B %: 24%-54 over 10 minutes] to afford the title compound (50.1 mg, 40% yield) as pink solid; 1H NMR (400 MHz, CD3OD) δ=7.72 (dd, J=5.6, 8.8 Hz, 1H), 7.42 (t, J=2.0 Hz, 1H), 7.28 (t, J=9.2 Hz, 1H), 7.19 (dd, J=2.0, 7.2 Hz, 1H), 5.55-5.16 (m, 1H), 4.27-3.95 (m, 5H), 3.71 (br dd, J=5.2, 17.6 Hz, 1H), 3.59-3.34 (m, 7H), 3.24-3.08 (m, 4H), 3.01 (s, 3H), 2.91-2.65 (m, 2H), 2.42-1.81 (m, 10H), 1.12 (dt, J=2.0, 7.2 Hz, 3H); LCMS (ESI, M+1): m/z=719.4.

### Example 36

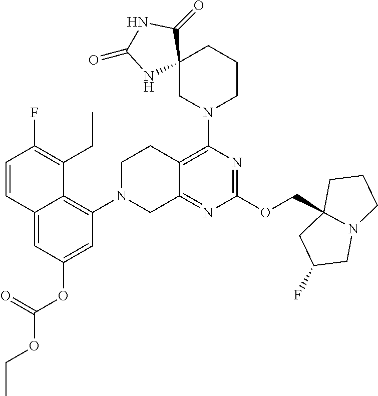 [SMILES: CCOC(=O)Oc1cc(N2CCc3c(nc(OC[C@@]45CCCN4C[C@H](F)C5)nc3N3CCC[C@]4(C3)NC(=O)NC4=O)C2)c2c(CC)c(F)ccc2c1]

### 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl ethyl carbonate

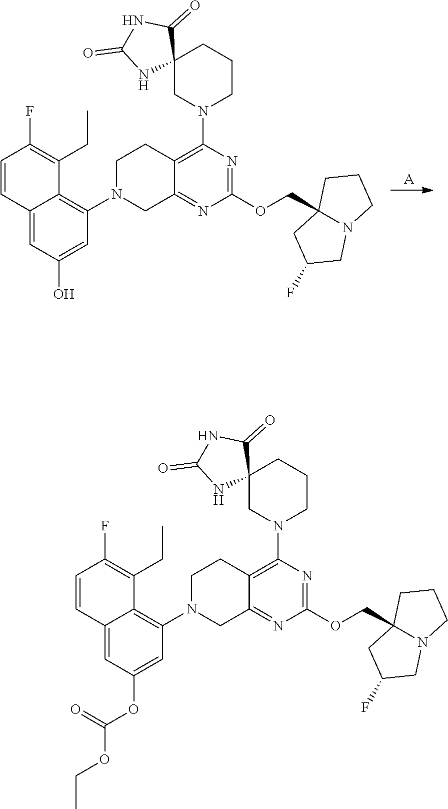

Step A. 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl ethyl carbonate: To a solution of (R)-7-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione (50.0 mg, 1.00 equiv) and TEA (15.6 mg, 2.00 equiv) in THF (1 mL) was added diethyl dicarbonate (12.5 mg, 1.00 equiv) at 0° C. The reaction was stirred at 0° C. for 0.5 hours. The mixture was diluted with water (5 mL) and extracted with ethyl acetate (3×4 mL). The combined organic layers were washed with brine (2×5 mL), dried over Na₂SO₄, filtered, concentrated under reduced pressure and purified with prep-HPLC (Xbridge Prep OBD C18 150 mm*30 mm*5 μm column (eluent: 30% to 60% CH₃CN and H₂O with 0.05% TFA). The collected fractions were neutralized with saturated NaHCO₃ (5 mL) and extracted with ethyl acetate (3×3 mL). The combined organic layers were washed with brine (2×5 mL), dried over Na₂SO₄, filtered and concentrated under reduced pressure to afford the title compound (5.00 mg, 8.5% yield) as a white solid; 1H NMR (400 MHz, DMSO-d6) δ=1.07 (br s, 3H) 1.23 (br s, 3H) 1.28-1.35 (m, 4H) 1.68-1.83 (m, 5H) 1.90-2.08 (m, 5H) 2.82 (br d, J=15.76 Hz, 2H) 2.88-3.00 (m, 2H) 3.05 (br s, 2H) 3.17 (br d, J=12.96 Hz, 2H) 3.71-3.87 (m, 2H) 3.90 (br s, 2H) 3.94-4.03 (m, 2H) 4.25-4.31 (m, 2H) 5.16-5.32 (m, 1H) 7.38-7.51 (m, 2H) 7.63-7.69 (m, 1H) 7.86 (br s, 1H) 8.52-8.62 (m, 1H) 10.72-10.83 (m, 1H); LCMS (ESI, M+1): m/z=720.2.

### Example 37

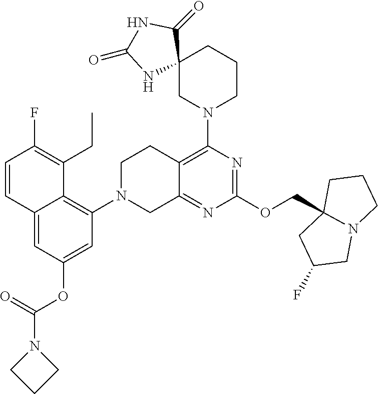 [SMILES: CCc1c(F)ccc2cc(OC(=O)N3CCC3)cc(N3CCc4c(nc(OC[C@@]56CCCN5C[C@H](F)C6)nc4N4CCC[C@]5(C4)NC(=O)NC5=O)C3)c12]

### 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl azetidine-1-carboxylate

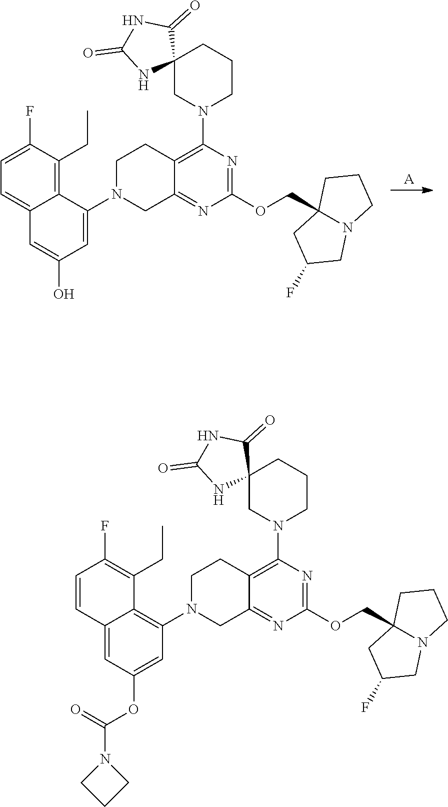

Step A: 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl azetidine-1-carboxylate: To a mixture of (R)-7-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione (100 mg, 1.0 equiv) and TEA (31.2 mg, 2.0 equiv) in DCM (1 mL) was added (4-nitrophenyl) carbonochloridate (31.1 mg, 1.0 equiv) at −10° C. under N2. After stirring at −10° C. for 20 minutes, azetidine (8.81 mg, 1.0 equiv) was added to above mixture. The reaction was warmed to 20° C. and stirred for 12 hours. The reaction mixture was diluted with water (3 mL) and extracted with dichloromethane (3×2 mL). The combined organic layers were dried over Na₂SO₄, concentrated and purified with prep-HPLC (column: Phenomenex luna C18 150×25 mm×10 um; mobile phase: [water (FA)-ACN]; B %: 23%-53%, 10 min) to afford the title compound (7.15 mg, 6.32% yield) as a white solid. SFC: ee=18.7%, Column: Chiralcel OD-3 50×4.6 mm I.D., 3 μm, Mobile phase: Phase A for CO₂, and Phase B for MeOH (0.05% DEA); Isocratic elution: 40% B in A, Flow rate: 3 mL/min; Detector: PDA; Column Temp: 35 C; Back Pressure: 100 Bar, tR1=0.659, tR2=1.849. 1H NMR (400 MHz, DMSO-d6) δ=10.80-10.69 (m, 1H), 8.58-8.49 (m, 1H), 7.82 (br s, 1H), 7.53 (d, J=2.0 Hz, 1H), 7.40 (s, 1H), 7.28-7.17 (m, 1H), 5.32-5.15 (m, 1H), 4.25-4.16 (m, 2H), 4.02-3.89 (m, 6H), 3.81 (m, 1H), 3.66 (m, 1H), 3.17 (m, 1H), 3.11-3.02 (m, 5H), 2.97 (br s, 1H), 2.87 (br s, 1H), 2.84-2.78 (m, 2H), 2.34-2.24 (m, 3H), 2.07 (m, 1H), 2.00-1.89 (m, 4H), 1.83-1.69 (m, 6H), 1.07 (m, 3H); F NMR (400 MHz, DMSO-d6) δ=−117.30, −172.08; LCMS (ESI, M+1): m/z=744.5.

### Example 38

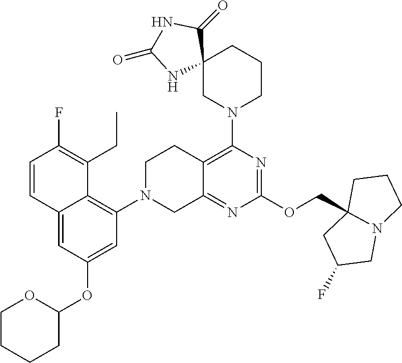 [SMILES: CCc1c(F)ccc2cc(OC3CCCCO3)cc(N3CCc4c(nc(OC[C@@]56CCCN5C[C@H](F)C6)nc4N4CCC[C@]5(C4)NC(=O)NC5=O)C3)c12]

### (5R)-7-(7-(8-ethyl-7-fluoro-3-((tetrahydro-2H-pyran-2-yl)oxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione

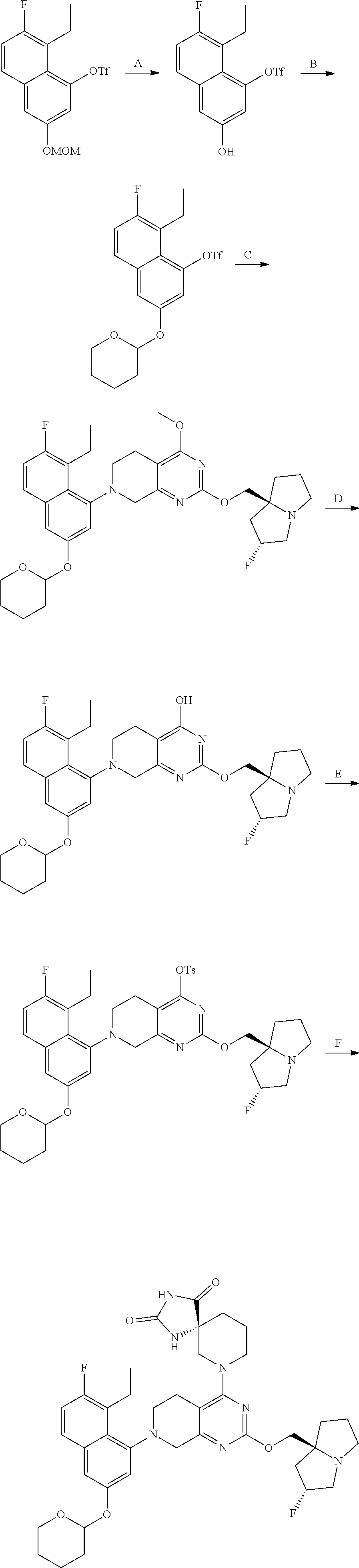

Step A. 8-ethyl-7-fluoro-3-hydroxynaphthalen-1-yl trifluoromethanesulfonate: To a solution of 8-ethyl-7-fluoro-3-(methoxymethoxy)naphthalen-1-yl trifluoromethanesulfonate (10.0 g, 1.0 equiv) in ACN (50 mL) was added HCl·dioxane (4 M, 50 mL) at 0° C. The reaction was stirred at 25° C. for 1 hour. The mixture was concentrated under reduced pressure and dissolved in water (20 mL). The residue was adjusted to pH=10 with 10% NaOH aqueous solution at 0° C. The mixture was extracted with DCM (3×100 mL). The combined organic layers were dried over anhydrous sodium sulfate and concentrated to afford the title compound (8.8 g, crude) as yellow solid.

Step B. 8-ethyl-7-fluoro-3-((tetrahydro-2H-pyran-2-yl)oxy)naphthalen-1-yl trifluoromethanesulfonate: To a mixture of 8-ethyl-7-fluoro-3-hydroxynaphthalen-1-yl trifluoromethanesulfonate (5.00 g, 1.0 equiv) and 3,4-dihydro-2H-pyran (2.49 g, 2.0 equiv) in DCM (50 mL) was added TsOH (127 mg, 0.05 equiv). The mixture was stirred at 0° C. for 0.5 hours. The reaction mixture was concentrated and purified with column chromatography [Petroleum ether/Ethyl acetate=I/O to 50/1] to afford the title compound (5.00 g, two steps yield: 74%) as yellow oil; ¹H NMR (400 MHz, METHANOL-d4) b=7.68-7.62 (m, 1H), 7.50-7.45 (m, 1H), 7.38-7.33 (m, 1H), 7.30-7.22 (m, 1H), 5.51 (s, 1H), 3.93-3.76 (m, 1H), 3.70-3.46 (m, 1H), 3.26-3.11 (m, 2H), 2.04-1.81 (m, 3H), 1.61-1.52 (m, 1H), 1.57 (m, 2H), 1.22-1.14 (m, 3H).

Step C. 7-(8-ethyl-7-fluoro-3-((tetrahydro-2H-pyran-2-yl)oxy)naphthalen-1-yl)-2-(((2R,7aS)-2-fluorotetrahydro-1H-pyrrolizin-7a(5H)-yl)methoxy)-4-methoxy-5,6,7,8-tetrahydropyrido[3,4-d]pyrimidine: To a mixture of 8-ethyl-7-fluoro-3-((tetrahydro-2H-pyran-2-yl)oxy)naphthalen-1-yl trifluoromethanesulfonate (1.00 g, 1.0 equiv) and 2-(((2R,7aS)-2-fluorotetrahydro-1H-pyrrolizin-7a(5H)-yl)methoxy)-4-methoxy-5,6,7,8-tetrahydropyrido[3,4-d]pyrimidine (916 mg, 1.2 equiv) in toluene (10 mL) were added Cs₂CO₃ (1.54 g, 2.0 equiv), Xantphos (342 mg, 0.25 equiv) and Pd₂(dba)₃ (352 mg, 0.15 equiv) under N₂ atmosphere. The reaction mixture was stirred at 110° C. for 12 hours under N₂ atmosphere. The reaction mixture was quenched with H₂O (50 mL) and extracted with EtOAc (3×100 mL). The combined organic layers were dried over anhydrous sodium sulfate, concentrated and purified with reversed phase flash chromatography [C18, 0.1% formic acid condition] to afford the title compound (1.00 g, 60% yield) as yellow solid; LCMS (ESI, M+1): m/z=595.3.

Step D. 7-(8-ethyl-7-fluoro-3-((tetrahydro-2H-pyran-2-yl)oxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-ol: To a solution of EtSH (0.780 g, 8.3 equiv) in DMAc (10 mL) was added NaH (484 mg, 60% purity, 8.0 equiv) at 0° C. The reaction mixture was stirred for 1 hour at 20° C. And then to the mixture was added 7-(8-ethyl-7-fluoro-3-((tetrahydro-2H-pyran-2-yl)oxy)naphthalen-1-yl)-2-(((2R,7aS)-2-fluorotetrahydro-1H-pyrrolizin-7a(5H)-yl)methoxy)-4-methoxy-5,6,7,8-tetrahydropyrido[3,4-d]pyrimidine (900 mg, 1.0 equiv). The reaction mixture was stirred at 60° C. for 1 hour. The reaction mixture was quenched by adding H₂O (50 mL) and extracted with EtOAc (3×100 mL). The combined organic layers were dried over anhydrous sodium sulfate and concentrated to afford the title compound (800 mg, crude) as yellow solid; LCMS (ESI, M+1): m/z=581.4.

Step E. 7-(8-ethyl-7-fluoro-3-((tetrahydro-2H-pyran-2-yl)oxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl 4-methylbenzenesulfonate: To a mixture of 7-(8-ethyl-7-fluoro-3-((tetrahydro-2H-pyran-2-yl)oxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-ol (500 mg, 1.0 equiv) and 4-methylbenzene-1-sulfonyl chloride (246 mg, 1.5 equiv) in DCM (5 mL) were added DIEA (334 mg, 3.0 equiv) and DMAP (10.5 mg, 0.1 equiv). The reaction mixture was stirred at 25° C. for 12 hours. The reaction mixture was quenched with H₂O (5 mL) and extracted with DCM (3×20 mL). The combined organic layers were dried over anhydrous sodium sulfate, concentrated and purified by column chromatography [Petroleum ether/Ethyl acetate=10/1 to 0/1] to afford the title compound (400 mg, 58% yield) as yellow solid; LCMS (ESI, M+1): m/z=735.2.

Step F. (5R)-7-(7-(8-ethyl-7-fluoro-3-((tetrahydro-2H-pyran-2-yl)oxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione: To a solution of 7-(8-ethyl-7-fluoro-3-((tetrahydro-2H-pyran-2-yl)oxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl 4-methylbenzenesulfonate (200 mg, 1.0 equiv) and DIEA (52.8 mg, 1.5 equiv) in DMF (1.0 mL) was added (R)-1,3,7-triazaspiro[4.5]decane-2,4-dione (92.1 mg, 2.0 equiv). The reaction mixture was stirred at 40° C. for 12 hours. The reaction mixture was concentrated and purified with prep-HPLC [Waters Xbridge C18 150×50 mm×10 m; A: water (NH₄HCO₃), B: ACN, B %: 64%-94% over 10 min] and [Waters Xbridge C18 150×25 mm×5 μm; A: water (NH₄HCO₃), B: ACN, B %: 58%-88% over 10 min] to afford the title compound (20.4 mg, 10% yield) as white solid; ¹H NMR (400 MHz, METHANOL-d4) δ=7.65-7.59 (m, 1H), 7.30-7.26 (m, 1H), 7.23-7.16 (m, 1H), 7.16-7.09 (m, 1H), 5.59-5.52 (m, 1H), 5.36-5.17 (m, 1H), 4.22-4.06 (m, 4H), 4.05-3.87 (m, 2H), 3.73-3.58 (m, 2H), 3.52-3.36 (m, 4H), 3.28-2.94 (m, 7H), 2.82-2.68 (m, 1H), 2.21-1.80 (m, 13H), 1.78-1.56 (m, 3H), 1.13-1.02 (m, 3H); LCMS (ESI, M+1): m/z=732.3.

### Example 39

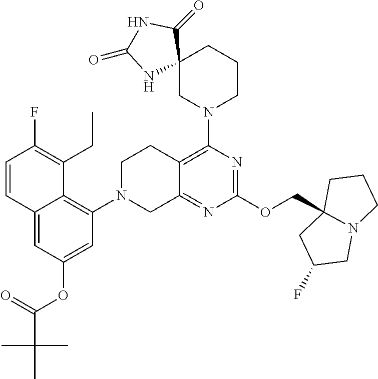 [SMILES: CCc1c(F)ccc2cc(OC(=O)C(C)(C)C)cc(N3CCc4c(nc(OC[C@@]56CCCN5C[C@H](F)C6)nc4N4CCC[C@]5(C4)NC(=O)NC5=O)C3)c12]

### 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl pivalate

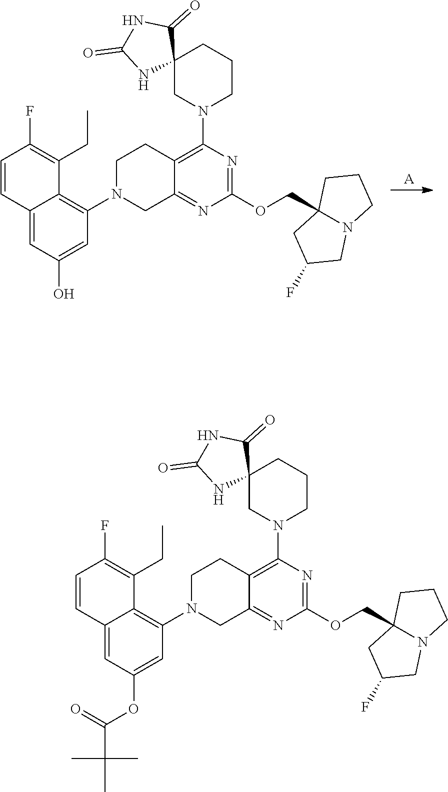

Step A. 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl pivalate: To a solution of (R)-7-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione (100 mg, 1.0 equiv) and TEA (62.0 mg, 4.0 equiv) in THF (2 mL) was added pivaloyl chloride (19.0 mg, 1.0 equiv) dropwise at −40° C. under N2 atmosphere. The reaction mixture was stirred at −40° C. for 2 hours. The mixture was quenched with anhydrous methanol (1 mL) at −40° C. and stirred for 10 minutes. The mixture was diluted with water (5 mL) and extracted with ethyl acetate (3×3 mL). The organic layer was dried over anhydrous Na₂SO₄, concentrated and purified with prep-HPLC [column: Phenomenex C18 75×30 mm×3 μm; mobile phase: water (FA)-ACN; B %: 25%-55% over 7 minutes] to afford the title compound (64 mg, 55% yield) as white solid; 1H NMR (400 MHz, CD3OD) δ=7.74 (dd, J=6.0, 8.8 Hz, 1H), 7.40 (t, J=2.4 Hz, 1H), 7.29 (t, J=9.2 Hz, 1H), 7.12 (dd, J=2.4, 7.6 Hz, 1H), 5.46-5.27 (m, 1H), 4.33-3.92 (m, 5H), 3.70 (dd, J=6.4, 17.6 Hz, 1H), 3.60-3.38 (m, 7H), 3.28-2.99 (m, 4H), 2.87-2.67 (m, 1H), 2.46-1.80 (m, 10H), 1.39 (d, J=2.0 Hz, 9H), 1.13 (dt, J=2.4, 7.2 Hz, 3H); LCMS (ESI, M+1): m/z=732.5.

### Example 40

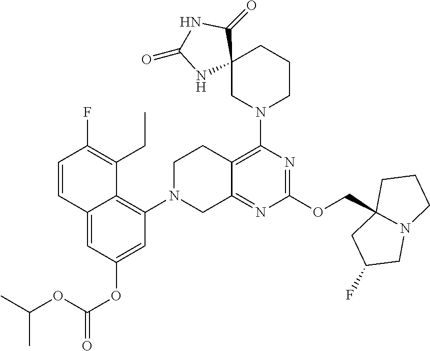 [SMILES: CCc1c(F)ccc2cc(OC(=O)OC(C)C)cc(N3CCc4c(nc(OC[C@@]56CCCN5C[C@H](F)C6)nc4N4CCC[C@]5(C4)NC(=O)NC5=O)C3)c12]

### 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl isopropyl carbonate

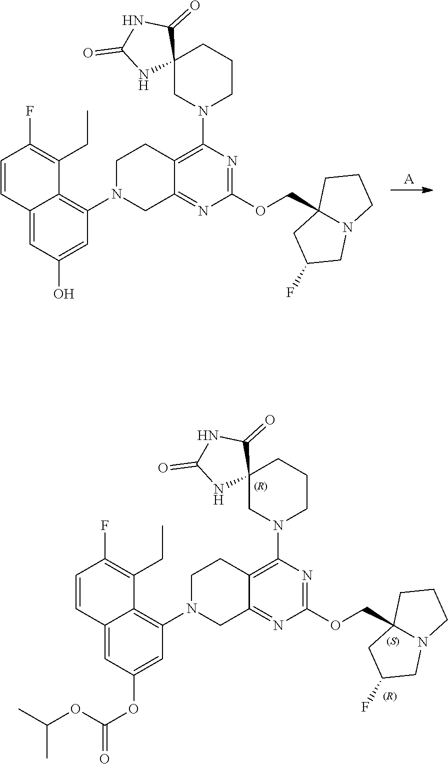

Step A. 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl isopropyl carbonate: To a solution of (R)-7-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione (200 mg, 1.00 equiv) and TEA (62.5 mg, 2.00 equiv) in THF (6 mL) was added isopropyl carbonochloridate (18.9 mg, 0.50 equiv) at −68° C. The reaction was stirred at −68° C. for 30 minutes. The mixture was quenched with NH₄Cl (5 mL) at 25° C., diluted with water (5 mL) and extracted with ethyl acetate (3×10 mL). The combined organic layers were washed with brine (10 mL), dried over Na₂SO₄, filtered, concentrated under reduced pressure and purified with prep-HPLC (Xtimate C18 150*40 mm*10 um; mobile phase: [water (NH₃)-ACN]; B %: 30%-60%, 7 min)) to afford the tittle compound (22.4 mg, 9.6% yield) as white solid; 1H NMR (400 MHz, DMSO-d6) δ=10.72 (br s, 1H), 8.63-8.47 (m, 1H), 7.86 (br dd, J=5.6, 8.8 Hz, 1H), 7.71-7.62 (m, 1H), 7.48-7.29 (m, 2H), 5.31-5.14 (m, 1H), 4.90 (qd, J=6.4, 12.5 Hz, 1H), 4.03-3.89 (m, 4H), 3.86-3.80 (m, 1H), 3.77-3.61 (m, 2H), 3.25-3.10 (m, 3H), 3.08-2.94 (m, 4H), 2.94-2.67 (m, 3H), 2.05 (br s, 1H), 2.01-1.86 (m, 4H), 1.86-1.63 (m, 6H), 1.36-1.29 (m, 6H), 1.07 (br t, J=7.2 Hz, 3H); LCMS (ESI, M+1): m/z=734.3.

### Example 41

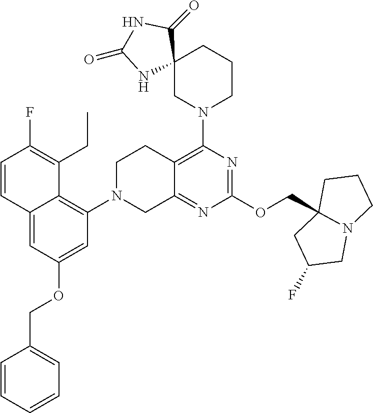 [SMILES: CCc1c(F)ccc2cc(OCc3ccccc3)cc(N3CCc4c(nc(OC[C@@]56CCCN5C[C@H](F)C6)nc4N4CCC[C@]5(C4)NC(=O)NC5=O)C3)c12]

### (R)-7-(7-(3-(benzyloxy)-8-ethyl-7-fluoronaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione

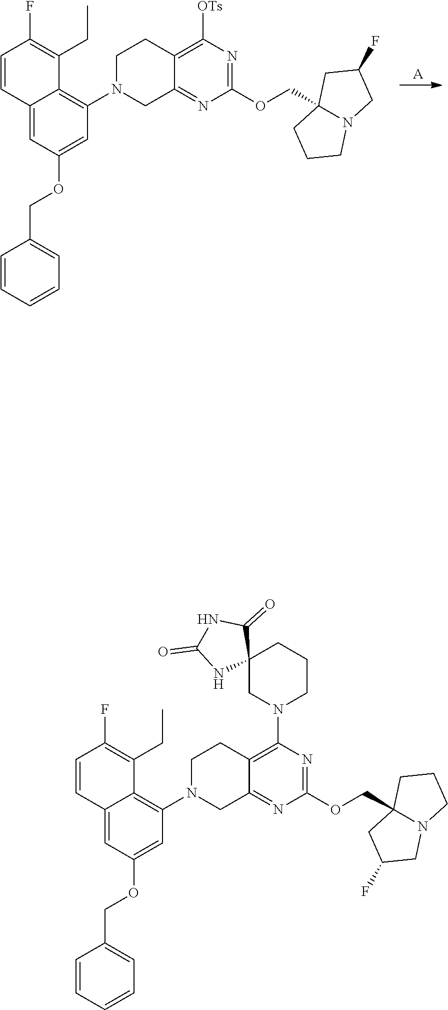

Step A. (R)-7-(7-(3-(benzyloxy)-8-ethyl-7-fluoronaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-1,3,7-tri azaspiro[4.5]decane-2,4-dione: To a mixture of 7-(3-(benzyloxy)-8-ethyl-7-fluoronaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl 4-methylbenzenesulfonate (80.0 mg, 1.0 equiv) and (R)-1,3,7-triazaspiro[4.5]decane-2,4-dione (27.4 mg, 1.5 equiv) in DMF (1 mL) were added DIEA (83.7 mg, 6.0 equiv) and 4 Å molecular sieve (10 mg). The reaction mixture was stirred at 60° C. for 12 hours. The mixture was filtered and purified with prep-HPLC (column: Phenomenex luna C18 150×25 mm×10 um; mobile phase: [water(FA)-ACN]; B %: 30%-60%, 8 min) to afford title compound (41.0 mg, 47% yield) as off-white solid; 1H NMR (400 MHz, Methanol-d4) δ=7.63 (br dd, J=6.0, 8.8 Hz, 1H), 7.48 (br d, J=6.8 Hz, 2H), 7.38 (br t, J=7.2 Hz, 2H), 7.34-7.28 (m, 1H), 7.23-7.15 (m, 2H), 7.09 (br d, J=9.2 Hz, 1H), 5.44-5.23 (m, 1H), 5.17 (s, 2H), 4.28-3.92 (m, 5H), 3.65 (br dd, J=4.4, 17.6 Hz, 1H), 3.55-3.33 (m, 7H), 3.26-2.98 (m, 4H), 2.83-2.67 (m, 1H), 2.46-2.22 (m, 2H), 2.21-1.80 (m, 8H), 1.17-1.06 (m, 3H); LCMS (ESI, M+1): m/z=738.4.

### Example 42

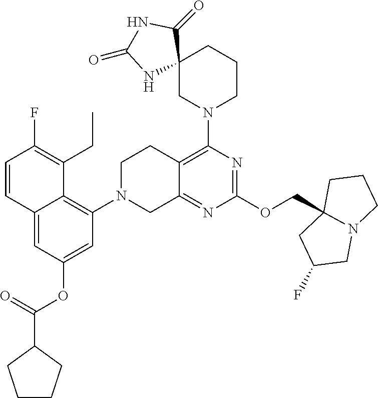 [SMILES: CCc1c(F)ccc2cc(OC(=O)C3CCCC3)cc(N3CCc4c(nc(OC[C@@]56CCCN5C[C@H](F)C6)nc4N4CCC[C@]5(C4)NC(=O)NC5=O)C3)c12]

### 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl cyclopentanecarboxylate

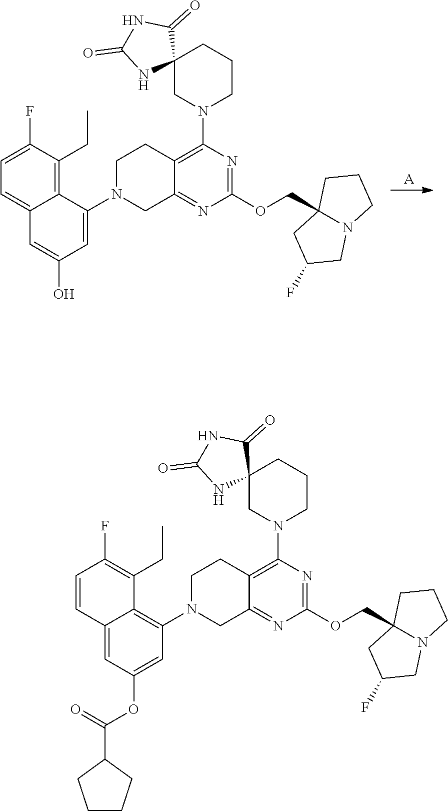

Step A: 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl cyclopentanecarboxylate: To a mixture of (R)-7-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione (100 mg, 1 equiv) and DIEA (59.9 mg, 3 equiv) in THF (1.0 mL) was added cyclopentanecarbonyl chloride (10.2 mg, 0.5 equiv) in one portion under N2. The mixture was stirred at −68° C. for 1.5 hours. The mixture was diluted with water (3 mL) and extracted with dichloromethane (3×2 mL). The combined organic layers were dried over Na₂SO₄, filtered, concentrated purified with prep-HPLC (column: Phenomenex Luna C18 150×25 mm×10 um; mobile phase: [water (FA)-ACN]; B %: 29%-59%, 10 min) to afford the title compound (12.1 mg, 10.0% yield) as off-white solid; 1H NMR (400 MHz, DMSO-d6) δ=10.77 (br s, 1H), 8.58-8.48 (m, 1H), 7.88-7.80 (m, 1H), 7.58-7.52 (m, 1H), 7.42 (t, J=9.3 Hz, 1H), 7.29-7.17 (m, 1H), 5.33-5.15 (m, 1H), 3.98-3.89 (m, 3H), 3.81 (br d, J=10.4 Hz, 1H), 3.67 (br d, J=17.6 Hz, 1H), 3.23 (br s, 4H), 3.14-3.01 (m, 6H), 2.97 (br s, 1H), 2.85-2.76 (m, 2H), 2.08-2.04 (m, 1H), 2.03-1.86 (m, 8H), 1.83-1.59 (m, 10H), 1.10-1.02 (m, 3H); F NMR (400 MHz, DMSO-d6) δ=116.97, −172.09; LCMS (ESI, M+1): m/z=744.5.

### Example 43

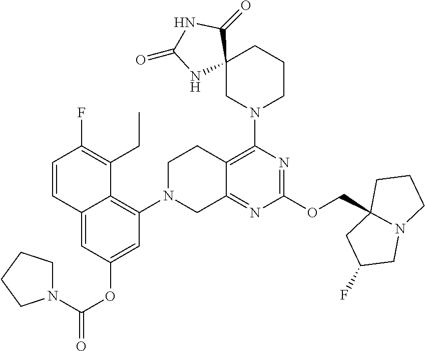 [SMILES: CCc1c(F)ccc2cc(OC(=O)N3CCCC3)cc(N3CCc4c(nc(OC[C@@]56CCCN5C[C@H](F)C6)nc4N4CCC[C@]5(C4)NC(=O)NC5=O)C3)c12]

### 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl pyrrolidine-1-carboxylate

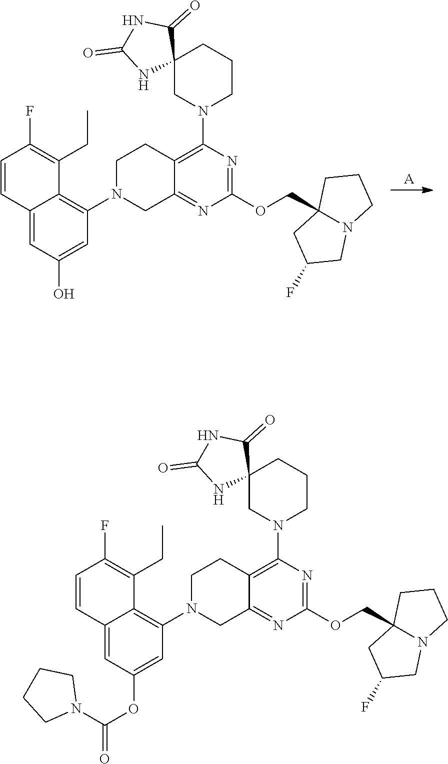

Step A. 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl pyrrolidine-1-carboxylate: To a solution of (R)-7-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione (100 mg, 1.0 equiv) in DMF (1 mL) were added K₂CO₃ (111 mg, 5.0 equiv) and pyrrolidine-1-carbonyl chloride (32.3 mg, 1.5 equiv) at 0° C. The reaction was stirred at 20° C. for 12 hours. The mixture was filtered, concentrated and purified with prep-HPLC [UniSil 3-100 C18 Ultra 150×25 mm×3 μm; A: water (FA), B: ACN; B %: 31%-51%, 7 min] to afford the title compound (31.1 mg, 36% yield) as white solid; 1H NMR (400 MHz, METHANOL-d4) δ=7.76-7.66 (m, 1H), 7.47-7.41 (m, 1H), 7.33-7.24 (m, 1H), 7.23-7.15 (m, 1H), 5.40-5.23 (m, 1H), 4.30-3.87 (m, 5H), 3.77-3.60 (m, 3H), 3.59-3.35 (m, 8H), 3.30-2.96 (m, 5H), 2.85-2.69 (m, 1H), 2.42-2.20 (m, 2H), 2.18-2.08 (m, 2H), 2.06-1.81 (m, 10H), 1.12 (t, J=7.2 Hz, 3H); LCMS (ESI, M+1): m/z=745.3.

### Example 44

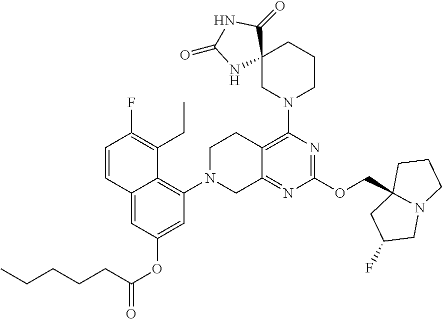 [SMILES: CCCCCC(=O)Oc1cc(N2CCc3c(nc(OC[C@@]45CCCN4C[C@H](F)C5)nc3N3CCC[C@]4(C3)NC(=O)NC4=O)C2)c2c(CC)c(F)ccc2c1]

### 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl hexanoate

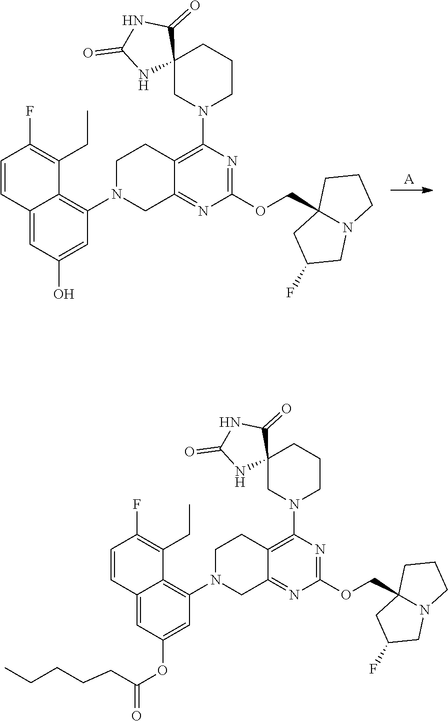

Step A. 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl hexanoate: To a solution of (5R)-9-[7-(8-ethyl-7-fluoro-3-hydroxy-1-naphthyl)-2-[[(2R,8S)-2-fluoro-1,2,3,5,6,7-hexahydropyrrolizin-8-yl]methoxy]-6,8-dihydro-5H-pyrido[3,4-d]pyrimidin-4-yl]-1,3,9-triazaspiro[4.5]decane-2,4-dione (100 mg, 1.0 equiv) and TEA (46.9 mg, 3.0 equiv) in DCM (2 mL) was added hexanoyl chloride (31.2 mg, 1.5 equiv) at 0° C. The reaction was stirred at 0° C. for 6 hours. The mixture was quenched by adding H₂O (5 mL) at 0° C. and extracted with DCM (3×5 mL). The combined organic layers were dried over Na₂SO₄, concentrated and purified by prep-HPLC [Waters Xbridge 150×25 mm×5 μm; A: water (NH₄HCO₃), B: ACN; B %: 70%-100%, 8 min] and [Waters Xbridge 150×25 mm×5 m; A: water (NH₄HCO₃), B: ACN; B %: 65%-95%, 8 min] to afford the title compound (4.89 mg, 4.0% yield) as off-white solid; 1H NMR (400 MHz, METHANOL-d4) δ=7.78-7.72 (m, 1H), 7.52-7.44 (m, 1H), 7.32-7.23 (m, 1H), 7.21-7.09 (m, 1H), 5.36-5.17 (m, 1H), 4.21-4.07 (m, 4H), 4.06-3.94 (m, 1H), 3.77-3.65 (m, 1H), 3.56-3.48 (m, 1H), 3.47-3.37 (m, 3H), 3.22-3.11 (m, 4H), 3.08-2.92 (m, 2H), 2.83-2.68 (m, 1H), 2.64-2.55 (m, 2H), 2.36-2.20 (m, 1H), 2.18-2.06 (m, 3H), 2.03-1.81 (m, 6H), 1.79-1.72 (m, 2H), 1.55-1.45 (m, 4H), 1.35-1.26 (m, 1H), 1.17-1.08 (m, 3H), 1.00-0.91 (m, 3H); LCMS (ESI, M+1): m/z=746.3.

### Example 45

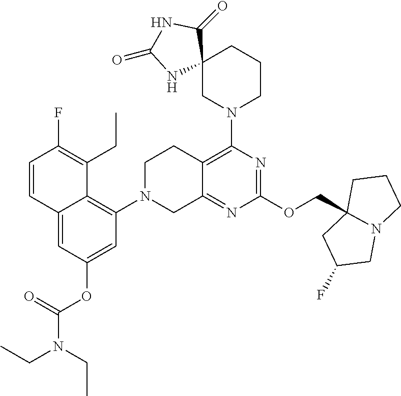 [SMILES: CCc1c(F)ccc2cc(OC(=O)N(CC)CC)cc(N3CCc4c(nc(OC[C@@]56CCCN5C[C@H](F)C6)nc4N4CCC[C@]5(C4)NC(=O)NC5=O)C3)c12]

### 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl diethylcarbamate

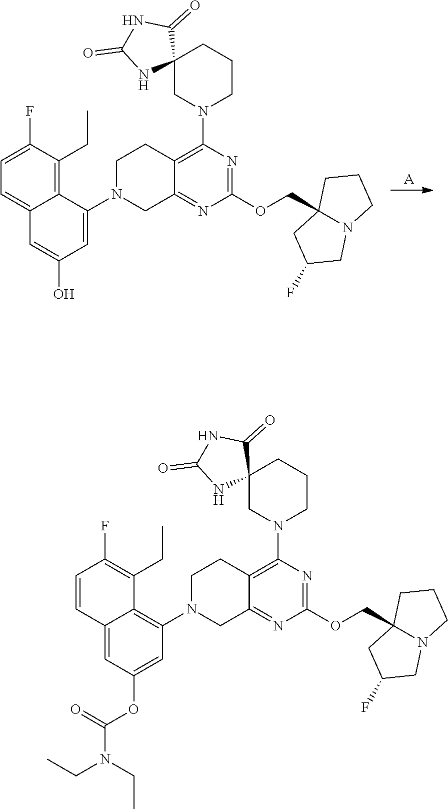

Step A. 4-(4-((R)-2,4-dioxo-1,3,7-triazaspiro[4.5]decan-7-yl)-2-(((2R,7aS)-2-fluorohexahydro-1H-pyrrolizin-7a-yl)methoxy)-5,6-dihydropyrido[3,4-d]pyrimidin-7(8H)-yl)-5-ethyl-6-fluoronaphthalen-2-yl diethylcarbamate: To a solution of (R)-7-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-1,3,7-triazaspiro[4.5]decane-2,4-dione (80.0 mg, 1.0 equiv) in DCM (4 mL) was added DMAP (15.1 mg, 1.0 equiv). After 5 minutes TEA (18.8 mg, 1.5 equiv) and diethylcarbamic chloride (25.1 mg, 1.5 equiv) were added to above mixture dropwise. The reaction was stirred at 40° C. for 5 hours. The mixture was quenched with water (4.0 mL) at 0-5° C. and extracted with DCM (2 mL×3). The combined organic layers was dried over anhydrous sodium sulfate, concentrated and purified with reversed-phase HPLC (column: Phenomenex luna C18 150×25 mm×10 um; mobile phase: [water (FA)-ACN]; B %: 33%-53%, 58 min) to afford the title compound (26.8 mg, 5.42% yield) as off-white solid; SFC: ee=23.5%, Column: Chiralcel OD-3 50×4.6 mm I.D., 3 um, Mobile phase: Phase A for CO₂, and Phase B for MeOH (0.05% DEA); Gradient elution: B in A from 5% to 40% Flow rate: 3 mL/min; Detector: PDA; Column Temp: 35 C; Back Pressure: 100 Bar, tR1=1.788, tR2=2.508; 1H NMR (400 MHz, DMSO-d6) δ=10.84-10.63 (m, 1H), 8.61-8.46 (m, 1H), 7.90-7.77 (m, 1H), 7.58-7.48 (m, 1H), 7.44-7.35 (m, 1H), 7.30-7.16 (m, 1H), 5.37-5.12 (m, 1H), 3.96-3.89 (m, 3H), 3.81 (br d, J=10.0 Hz, 1H), 3.69-3.65 (m, 1H), 3.49-3.46 (m, 2H), 3.19 (br d, J=12.4 Hz, 2H), 3.11-3.02 (m, 4H), 2.97 (br s, 1H), 2.85-2.76 (m, 2H), 2.71-2.57 (m, 2H), 2.11-2.04 (m, 2H), 2.02-1.88 (m, 5H), 1.86-1.78 (m, 2H), 1.78-1.67 (m, 4H), 1.23 (br d, J=6.4 Hz, 4H), 1.16-1.11 (m, 3H), 1.07 (br t, J=7.2 Hz, 3H); F NMR (400 MHz, DMSO-d6) δ=−117.423, −172.136; LCMS (ESI, M+1): m/z=747.1.

### Example 46

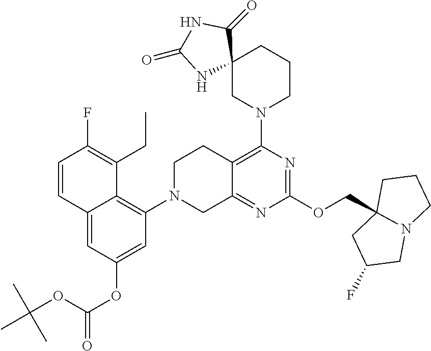 [SMILES: CCc1c(F)ccc2cc(OC(=O)OC(C)(C)C)cc(N3CCc4c(nc(OC[C@@]56CCCN5C[C@H](F)C6)nc4N4CCC[C@]5(C4)NC(=O)NC5=O)C3)c12]

### tert-butyl (4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl) carbonate

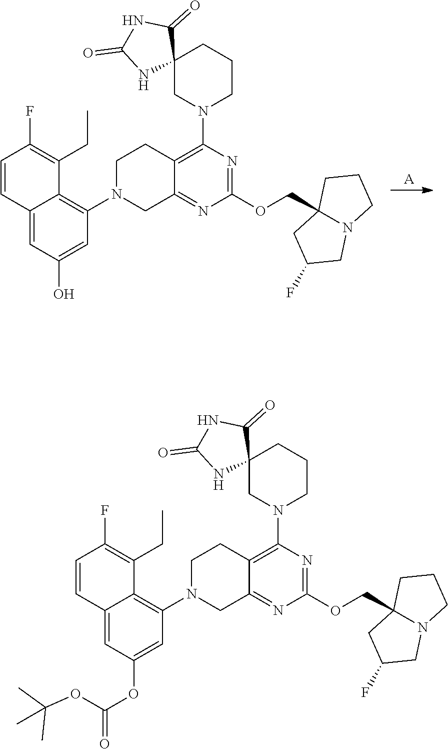

Step A. tert-butyl 4-(R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl) carbonate: To a solution of (R)-7-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione (50.0 mg, 1.0 equiv) and TEA (15.6 mg, 2.00 equiv) in THF (2.00 mL) was added (Boc)₂O (33.7 mg, 2.0 equiv) at 0° C. The reaction was stirred at 25° C. for 12 hours. The mixture was quenched with NH₄Cl (2.00 mL) at 25° C., diluted with water (5.00 mL) and extracted with ethyl acetate (2×10 mL). The combined organic layers were washed with brine (10 mL), dried over Na₂SO₄, filtered, concentrated under reduced pressure and purified with prep-HPLC (Xtimate C18 150*40 mm*10 um; mobile phase: [water (TFA)-ACN]; B %: 30%-60%, 10 min). The collected fractions were neutralized with saturated aqueous of NaHCO₃ (2 mL) and extracted with ethyl acetate (2×10 mL). The combined organic layers were dried over Na₂SO₄, filtered and concentrated under reduced pressure to afford the tittle compound (7.58 mg, 13% yield) as white solid. 1H NMR (400 MHz, DMSO-d6) δ=10.86-10.67 (m, 1H), 8.67-8.47 (m, 1H), 7.87 (br s, 1H), 7.71-7.59 (m, 1H), 7.43 (br t, J=8.8 Hz, 1H), 7.37-7.23 (m, 1H), 5.32-5.13 (m, 1H), 4.10-3.51 (m, 6H), 3.28-2.94 (m, 8H), 2.94-2.62 (m, 3H), 2.11-1.88 (m, 5H), 1.86-1.59 (m, 6H), 1.57-1.48 (m, 9H), 1.07 (br s, 3H); LCMS (ESI, M+1): m/z=748.5.

### Example 47

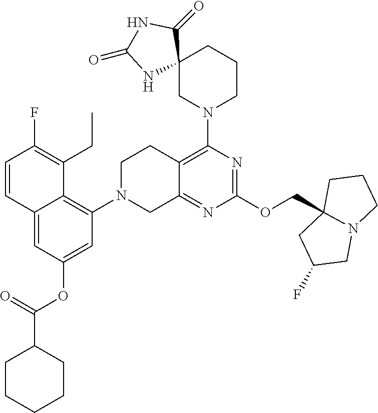 [SMILES: CCc1c(F)ccc2cc(OC(=O)C3CCCCC3)cc(N3CCc4c(nc(OC[C@@]56CCCN5C[C@H](F)C6)nc4N4CCC[C@]5(C4)NC(=O)NC5=O)C3)c12]

### 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl cyclohexanecarboxylate

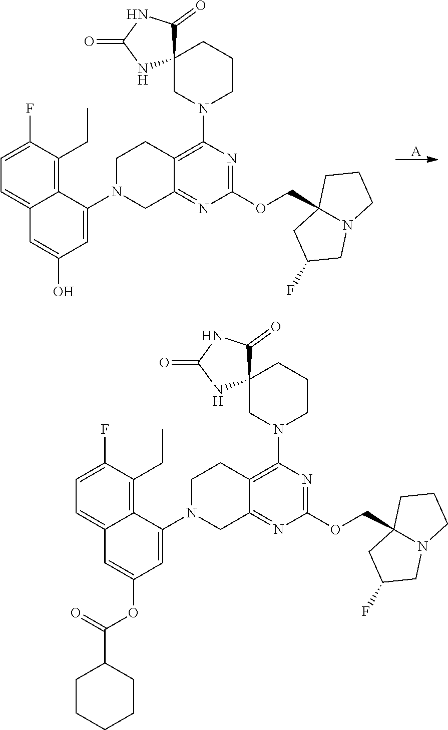

Step A. 4-(4-((R)-2,4-dioxo-1,3,7-triazaspiro[4.5]decan-7-yl)-2-(((2R,7aS)-2-fluorohexahydro-1H-pyrrolizin-7a-yl)methoxy)-5,6-dihydropyrido[3,4-d]pyrimidin-7(8H)-yl)-5-ethyl-6-fluoronaphthalen-2-yl cyclohexanecarboxylate: To a solution of (R)-7-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-1,3,7-triazaspiro[4.5]decane-2,4-dione (80.0 mg, 1.0 equiv) and DIEA (47.9 mg, 3.0 equiv) in THF (0.80 mL) was added cyclohexanecarbonyl chloride (9.05 mg, 0.50 equiv) at −68° C. The reaction was stirred at −68° C. for 1 hour. The mixture was quenched with water (2 mL) at 0-5° C. and extracted with DCM (1 mL×3). The combined organic layers were dried over anhydrous sodium sulfate, filtered, concentrated and purified with reversed-phase HPLC (column: Phenomenex luna C18 150×25 mm×10 um; mobile phase: [water (FA)-ACN]; B %: 31%-61%, 10 min) to afford the title compound (29.9 mg, 10.3% yield) as off-white solid; SFC: ee=21.572%, Column: Chiralcel OD-3 50×4.6 mm I.D., 3 um, Mobile phase: Phase A for CO₂, and Phase B for MeOH+ACN(0.05% DEA); Isocratic elution: 40% B in A, Flow rate: 3 mL/min; Detector: PDA; Column Temp: 35 C; Back Pressure: 100 Bar, tR1=0.507, tR2=1.126; 1H NMR (400 MHz, DMSO-d6) δ=10.87-10.63 (m, 1H), 8.60-8.47 (m, 1H), 7.90-7.80 (m, 1H), 7.57-7.49 (m, 1H), 7.46-7.37 (m, 1H), 7.28-7.14 (m, 1H), 5.36-5.13 (m, 1H), 4.05-3.85 (m, 4H), 3.81 (br d, J=10.4 Hz, 1H), 3.71-3.63 (m, 1H), 3.13-3.02 (m, 4H), 2.99-2.95 (m, 1H), 2.94-2.85 (m, 1H), 2.85-2.74 (m, 2H), 2.72-2.57 (m, 2H), 2.10-2.00 (m, 3H), 1.99 (br s, 1H), 1.93 (br s, 3H), 1.81 (br s, 2H), 1.74 (m, 6H), 1.68-1.46 (m, 4H), 1.44-1.16 (m, 4H), 1.12-1.03 (m, 3H); F NMR (400 MHz, DMSO-d6) δ=−116.943; −172.150; LCMS (ESI, M+1): m/z=758.4.

### Example 48

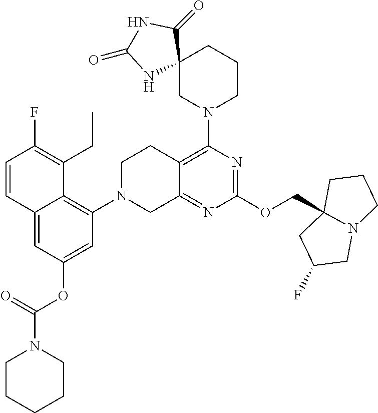 [SMILES: CCc1c(F)ccc2cc(OC(=O)N3CCCCC3)cc(N3CCc4c(nc(OC[C@@]56CCCN5C[C@H](F)C6)nc4N4CCC[C@]5(C4)NC(=O)NC5=O)C3)c12]

### 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl piperidine-1-carboxylate

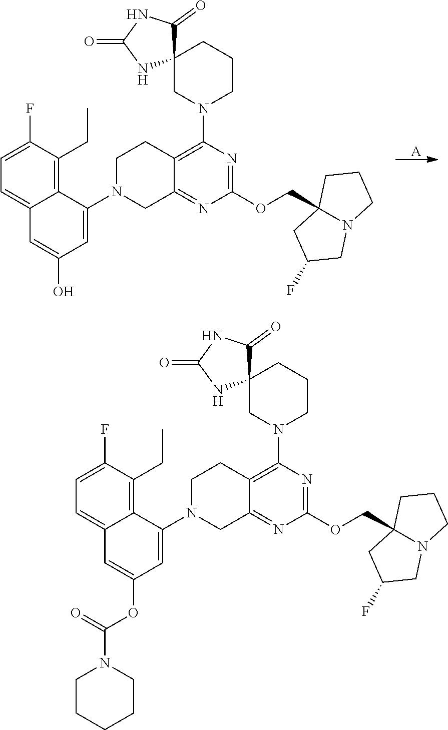

Step A. 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl piperidine-1-carboxylate: To a solution of (R)-7-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione (50 mg, 1.0 equiv) and K₂CO₃ (53.3 mg, 5.0 equiv) in DMF (0.5 mL) was added piperidine-1-carbonyl chloride (13.7 mg, 1.2 equiv). The reaction mixture was stirred at 20° C. for 12 hours. The reaction mixture was filtered, concentrated and purified with prep-HPLC [Phenomenex C18 150×25 mm×10 μm; A: water (FA), B: ACN, B %: 26%-56% over 10 min] to afford the title compound (19.2 mg, 33% yield) as white solid; 1H NMR (400 MHz, METHANOL-d4) δ=7.73 (dd, J=6.0, 8.8 Hz, 1H), 7.41 (t, J=2.4 Hz, 1H), 7.28 (t, J=9.6 Hz, 1H), 7.17 (dd, J=2.4, 7.2 Hz, 1H), 5.45-5.23 (m, 1H), 4.28-3.93 (m, 5H), 3.80-3.62 (m, 3H), 3.58-3.35 (m, 9H), 3.25-2.98 (m, 4H), 2.85-2.67 (m, 1H), 2.46-2.21 (m, 2H), 2.21-2.08 (m, 2H), 2.07-1.78 (m, 6H), 1.75-1.58 (m, 6H), 1.12 (dt, J=2.4, 7.2 Hz, 3H); LCMS (ESI, M+1): m/z=759.6.

### Example 49

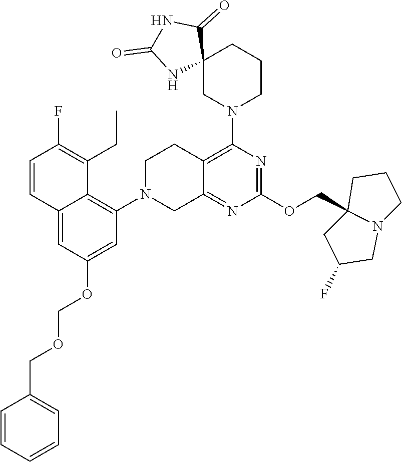 [SMILES: CCc1c(F)ccc2cc(OCOCc3ccccc3)cc(N3CCc4c(nc(OC[C@@]56CCCN5C[C@H](F)C6)nc4N4CCC[C@]5(C4)NC(=O)NC5=O)C3)c12]

### (R)-7-(7-(3-((benzyloxy)methoxy)-8-ethyl-7-fluoronaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione

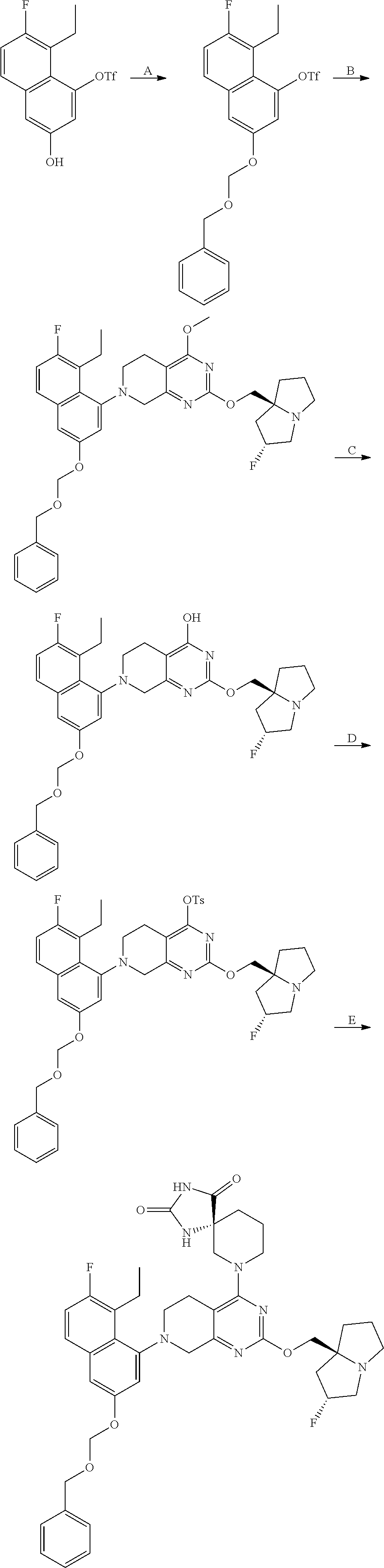

Step A. 3-((benzyloxy)methoxy)-8-ethyl-7-fluoronaphthalen-1-yl trifluoromethanesulfonate: To a solution of 8-ethyl-7-fluoro-3-hydroxy-8,8a-dihydronaphthalen-1-yl trifluoromethanesulfonate (2.00 g, 1.0 equiv) in dichloromethane (15 mL) were added DIEA (2.28 g, 3.0 equiv) and ((chloromethoxy)methyl)benzene (1.38 g, 1.5 equiv) at 0° C. The mixture was stirred at 25° C. for 1 hour. The mixture was diluted with water (10 mL) and extracted with dichloromethane (2×10 mL). The organic layer was dried over anhydrous Na₂SO₄, concentrated and purified with column chromatography [SiO₂, petroleum ether/ethyl acetate=I/O to 5/1] to afford the title compound (2.40 g, 88% yield) as colorless oil; 1H NMR (400 MHz, CD3OD) δ=7.72 (dd, J=5.6, 9.2 Hz, 1H), 7.57 (d, J=2.4 Hz, 1H), 7.38-7.34 (m, 1H), 7.33-7.28 (m, 4H), 7.27-7.23 (m, 2H), 5.42 (s, 2H), 4.74 (s, 2H), 3.27-3.17 (m, 2H), 1.20 (t, J=7.6 Hz, 3H).

Step B. 7-(3-((benzyloxy)methoxy)-8-ethyl-7-fluoronaphthalen-1-yl)-2-(((2R,7aS)-2-fluorotetrahydro-1H-pyrrolizin-7a(5H)-yl)methoxy)-4-methoxy-5,6,7,8-tetrahydropyrido[3,4-d]pyrimidine: To a mixture of 2-(((2R,7aS)-2-fluorotetrahydro-1H-pyrrolizin-7a(5H)-yl)methoxy)-4-methoxy-5,6,7,8-tetrahydropyrido[3,4-d]pyrimidine (600 mg, 1.0 equiv) and 3-((benzyloxy)methoxy)-8-ethyl-7-fluoronaphthalen-1-yl trifluoromethanesulfonate (1.02 g, 1.2 equiv) in dioxane (5 mL) were added Xantphos Pd G4 (179 mg, 0.1 equiv) and Cs₂CO₃ (1.82 g, 3.0 equiv). The reaction mixture was stirred at 90° C. for 12 hours under N₂ atmosphere. The mixture was diluted with water (10 mL) and extracted with ethyl acetate (2×10 mL). The organic layer was dried over anhydrous Na₂SO₄, concentrated and purified with reversed phase flash [C18, 0.1% formic acid condition] to afford the title compound (160 mg, 13% yield) as yellow oil; LCMS (ESI, M+1): m/z=631.5.

Step C. 7-(3-((benzyloxy)methoxy)-8-ethyl-7-fluoronaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-ol: To a mixture of NaH (20.1 mg, 60% purity, 2.0 equiv) in DMAC (2 mL) was added EtSH (87.2 mg, 5.5 equiv) slowly at 0° C. under N₂ atmosphere. The mixture was stirred at 25° C. for 10 minutes. 7-(3-((benzyloxy)methoxy)-8-ethyl-7-fluoronaphthalen-1-yl)-2-(((2R,7aS)-2-fluorotetrahydro-1H-pyrrolizin-7a(5H)-yl)methoxy)-4-methoxy-5,6,7,8-tetrahydropyrido[3,4-d]pyrimidine (160 mg, 1.0 equiv) was added at 0° C. and the reaction mixture was stirred at 60° C. for 1 hour. The mixture was quenched with saturated NH₄Cl aqueous (10 mL) at 0° C. and extracted with ethyl acetate (3×10 mL). The organic layer was washed with brine (10 mL), dried over anhydrous Na₂SO₄ and concentrated to afford the title compound (220 mg, crude) as yellow solid.

Step D. 7-(3-((benzyloxy)methoxy)-8-ethyl-7-fluoronaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl 4-methylbenzenesulfonate: To a solution of 7-(3-((benzyloxy)methoxy)-8-ethyl-7-fluoronaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-ol (200 mg, 1.0 equiv) and DIEA (126 mg, 3.0 equiv) in dichloromethane (2 mL) were added DMAP (3.96 mg, 0.1 equiv) and 4-methylbenzenesulfonyl chloride (92.7 mg, 1.5 equiv) at 0° C. The reaction mixture was stirred at 25° C. for 2 hours. The mixture was diluted with water (5 mL) and extracted with dichloromethane (2×5 mL). The organic layer was washed with brine (5 mL), dried over anhydrous Na₂SO₄ and concentrated to afford the title compound (80.0 mg, crude) as yellow oil.

Step E. (R)-7-(7-(3-((benzyloxy)methoxy)-8-ethyl-7-fluoronaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione: To a solution of 7-(3-((benzyloxy)methoxy)-8-ethyl-7-fluoronaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl 4-methylbenzenesulfonate (70 mg, 1.0 equiv) and (R)-1,3,7-triazaspiro[4.5]decane-2,4-dione (30.7 mg, 2.0 equiv) in DMF (0.5 mL) were added DIEA (23.5 mg, 2.0 equiv) and 4 Å molecular sieve (10 mg). The reaction mixture was stirred at 40° C. for 12 hours. The mixture was filtered and purified with reversed phase flash [C18, 0.1% formic acid condition] and prep-HPLC[column: Waters Xbridge 150×25 mm×5 m; A: water (NH₄HCO₃), B: (ACN), B %: 65%-95% over 8 minutes] to afford the title compound (18.4 mg, 26% yield); ¹H NMR (400 MHz, CD3OD) δ=7.62 (dd, J=6.0, 8.8 Hz, 1H), 7.38-7.08 (m, 8H), 5.41 (d, J=4.0 Hz, 2H), 5.35-5.16 (m, 1H), 4.75 (d, J=3.2 Hz, 2H), 4.27-3.87 (m, 5H), 3.65 (br d, J=17.6 Hz, 1H), 3.48 (br d, J=9.6 Hz, 1H), 3.45-3.35 (m, 3H), 3.30-2.93 (m, 7H), 2.83-2.66 (m, 1H), 2.37-2.06 (m, 4H), 2.04-1.75 (m, 6H), 1.11 (dt, J=3.2, 7.2 Hz, 3H); LCMS (ESI, M+1): m/z=768.5.

### Example 50

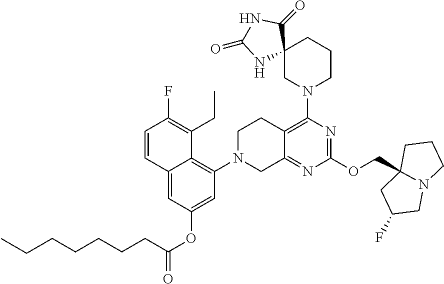 [SMILES: CCCCCCCC(=O)Oc1cc(N2CCc3c(nc(OC[C@@]45CCCN4C[C@H](F)C5)nc3N3CCC[C@]4(C3)NC(=O)NC4=O)C2)c2c(CC)c(F)ccc2c1]

### 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl octanoate

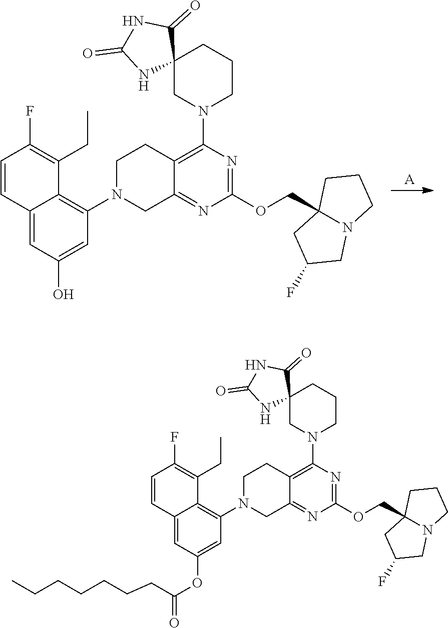

Step A. 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl octanoate: To a solution of (R)-7-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione (50.0 mg, 1.0 equiv) and TEA (15.6 mg, 2.0 equiv) in THF (2 mL) was added octanoyl chloride (25.1 mg, 2.0 equiv) at 0° C. The reaction was stirred at 25° C. for 12 hours. The mixture was diluted with water (3 mL) at 25° C. and extracted with ethyl acetate (3×10 mL). The combined organic layers were washed with brine (10 mL), dried over Na₂SO₄, filtered, concentrated under reduced pressure and purified with prep-HPLC (Xbridge Prep OBD C18 150 mm*30 mm*5 um column (eluent: 30% to 60% CH₃CN and H₂O with 0.05% TFA). The collected fractions were neutralized with aqueous of NaHCO₃ (2 M, 2 mL), extracted with ethyl acetate (3×10 mL), dried over Na₂SO₄, filtered and concentrated under reduced pressure to afford the title compound (4.02 mg, 6.8% yield) as white solid; 1H NMR (400 MHz, DMSO-d6) δ=10.78 (br s, 1H), 8.66-8.47 (m, 1H), 7.85 (br s, 1H), 7.57-7.50 (m, 1H), 7.42 (br t, J=9.0 Hz, 1H), 7.29-7.20 (m, 1H), 5.31-5.16 (m, 1H), 4.06-3.87 (m, 4H), 3.86-3.79 (m, 1H), 3.74-3.62 (m, 1H), 3.21-2.94 (m, 7H), 2.92-2.71 (m, 3H), 2.62 (br d, J=7.2 Hz, 2H), 2.09-1.89 (m, 6H), 1.83-1.62 (m, 8H), 1.28 (br s, 6H), 1.07 (br s, 3H), 0.86 (br d, J=7.2 Hz, 5H); LCMS (ESI, M+1): m/z=774.2.

### Example 51

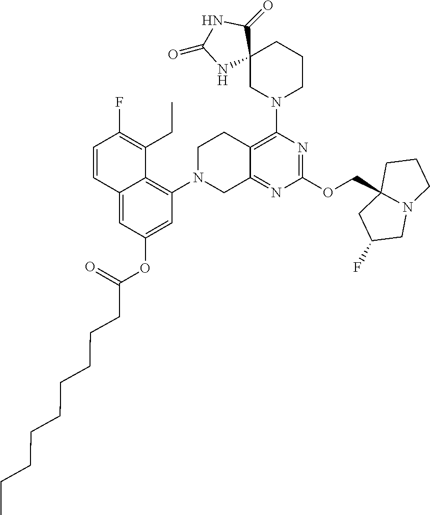 [SMILES: CCCCCCCCCC(=O)Oc1cc(N2CCc3c(nc(OC[C@@]45CCCN4C[C@H](F)C5)nc3N3CCC[C@]4(C3)NC(=O)NC4=O)C2)c2c(CC)c(F)ccc2c1]

### 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl decanoate

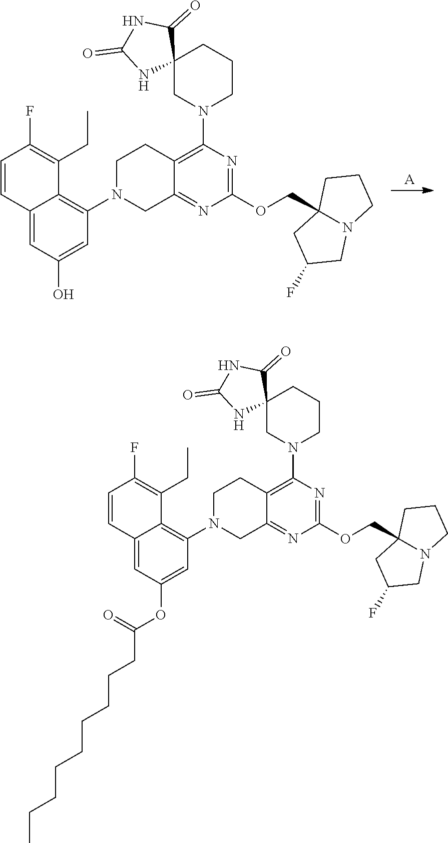

Step A. 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl decanoate: To a solution of (R)-7-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione (100 mg, 1.0 equiv) and TEA (63 mg, 4.0 equiv) in THF (1.5 mL) was added decanoyl chloride (24 mg, 0.8 equiv) dropwise at −40° C. under N2 atmosphere. The reaction mixture was stirred at −40° C. for 2 hours. The mixture was quenched with anhydrous methanol (1 mL) at −40° C. and stirred for 10 minutes. The mixture was diluted with water (5 mL) and extracted with ethyl acetate (3×3 mL). The organic layer was dried over anhydrous Na₂SO₄, concentrated and purified with prep-HPLC [column: Phenomenex C18 75×30 mm×3 m; mobile phase: water (FA)-ACN; B %: 35%-65% over 5 minutes] to afford the title compound (30 mg, 24% yield) as pink solid; 1H NMR (400 MHz, CD3OD) δ=7.71 (dd, J=6.0, 8.8 Hz, 1H), 7.41 (dd, J=2.4, 3.6 Hz, 1H), 7.27 (t, J=9.6 Hz, 1H), 7.14 (dd, J=2.4, 13.2 Hz, 1H), 5.40-5.16 (m, 1H), 4.26-3.90 (m, 5H), 3.69 (br d, J=17.6 Hz, 1H), 3.55-3.32 (m, 5H), 3.29-2.96 (m, 6H), 2.80-2.56 (m, 3H), 2.36-1.67 (m, 12H), 1.50-1.23 (m, 12H), 1.12 (dt, J=1.6, 7.2 Hz, 3H), 0.96-0.81 (m, 3H); LCMS (ESI, M+1): m/z=802.6.

### Example 52

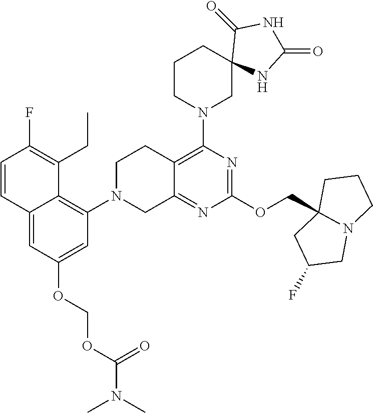 [SMILES: CCc1c(F)ccc2cc(OCOC(=O)N(C)C)cc(N3CCc4c(nc(OC[C@@]56CCCN5C[C@H](F)C6)nc4N4CCC[C@]5(C4)NC(=O)NC5=O)C3)c12]

### ((4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl)oxy)methyl dimethylcarbamate

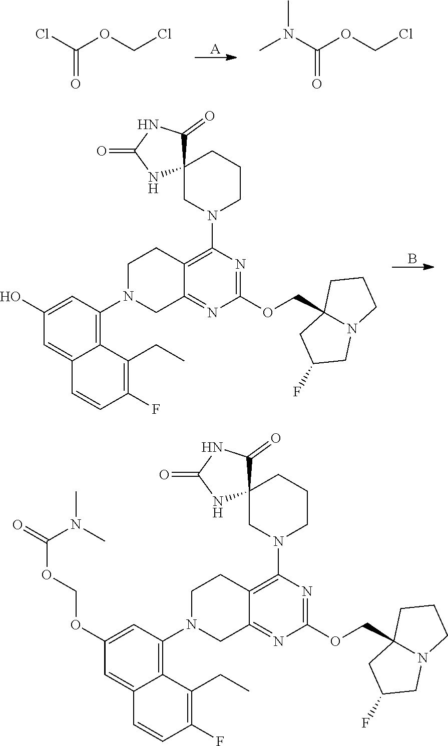

Step A. chloromethyl dimethylcarbamate: To a solution of chloromethyl carbonochloridate (2.79 g, 1.0 equiv) and DIEA (8.39 g, 3.0 equiv) in THE (10 mL) was added dimethylamine (2 M, 6.49 mL, 0.60 equiv) at −60° C. slowly. The reaction was stirred at −60° C. for 0.5 hours. The mixture was quenched with water (30 mL) at 0° C. and extracted with ethyl acetate (4×30 mL). The combined organic layers were washed with brine (30 mL), dried over anhydrous sodium sulfate, concentrated and purified with column chromatography [SiO2, Petroleum ether/Ethyl acetate=50/1 to 10/1] to afford the title compound (600 mg, 20% yield) as yellow oil; 1H NMR (400 MHz, CHLOROFORM-d) δ=6.05-5.52 (m, 2H), 3.11-2.83 (m, 6H).

Step B. ((4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl)oxy)methyl dimethylcarbamate: To a mixture of (R)-7-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione (200 mg, 1.0 equiv) and K₂CO₃ (128 mg, 3.0 equiv) in DMF (1.0 mL) was added chloromethyl dimethylcarbamate (33.9 mg, 0.80 equiv). The reaction was stirred at 20° C. for 12 hours. The mixture was diluted with water (10 mL) and extracted with ethyl acetate (3×10 mL). The combined organic layers were washed with brine (20 mL), dried over anhydrous sodium sulfate, concentrated and purified with prep-TLC [SiO2, Dichloromethane:Methanol=10/1] and prep-HPLC [Phenomenex luna C18 150×25 mm×10 μm; A: water (FA), B: ACN; B %: 15%-45% over 10 min]. The product was further purified with SFC [DAICEL CHIRALPAK AS 250 mm×30 mm, 10 m; A: [ACN, B: EtOH (0.1% NH₃H₂O), B %: 30%-30% over 3.8 min] and prep-HPLC [Phenomenex luna C18 150×25 mm×10 μm; A: water (FA), B: ACN; B %: 16%-46% over 10 min] to afford the title compound (3.44 mg, 1.5% yield, 0.18 HCOOH) as white solid; 1H NMR (400 MHz, DMSO-d6) δ=11.00-10.44 (m, 1H), 8.63-8.47 (m, 1H), 7.84-7.72 (m, 1H), 7.46-7.28 (m, 2H), 7.24-7.06 (m, 1H), 5.90-5.77 (m, 2H), 5.36-5.11 (m, 1H), 4.05-3.74 (m, 6H), 3.73-3.55 (m, 2H), 3.21 (br s, 3H), 3.09-2.91 (m, 5H), 2.80-2.78 (m, 8H), 2.10-1.88 (m, 5H), 1.84-1.69 (m, 5H), 1.11-1.00 (m, 3H); LCMS (ESI, M+1): m/z=749.4.

### Example 53

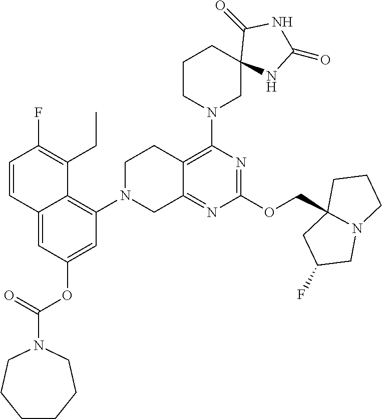 [SMILES: CCc1c(F)ccc2cc(OC(=O)N3CCCCCC3)cc(N3CCc4c(nc(OC[C@@]56CCCN5C[C@H](F)C6)nc4N4CCC[C@]5(C4)NC(=O)NC5=O)C3)c12]

### 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl azepane-1-carboxylate

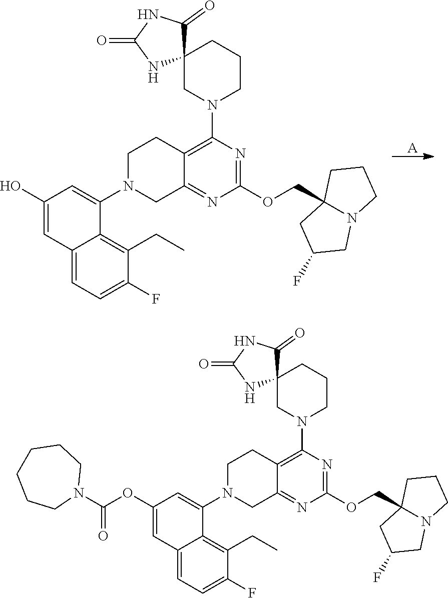

Step A. 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl azepane-1-carboxylate: To a solution of (R)-7-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione (100 mg, 1.0 equiv) and TEA (31.2 mg, 2.0 equiv) in DCM (1.0 mL) was added 4-nitrophenyl carbonochloridate (31.1 mg, 1.0 equiv) at −10° C. The mixture was stirred at this temperature for 0.5 hours, and then azepane (76.6 mg, 5.0 equiv) was added at −10° C. The reaction was stirred at 20° C. for 12 hours. The mixture was diluted with water (5.0 mL) and extracted with DCM (5.0 mL×3). The combined organic layers were dried over anhydrous sodium sulfate, concentrated and purified with prep-HPLC [Waters Xbridge 150×25 mm×5 μm; A: water (NH₄HCO₃), B: ACN, B %: 60%-90% over 9 min] to afford the title compound (8.35 mg, 6.7% yield) as white solid; 1H NMR (400 MHz, DMSO-d6) δ=10.87-10.70 (m, 1H), 8.67-8.44 (m, 1H), 7.96-7.81 (m, 1H), 7.59-7.50 (m, 1H), 7.41 (t, J=9.2 Hz, 1H), 7.32-7.12 (m, 1H), 5.75-5.13 (m, 1H), 4.05-3.70 (m, 5H), 3.67-3.53 (m, 3H), 3.50-3.40 (m, 4H), 3.31-2.97 (m, 7H), 2.95-2.67 (m, 3H), 2.11-1.99 (m, 2H), 1.99-1.92 (m, 2H), 1.92-1.61 (m, 10H), 1.60-1.48 (m, 4H), 1.11-1.03 (m, 3H); LCMS (ESI, M+1): m/z=773.4.

### Example 54

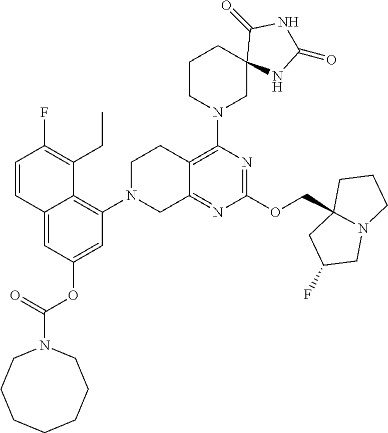 [SMILES: CCc1c(F)ccc2cc(OC(=O)N3CCCCCCC3)cc(N3CCc4c(nc(OC[C@@]56CCCN5C[C@H](F)C6)nc4N4CCC[C@]5(C4)NC(=O)NC5=O)C3)c12]

### 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl azocane-1-carboxylate

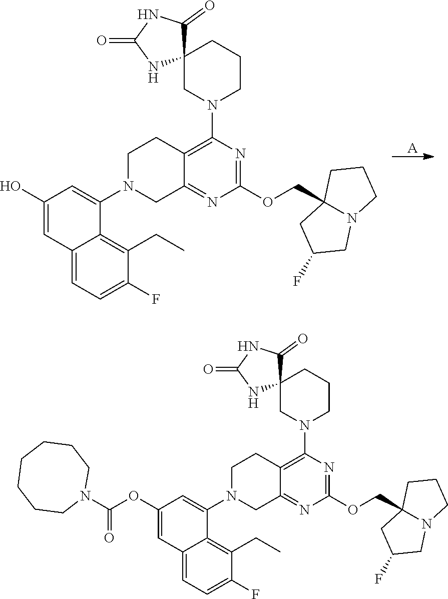

Step A. 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl azocane-1-carboxylate: To a solution of (R)-7-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione (150 mg, 1.0 equiv) and TEA (46.7 mg, 2.0 equiv) in DCM (1.0 mL) was added 4-nitrophenyl carbonochloridate (46.7 mg, 1.0 equiv) at −10° C. The mixture was stirred at this temperature for 0.5 hours, and then azocane (26.2 mg, 1.0 equiv) was added at −10° C. The reaction was stirred at 20° C. for 12 hours. The mixture was diluted with water (5.0 mL) and extracted with DCM (3×5 mL). The combined organic layers were dried over anhydrous sodium sulfate, concentrated and purified with prep-HPLC [Waters Xbridge 150×25 mm×5 μm; A: water (NH₄HCO₃), B: ACN, B %: 64%-94% over 9 min] to afford the title compound (28.2 mg, 15% yield) as off-white solid; 1H NMR (400 MHz, DMSO-d6) δ=10.89-10.64 (m, 1H), 8.65-8.42 (m, 1H), 7.98-7.76 (m, 1H), 7.56-7.46 (m, 1H), 7.40 (t, J=9.2 Hz, 1H), 7.26-7.08 (m, 1H), 5.39-5.09 (m, 1H), 4.10-3.75 (m, 5H), 3.75-3.49 (m, 3H), 3.48-3.36 (m, 4H), 3.30-2.94 (m, 7H), 2.94-2.65 (m, 3H), 2.12-1.98 (m, 2H), 1.97-1.91 (m, 2H), 1.90-1.64 (m, 10H), 1.63-1.44 (m, 6H), 1.16-1.02 (m, 3H); LCMS (ESI, M+1): m/z=787.4.

### Example 55

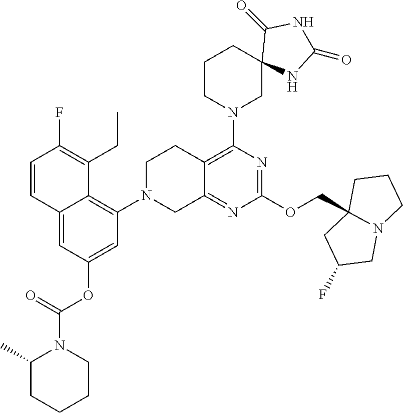 [SMILES: CCc1c(F)ccc2cc(OC(=O)N3CCCC[C@@H]3C)cc(N3CCc4c(nc(OC[C@@]56CCCN5C[C@H](F)C6)nc4N4CCC[C@]5(C4)NC(=O)NC5=O)C3)c12]

### 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl(S)-2-methylpiperidine-1-carboxylate

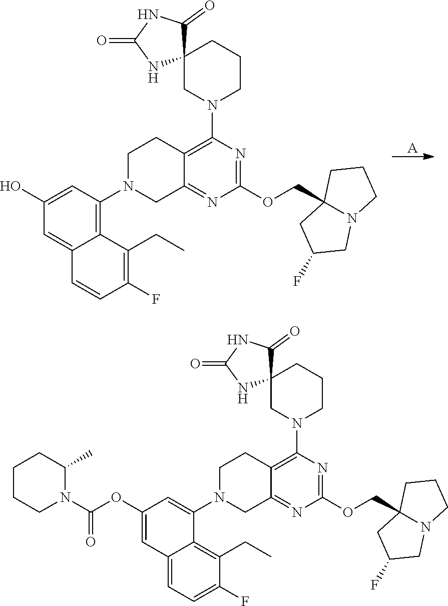

Step A. 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl (S)-2-methylpiperidine-1-carboxylate. To a solution of (R)-7-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione (100 mg, 1.0 equiv) and TEA (31.2 mg, 2.0 equiv) in DCM (3.0 mL) was added 4-nitrophenyl carbonochloridate (31.1 mg, 1.0 equiv) at −10° C. The mixture was stirring at −10° C. for 30 minutes, and then (S)-2-methylpiperidine (15.3 mg, 1.0 equiv) was added into the mixture. The reaction was stirred at 20° C. for 12 hours. The mixture was diluted with water (5.0 mL) and extracted with dichloromethane (5.0 mL×3). The combined organic layers were washed with brine (20 mL), dried over anhydrous sodium sulfate, concentrated and purified with prep-HPLC [Phenomenex luna C18 150×25 mm×10 μm; A: water (FA), B: ACN; B %: 26%-56% over 10 min] and [Waters Xbridge 150×25 mm×5 μm; A: water (NH₄HCO₃), B: ACN]; B %: 61%-91% over 9 min] to afford the title compound (23.3 mg, 19% yield) as white solid; 1H NMR (400 MHz, DMSO-d6) δ=10.79-10.53 (m, 1H), 8.63-8.46 (m, 1H), 7.89-7.78 (m, 1H), 7.59-7.48 (m, 1H), 7.42-4.40 (m, 1H), 7.31-7.15 (m, 1H), 5.35-5.11 (m, 1H), 4.56-4.36 (m, 1H), 3.81 (s, 6H), 3.71-3.64 (m, 1H), 3.22-3.13 (m, 2H), 3.14-3.11 (m, 6H), 2.87-2.56 (m, 6H), 2.11-2.04 (m, 1H), 2.03-1.87 (m, 4H), 1.83-1.80 (m, 1H), 1.70-1.67 (m, 6H), 1.61-1.53 (m, 2H), 1.48-1.35 (m, 1H), 1.25-1.23 (m, 3H), 1.09-1.05 (m, 3H); LCMS (ESI, M+1): m/z=773.5.

### Example 56

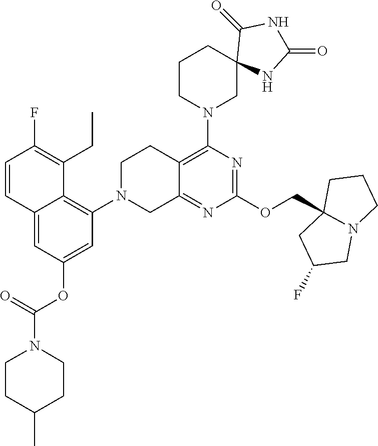 [SMILES: CCc1c(F)ccc2cc(OC(=O)N3CCC(C)CC3)cc(N3CCc4c(nc(OC[C@@]56CCCN5C[C@H](F)C6)nc4N4CCC[C@]5(C4)NC(=O)NC5=O)C3)c12]

### 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl 4-methylpiperidine-1-carboxylate

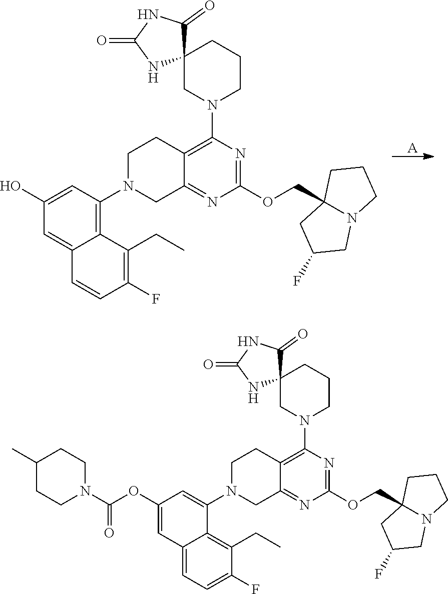

Step A. 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl 4-methylpiperidine-1-carboxylate: To a solution of (R)-7-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione (100 mg, 1.0 equiv) and TEA (31.2 mg, 2.0 equiv) in DCM (3.0 mL) was added 4-nitrophenyl carbonochloridate (31.1 mg, 1.0 equiv) at −10° C. The mixture was stirring at −10° C. for 30 minutes, and then 4-methylpiperidine (15.3 mg, 1.0 equiv) was added into the mixture. The reaction was stirred at 20° C. for 12 hours. The mixture was diluted with water (5.0 mL) and extracted with dichloromethane (5.0 mL×3). The combined organic layers were washed with brine (20 mL), dried over anhydrous sodium sulfate, concentrated and purified with prep-HPLC [Phenomenex luna C18 150×25 mm×10 μm; A: water (FA), B: ACN; B %: 27%-57% over 10 min] and [Waters Xbridge 150×25 mm×5 μm; A: water (NH₄HCO₃), B: ACN]; B %: 63%-93% over 10 min] to afford the title compound (11.1 mg, 9.1% yield) as white solid; 1H NMR (400 MHz, DMSO-d6) δ=10.87-10.11 (m, 1H), 8.59-8.42 (m, 1H), 7.85-7.80 (m, 1H), 7.58-7.49 (m, 1H), 7.42-7.38 (m, 1H), 7.29-7.15 (m, 1H), 5.37-5.10 (m, 1H), 4.24-4.12 (m, 1H), 4.02-3.80 (m, 6H), 3.73-3.66 (m, 1H), 3.21-3.15 (m, 2H), 3.09-2.95 (m, 6H), 2.93-2.75 (m, 6H), 2.06-2.01 (m, 1H), 1.99-1.86 (m, 4H), 1.93-1.81 (m, 8H), 1.25-1.21 (m, 1H), 1.12-1.02 (m, 4H), 0.99-0.91 (m, 3H); LCMS (ESI, M+1): m/z=773.3.

### Example 57

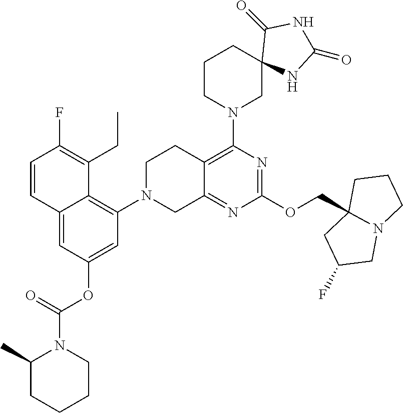 [SMILES: CCc1c(F)ccc2cc(OC(=O)N3CCCC[C@H]3C)cc(N3CCc4c(nc(OC[C@@]56CCCN5C[C@H](F)C6)nc4N4CCC[C@]5(C4)NC(=O)NC5=O)C3)c12]

### 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl (R)-2-methylpiperidine-1-carboxylate

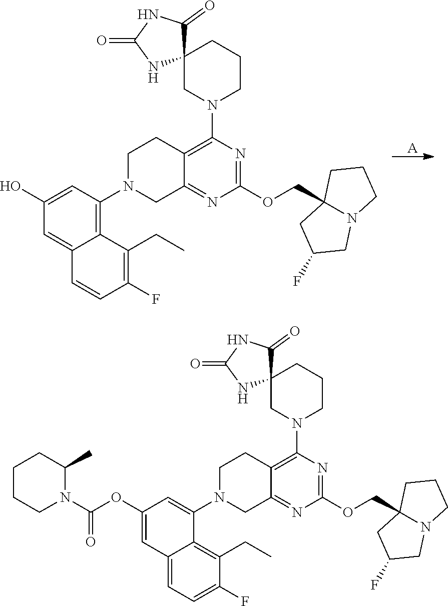

Step A. 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl (R)-2-methylpiperidine-1-carboxylate: To a solution of (R)-7-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione (100 mg, 1.0 equiv) and TEA (31.2 mg, 2.0 equiv) in DCM (1.0 mL) was added 4-nitrophenyl carbonochloridate (31.1 mg, 1.0 equiv) at −10° C. The mixture was stirred at −10° C. for 0.5 hours, and then (R)-2-methylpiperidine (76.7 mg, 5.0 equiv) was added. The reaction was stirred at 20° C. for 12 hours. The mixture was diluted with water (5.0 mL) and extracted with DCM (5.0 mL×3). The combined organic layers were dried over anhydrous sodium sulfate, concentrated and purified with prep-HPLC [Waters Xbridge 150×25 mm×5 μm; A: water (NH₄HCO₃), B: ACN, B %: 61%-91% over 9 min] to afford the title compound (24.7 mg, 20% yield) as white solid; 1H NMR (400 MHz, DMSO-d6) δ=10.85-10.52 (m, 1H), 8.68-8.35 (m, 1H), 7.93-7.75 (m, 1H), 7.60-7.48 (m, 1H), 7.40 (t, J=9.2 Hz, 1H), 7.31-7.14 (m, 1H), 5.39-5.09 (m, 1H), 4.57-4.37 (m, 1H), 4.11-3.59 (m, 7H), 3.42 (br s, 1H), 3.27-2.95 (m, 8H), 2.92-2.74 (m, 3H), 2.68-2.62 (m, 1H), 2.09-1.92 (m, 4H), 1.92-1.70 (m, 6H), 1.69-1.35 (m, 6H), 1.32-1.22 (m, 3H), 1.15-0.97 (m, 3H); LCMS (ESI, M+1): m/z=773.4.

### Example 58

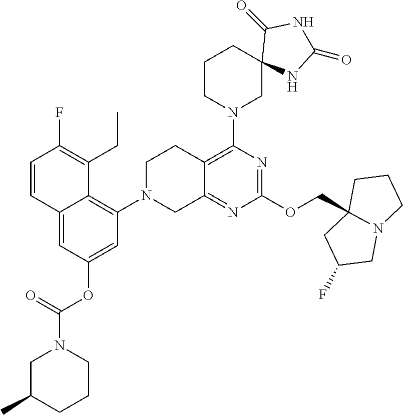 [SMILES: CCc1c(F)ccc2cc(OC(=O)N3CCC[C@@H](C)C3)cc(N3CCc4c(nc(OC[C@@]56CCCN5C[C@H](F)C6)nc4N4CCC[C@]5(C4)NC(=O)NC5=O)C3)c12]

### 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl (R)-3-methylpiperidine-1-carboxylate

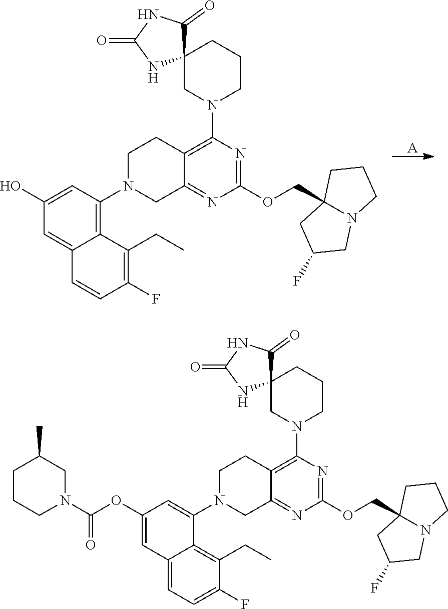

Step A: 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl (R)-3-methylpiperidine-1-carboxylate: To a solution of (R)-7-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione (100 mg, 1.0 equiv) and TEA (31.2 mg, 2.0 equiv) in DCM (1.0 mL) was added (4-nitrophenyl) carbonochloridate (31.1 mg, 1.0 equiv) at −10° C. The mixture was stirring at −10° C. for 20 minutes, and then (R)-3-methylpiperidine hydrochloride (31.29 mg, 1.5 equiv) was added. The reaction was stirred at 20° C. for 12 hours. The mixture was diluted with water (5.0 mL) and extracted with DCM (5.0 mL×3). The combined organic layers were dried over anhydrous sodium sulfate, concentrated and purified with prep-HPLC (Phenomenex luna C18 150*25 mm*10 um; mobile phase: [water(FA)-ACN]; B %: 26%-56%, 10 min) to afford the title compound (30.2 mg, 23.5% yield, 0.33 HCOOH) as a white solid. 1H NMR (400 MHz, DMSO-d6) δ=10.90-10.65 (m, 1H), 8.63-8.49 (m, 1H), 7.89-7.80 (m, 1H), 7.58-7.50 (m, 1H), 7.41 (t, J=9.2 Hz, 1H), 7.32-7.15 (m, 1H), 5.34-5.15 (m, 1H), 4.04-3.63 (m, 8H), 3.27-3.11 (m, 5H), 3.10-2.94 (m, 5H), 2.94-2.67 (m, 4H), 2.16-1.87 (m, 5H), 1.86-1.52 (m, 9H), 1.22-1.12 (m, 1H), 1.08 (br t, J=7.2 Hz, 3H), 0.91 (br s, 3H); LCMS (ESI, M+1): m/z=773.4.

### Example 59

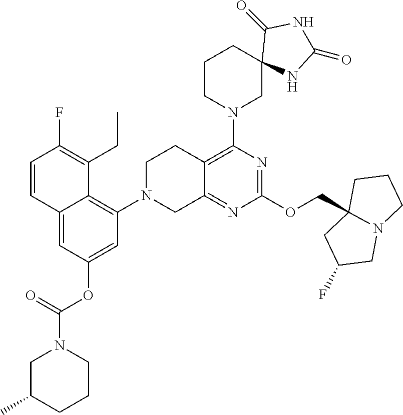 [SMILES: CCc1c(F)ccc2cc(OC(=O)N3CCC[C@H](C)C3)cc(N3CCc4c(nc(OC[C@@]56CCCN5C[C@H](F)C6)nc4N4CCC[C@]5(C4)NC(=O)NC5=O)C3)c12]

### 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl (S)-3-methylpiperidine-1-carboxylate

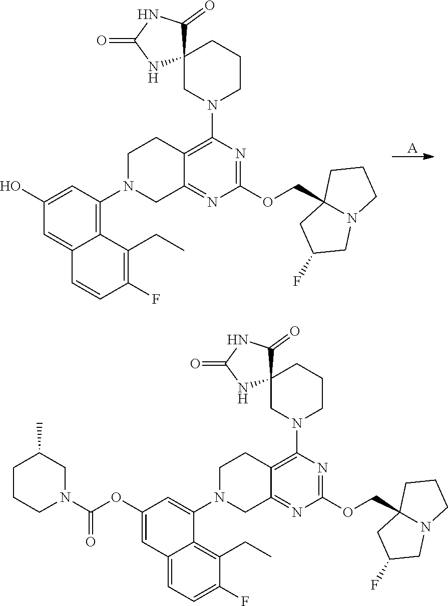

Step A: 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl (S)-3-methylpiperidine-1-carboxylate: To a solution of (R)-7-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione (100 mg, 1.0 equiv) and TEA (31.2 mg, 2.0 equiv) in DCM (1.0 mL) was added (4-nitrophenyl) carbonochloridate (31.1 mg, 1.0 equiv) at −10° C. The mixture was stirred at −10° C. for 20 minutes, and then (S)-3-methylpiperidine hydrochloride (31.29 mg, 1.5 equiv) was added. The reaction was stirred at 20° C. for 12 hours. The mixture was diluted with water (5.0 mL) and extracted with DCM (5.0 mL×3). The combined organic layers were dried over anhydrous sodium sulfate, concentrated and purified with prep-HPLC (column: Phenomenex luna C18 150*25 mm*10 um; mobile phase: [water(FA)-ACN]; gradient: 27%-57% B over 10 min) to afford the title compound (30.0 mg, 23.2% yield, 0.2 HCOOH) as a white solid. 1H NMR (400 MHz, DMSO-d6) δ=10.87 (s, 1H), 8.62-8.50 (m, 1H), 7.88-7.78 (m, 1H), 7.60-7.49 (m, 1H), 7.46-7.36 (m, 1H), 7.31-7.16 (m, 1H), 5.35-5.13 (m, 1H), 4.03-3.63 (m, 8H), 3.14 (br s, 5H), 3.10-2.94 (m, 5H), 2.92-2.66 (m, 4H), 2.10-1.87 (m, 5H), 1.86-1.57 (m, 9H), 1.22-1.12 (m, 1H), 1.11-1.03 (m, 3H), 0.95-0.86 (m, 3H); LCMS (ESI, M+1): m/z=773.4.

### Example 60

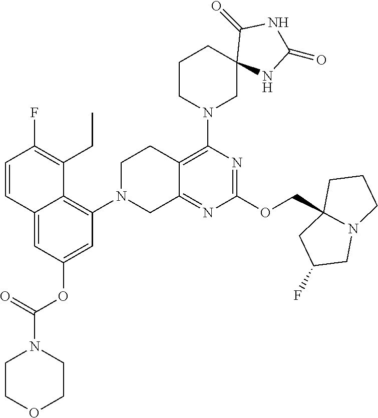 [SMILES: CCc1c(F)ccc2cc(OC(=O)N3CCOCC3)cc(N3CCc4c(nc(OC[C@@]56CCCN5C[C@H](F)C6)nc4N4CCC[C@]5(C4)NC(=O)NC5=O)C3)c12]

### 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl morpholine-4-carboxylate

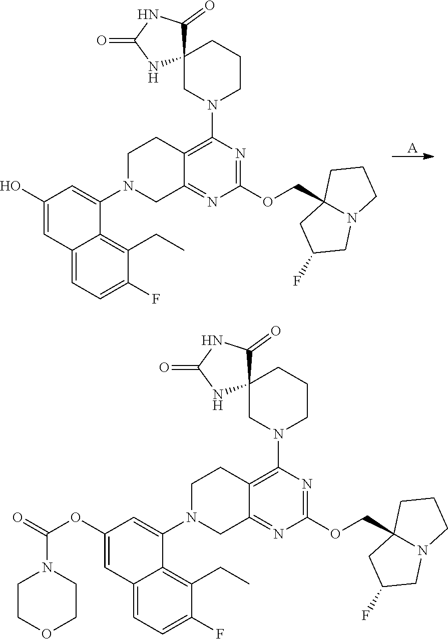

Step A. 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl morpholine-4-carboxylate: To a mixture of (R)-7-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione (100 mg, 1.0 equiv) and morpholine-4-carbonyl chloride (23.1 mg, 1.0 equiv) in DMF (2 mL) was added K₂CO₃ (64.0 mg, 3.0 equiv). The reaction was stirred at 20° C. for 4 hours. The mixture was diluted with water (10 mL) and extracted with ethyl acetate (2×10 mL). The combined organic layers were washed with brine (10 mL), dried over anhydrous sodium sulfate, concentrated and purified with prep-HPLC [column: YMC-Actus Triart C18 150×30 mm×7 μm; mobile phase: water(FA)-ACN; gradient: 25%-55% B over 10 minutes] to afford the title compound (61.2 mg, 49% yield, HCOOH) as off-white solid; 1H NMR (400 MHz, DMSO-d6) δ=10.88-10.63 (m, 1H), 8.66-8.43 (m, 1H), 8.14 (s, 1H), 7.83 (m, 1H), 7.59-7.53 (m, 1H), 7.41 (t, J=9.2 Hz, 1H), 7.33-7.20 (m, 1H), 5.36-5.14 (m, 1H), 4.07-3.77 (m, 5H), 3.48-2.77 (m, 21H), 2.08-1.69 (m, 10H), 1.07 (br t, J=7.2 Hz, 3H); LCMS (ESI, M+1): m/z=761.4.

### Example 61

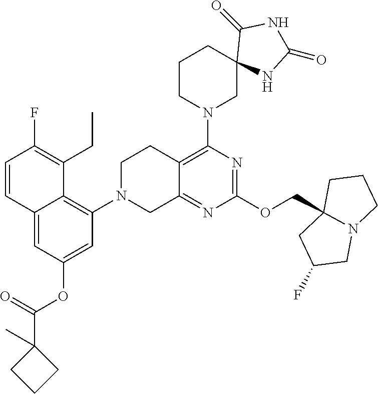 [SMILES: CCc1c(F)ccc2cc(OC(=O)C3(C)CCC3)cc(N3CCc4c(nc(OC[C@@]56CCCN5C[C@H](F)C6)nc4N4CCC[C@]5(C4)NC(=O)NC5=O)C3)c12]

### 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl 1-methylcyclobutane-1-carboxylate

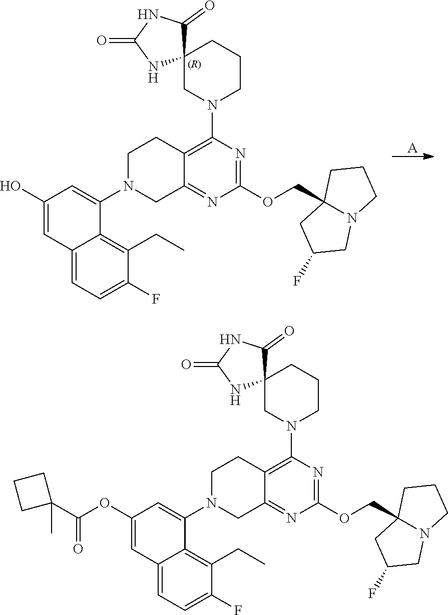

Step A. 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl 1-methylcyclobutane-1-carboxylate: To a solution of (R)-7-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione (50.0 mg, 1.0 equiv) and TEA (37.0 mg, 5.0 equiv) in DCM (1 mL) were added 1-methylcyclobutanecarboxylic acid (47.3 mg, 5.7 equiv) and isobutyl carbonochloridate (47.3 mg, 4.7 equiv). The reaction was stirred at 25° C. for 12 hours. The mixture was quenched with saturated NaHCO₃ aqueous solution (5 mL) and extracted with Ethyl acetate (3×10 mL). The combined organic layers were washed with brine (3×5 mL), dried over anhydrous sodium sulfate, concentrated and purified with by prep-HPLC [Phenomenex luna C18 150×25 mm×10 um; mobile phase: water(FA)-ACN; gradient: 25%-55% B over 10 min] to afford the title compound (10.2 mg, 18% yield, HCOOH) as off-white solid; 1H NMR (400 MHz, DMSO-d6) δ=10.00-9.87 (m, 1H), 7.78-7.65 (m, 1H), 7.33 (s, 1H), 7.08-7.07 (m, 1H), 7.02 (br s, 1H), 6.86-6.70 (m, 1H), 6.64-6.54 (m, 1H), 6.52-6.35 (m, 1H), 4.50-4.32 (m, 1H), 3.23-2.79 (m, 7H), 2.39-1.73 (m, 13H), 1.26-1.06 (m, 8H), 0.99-0.88 (m, 4H), 0.74-0.70 (m, 2H), 0.24 (br s, 2H), 0.12 (br d, J=6.0 Hz, 2H); LCMS (ESI, M+1): m/z=744.6.

### Example 62

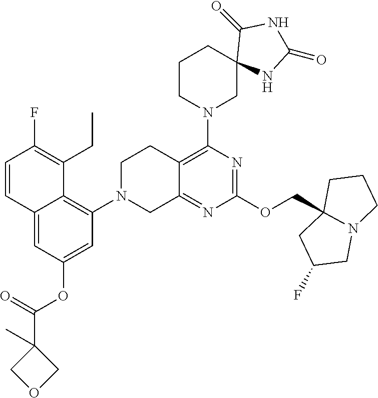 [SMILES: CCc1c(F)ccc2cc(OC(=O)C3(C)COC3)cc(N3CCc4c(nc(OC[C@@]56CCCN5C[C@H](F)C6)nc4N4CCC[C@]5(C4)NC(=O)NC5=O)C3)c12]

### 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl 3-methyloxetane-3-carboxylate

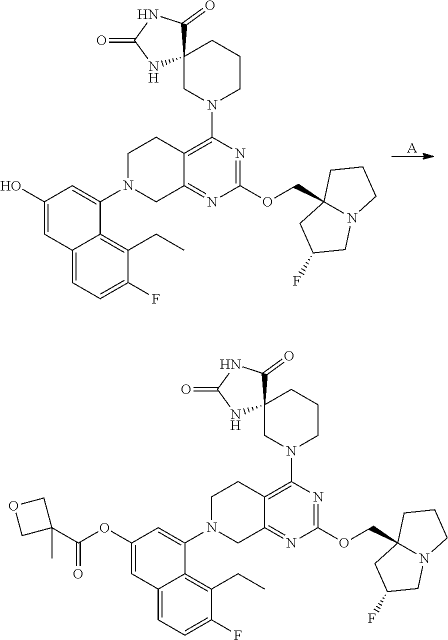

Step A. 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl 3-methyloxetane-3-carboxylate: To a solution of 3-methyloxetane-3-carboxylic acid (20.0 mg, 1.0 equiv) in DCM (1 mL) were added TEA (87.2 mg, 5.0 equiv) and isobutyl carbonochloridate (23.5 mg, 1.0 equiv). The reaction was stirred at 0° C. for 0.5 hours and (R)-7-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione (112 mg, 1.0 equiv) was added. The reaction was stirred at 0° C. for 0.5 hours. The mixture was diluted with water (10 mL) and extracted with ethyl acetate (2×10 mL). The combined organic layers were washed with brine (10 mL), dried over anhydrous sodium sulfate, concentrated and purified with prep-HPLC [column: YMC-Actus Triart C18 150×30 mm×7 μm; mobile phase: water (formic acid v/v, B: ACN); gradient: 25%-55% B over 10 minutes] to afford the title compound (11.8 mg, 8.7% yield, HCOOH) as pink solid; 1H NMR (400 MHz, DMSO-d6) δ=10.87-10.68 (m, 1H), 8.65-8.46 (m, 1H), 8.21 (s, 1H), 7.90-7.81 (m, 1H), 7.69-7.59 (m, 1H), 7.49-7.30 (m, 2H), 5.36-5.12 (m, 1H), 5.06-4.98 (m, 2H), 4.52-4.44 (m, 2H), 4.06-3.65 (m, 7H), 3.22-2.71 (m, 11H), 2.07-1.70 (m, 13H), 1.08 (dt, J=2.4, 7.2 Hz, 3H); LCMS (ESI, M+1): m/z=746.3.

### Example 63

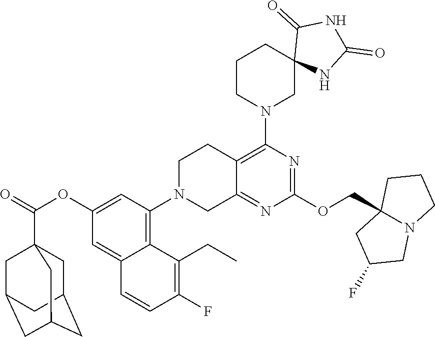 [SMILES: CCc1c(F)ccc2cc(OC(=O)C34CC5CC(CC(C5)C3)C4)cc(N3CCc4c(nc(OC[C@@]56CCCN5C[C@H](F)C6)nc4N4CCC[C@]5(C4)NC(=O)NC5=O)C3)c12]

### 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl (3r,5r,7r)-adamantane-1-carboxylate

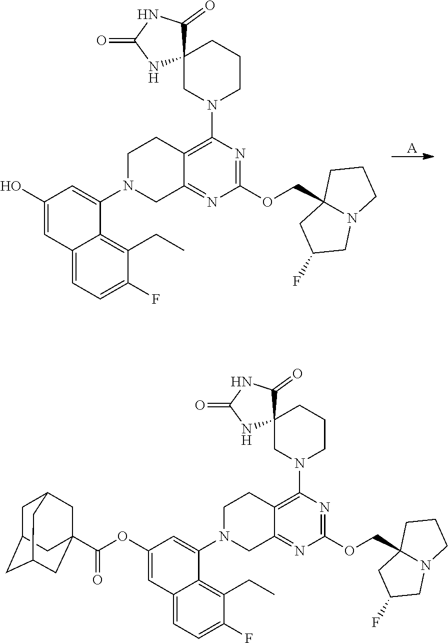

Step A. 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl (3r,5r,7r)-adamantane-1-carboxylate: To a solution of (R)-7-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione (2.0 g, 1.0 equiv) in DMF (100 mL) were added K₂CO₃ (3.41 g, 8.0 equiv) and adamantane-1-carbonyl chloride (3.07 g, 5.0 equiv). The reaction was stirred at 40° C. for 18 hours. The mixture was quenched with saturated aqueous NaHCO3 (30 mL) and extracted with ethyl acetate (3×30 mL). The combined organic layers were dried over Na₂SO₄, concentrated and purified with prep-HPLC [column: Waters Xbridge Prep OBD C18 150×40 mm×10 μm; A: water (NH₄HCO₃), B: ACN; B %: 65%-95% over 15 min] to afford the title compound (395 mg, 15% yield) as an off-white solid; 1H NMR (400 MHz, DMSO-d6) δ=10.85-10.67 (m, 1H), 8.60-8.46 (m, 1H), 7.91-7.79 (m, 1H), 7.56-7.37 (m, 2H), 7.28-7.12 (m, 1H), 5.34-5.13 (m, 1H), 4.02-3.64 (m, 6H), 3.49-3.35 (m, 2H), 3.29-3.00 (m, 6H), 2.99-2.60 (m, 4H), 2.09-2.01 (m, 10H), 2.00-1.87 (m, 4H), 1.82 (br s, 1H), 1.79-1.67 (m, 10H), 1.07 (br t, J=7.1 Hz, 3H); LCMS (ESI, M+1): m/z=810.4.

### Example 64

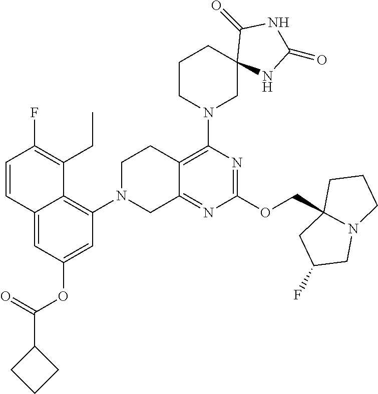 [SMILES: CCc1c(F)ccc2cc(OC(=O)C3CCC3)cc(N3CCc4c(nc(OC[C@@]56CCCN5C[C@H](F)C6)nc4N4CCC[C@]5(C4)NC(=O)NC5=O)C3)c12]

### 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl cyclobutanecarboxylate

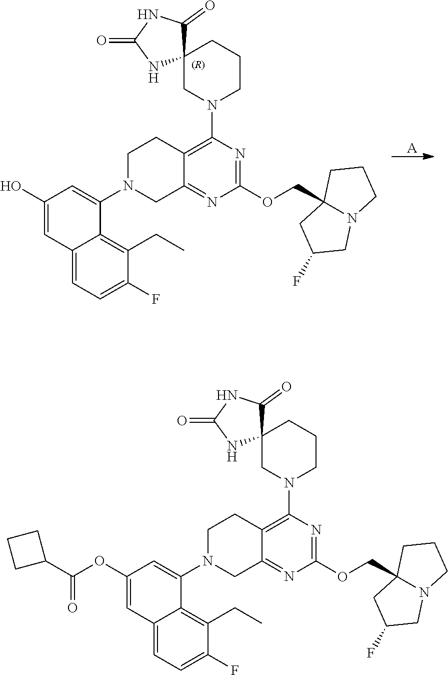

Step A. 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl cyclobutanecarboxylate: To a solution of (R)-7-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione (200 mg, 1.0 equiv) in DMF (3 mL) were added cyclobutanecarbonyl chloride (36.6 mg, 1.0 equiv) and K₂CO₃ (128 mg, 3.0 equiv). The reaction was stirred at 25° C. for 12 hours. The mixture was diluted with H₂O (15 mL) and extracted with ethyl acetate (2×10 mL). The combined organic layers were washed with brine (20 mL), dried over anhydrous Na₂SO₄, concentrated and purified with prep-HPLC [column: Waters Xbridge 150×25 mm×5 um; mobile phase: water (ammonia hydroxide v/v, B: ACN); gradient: 56%-86% B over 9 min] to afford the title compound (26.0 mg, 11% yield) as off-white solid; 1H NMR (400 MHz, DMSO-d6) δ=10.84-10.68 (m, 1H), 8.66-8.43 (m, 1H), 7.86-7.82 (m, 1H), 7.61-7.53 (m, 1H), 7.42 (t, J=9.2 Hz, 1H), 7.34-7.13 (m, 1H), 5.35-5.08 (m, 1H), 4.04-3.63 (m, 6H), 3.55-3.41 (m, 2H), 3.30-2.93 (m, 8H), 2.92-2.58 (m, 3H), 2.41-2.25 (m, 4H), 2.10-1.86 (m, 7H), 1.83-1.68 (m, 5H), 1.07 (br t, J=7.2 Hz, 3H); LCMS (ESI, M+1): m/z=730.4.

### Example 65

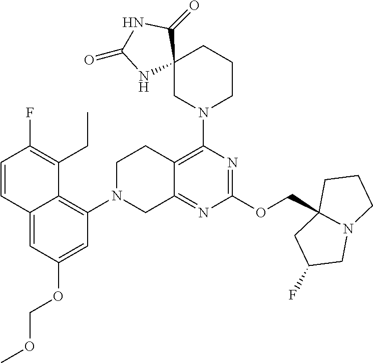 [SMILES: CCc1c(F)ccc2cc(OCOC)cc(N3CCc4c(nc(OC[C@@]56CCCN5C[C@H](F)C6)nc4N4CCC[C@]5(C4)NC(=O)NC5=O)C3)c12]

### 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl bis(2-methoxyethyl)carbamate

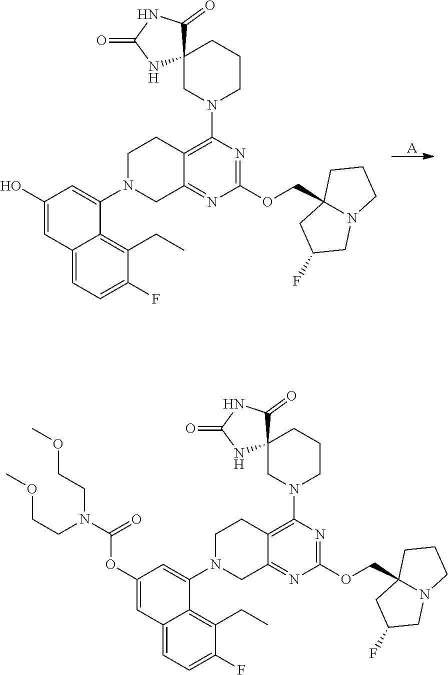

Step A. 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl bis(2-methoxyethyl)carbamate: To a solution of (R)-7-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione (36.0 mg, 1.0 equiv) in DCM (1 mL) were added (4-nitrophenyl) carbonochloridate (13.4 mg, 1.2 equiv) and TEA (16.9 mg, 3.0 equiv). The reaction was degassed and purged with nitrogen for three times and stirred at −10° C. for 0.5 hours. 2-methoxy-N-(2-methoxyethyl) ethanamine (5.74 mg, 1.0 equiv) was added dropwise at 20° C. and the reaction was stirred at 20° C. for 12 hours. The mixture was diluted with water (50 mL) and extracted with ethyl acetate (3×10 mL). The combined organic layers were washed with brine (50 mL), dried over anhydrous sodium sulfate, concentrated and purified with prep-HPLC [column: Waters Xbridge 150×25 mm×5 μm; mobile phase: water (ammonia hydroxide v/v, B: ACN); gradient: 40%-70% B over minutes] to afford the title compound (8.92 mg, 9% yield) as off-white solid; 1H NMR (400 MHz, DMSO-d6) δ=11.08-10.23 (m, 1H), 8.27-7.91 (m, 1H), 7.89-7.76 (m, 1H), 7.58-7.49 (m, 1H), 7.46-7.36 (m, 1H), 7.32-7.13 (m, 1H), 5.39-5.11 (m, 1H), 4.08-3.77 (m, 5H), 3.73-3.55 (m, 5H), 3.55-3.46 (m, 4H), 3.32-3.28 (m, 7H), 2.55 (s, 11H), 2.13-1.58 (m, 10H), 1.20-0.96 (m, 3H); LCMS (ESI, M+1): m/z=807.6.

### Example 66

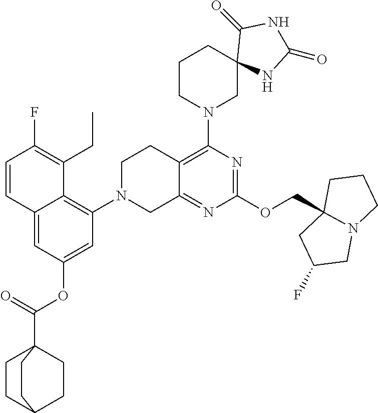 [SMILES: CCc1c(F)ccc2cc(OC(=O)C34CCC(CC3)CC4)cc(N3CCc4c(nc(OC[C@@]56CCCN5C[C@H](F)C6)nc4N4CCC[C@]5(C4)NC(=O)NC5=O)C3)c12]

### 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl bicyclo[2.2.2]octane-1-carboxylate

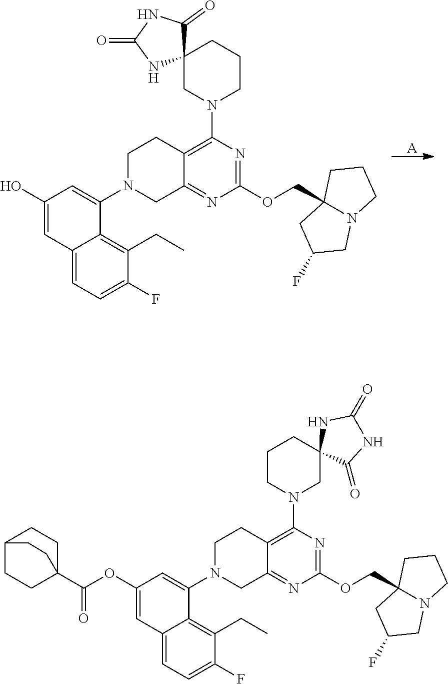

Step A. 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl bicyclo[2.2.2]octane-1-carboxylate: To a solution of bicyclo[2.2.2]octane-1-carboxylic acid (59.5 mg, 2.5 equiv) in DCM (2 mL) were added isobutyl carbonochloridate (37.9 mg, 1.8 equiv) and TEA (78.1 mg, 5.0 equiv) at 0° C. The reaction was stirred at 25° C. for 12 hours. (R)-7-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione (100 mg, 1.0 equiv) was added. The reaction was stirred at 40° C. for 12 hours. The mixture was diluted with H₂O (15 mL) and extracted with ethyl acetate (2×20 mL). The combined organic layers were washed with brine (20 mL), dried over anhydrous sodium sulfate, concentrated and purified with prep-HPLC [column: Phenomenex luna C18 150×25 mm×10 μm; mobile phase: water (formic acid v/v, B: ACN); gradient: 38%-68% B over 9 min] to afford the title compound (26.0 mg, 11% yield, HCOOH) as off-white solid; 1H NMR (400 MHz, DMSO-d6) δ=10.84-10.68 (m, 1H), 8.66-8.43 (m, 1H), 7.84 (m, 1H), 7.61-7.53 (m, 1H), 7.42 (t, J=9.2 Hz, 1H), 7.34-7.13 (m, 1H), 5.35-5.08 (m, 1H), 4.04-3.63 (m, 6H), 3.55-3.41 (m, 2H), 3.30-2.93 (m, 8H), 2.92-2.58 (m, 3H), 2.41-2.25 (m, 4H), 2.10-1.86 (m, 7H), 1.83-1.68 (m, 5H), 1.07 (br t, J=7.2 Hz, 3H); LCMS (ESI, M+1): m/z=784.6.

### Example 67

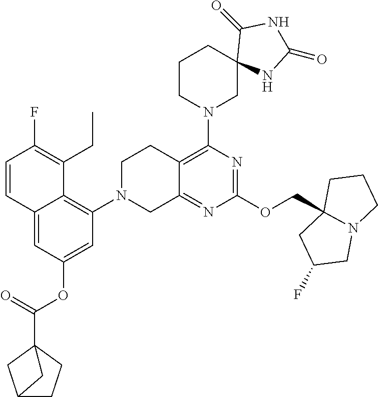 [SMILES: CCc1c(F)ccc2cc(OC(=O)C34CCC(C3)C4)cc(N3CCc4c(nc(OC[C@@]56CCCN5C[C@H](F)C6)nc4N4CCC[C@]5(C4)NC(=O)NC5=O)C3)c12]

### 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl bicyclo[2.1.1]hexane-1-carboxylate

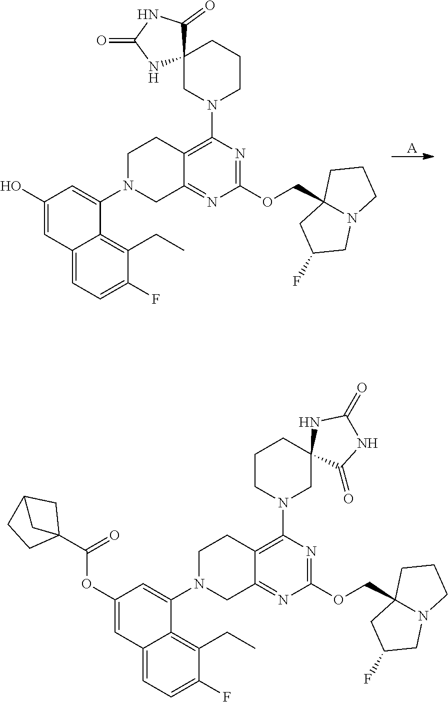

Step A. 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl bicyclo[2.1.1]hexane-1-carboxylate: To a solution of bicyclo[2.1.1]hexane-1-carboxylic acid (38.9 mg, 2.0 equiv) in DCM (0.5 mL) were added isobutyl carbonochloridate (37.9 mg, 1.8 equiv) and TEA (78.1 mg, 5.0 equiv) at 0° C. The reaction was stirred at 25° C. for 12 hours and (5R)-9-[7-(8-ethyl-7-fluoro-3-hydroxy-1-naphthyl)-2-[[(2R,8S)-2-fluoro-1,2,3,5,6,7-hexahydropyrrolizin-8-yl]methoxy]-6,8-dihydro-5H-pyrido[3,4-d]pyrimidin-4-yl]-1,3,9-triazaspiro[4.5]decane-2,4-dione (100 mg, 1.0 equiv) was added. The reaction was stirred at 25° C. for 24 hours. The mixture was diluted with water (10 mL) and extracted with ethyl acetate (3×10 mL). The combined organic layers were washed with brine (10 mL), dried over anhydrous sodium sulfate, concentrated and purified with prep-HPLC [column: Waters Xbridge 150×25 mm×5 um; mobile phase: water (ammonium bicarbonate v/v)-ACN; gradient: 63%-93% B over 9 minutes] to afford the title compound (9.34 mg, 7.4% yield) as white solid; 1H NMR (400 MHz, METHANOL-d4) δ=7.74 (dd, J=6.0, 8.4 Hz, 1H), 7.45-7.40 (m, 1H), 7.32-7.26 (m, 1H), 7.17-7.12 (m, 1H), 5.33-5.18 (m, 1H), 4.23-4.16 (m, 1H), 4.15-4.11 (m, 2H), 4.10-4.06 (m, 1H), 4.06-4.01 (m, 1H), 3.69 (br d, J=17.2 Hz, 1H), 3.57-3.48 (m, 2H), 3.45-3.38 (m, 3H), 3.24-3.17 (m, 4H), 3.04-2.94 (m, 2H), 2.83-2.75 (m, 1H), 2.53 (br s, 1H), 2.21-2.11 (m, 3H), 2.06 (br s, 5H), 1.96-1.82 (m, 8H), 1.48 (br s, 2H), 1.16-1.10 (m, 3H); LCMS (ESI, M+1): m/z=756.4.

### Example 68

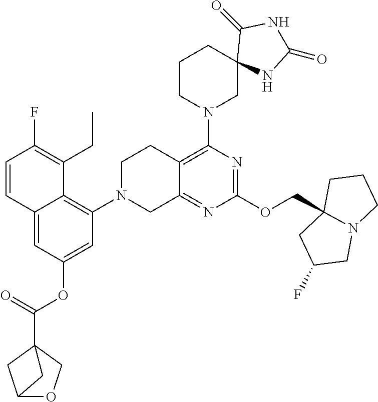 [SMILES: CCc1c(F)ccc2cc(OC(=O)C34COC(C3)C4)cc(N3CCc4c(nc(OC[C@@]56CCCN5C[C@H](F)C6)nc4N4CCC[C@]5(C4)NC(=O)NC5=O)C3)c12]

### 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl 2-oxabicyclo[2.1.1]hexane-4-carboxylate

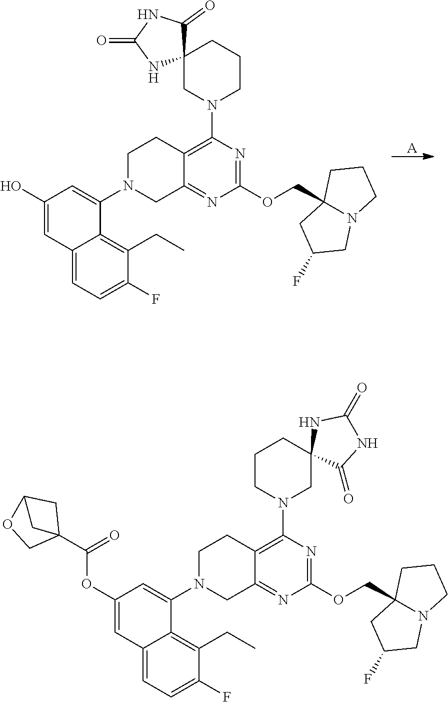

Step A. 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl 2-oxabicyclo[2.1.1]hexane-4-carboxylate: To a solution of 2-oxabicyclo[2.1.1]hexane-4-carboxylic acid (70.0 mg, 2.0 equiv) in DCM (1 mL) were added isobutyl carbonochloridate (67.2 mg, 1.8 equiv) and TEA (138 mg, 5.0 equiv) at 0° C. The reaction was stirred at 25° C. for 12 hours and then (R)-7-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione (177 mg, 1.0 equiv) was added. The reaction was stirred at 25° C. for 3 hours. The mixture was diluted with water (10 mL) and extracted with ethyl acetate (2×10 mL). The combined organic layers were washed with brine (10 mL), dried over anhydrous sodium sulfate, concentrated and purified with prep-HPLC [column: Phenomenex luna C18 150×25 mm×10 μm; mobile phase: water (formic acid v/v, B: ACN); gradient: 25%-55% B over 9 min] to afford the title compound (82.9 mg, 37% yield, HCOOH) as off-white solid; 1H NMR (400 MHz, DMSO-d6) δ=10.82-10.72 (m, 1H), 8.62-8.49 (m, 1H), 8.17 (s, 1H), 7.91-7.82 (m, 1H), 7.66-7.59 (m, 1H), 7.44 (t, J=9.6 Hz, 1H), 7.38-7.26 (m, 1H), 5.34-5.15 (m, 1H), 4.60-4.58 (m, 1H), 4.07-3.80 (m, 7H), 3.76-3.62 (m, 2H), 3.20 (br d, J=13.2 Hz, 1H), 3.15-3.05 (m, 4H), 2.94-2.75 (m, 3H), 2.34-2.28 (m, 2H), 2.12-1.64 (m, 14H), 1.12-1.04 (m, 3H); LCMS (ESI, M+1): m/z=758.3.

### Example 69

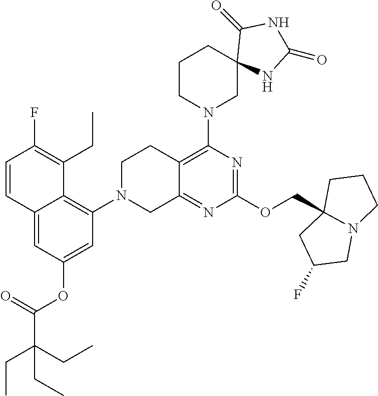 [SMILES: CCc1c(F)ccc2cc(OC(=O)C(CC)(CC)CC)cc(N3CCc4c(nc(OC[C@@]56CCCN5C[C@H](F)C6)nc4N4CCC[C@]5(C4)NC(=O)NC5=O)C3)c12]

### 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl 2,2-diethylbutanoate

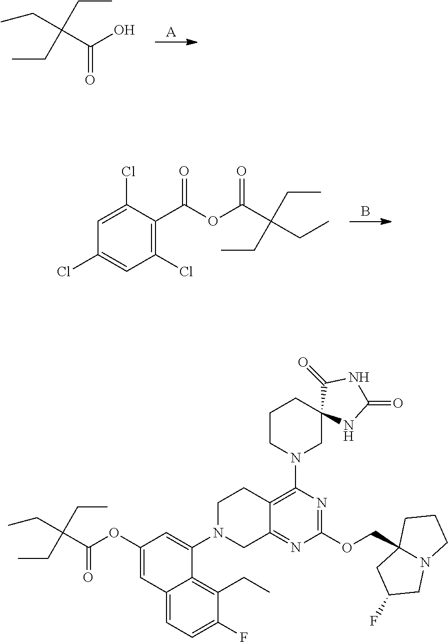

Step A. 2,4,6-trichlorobenzoic 2,2-diethylbutanoic anhydride: To a solution of 2,2-diethylbutanoic acid (70.0 mg, 1.0 equiv) in DCM (2 mL) were added TEA (98.2 mg, 2.0 equiv) and 2,4,6-trichlorobenzoyl chloride (118 mg, 1.0 equiv). The reaction was stirred at 30° C. for 12 hours. The mixture was diluted with water (10 mL) and extracted with ethyl acetate (2×10 mL). The combined organic layers were washed with brine (10 mL), dried over anhydrous sodium sulfate, concentrated and purified with prep-TLC [SiO₂, petroleum ether/ethyl acetate=50/1] to afford the title compound (166 mg, 49% yield) as white solid; 1HNMR (400 MHz, METHANOL-d4) δ=7.54 (s, 2H), 1.62-1.55 (m, 6H), 0.76 (t, J=7.6 Hz, 9H).

Step B. 4-(4-((R)-2,4-dioxo-1,3,7-triazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl 2,2-diethylbutanoate: To a solution of 2,4,6-trichlorobenzoic 2,2-diethylbutanoic anhydride (120 mg, 1.0 equiv) in toluene (6 mL) were added DMAP (83.4 mg, 2.0 equiv) and (R)-7-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione (221 mg, 1.0 equiv). The reaction was stirred at 90° C. for 12 hours. The mixture was diluted with water (15 mL) and extracted with ethyl acetate (2×10 mL). The combined organic layers were washed with brine (10 mL), dried over anhydrous sodium sulfate, concentrated and purified with prep-HPLC [column: Waters Xbridge 150×25 mm×5 μm; mobile phase: water (NH₄HCO₃)-ACN; gradient: 70%-100% B over 9 min] and SFC [column: DAICEL CHIRALCEL OD (250 mm×30 mm, 10 μm); mobile phase: CO₂-EtOH (0.1% NH₃·H₂O); B %: 50%, isocratic elution mode] to afford the title compound (6.30 mg, 2.3% yield) as off-white solid; 1HNMR (400 MHz, METHANOL-d4) δ=7.75 (td, J=5.6, 8.4 Hz, 1H), 7.36 (dd, J=2.0, 6.0 Hz, 1H), 7.29 (t, J=9.2 Hz, 1H), 7.08-6.97 (m, 1H), 5.40-5.17 (m, 1H), 4.21-3.94 (m, 5H), 3.78-3.35 (m, 6H), 3.22-2.96 (m, 6H), 2.84-2.63 (m, 1H), 2.29-1.85 (m, 10H), 1.81-1.73 (m, 6H), 1.16-1.09 (m, 3H), 0.92 (q, J=7.2 Hz, 9H); LCMS (ESI, M+1): m/z=774.5.

### Example 70

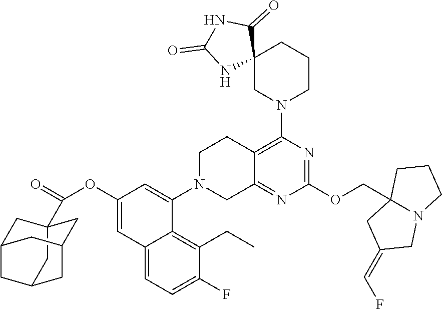 [SMILES: CCc1c(F)ccc2cc(OC(=O)C34CC5CC(CC(C5)C3)C4)cc(N3CCc4c(nc(OCC56CCCN5C/C(=C\F)C6)nc4N4CCC[C@]5(C4)NC(=O)NC5=O)C3)c12]

### 4-(4-((R)-2,4-dioxo-1,3,7-triazaspiro[4.5]decan-7-yl)-2-(((S,Z)-2-(fluoromethylene)tetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl (3r,5r,7r)-adamantane-1-carboxylate

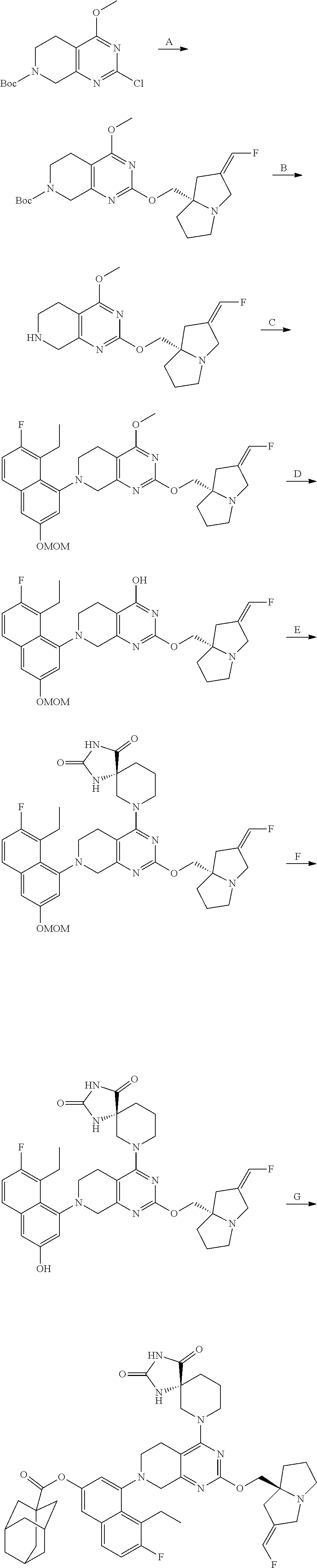

Step A. tert-butyl (S,Z)-2-((2-(fluoromethylene)tetrahydro-1H-pyrrolizin-7a(5H)-yl)methoxy)-4-methoxy-5,8-dihydropyrido[3,4-d]pyrimidine-7(6H)-carboxylate: To a mixture of tert-butyl 2-chloro-4-methoxy-5,8-dihydropyrido[3,4-d]pyrimidine-7(6H)-carboxylate (7.0 g, 1.0 equiv), (S,Z)-(2-(fluoromethylene)tetrahydro-1H-pyrrolizin-7a(5H)-yl)methanol (4.40 g, 1.1 equiv) and Cs2CO3 (22.8 g, 3.0 equiv) in toluene (70.0 mL) was added 2,2′-bis(diphenylphosphaneyl)-1,1′-binaphthalene (2.91 g, 0.2 equiv). The mixture was degassed and purged with N2 for 3 times. diacetoxypalladium (524 mg, 0.10 equiv) was added into the mixture. The reaction was degassed and purged with N2 for 3 times. The reaction was stirred at 110° C. for 3 hours. The mixture was filtered, concentrated, and purified with prep-HPLC [Phenomenex luna C18 250×70 mm, 10 um; A: water (FA), B: ACN, B %: 22%-50% over 18 min] to afford the title compound (9.20 g, 91% yield) as yellow oil; LCMS (ESI, M+1): m/z=435.2.

Step B. (S,Z)-2-((2-(fluoromethylene)tetrahydro-1H-pyrrolizin-7a(5H)-yl)methoxy)-4-methoxy-5,6,7,8-tetrahydropyrido[3,4-d]pyrimidine: A solution of tert-butyl (S,Z)-2-((2-(fluoromethylene)tetrahydro-1H-pyrrolizin-7a(5H)-yl)methoxy)-4-methoxy-5,8-dihydropyrido[3,4-d]pyrimidine-7(6H)-carboxylate (8.00 g, 1.0 equiv) in HCl-MeOH (4 M, 30 mL, 6.5 equiv) was stirred at 25° C. for 0.5 hours. The mixture was concentrated. The residue was diluted with water (30.0 mL). The pH of the mixture was adjusted to 11 with sodium hydroxide solution (15 ml). The mixture was extracted with ethyl acetate (3×100 mL). The combined organic layers were dried over anhydrous sodium sulfate and concentrated to afford the title compound (6.00 g, 88% yield) as yellow oil; LCMS (ESI, M+1): m/z=335.0.

Step C. (S,Z)-7-(8-ethyl-7-fluoro-3-(methoxymethoxy)naphthalen-1-yl)-2-((2-(fluoromethylene)tetrahydro-1H-pyrrolizin-7a(5H)-yl)methoxy)-4-methoxy-5,6,7,8-tetrahydropyrido[3,4-d]pyrimidine: To a solution of (S,Z)-2-((2-(fluoromethylene)tetrahydro-1H-pyrrolizin-7a(5H)-yl)methoxy)-4-methoxy-5,6,7,8-tetrahydropyrido[3,4-d]pyrimidine (6.00 g, 1.0 equiv) and 8-ethyl-7-fluoro-3-(methoxymethoxy)naphthalen-1-yl trifluoromethanesulfonate (8.92 g, 1.3 equiv) in dioxane (100 mL) was added Cs₂CO₃ (17.5 g, 3.0 equiv). The mixture was degassed and purged with N2 for 3 times. 1,3-bis[2,6-bis(1-propylbutyl)phenyl]-4,5-dichloro-2H-imidazol-1-ium-2-ide;3-chloropyridine;dichloropalladium (872 mg, 0.05 equiv) was added to the mixture. The reaction was degassed and purged with nitrogen 3 times. The reaction was stirred at 90° C. for 16 hours. The mixture was filtered and purified with reversed phase flash chromatography [C18, 0.1% formic acid condition] to afford the title compound (6.00 g, 57.2% yield) as yellow oil; LCMS (ESI, M+1): m/z=567.2.

Step D. (S,Z)-7-(8-ethyl-7-fluoro-3-(methoxymethoxy)naphthalen-1-yl)-2-((2-(fluoromethylene)tetrahydro-1H-pyrrolizin-7a(5H)-yl)methoxy)-5,6,7,8-tetrahydropyrido[3,4-d]pyrimidin-4-ol: To a solution of (S,Z)-7-(8-ethyl-7-fluoro-3-(methoxymethoxy)naphthalen-1-yl)-2-((2-(fluoromethylene)tetrahydro-1H-pyrrolizin-7a(5H)-yl)methoxy)-4-methoxy-5,6,7,8-tetrahydropyrido[3,4-d]pyrimidine (5.00 g, 1.0 equiv) in DMAC (30.0 mL) was added NaSEt (2.23 g, 3.0 equiv). The reaction was stirred at 60° C. for 1 hour. The mixture was quenched with water (30 mL) and extracted with EtOAc (3×50.0 mL). The combined organic layers were dried over anhydrous sodium sulfate, concentrated, and purified with reversed phase flash chromatography [C18, 0.1% formic acid condition] to afford the title compound (4.50 g, 86% yield) as yellow oil; LCMS (ESI, M+1): m/z=553.1.

Step E. (R)-7-(7-(8-ethyl-7-fluoro-3-(methoxymethoxy)naphthalen-1-yl)-2-(((S,Z)-2-(fluoromethylene)tetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione: To a solution of (S,Z)-7-(8-ethyl-7-fluoro-3-(methoxymethoxy)naphthalen-1-yl)-2-((2-(fluoromethylene)tetrahydro-1H-pyrrolizin-7a(5H)-yl)methoxy)-5,6,7,8-tetrahydropyrido[3,4-d]pyrimidin-4-ol (6.00 g, 1.0 equiv) in DMSO (60 mL) were added TEA (3.30 g, 3.0 equiv) and PyBOP (8.48 g, 1.5 equiv). The reaction was stirred at 25° C. for 0.5 hours. Then (R)-1,3,7-triazaspiro[4.5]decane-2,4-dione (2.76 g, 1.5 equiv) was added into the mixture. The reaction was stirred at 25° C. for 12 hours. The mixture was diluted with water (500 mL) and extracted with EtOAc (3×300 mL). The combined organic layers were washed with brine (500 mL), dried over anhydrous sodium sulfate, concentrated, and purified with column chromatography [SiO₂, petroleum ether/ethyl acetate=2/1 to 0/1] to afford the title compound (7.2 g, 90% yield) as red solid; LCMS (ESI, M+1): m/z=704.3.

Step F. (R)-7-(7-(8-ethyl-7-fluoro-3-hydroxynaphthalen-1-yl)-2-(((S,Z)-2-(fluoromethylene)tetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione: To a solution of (R)-7-(7-(8-ethyl-7-fluoro-3-(methoxymethoxy)naphthalen-1-yl)-2-(((S,Z)-2-(fluoromethylene)tetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione (1.00 g, 1.0 equiv) in DCM (5 mL) was added TFA (7.68 g, 49.1 equiv). The reaction was stirred at 15° C. for 0.1 hours. The mixture was diluted with DCM (10 mL) and quenched with TEA until pH=8 at −40° C. The mixture was diluted with water (20 mL) and extracted with DCM (3×15 mL). The combined organic layers were washed with brine (20 mL), dried over anhydrous sodium sulfate, concentrated and purified with prep-HPLC [column: Waters Xbridge Prep OBD C18 150×40 mm×10 um; mobile phase: [water (NH₄HCO₃)-ACN]; gradient: 35%-65% B over 12 min] to afford the title compound (175 mg, 19% yield) as red solid; LCMS (ESI, M+1): m/z=660.3.

Step G. 4-(4-((R)-2,4-dioxo-1,3,7-tri azaspiro[4.5]decan-7-yl)-2-(((S,Z)-2-(fluoromethylene)tetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl (3r,5r,7r)-adamantane-1-carboxylate: To a solution of (R)-7-(7-(8-ethyl-7-fluoro-3-hydroxynaphthalen-1-yl)-2-(((S,Z)-2-(fluoromethylene)tetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione (170 mg, 1.0 equiv) in DMF (2 mL) were added K₂CO₃ (125 mg, 15 equiv) and (3r,5r,7r)-adamantane-1-carbonyl chloride (307 mg, 10 equiv). The reaction was stirred at 50° C. for 5 hours. The mixture was diluted with water (10 mL) and extracted with ethyl acetate (3×10 mL). The combined organic layers were washed with brine (10 mL), dried over anhydrous sodium sulfate, concentrated and purified with prep-HPLC [column: Phenomenex luna C18 150×25 mm×10 μm; mobile phase: water(FA)-ACN; gradient: 35%-65% B over 10 min] to afford the title compound (18.2 mg, 16% yield, 0.24 HCOOH) as off-white solid; ¹H NMR (400 MHz, METHANOL-d4) δ=7.79-7.73 (m, 1H), 7.41 (t, J=2.4 Hz, 1H), 7.31 (t, J=9.2 Hz, 1H), 7.12 (dd, J=2.0, 7.6 Hz, 1H), 6.90-6.61 (m, 1H), 4.38-4.34 (m, 1H), 4.31-4.20 (m, 1H), 4.19-3.93 (m, 4H), 3.86-3.69 (m, 2H), 3.58-3.38 (m, 6H), 3.25-3.14 (m, 2H), 3.11-2.89 (m, 2H), 2.87-2.71 (m, 2H), 2.64-2.54 (m, 1H), 2.29-2.19 (m, 1H), 2.12 (brs, 10H), 2.06-1.92 (m, 4H), 1.90-1.82 (m, 7H), 1.19-1.12 (m, 3H); LCMS (ESI, M+1): m/z=822.5.

### Example 71

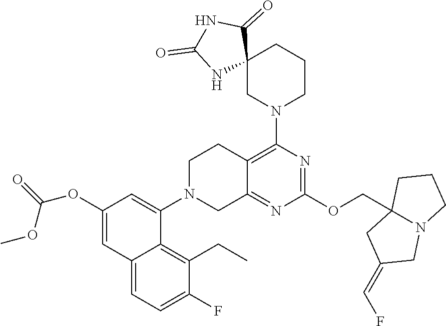 [SMILES: CCc1c(F)ccc2cc(OC(=O)OC)cc(N3CCc4c(nc(OCC56CCCN5C/C(=C\F)C6)nc4N4CCC[C@]5(C4)NC(=O)NC5=O)C3)c12]

### 4-(4-((R)-2,4-dioxo-1,3,7-triazaspiro[4.5]decan-7-yl)-2-(((S,Z)-2-(fluoromethylene)tetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl methyl carbonate

Compound of Example 71 can be prepared by one skilled in the art by methods similar to those described for preparation of compounds of Examples 30.

### Example 72

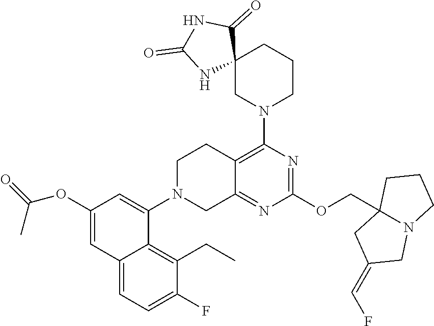 [SMILES: CCc1c(F)ccc2cc(OC(C)=O)cc(N3CCc4c(nc(OCC56CCCN5C/C(=C\F)C6)nc4N4CCC[C@]5(C4)NC(=O)NC5=O)C3)c12]

### 4-(4-(CAN-2,4-dioxo-1,3,7-triazaspiro[4.5]decan-7-yl)-2-(((S,Z)-2-(fluoromethylene)tetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl acetate

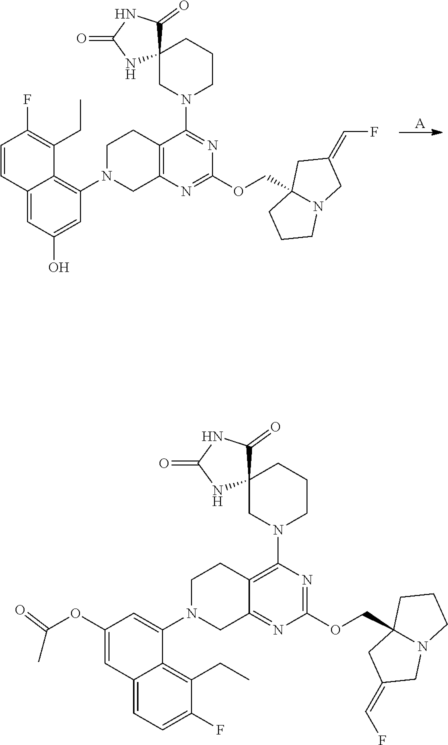

Step A. 4-(4-(CAN-2,4-dioxo-1,3,7-triazaspiro[4.5]decan-7-yl)-2-(((S,Z)-2-(fluoromethylene)tetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl acetate: To a solution of CAN-7-(7-(8-ethyl-7-fluoro-3-hydroxynaphthalen-1-yl)-2-(((S,Z)-2-(fluoromethylene)tetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione (30.0 mg, 1.0 equiv) in DMF (3 mL) were added K₂CO₃ (16.3 mg, 3.0 equiv) and acetyl chloride (6.18 mg, 2.0 equiv). The reaction was stirred at 50° C. for 12 hours. The mixture was diluted with water (10 mL) and extracted with EtOAc (2×10 mL). The combined organic layers were washed with brine (10 mL), dried over anhydrous sodium sulfate, concentrated and purified with prep-HPLC [column: waters Xbridge 150×25 mm×5 um; mobile phase: [water (NH₄HCO₃)-ACN]; gradient: 45%-75% B over 9 min] to afford the title compound (5.65 mg, 20% yield) as yellow solid; 1H NMR (400 MHz, METHANOL-d4) δ=7.73 (dd, J=6.0, 8.8 Hz, 1H), 7.43 (dd, J=2.4, 4.8 Hz, 1H), 7.29 (t, J=9.2 Hz, 1H), 7.17 (dd, J=2.4, 16.2 Hz, 1H), 6.75-6.47 (m, 1H), 4.20-3.90 (m, 5H), 3.86-3.76 (m, 1H), 3.70 (dd, J=10.8, 17.6 Hz, 1H), 3.53-3.37 (m, 5H), 3.26-2.98 (m, 4H), 2.82-2.62 (m, 3H), 2.42-2.35 (m, 1H), 2.31 (d, J=4.0 Hz, 3H), 2.16-2.04 (m, 2H), 2.01-1.75 (m, 6H), 1.12 (dt, J=2.4, 7.2 Hz, 3H); LCMS (ESI, M+1): m/z=702.4.

### Example 73

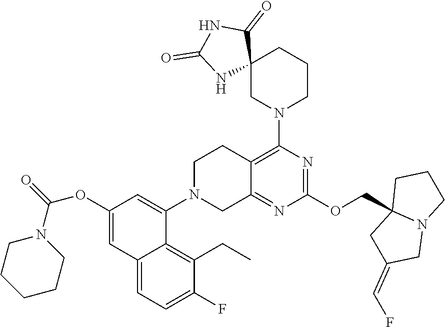 [SMILES: CCc1c(F)ccc2cc(OC(=O)N3CCCCC3)cc(N3CCc4c(nc(OC[C@@]56CCCN5C/C(=C\F)C6)nc4N4CCC[C@]5(C4)NC(=O)NC5=O)C3)c12]

### 4-(4-(CAN-2,4-dioxo-1,3,7-triazaspiro[4.5]decan-7-yl)-2-(((S,Z)-2-(fluoromethylene)tetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl piperidine-1-carboxylate

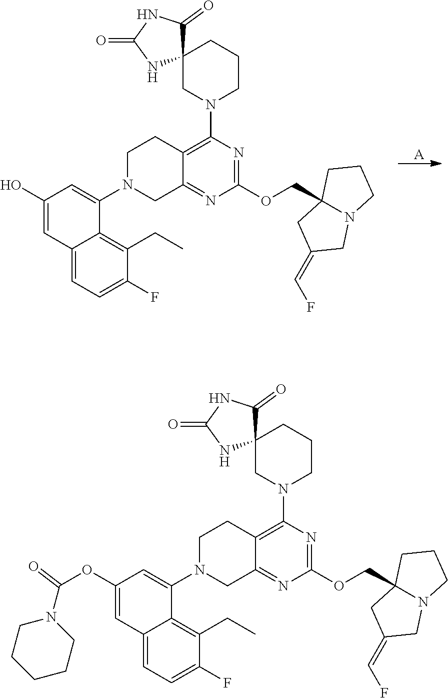

Step A. 4-(4-(CAN-2,4-dioxo-1,3,7-triazaspiro[4.5]decan-7-yl)-2-(((S,Z)-2-(fluoromethylene)tetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl piperidine-1-carboxylate: To a solution of CAN-7-(7-(8-ethyl-7-fluoro-3-hydroxynaphthalen-1-yl)-2-(((S,Z)-2-(fluoromethylene)tetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione (40.0 mg, 1.0 equiv) in DMF (4 mL) were added K₂CO₃ (83.8 mg, 10.0 equiv) and piperidine-1-carbonyl chloride (89.5 mg, 10.0 equiv). The reaction was stirred at 50° C. for 12 hours. The mixture was diluted with water (10 mL) and extracted with EtOAc (2×10 mL). The combined organic layers were washed with brine (10 mL), dried over anhydrous sodium sulfate, concentrated and purified with prep-HPLC [column: Waters Xbridge 150×25 mm×5 um; mobile phase: [water(NH₄HCO₃)-ACN]; gradient: 52%-82% B over 9 min] to afford the title compound (3.49 mg, 7.3% yield) as yellow solid; 1H NMR (400 MHz, METHANOL-d4) δ=7.72 (dd, J=6.0, 8.8 Hz, 1H), 7.45-7.37 (m, 1H), 7.28 (t, J=9.6 Hz, 1H), 7.17 (dd, J=2.0, 12.4 Hz, 1H), 6.76-6.47 (m, 1H), 4.25-4.04 (m, 5H), 3.86-3.66 (m, 4H), 3.52 (br s, 3H), 3.46-3.39 (m, 3H), 3.24-3.10 (m, 3H), 3.03 (br s, 1H), 2.81-2.64 (m, 3H), 2.39 (br d, J=14.8 Hz, 1H), 2.16-2.06 (m, 2H), 2.03-1.75 (m, 7H), 1.68 (br d, J=11.2 Hz, 6H), 1.14-1.10 (m, 3H); LCMS (ESI, M+1): m/z=771.5.

### Example 74

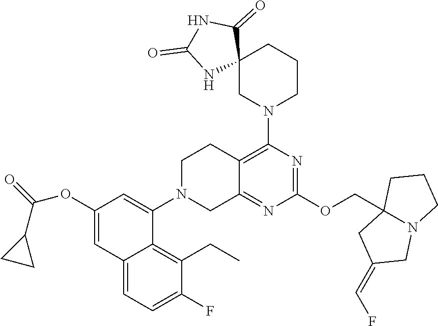 [SMILES: CCc1c(F)ccc2cc(OC(=O)C3CC3)cc(N3CCc4c(nc(OCC56CCCN5C/C(=C\F)C6)nc4N4CCC[C@]5(C4)NC(=O)NC5=O)C3)c12]

### 4-(4-(CAN-2,4-dioxo-1,3,7-triazaspiro[4.5]decan-7-yl)-2-(((S,Z)-2-(fluoromethylene)tetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl cyclopropanecarboxylate

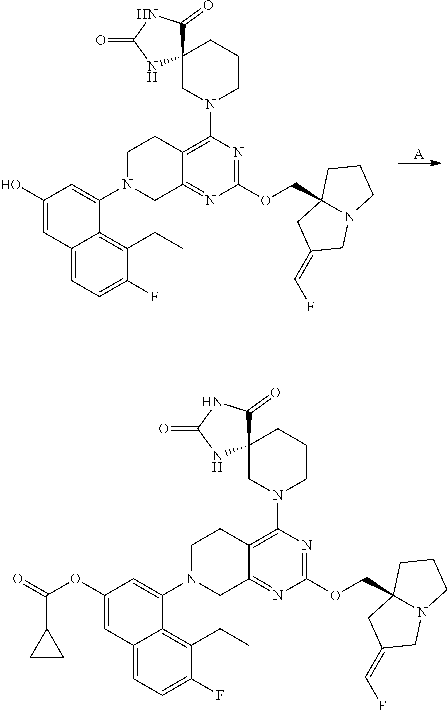

Step A. 4-(4-(CAN-2,4-dioxo-1,3,7-triazaspiro[4.5]decan-7-yl)-2-(((S,Z)-2-(fluoromethylene)tetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl cyclopropanecarboxylate: To a solution of CAN-7-(7-(8-ethyl-7-fluoro-3-hydroxynaphthalen-1-yl)-2-(((S,Z)-2-(fluoromethylene)tetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione (50.0 mg, 1.0 equiv) in DMF (4 mL) were added K₂CO₃ (157 mg, 15.0 equiv) and cyclopropanecarbonyl chloride (79.2 mg, 10.0 equiv). The reaction was stirred at 50° C. for 5 hours. The mixture was diluted with water (10 mL) and extracted with EtOAc (2×10 mL). The combined organic layers were washed with brine (10 mL), dried over anhydrous sodium sulfate, concentrated and purified with prep-HPLC [column: Waters Xbridge 150×25 mm×5 um; mobile phase: [water(NH₄HCO₃)-ACN]; gradient: 50%-80% B over 9 min] to afford the title compound (6.18 mg, 11% yield) as yellow solid; 1H NMR (400 MHz, METHANOL-d4) δ=7.78-7.66 (m, 1H), 7.42 (br s, 1H), 7.29 (br t, J=9.2 Hz, 1H), 7.21-7.10 (m, 1H), 6.75-6.46 (m, 1H), 4.26-4.01 (m, 5H), 3.96-3.81 (m, 1H), 3.76-3.65 (m, 1H), 3.52-3.38 (m, 5H), 3.22-2.98 (m, 4H), 2.80-2.62 (m, 3H), 2.44-2.35 (m, 1H), 2.16-2.06 (m, 2H), 2.00-1.77 (m, 7H), 1.20-1.00 (m, 7H); LCMS (ESI, M+1): m/z=728.2.

### Example 75

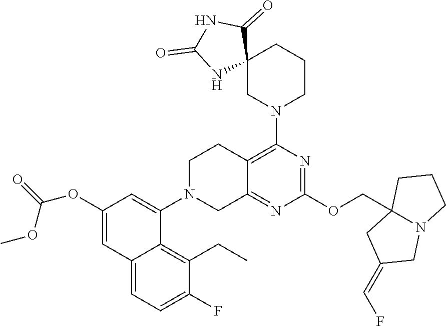 [SMILES: CCc1c(F)ccc2cc(OC(=O)OC)cc(N3CCc4c(nc(OCC56CCCN5C/C(=C\F)C6)nc4N4CCC[C@]5(C4)NC(=O)NC5=O)C3)c12]

### 4-(4-(CAN-2,4-dioxo-1,3,7-triazaspiro[4.5]decan-7-yl)-2-(((S,Z)-2-(fluoromethylene)tetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl propionate

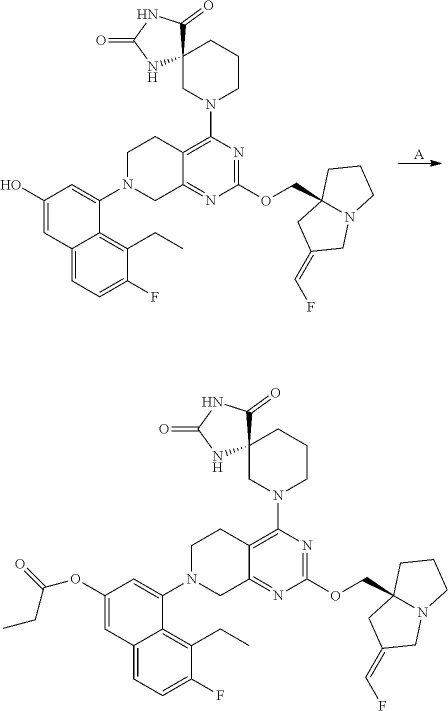

Step A. 4-(4-(CAN-2,4-dioxo-1,3,7-triazaspiro[4.5]decan-7-yl)-2-(((S,Z)-2-(fluoromethylene)tetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl propionate: To a solution of CAN-7-(7-(8-ethyl-7-fluoro-3-hydroxynaphthalen-1-yl)-2-(((S,Z)-2-(fluoromethylene)tetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione (30.0 mg, 1.0 equiv) in DMF (4 mL) were added K₂CO₃ (94.3 mg, 15.0 equiv) and propionyl chloride (42.1 mg, 10.0 equiv). The reaction was stirred at 50° C. for 5 hours. The mixture was diluted with water (10 mL) and extracted with EtOAc (2×10 mL). The combined organic layers were washed with brine (10 mL), dried over anhydrous sodium sulfate, concentrated and purified with prep-HPLC [column: Waters Xbridge 150×25 mm×5 um; mobile phase: [water(NH₄HCO₃)-ACN]; gradient: 48%-78% B over 9 min] to afford the title compound (4.55 mg, 13% yield) as pink solid; 1H NMR (400 MHz, METHANOL-d4) δ=7.73 (dd, J=5.6, 8.8 Hz, 1H), 7.43 (dd, J=2.4, 4.0 Hz, 1H), 7.29 (t, J=9.2 Hz, 1H), 7.17 (dd, J=2.0, 13.6 Hz, 1H), 6.77-6.47 (m, 1H), 4.18-4.07 (m, 4H), 3.97-3.60 (m, 3H), 3.54-3.38 (m, 5H), 3.23-2.99 (m, 4H), 2.82-2.60 (m, 5H), 2.39 (br d, J=15.6 Hz, 1H), 2.12-2.07 (m, 2H), 2.02-1.77 (m, 6H), 1.29-1.23 (m, 3H), 1.15-1.11 (m, 3H); LCMS (ESI, M+1): m/z=716.2.

### Example 76

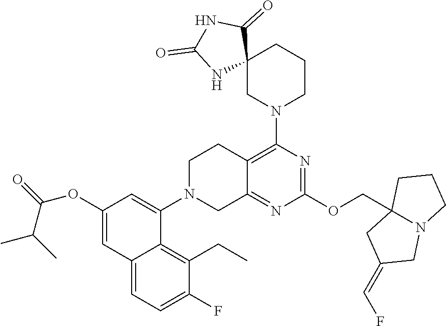 [SMILES: CCc1c(F)ccc2cc(OC(=O)C(C)C)cc(N3CCc4c(nc(OCC56CCCN5C/C(=C\F)C6)nc4N4CCC[C@]5(C4)NC(=O)NC5=O)C3)c12]

### 4-(4-(CAN-2,4-dioxo-1,3,7-triazaspiro[4.5]decan-7-yl)-2-(((S,Z)-2-(fluoromethylene)tetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl isobutyrate

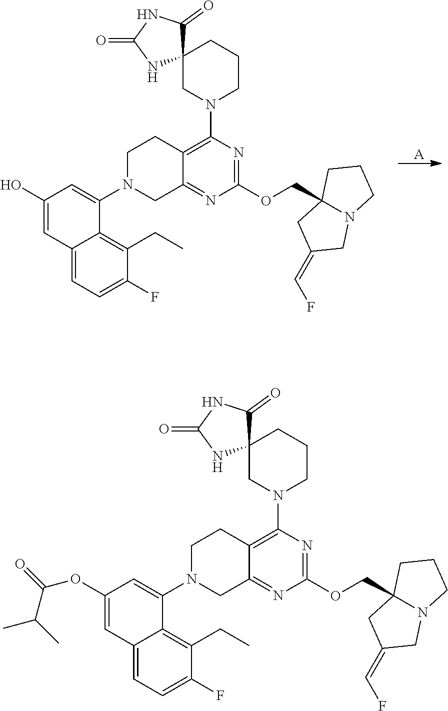

Step A. 4-(4-(CAN-2,4-dioxo-1,3,7-triazaspiro[4.5]decan-7-yl)-2-(((S,Z)-2-(fluoromethylene)tetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl isobutyrate: To a solution of CAN-7-(7-(8-ethyl-7-fluoro-3-hydroxynaphthalen-1-yl)-2-(((S,Z)-2-(fluoromethylene)tetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione (40.0 mg, 1.0 equiv) in DMF (2 mL) was added K₂CO₃ (29.3 mg, 3.5 equiv) and 2-methylpropanoyl chloride (32.3 mg, 5.0 equiv). The reaction was stirred at 50° C. for 12 hours. The mixture was diluted with water (10 mL) and extracted with EtOAc (2×10 mL). The combined organic layers were washed with brine (10 mL), dried over anhydrous sodium sulfate, concentrated and purified with prep-HPLC [column: waters Xbridge 150×25 mm×5 um; mobile phase: [water (NH₄HCO₃)-ACN]; gradient: 55%-85% B over 9 min] to afford the title compound (10.2 mg, 23% yield) as yellow solid; 1H NMR (400 MHz, METHANOL-d4) δ=7.77-7.69 (m, 1H), 7.42 (dd, J=2.4, 4.4 Hz, 1H), 7.29 (t, J=9.2 Hz, 1H), 7.15 (dd, J=2.4, 13.2 Hz, 1H), 6.75-6.48 (m, 1H), 4.23-3.91 (m, 5H), 3.86-3.76 (m, 1H), 3.69 (dd, J=5.2, 17.6 Hz, 1H), 3.54-3.38 (m, 5H), 3.27-2.97 (m, 4H), 2.87 (dtd, J=2.4, 7.2, 14.0 Hz, 1H), 2.82-2.62 (m, 3H), 2.39 (br d, J=15.2 Hz, 1H), 2.16-2.05 (m, 2H), 2.02-1.76 (m, 6H), 1.33 (dd, J=3.2, 7.2 Hz, 6H), 1.13 (dt, J=2.8, 7.2 Hz, 3H); LCMS (ESI, M+1): m/z=730.4.

### Example 77

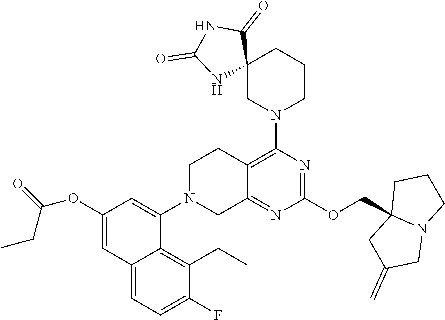 [SMILES: C=C1CN2CCC[C@@]2(COc2nc3c(c(N4CCC[C@]5(C4)NC(=O)NC5=O)n2)CCN(c2cc(OC(=O)CC)cc4ccc(F)c(CC)c24)C3)C1]

### 4-(4-(CAN-2,4-dioxo-1,3,7-triazaspiro[4.5]decan-7-yl)-2-(((S)-2-methylenetetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl propionate

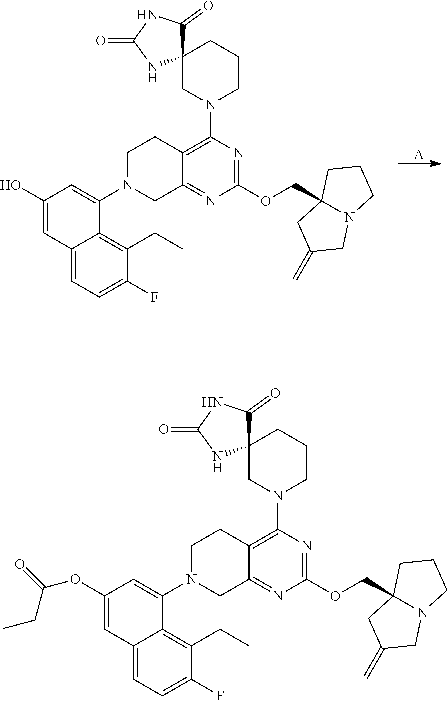

Step A. 4-(4-(CAN-2,4-dioxo-1,3,7-triazaspiro[4.5]decan-7-yl)-2-(((S)-2-methylenetetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl propionate: To a solution of CAN-7-(7-(8-ethyl-7-fluoro-3-hydroxynaphthalen-1-yl)-2-(((S)-2-methylenetetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione (100 mg, 1.0 equiv) in DMF (1.0 mL) were added K₂CO₃ (129 mg, 6.0 equiv) and propionyl chloride (57.7 mg, 4.0 equiv). The reaction was stirred at 50° C. for 12 hours. The mixture was diluted with water (3 mL) and extracted with ethyl acetate (4×5 mL). The combined organic layers were dried over anhydrous sodium sulfate, concentrated and purified by reversed phase flash chromatography [C18, 0.1% NH₃·H₂O condition] to afford the title compound (50 mg, 44% yield) as white solid; 1H NMR (400 MHz, DMSO-d6) δ 10.90-10.61 (m, 1H), 8.65-8.45 (m, 1H), 7.89-7.78 (m, 1H), 7.60-7.50 (m, 1H), 7.42 (t, J=9.2 Hz, 1H), 7.34-7.15 (m, 1H), 4.90-4.79 (m, 2H), 4.05-3.73 (m, 5H), 3.73-3.59 (m, 1H), 3.56-3.38 (m, 3H), 3.30-3.21 (m, 2H), 3.21-3.00 (m, 4H), 2.99-2.76 (m, 3H), 2.72-2.54 (m, 3H), 2.34-2.24 (m, 1H), 2.06-1.85 (m, 3H), 1.84-1.68 (m, 4H), 1.66-1.56 (m, 1H), 1.20-1.12 (m, 3H), 1.11-1.02 (m, 3H); LCMS (ESI, M+1): m/z=698.4.

### Example 78

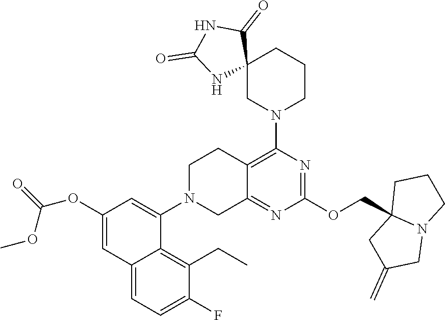 [SMILES: C=C1CN2CCC[C@@]2(COc2nc3c(c(N4CCC[C@]5(C4)NC(=O)NC5=O)n2)CCN(c2cc(OC(=O)OC)cc4ccc(F)c(CC)c24)C3)C1]

### 4-(4-(CAN-2,4-dioxo-1,3,7-triazaspiro[4.5]decan-7-yl)-2-(((S)-2-methylenetetrahydro-JH-pyrrolizin-7a(5H)-yl)methoxy)-5,8-dihydropyrido[3,4-d]pyrimidin-7(6H)-yl)-5-ethyl-6-fluoronaphthalen-2-yl methyl carbonate

Compound of Example 78 can be prepared by one skilled in the art by methods similar to those described for preparation of compounds of Examples 30.

### Example 79

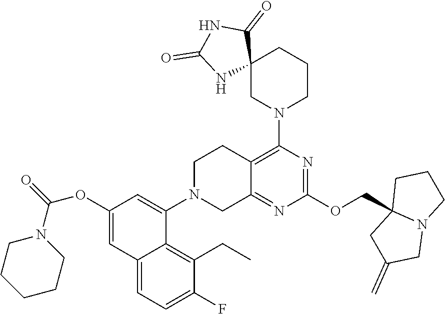 [SMILES: C=C1CN2CCC[C@@]2(COc2nc3c(c(N4CCC[C@]5(C4)NC(=O)NC5=O)n2)CCN(c2cc(OC(=O)N4CCCCC4)cc4ccc(F)c(CC)c24)C3)C1]

### 4-(4-(CAN-2,4-dioxo-1,3,7-triazaspiro[4.5]decan-7-yl)-2-(((S)-2-methylenetetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl piperidine-1-carboxylate

Compound of Example 79 can be prepared by one skilled in the art by methods similar to those described for preparation of compounds of Examples 73.

### Example 80

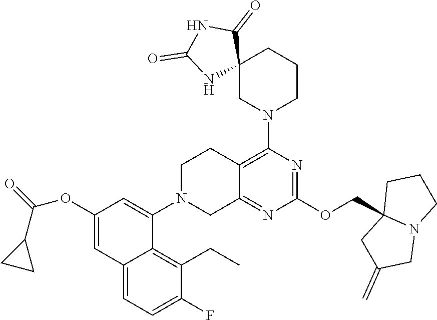 [SMILES: C=C1CN2CCC[C@@]2(COc2nc3c(c(N4CCC[C@]5(C4)NC(=O)NC5=O)n2)CCN(c2cc(OC(=O)C4CC4)cc4ccc(F)c(CC)c24)C3)C1]

### 4-(4-(CAN-2,4-dioxo-1,3,7-triazaspiro[4.5]decan-7-yl)-2-(((S)-2-methylenetetrahydro-1H-pyrrolizin-7a(5H)-yl)methoxy)-5,8-dihydropyrido[3,4-d]pyrimidin-7(6H)-yl)-5-ethyl-6-fluoronaphthalen-2-yl cyclopropanecarboxylate

Compound of Example 80 can be prepared by one skilled in the art by methods similar to those described for preparation of compounds of Examples 77.

### Example A

### Whole Blood Stability Assay

This Example illustrates that exemplary compounds of the present invention is cleavage in mouse whole blood.

Test Compounds and Stock Solutions: Stock Conc. 10 mM; Final Conc. 2 μM

### Test Compound and Control Working Solution Preparation:

Intermediate solution: 5 μL of compound stock solution (10 mM in dimethyl sulfoxide (DMSO)) were diluted with 45 1 of DMSO (intermediate solution concentration: 1 mM, 100% DMSO)
Propantheline bromide (control) intermediate solution: 5 μL of propantheline stock solution (10 mM in ultra-pure water) were diluted with 45 μL of ultra-pure water (intermediate solution concentration: 1 mM)
Working solution 1: 20 μL of test compound intermediate solution (1 mM) were diluted with 180 μL of 45% ACN/H₂O (working solution concentration: 100 PM, 40.5% ACN, 10% DMSO)
Working solution 2: 20 μL of control intermediate solution (1 mM) were diluted with 180 μL of 45% ACN/H₂O (working solution concentration: 100 μM, 40.5% ACN, 10% DMSO)
Species/Matrix: CD-1 Mouse Blood
No. of Individuals: n>3 Male
Anticoagulant Used: EDTA-K2

### Method

The fresh blood was collected on the day of experiment and pre-warmed in a water bath at 37° C.
Using an Apricot automation workstation, 98 μL/well of blank blood were added to all 96-well reaction plates (Blank, T0, T10, T30, T60 and T120).
An Apricot automation workstation was used to add 2 μL/well of working solution (100 μM) to all reaction plates except Blank (T0, T10, T30, T60 and T120).
All reaction plates containing mixtures of compound and blood were incubated at 37° C. in water bath.
The reaction plates were incubated at 37° C., and timer was started.
At the end of incubation, mixed 100 μL water with 100 μL spiked blood samples, then added 800 μL of stop solution (200 ng/mL tolbutamide and 200 ng/mL labetalol in ACN) to precipitate protein. Mixed thoroughly.
Each plate was sealed and shaken for 20 minutes.
After shaking, each plate was centrifuged at 4000 rpm and 4° C. for 20 minutes.
After centrifugation, an Apricot automation workstation was used to transfer 150 μL of supernatant from each reaction plate to its corresponding bioanalysis plate.
Each bioanalysis plate was submitted to LC-MS/MS analysis.

### Data Analysis

The % remaining of test compound after incubation in blood was calculated using following equation:

$$\% Remaining = 100 \times ( PAR at appointed incubation time / PAR at T 0 time )$$

where PAR is the peak area ratio of analyte versus internal standard (IS)
The appointed incubation time points are TO (0 min), Tn (n=0, 10, 30, 60 and 120 min)

TABLE 1
Mouse Whole Blood Stability

| Ex. No. | t_{1/2} (min) |
|---|---|
| 1 | 3 |
| 2 | 1 |
| 3 | 3 |
| 4 | 5 |
| 5 | 2 |
| 6 | >289 |
| 7 | >289 |
| 9 | 1 |
| 11 | <5 |
| 12 | <5 |
| 13 | 6 |
| 15 | <5 |
| 17 | 1 |
| 18 | <5 |
| 19 | 3 |
| 20 | >289 |
| 21 | 1 |
| 22 | >289 |
| 24 | 1 |
| 25 | 1 |
| 27 | 2 |
| 29 | 2 |
| 30 | 1 |
| 32 | 2 |
| 33 | <5 |
| 34 | 1 |
| 35 | >289 |
| 36 | 3 |
| 37 | 189 |
| 39 | 1 |
| 40 | 2 |
| 42 | 1 |
| 43 | >289 |
| 44 | 1 |
| 45 | >289 |
| 46 | 13 |
| 47 | 4 |
| 48 | 106 |
| 50 | 13 |
| 51 | 4 |
| 52 | >289 |
| 53 | >289 |
| 54 | >289 |
| 55 | >289 |
| 56 | >289 |
| 57 | >289 |
| 58 | >289 |
| 59 | >289 |
| 60 | 79 |
| 61 | 2 |
| 62 | 1 |
| 63 | 13 |
| 64 | 2 |
| 66 | 7 |
| 67 | 1 |
| 72 | 2 |
| 73 | 147 |
| 76 | 4 |

The formation of parent compounds was observed for these having a short half-life.

### Example B

### Plasma Stability Assay

This Example illustrates that exemplary compounds of the present invention is cleavage in human plasma.

Test Compounds and Stock Solutions: Stock Conc. 10 mM; Final Conc. 2 μM

### Test Compound and Control Working Solution Preparation:

Test compound intermediate solution: 10 μL of compound stock solution (10 mM in dimethyl sulfoxide (DMSO)) were diluted with 90 μL of DMSO (intermediate solution concentration: 1 mM, 100% DMSO)
Control intermediate solution: 10 μL of control stock solution (10 mM in dimethyl sulfoxide (DMSO)) were diluted with 90 μL of DMSO (intermediate solution concentration: 1 mM, 100% DMSO)
Species/Matrix: Human Plasma
No. of Individuals: 3 Male & 3 Female
Anticoagulant Used: Heparin

### Method

The pooled frozen plasma was thawed in a water bath at 37° C. prior to experiment. Plasma was centrifuged at 4000 rpm for 5 min and the clots were removed if any.
Using an Apricot automation workstation, 98 μL/well of blank plasma were added to all 96-well reaction plates. (Blank, T0, T10, T30, T60 and T120)
An Apricot automation workstation was used to add 2 μL/well of working solution (100 μM) to all reaction plates except Blank. (T0, T10, T30, T60 and T120)
All reaction plates containing mixtures of compound and plasma were incubated at 37° C. in water bath.
The reaction plates were incubated at 37° C., and timer was started.
At the end of incubation, added 500 μL of stop solution (200 ng/mL tolbutamide and 200 ng/mL labetalol in ACN) to precipitate protein. Mixed thoroughly.
Each plate was sealed and shaken for 20 minutes.
After shaking, each plate was centrifuged at 4000 rpm and 4° C. for 20 minutes
After centrifugation, an Apricot automation workstation was used to transfer 50 μL supernatant into 100 μL HPLC water.
Each bioanalysis plate was sealed and shaken for 10 minutes prior to LC-MS/MS analysis Data Analysis
The % remaining of test compound after incubation in blood was calculated using following equation:

$$\% Remaining = 100 \times ( PAR at appointed incubation time / PAR at T 0 time )$$

where PAR is the peak area ratio of analyte versus internal standard (IS)
The appointed incubation time points are TO (0 min), Tn (n=0, 10, 30, 60 and 120 min)

TABLE 2
Human Plasma Stability

| Ex. No. | t_{1/2} (min) |
|---|---|
| 1 | 23 |
| 2 | 13 |
| 3 | 27 |
| 4 | 17 |
| 5 | 67 |
| 6 | >289 |
| 7 | >289 |
| 9 | 9 |
| 11 | 46 |
| 12 | 26 |
| 13 | 16 |
| 15 | 146 |
| 17 | 41 |
| 18 | 44 |
| 19 | >289 |
| 20 | >289 |
| 21 | 41 |
| 22 | >289 |
| 24 | 65 |
| 25 | 77 |
| 27 | 12 |
| 29 | 33 |
| 30 | 27 |
| 32 | 51 |
| 33 | 62 |
| 34 | 210 |
| 35 | >289 |
| 36 | 33 |
| 37 | >289 |
| 39 | >289 |
| 40 | 95 |
| 42 | 79 |
| 43 | >289 |
| 44 | 142 |
| 45 | >289 |
| 46 | 116 |
| 47 | 223 |
| 48 | >289 |
| 50 | 235 |
| 51 | 235 |
| 52 | >289 |
| 53 | >289 |
| 54 | >289 |
| 55 | >289 |
| 56 | >289 |
| 57 | >289 |
| 58 | >289 |
| 59 | >289 |
| 60 | >289 |
| 61 | >289 |
| 62 | 38 |
| 63 | >289 |
| 64 | 33 |
| 65 | >289 |
| 66 | >289 |
| 67 | 267 |

The formation of parent compounds was observed for these having a short half-life.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. A prodrug of a compound of the following structure: 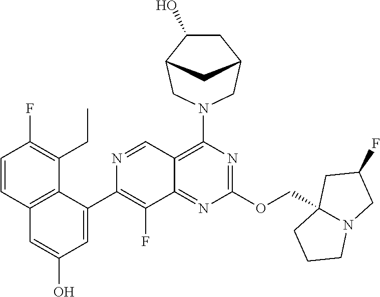 [SMILES: CCc1c(F)ccc2cc(O)cc(-c3ncc4c(N5C[C@@H]6C[C@H](C5)[C@H](O)C6)nc(OC[C@@]56CCCN5C[C@H](F)C6)nc4c3F)c12]
or pharmaceutically acceptable salt thereof.

2. The prodrug compound or salt of claim 1, wherein the compound has the following structure: 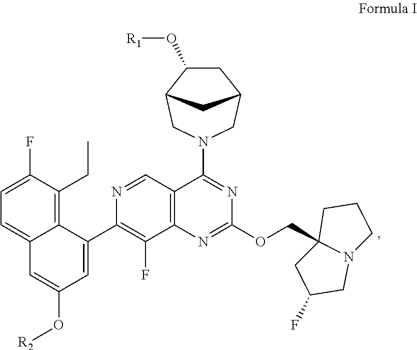
or a pharmaceutically acceptable salt thereof,
wherein
R₁ and R₂ are independently selected from the group consisting of:
R₃—*, R₃—O—CH₂—*, 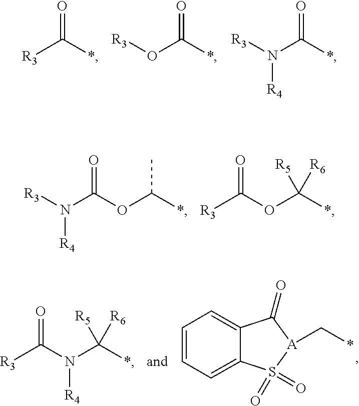
with the proviso that only one of R₁ and R₂ can be H;
A is CH or N;
R₃ is C₁-C₂₀ alkyl, (C₁-C₄ alkyl)₀₋₁-cycloalkyl, (C₁-C₄ alkyl)₀₋₁-heterocyclyl or (C₁-C₄ alkyl)₀₋₁-aryl, where R₃ is optionally substituted with C₁-C₄ alkyl or —O— C₁-C₄ alkyl; R₄ is H or R₃; and
R₅ and R₆ are independently selected from the group consisting of H and C₁-C₆ alkyl.

3. The compound of claim 2, wherein R₁ is H and R₂ is selected from the group consisting of: R₃—*, R₃—O—CH₂—*,

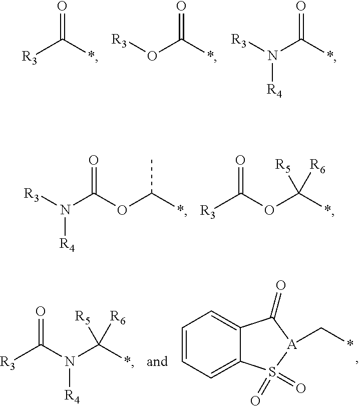

4. The compound of claim 2, wherein R₂ is H and R₁ is selected from the group consisting of: R₃—*, R₃—O—CH₂—*,

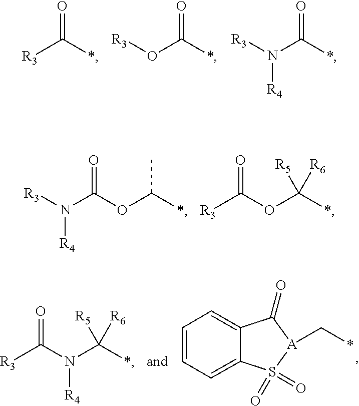

5. The prodrug compound or salt of claim 2, wherein the compound is selected from the group consisting of: 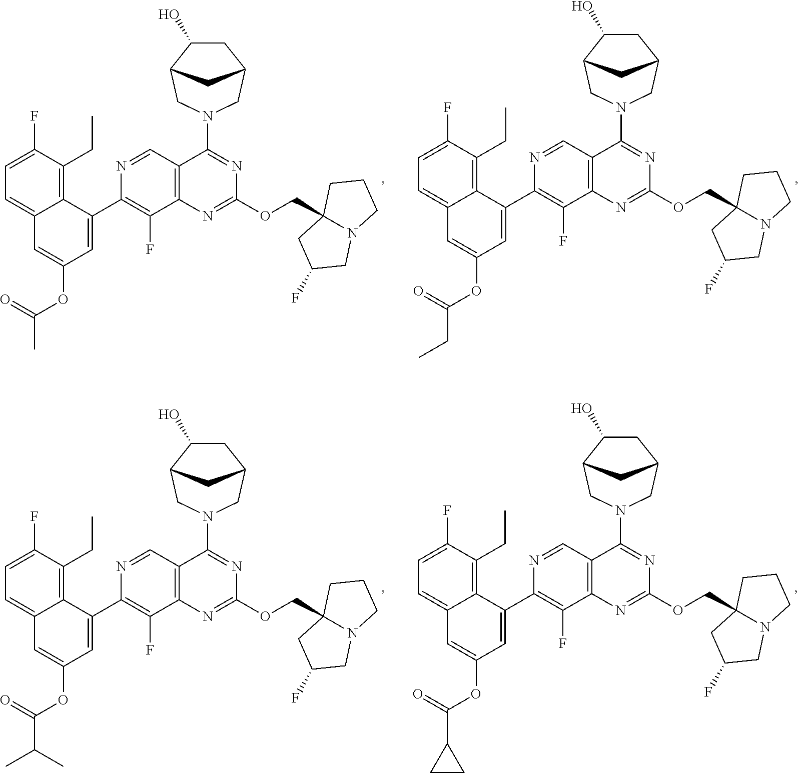 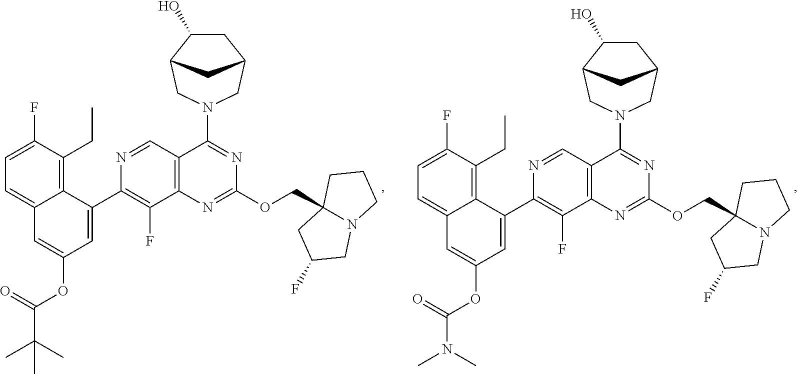 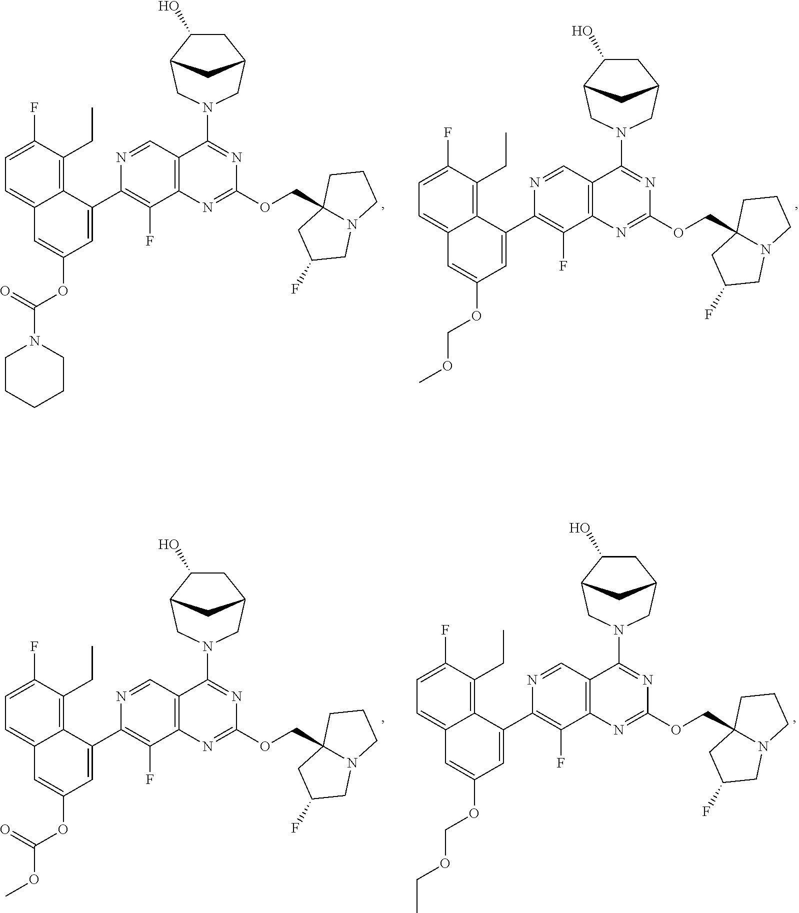 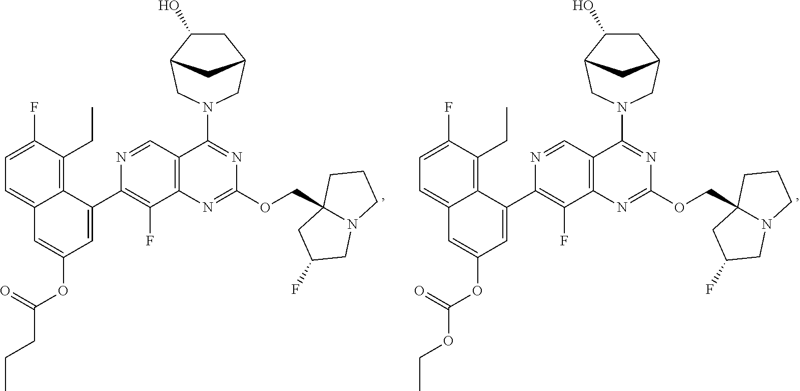 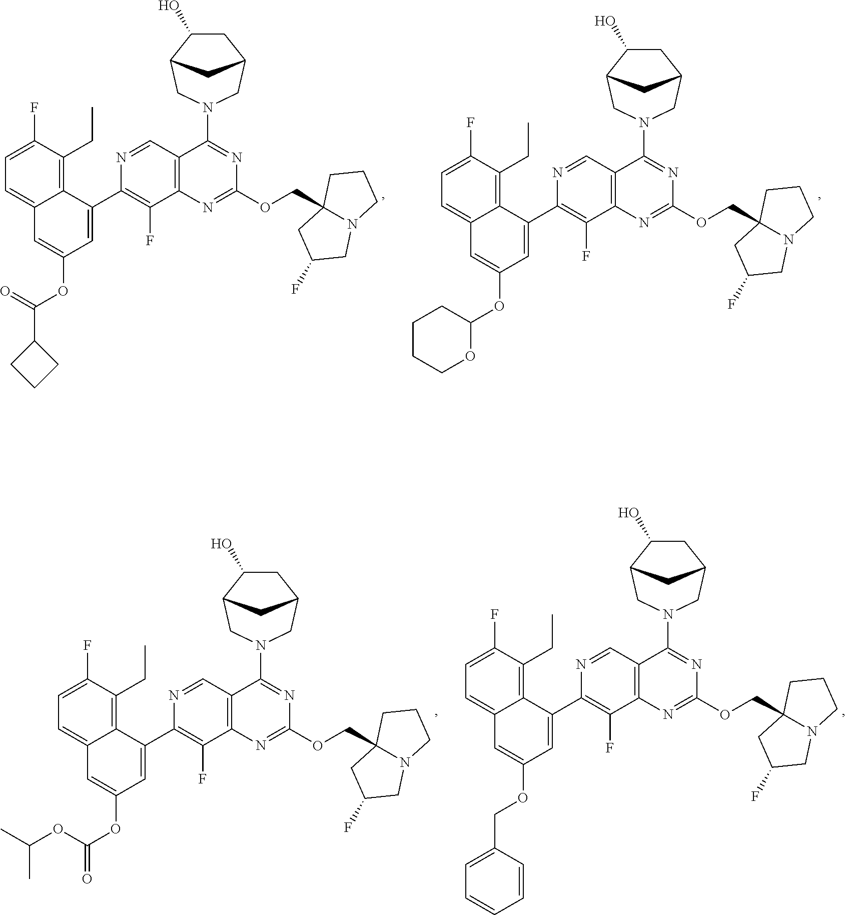 [SMILES: CCc1c(F)ccc2cc(OC(=O)C3CCC3)cc(-c3ncc4c(N5C[C@@H]6C[C@H](C5)[C@H](O)C6)nc(OC[C@@]56CCCN5C[C@H](F)C6)nc4c3F)c12 ; CCc1c(F)ccc2cc(OC3CCCCO3)cc(-c3ncc4c(N5C[C@@H]6C[C@H](C5)[C@H](O)C6)nc(OC[C@@]56CCCN5C[C@H](F)C6)nc4c3F)c12 ; CCc1c(F)ccc2cc(OC(=O)OC(C)C)cc(-c3ncc4c(N5C[C@@H]6C[C@H](C5)[C@H](O)C6)nc(OC[C@@]56CCCN5C[C@H](F)C6)nc4c3F)c12 ; CCc1c(F)ccc2cc(OCc3ccccc3)cc(-c3ncc4c(N5C[C@@H]6C[C@H](C5)[C@H](O)C6)nc(OC[C@@]56CCCN5C[C@H](F)C6)nc4c3F)c12] 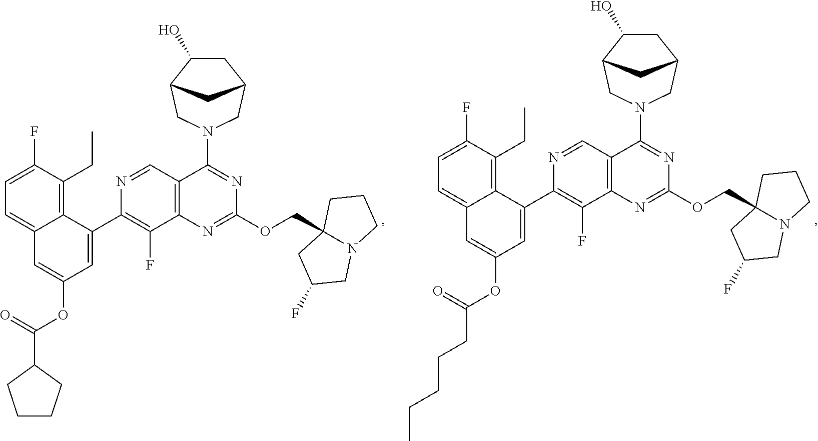 [SMILES: CCc1c(F)ccc2cc(OC(=O)C3CCCC3)cc(-c3ncc4c(N5C[C@@H]6C[C@H](C5)[C@H](O)C6)nc(OC[C@@]56CCCN5C[C@H](F)C6)nc4c3F)c12 ; CCCCCC(=O)Oc1cc(-c2ncc3c(N4C[C@@H]5C[C@H](C4)[C@H](O)C5)nc(OC[C@@]45CCCN4C[C@H](F)C5)nc3c2F)c2c(CC)c(F)ccc2c1] 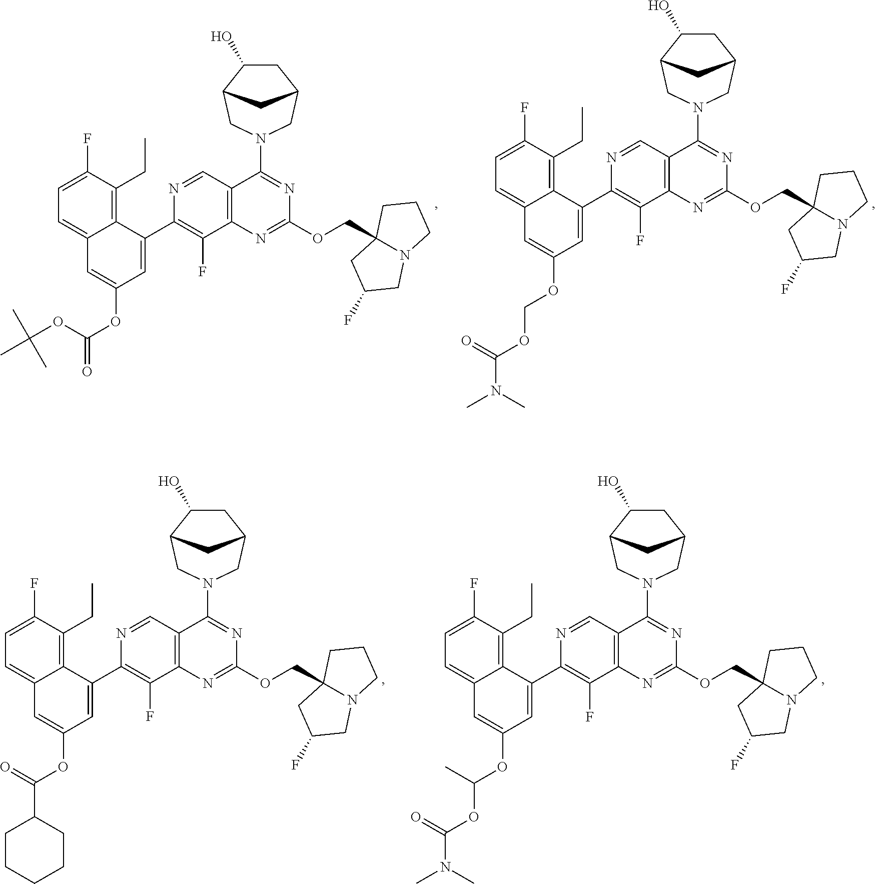 [SMILES: CCc1c(F)ccc2cc(OC(=O)OC(C)(C)C)cc(-c3ncc4c(N5C[C@@H]6C[C@H](C5)[C@H](O)C6)nc(OC[C@@]56CCCN5C[C@H](F)C6)nc4c3F)c12 ; CCc1c(F)ccc2cc(OCOC(=O)N(C)C)cc(-c3ncc4c(N5C[C@@H]6C[C@H](C5)[C@H](O)C6)nc(OC[C@@]56CCCN5C[C@H](F)C6)nc4c3F)c12 ; CCc1c(F)ccc2cc(OC(=O)C3CCCCC3)cc(-c3ncc4c(N5C[C@@H]6C[C@H](C5)[C@H](O)C6)nc(OC[C@@]56CCCN5C[C@H](F)C6)nc4c3F)c12 ; CCc1c(F)ccc2cc(OC(C)OC(=O)N(C)C)cc(-c3ncc4c(N5C[C@@H]6C[C@H](C5)[C@H](O)C6)nc(OC[C@@]56CCCN5C[C@H](F)C6)nc4c3F)c12] 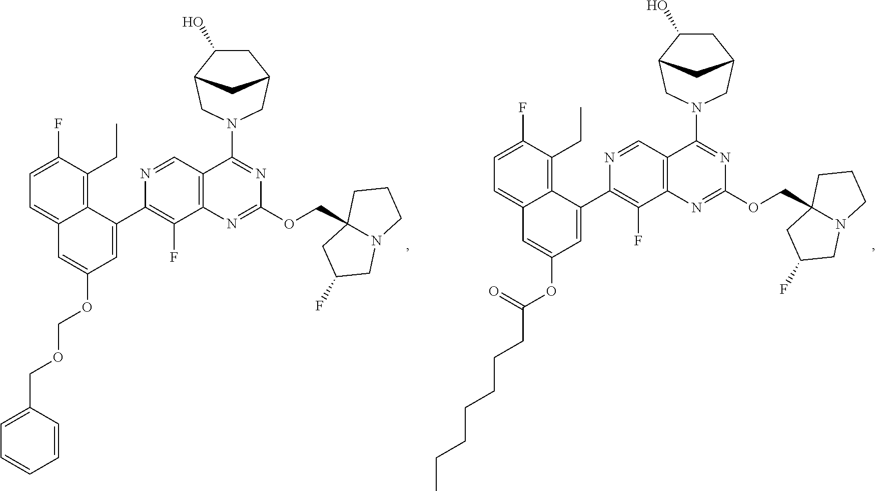 [SMILES: CCc1c(F)ccc2cc(OCOCc3ccccc3)cc(-c3ncc4c(N5C[C@@H]6C[C@H](C5)[C@H](O)C6)nc(OC[C@@]56CCCN5C[C@H](F)C6)nc4c3F)c12 ; CCCCCCCC(=O)Oc1cc(-c2ncc3c(N4C[C@@H]5C[C@H](C4)[C@H](O)C5)nc(OC[C@@]45CCCN4C[C@H](F)C5)nc3c2F)c2c(CC)c(F)ccc2c1] 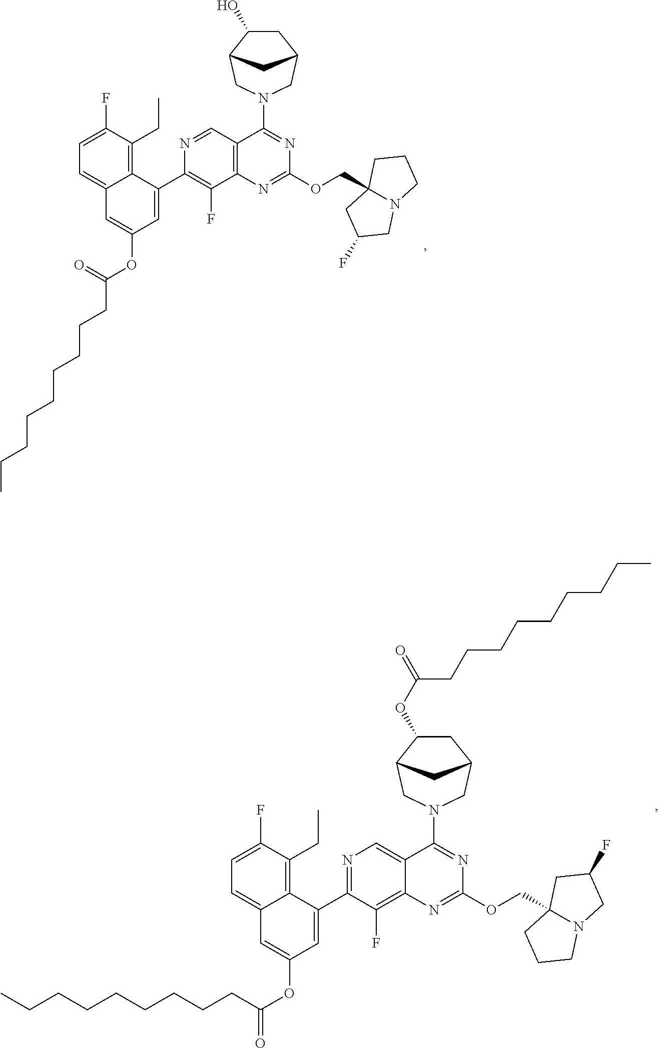 [SMILES: CCCCCCCCCC(=O)Oc1cc(-c2ncc3c(N4C[C@@H]5C[C@H](C4)[C@H](O)C5)nc(OC[C@@]45CCCN4C[C@H](F)C5)nc3c2F)c2c(CC)c(F)ccc2c1 ; CCCCCCCCCC(=O)Oc1cc(-c2ncc3c(N4C[C@@H]5C[C@H](C4)[C@H](OC(=O)CCCCCCCCC)C5)nc(OC[C@@]45CCCN4C[C@H](F)C5)nc3c2F)c2c(CC)c(F)ccc2c1]
and pharmaceutically acceptable salts thereof:

6. The prodrug compound or salt of claim 1, wherein the compound has the following structure: 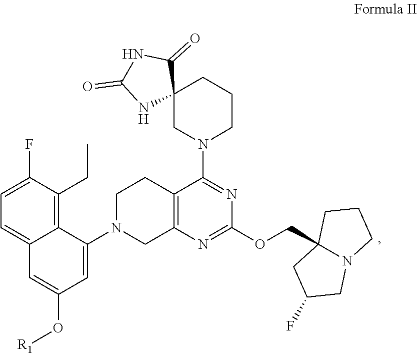
or a pharmaceutically acceptable salt thereof, wherein
R₁ is selected from the group consisting of:
R₃—*, R₃—O—CH₂—*, 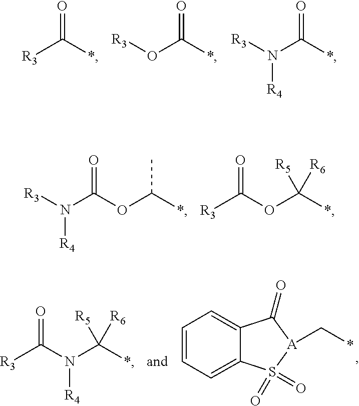
A is CH or N;
R₃ is C₁-C₂₀ alkyl, (C₁-C₄ alkyl)₀₋₁-cycloalkyl, (C₁-C₄ alkyl)₀₋₁-heterocyclyl or (C₁-C₄ alkyl)₀₋₁-aryl, where R₃ is optionally substituted with C₁-C₄ alkyl or —O— C₁-C₄ alkyl;
R₄ is H or R₃; and
R₅ and R₆ are independently selected from the group consisting of H and C₁-C₆ alkyl.

7. The compound or salt of claim 6, wherein A is CH.

8. The compound or salt of claim 6, wherein R₄ is R₃.

9. The compound or salt of claim 6, wherein the compound is selected from the group consisting of: 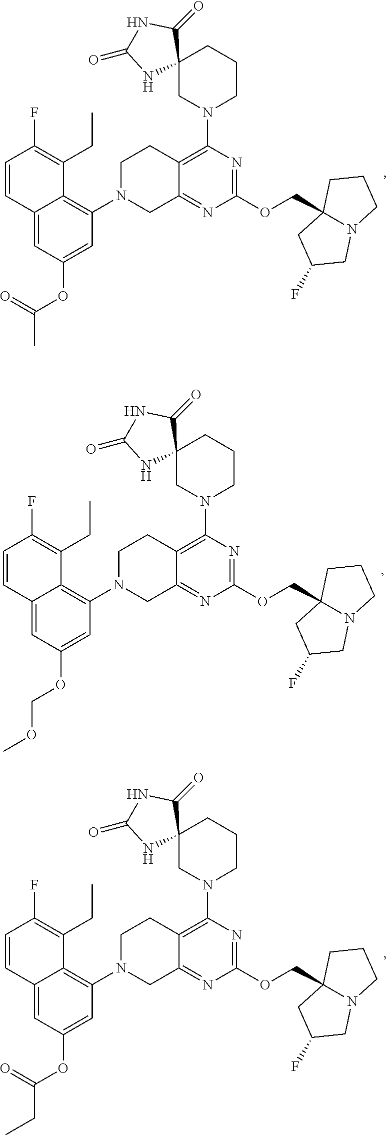 [SMILES: CCc1c(F)ccc2cc(OC(C)=O)cc(N3CCc4c(nc(OC[C@@]56CCCN5C[C@H](F)C6)nc4N4CCC[C@]5(C4)NC(=O)NC5=O)C3)c12 ; CCc1c(F)ccc2cc(OCOC)cc(N3CCc4c(nc(OC[C@@]56CCCN5C[C@H](F)C6)nc4N4CCC[C@]5(C4)NC(=O)NC5=O)C3)c12 ; CCC(=O)Oc1cc(N2CCc3c(nc(OC[C@@]45CCCN4C[C@H](F)C5)nc3N3CCC[C@]4(C3)NC(=O)NC4=O)C2)c2c(CC)c(F)ccc2c1] 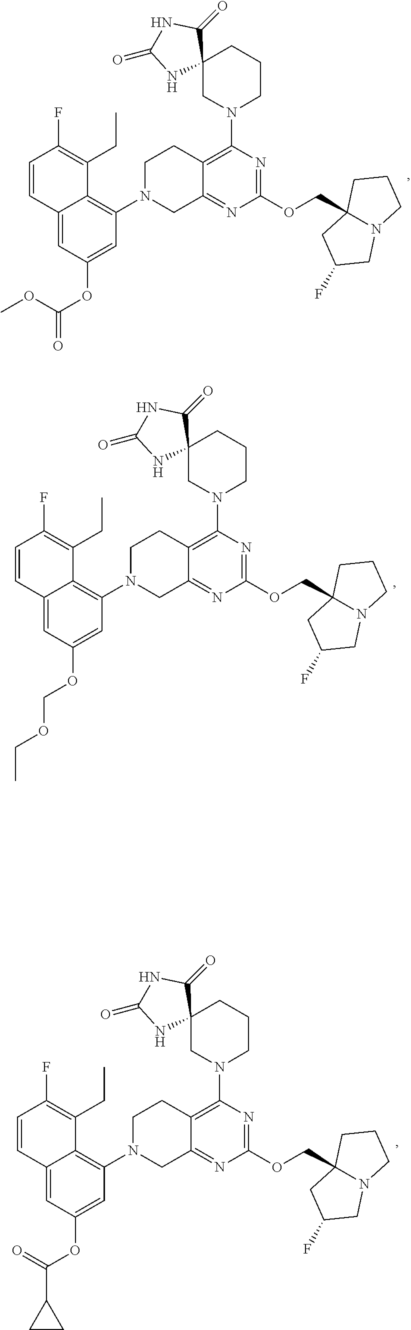 [SMILES: CCc1c(F)ccc2cc(OC(=O)OC)cc(N3CCc4c(nc(OC[C@@]56CCCN5C[C@H](F)C6)nc4N4CCC[C@]5(C4)NC(=O)NC5=O)C3)c12 ; CCOCOc1cc(N2CCc3c(nc(OC[C@@]45CCCN4C[C@H](F)C5)nc3N3CCC[C@]4(C3)NC(=O)NC4=O)C2)c2c(CC)c(F)ccc2c1 ; CCc1c(F)ccc2cc(OC(=O)C3CC3)cc(N3CCc4c(nc(OC[C@@]56CCCN5C[C@H](F)C6)nc4N4CCC[C@]5(C4)NC(=O)NC5=O)C3)c12] 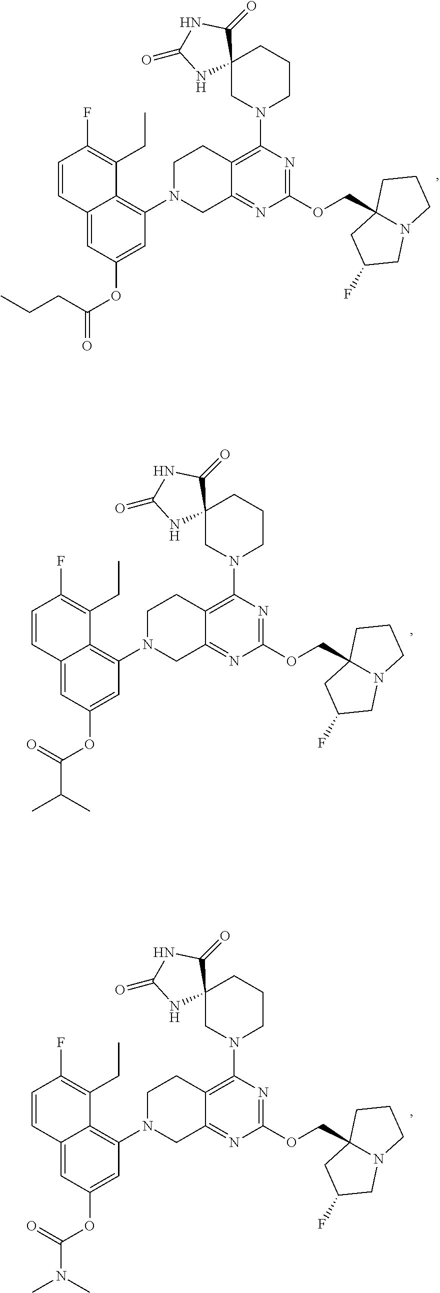 [SMILES: CCCC(=O)Oc1cc(N2CCc3c(nc(OC[C@@]45CCCN4C[C@H](F)C5)nc3N3CCC[C@]4(C3)NC(=O)NC4=O)C2)c2c(CC)c(F)ccc2c1 ; CCc1c(F)ccc2cc(OC(=O)C(C)C)cc(N3CCc4c(nc(OC[C@@]56CCCN5C[C@H](F)C6)nc4N4CCC[C@]5(C4)NC(=O)NC5=O)C3)c12 ; CCc1c(F)ccc2cc(OC(=O)N(C)C)cc(N3CCc4c(nc(OC[C@@]56CCCN5C[C@H](F)C6)nc4N4CCC[C@]5(C4)NC(=O)NC5=O)C3)c12] 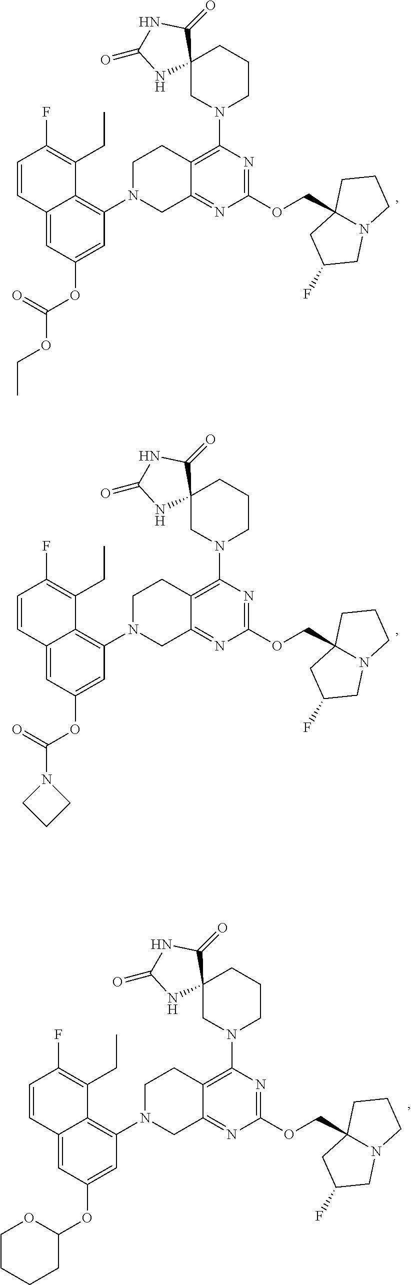 [SMILES: CCOC(=O)Oc1cc(N2CCc3c(nc(OC[C@@]45CCCN4C[C@H](F)C5)nc3N3CCC[C@]4(C3)NC(=O)NC4=O)C2)c2c(CC)c(F)ccc2c1 ; CCc1c(F)ccc2cc(OC(=O)N3CCC3)cc(N3CCc4c(nc(OC[C@@]56CCCN5C[C@H](F)C6)nc4N4CCC[C@]5(C4)NC(=O)NC5=O)C3)c12 ; CCc1c(F)ccc2cc(OC3CCCCO3)cc(N3CCc4c(nc(OC[C@@]56CCCN5C[C@H](F)C6)nc4N4CCC[C@]5(C4)NC(=O)NC5=O)C3)c12] 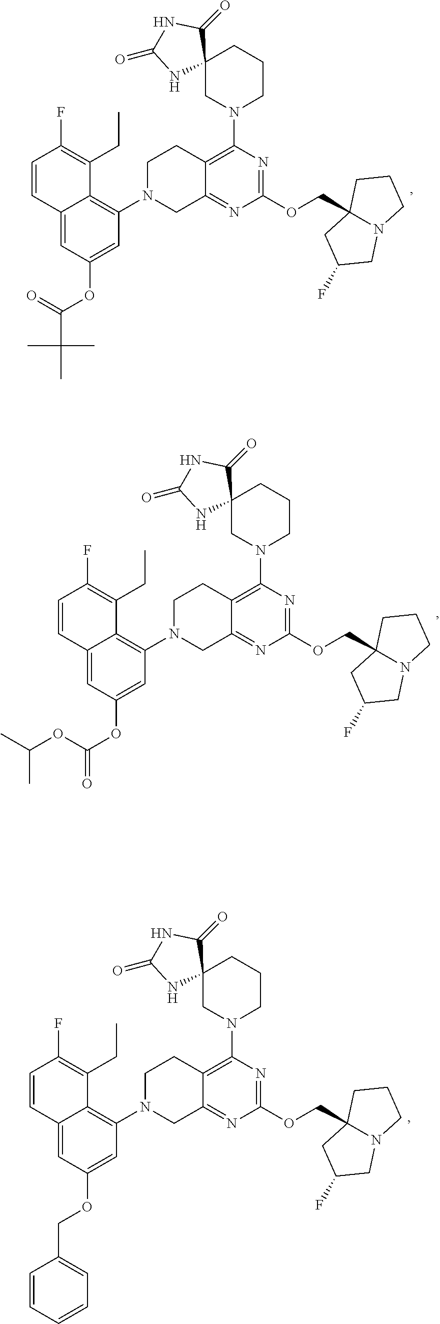 [SMILES: CCc1c(F)ccc2cc(OC(=O)C(C)(C)C)cc(N3CCc4c(nc(OC[C@@]56CCCN5C[C@H](F)C6)nc4N4CCC[C@]5(C4)NC(=O)NC5=O)C3)c12 ; CCc1c(F)ccc2cc(OC(=O)OC(C)C)cc(N3CCc4c(nc(OC[C@@]56CCCN5C[C@H](F)C6)nc4N4CCC[C@]5(C4)NC(=O)NC5=O)C3)c12 ; CCc1c(F)ccc2cc(OCc3ccccc3)cc(N3CCc4c(nc(OC[C@@]56CCCN5C[C@H](F)C6)nc4N4CCC[C@]5(C4)NC(=O)NC5=O)C3)c12] 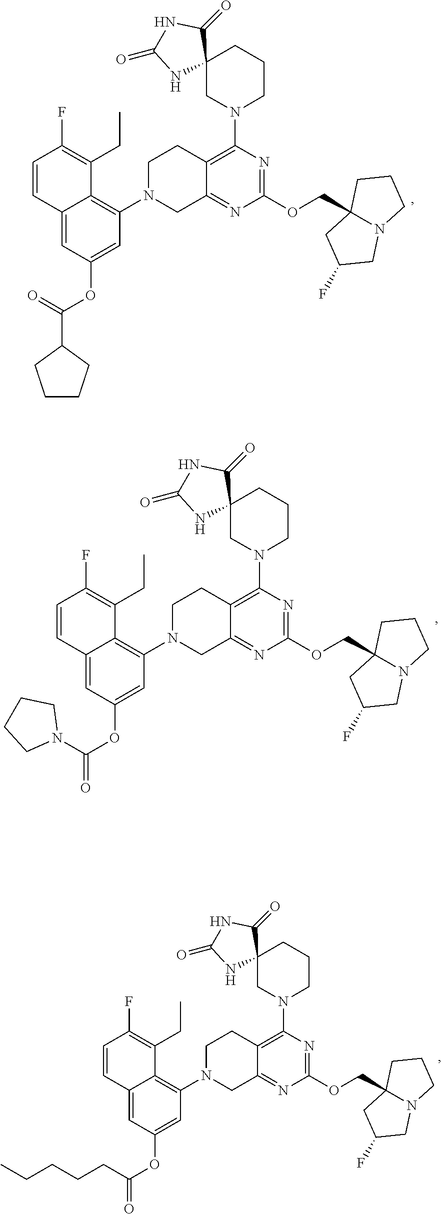 [SMILES: CCc1c(F)ccc2cc(OC(=O)C3CCCC3)cc(N3CCc4c(nc(OC[C@@]56CCCN5C[C@H](F)C6)nc4N4CCC[C@]5(C4)NC(=O)NC5=O)C3)c12 ; CCc1c(F)ccc2cc(OC(=O)N3CCCC3)cc(N3CCc4c(nc(OC[C@@]56CCCN5C[C@H](F)C6)nc4N4CCC[C@]5(C4)NC(=O)NC5=O)C3)c12 ; CCCCCC(=O)Oc1cc(N2CCc3c(nc(OC[C@@]45CCCN4C[C@H](F)C5)nc3N3CCC[C@]4(C3)NC(=O)NC4=O)C2)c2c(CC)c(F)ccc2c1] 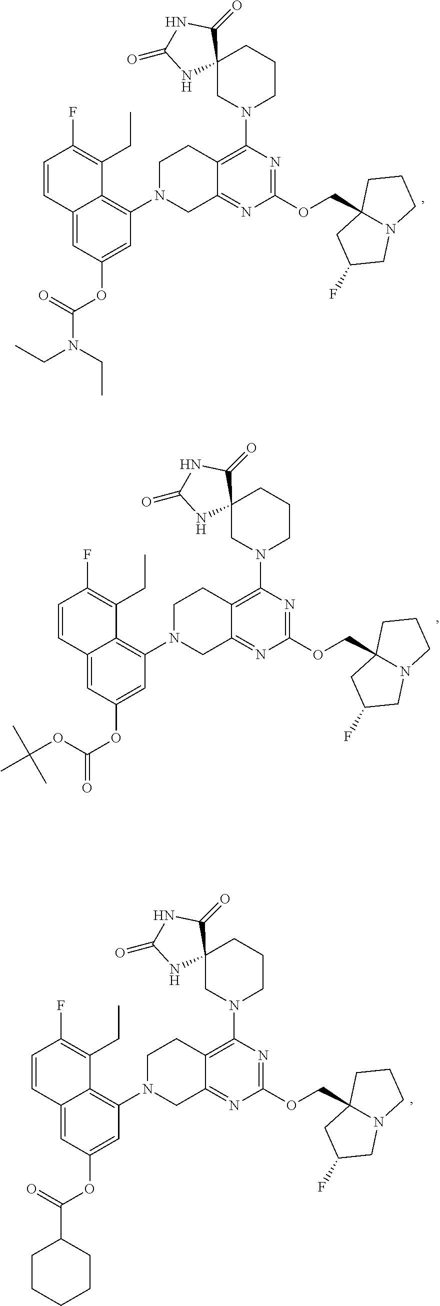 [SMILES: CCc1c(F)ccc2cc(OC(=O)N(CC)CC)cc(N3CCc4c(nc(OC[C@@]56CCCN5C[C@H](F)C6)nc4N4CCC[C@]5(C4)NC(=O)NC5=O)C3)c12 ; CCc1c(F)ccc2cc(OC(=O)OC(C)(C)C)cc(N3CCc4c(nc(OC[C@@]56CCCN5C[C@H](F)C6)nc4N4CCC[C@]5(C4)NC(=O)NC5=O)C3)c12 ; CCc1c(F)ccc2cc(OC(=O)C3CCCCC3)cc(N3CCc4c(nc(OC[C@@]56CCCN5C[C@H](F)C6)nc4N4CCC[C@]5(C4)NC(=O)NC5=O)C3)c12] 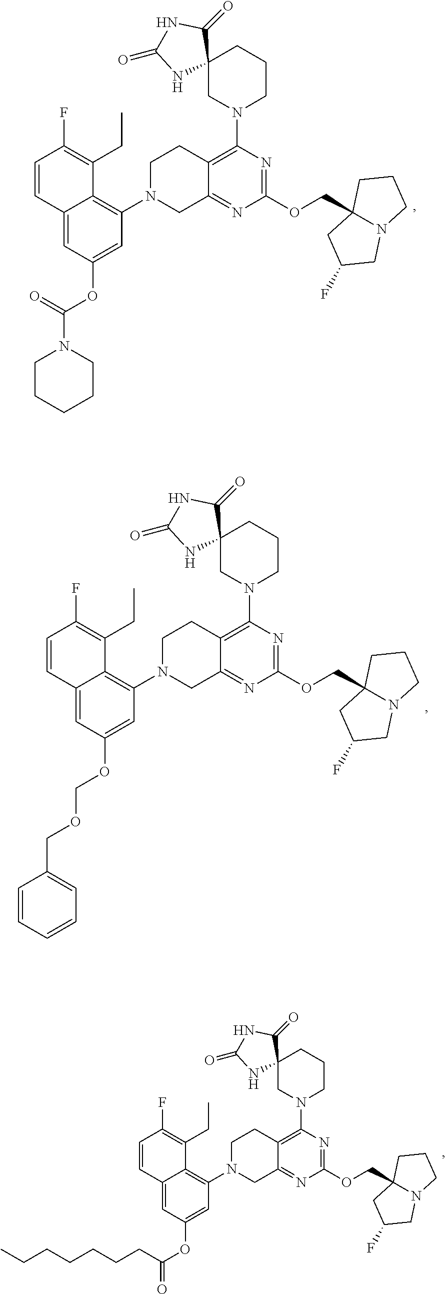 [SMILES: CCc1c(F)ccc2cc(OC(=O)N3CCCCC3)cc(N3CCc4c(nc(OC[C@@]56CCCN5C[C@H](F)C6)nc4N4CCC[C@]5(C4)NC(=O)NC5=O)C3)c12 ; CCc1c(F)ccc2cc(OCOCc3ccccc3)cc(N3CCc4c(nc(OC[C@@]56CCCN5C[C@H](F)C6)nc4N4CCC[C@]5(C4)NC(=O)NC5=O)C3)c12 ; CCCCCCCC(=O)Oc1cc(N2CCc3c(nc(OC[C@@]45CCCN4C[C@H](F)C5)nc3N3CCC[C@]4(C3)NC(=O)NC4=O)C2)c2c(CC)c(F)ccc2c1] 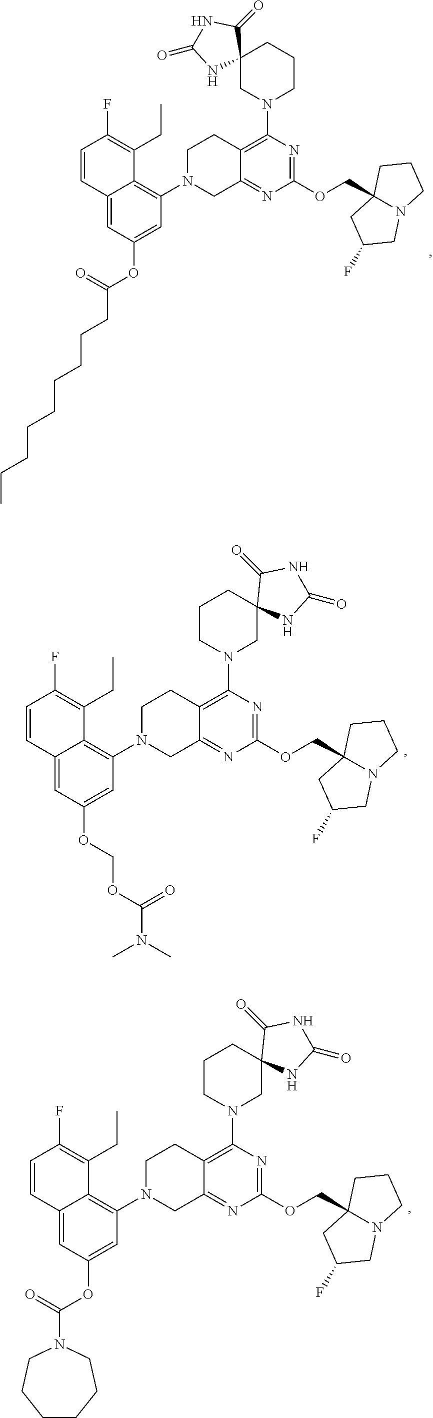 [SMILES: CCCCCCCCCC(=O)Oc1cc(N2CCc3c(nc(OC[C@@]45CCCN4C[C@H](F)C5)nc3N3CCC[C@]4(C3)NC(=O)NC4=O)C2)c2c(CC)c(F)ccc2c1 ; CCc1c(F)ccc2cc(OCOC(=O)N(C)C)cc(N3CCc4c(nc(OC[C@@]56CCCN5C[C@H](F)C6)nc4N4CCC[C@]5(C4)NC(=O)NC5=O)C3)c12 ; CCc1c(F)ccc2cc(OC(=O)N3CCCCCC3)cc(N3CCc4c(nc(OC[C@@]56CCCN5C[C@H](F)C6)nc4N4CCC[C@]5(C4)NC(=O)NC5=O)C3)c12] 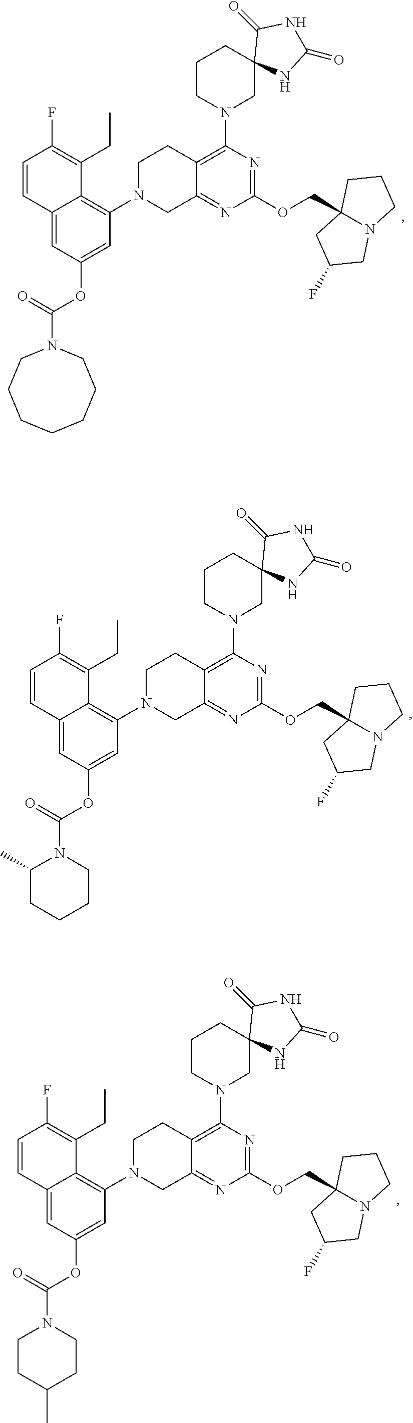 [SMILES: CCc1c(F)ccc2cc(OC(=O)N3CCCCCCC3)cc(N3CCc4c(nc(OC[C@@]56CCCN5C[C@H](F)C6)nc4N4CCC[C@]5(C4)NC(=O)NC5=O)C3)c12 ; CCc1c(F)ccc2cc(OC(=O)N3CCCC[C@@H]3C)cc(N3CCc4c(nc(OC[C@@]56CCCN5C[C@H](F)C6)nc4N4CCC[C@]5(C4)NC(=O)NC5=O)C3)c12 ; CCc1c(F)ccc2cc(OC(=O)N3CCC(C)CC3)cc(N3CCc4c(nc(OC[C@@]56CCCN5C[C@H](F)C6)nc4N4CCC[C@]5(C4)NC(=O)NC5=O)C3)c12] 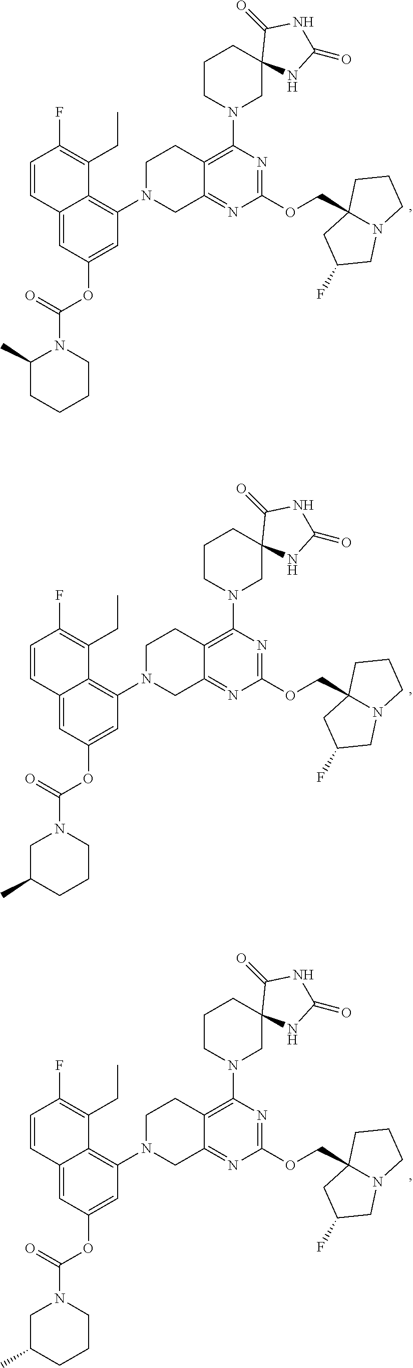 [SMILES: CCc1c(F)ccc2cc(OC(=O)N3CCCC[C@H]3C)cc(N3CCc4c(nc(OC[C@@]56CCCN5C[C@H](F)C6)nc4N4CCC[C@]5(C4)NC(=O)NC5=O)C3)c12 ; CCc1c(F)ccc2cc(OC(=O)N3CCC[C@@H](C)C3)cc(N3CCc4c(nc(OC[C@@]56CCCN5C[C@H](F)C6)nc4N4CCC[C@]5(C4)NC(=O)NC5=O)C3)c12 ; CCc1c(F)ccc2cc(OC(=O)N3CCC[C@H](C)C3)cc(N3CCc4c(nc(OC[C@@]56CCCN5C[C@H](F)C6)nc4N4CCC[C@]5(C4)NC(=O)NC5=O)C3)c12] 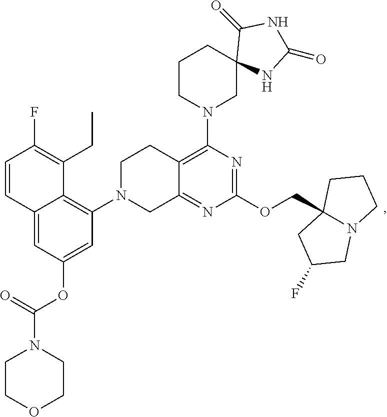 [SMILES: CCc1c(F)ccc2cc(OC(=O)N3CCOCC3)cc(N3CCc4c(nc(OC[C@@]56CCCN5C[C@H](F)C6)nc4N4CCC[C@]5(C4)NC(=O)NC5=O)C3)c12] 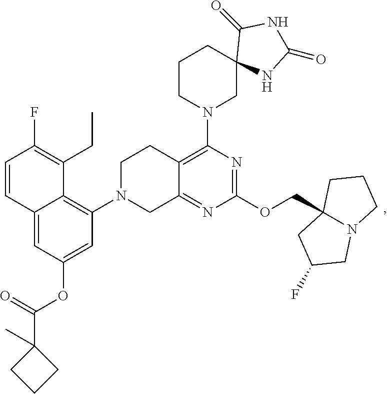 [SMILES: CCc1c(F)ccc2cc(OC(=O)C3(C)CCC3)cc(N3CCc4c(nc(OC[C@@]56CCCN5C[C@H](F)C6)nc4N4CCC[C@]5(C4)NC(=O)NC5=O)C3)c12] 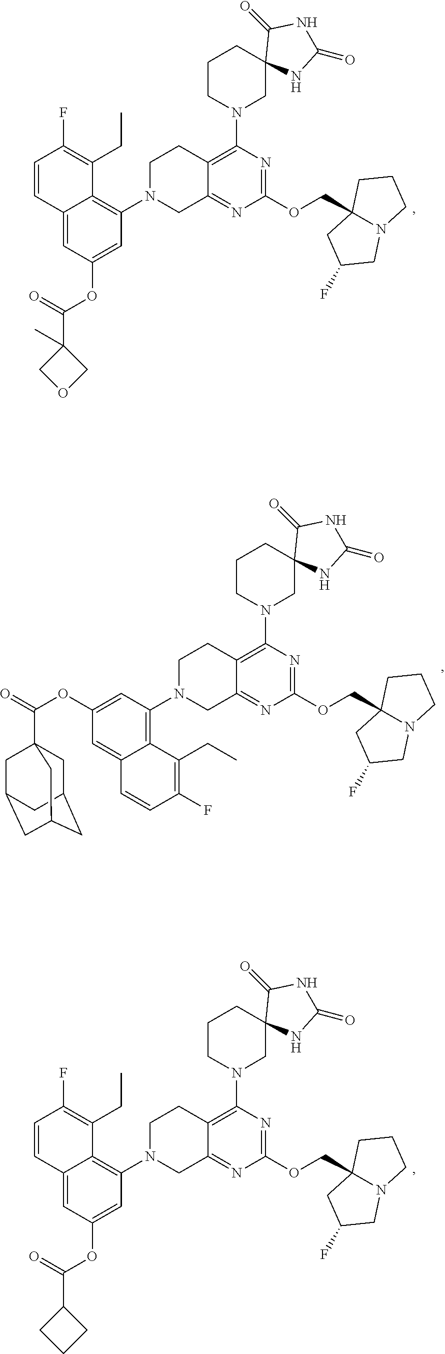 [SMILES: CCc1c(F)ccc2cc(OC(=O)C3(C)COC3)cc(N3CCc4c(nc(OC[C@@]56CCCN5C[C@H](F)C6)nc4N4CCC[C@]5(C4)NC(=O)NC5=O)C3)c12 ; CCc1c(F)ccc2cc(OC(=O)C34CC5CC(CC(C5)C3)C4)cc(N3CCc4c(nc(OC[C@@]56CCCN5C[C@H](F)C6)nc4N4CCC[C@]5(C4)NC(=O)NC5=O)C3)c12 ; CCc1c(F)ccc2cc(OC(=O)C3CCC3)cc(N3CCc4c(nc(OC[C@@]56CCCN5C[C@H](F)C6)nc4N4CCC[C@]5(C4)NC(=O)NC5=O)C3)c12] 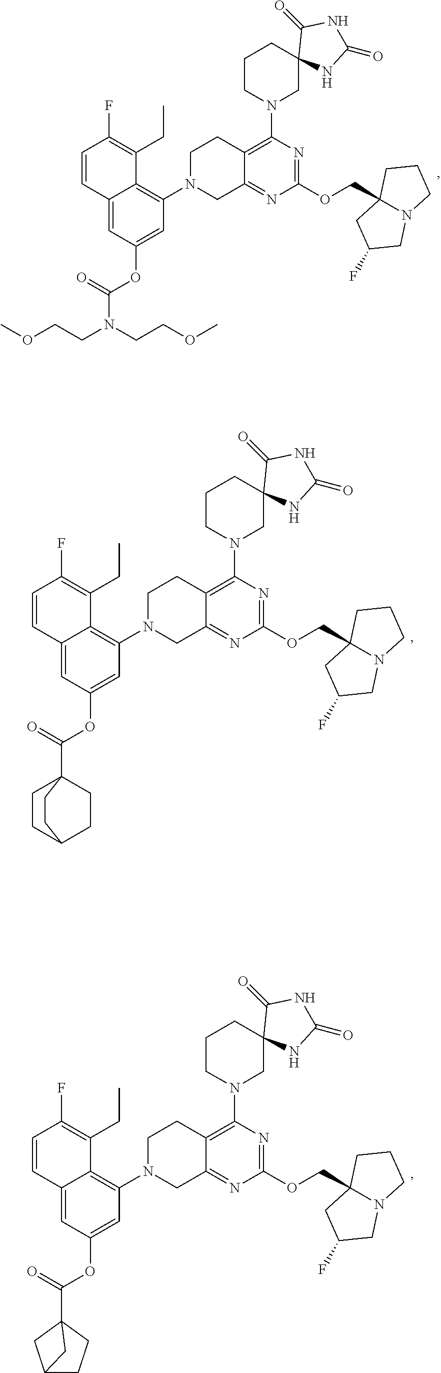 [SMILES: CCc1c(F)ccc2cc(OC(=O)N(CCOC)CCOC)cc(N3CCc4c(nc(OC[C@@]56CCCN5C[C@H](F)C6)nc4N4CCC[C@]5(C4)NC(=O)NC5=O)C3)c12 ; CCc1c(F)ccc2cc(OC(=O)C34CCC(CC3)CC4)cc(N3CCc4c(nc(OC[C@@]56CCCN5C[C@H](F)C6)nc4N4CCC[C@]5(C4)NC(=O)NC5=O)C3)c12 ; CCc1c(F)ccc2cc(OC(=O)C34CCC(C3)C4)cc(N3CCc4c(nc(OC[C@@]56CCCN5C[C@H](F)C6)nc4N4CCC[C@]5(C4)NC(=O)NC5=O)C3)c12] 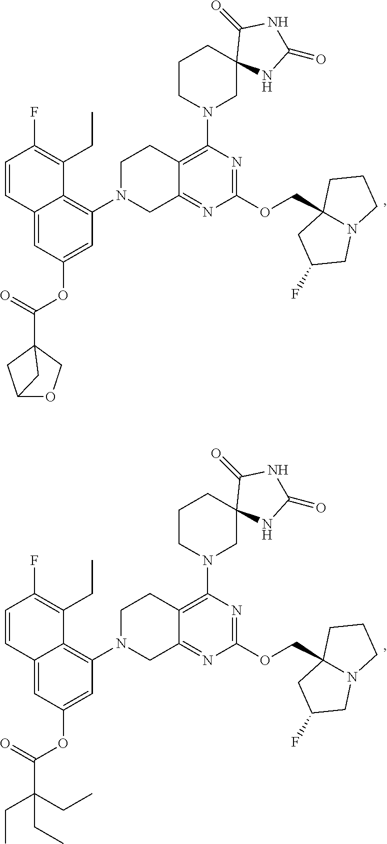 [SMILES: CCc1c(F)ccc2cc(OC(=O)C34COC(C3)C4)cc(N3CCc4c(nc(OC[C@@]56CCCN5C[C@H](F)C6)nc4N4CCC[C@]5(C4)NC(=O)NC5=O)C3)c12 ; CCc1c(F)ccc2cc(OC(=O)C(CC)(CC)CC)cc(N3CCc4c(nc(OC[C@@]56CCCN5C[C@H](F)C6)nc4N4CCC[C@]5(C4)NC(=O)NC5=O)C3)c12]
and pharmaceutically acceptable salts thereof.

10. The prodrug compound or salt of claim 1, wherein the compound has the following structure: 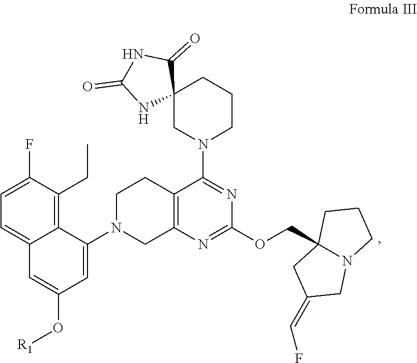
or a pharmaceutically acceptable salt thereof, wherein
R₁ is selected from the group consisting of:
R₃—*, R₃—O—CH₂—*, 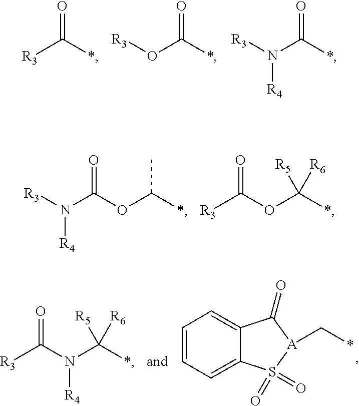
A is CH or N;
R₃ is C₁-C₂₀ alkyl, (C₁-C₄ alkyl)₀₋₁-cycloalkyl, (C₁-C₄ alkyl)₀₋₁-heterocyclyl or (C₁-C₄ alkyl)₀₋₁-aryl, where R₃ is optionally substituted with C₁-C₄ alkyl or —O— C₁-C₄ alkyl;
R₄ is H or R₃; and
R₅ and R₆ are independently selected from the group consisting of H and C₁-C₆ alkyl.

11. The compound or salt of claim 10, wherein A is CH.

12. The compound or salt of claim 10, wherein R₄ is R₃.

13. The compound or salt of claim 10, wherein the compound is selected from the group consisting of: 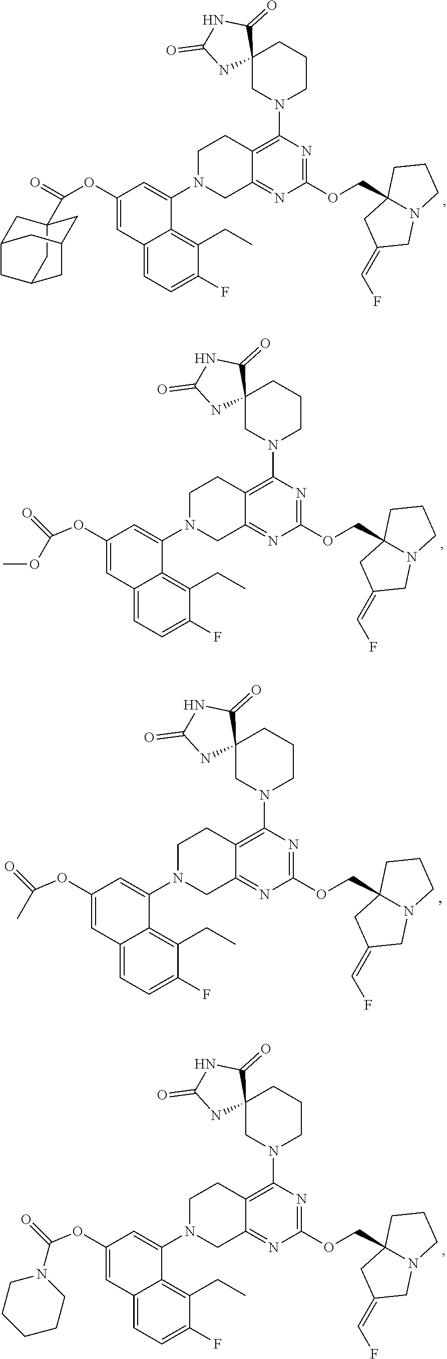 [SMILES: CCc1c(F)ccc2cc(OC(=O)C34CC5CC(CC(C5)C3)C4)cc(N3CCc4c(nc(OC[C@@]56CCCN5C/C(=C\F)C6)nc4N4CCC[C@]5(C4)NC(=O)NC5=O)C3)c12 ; CCc1c(F)ccc2cc(OC(=O)OC)cc(N3CCc4c(nc(OC[C@@]56CCCN5C/C(=C\F)C6)nc4N4CCC[C@]5(C4)NC(=O)NC5=O)C3)c12 ; CCc1c(F)ccc2cc(OC(C)=O)cc(N3CCc4c(nc(OC[C@@]56CCCN5C/C(=C\F)C6)nc4N4CCC[C@]5(C4)NC(=O)NC5=O)C3)c12 ; CCc1c(F)ccc2cc(OC(=O)N3CCCCC3)cc(N3CCc4c(nc(OC[C@@]56CCCN5C/C(=C\F)C6)nc4N4CCC[C@]5(C4)NC(=O)NC5=O)C3)c12] 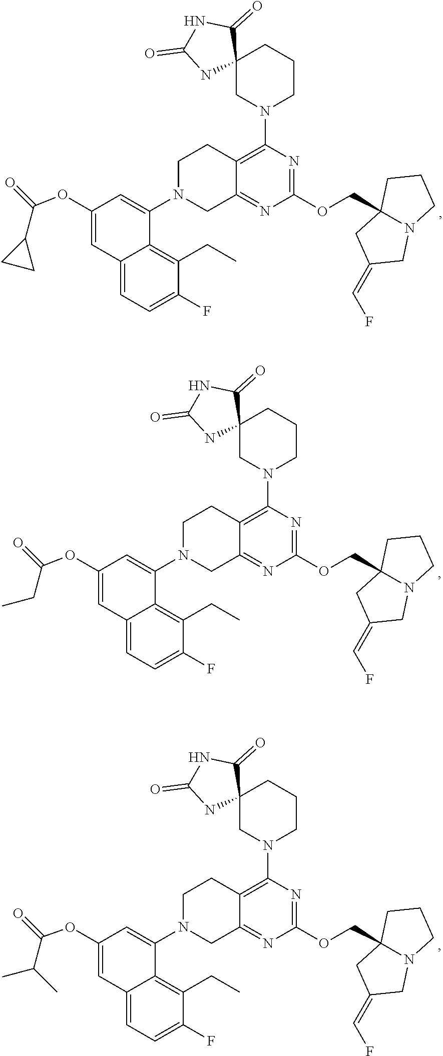 [SMILES: CCc1c(F)ccc2cc(OC(=O)C3CC3)cc(N3CCc4c(nc(OC[C@@]56CCCN5C/C(=C\F)C6)nc4N4CCC[C@]5(C4)NC(=O)NC5=O)C3)c12 ; CCC(=O)Oc1cc(N2CCc3c(nc(OC[C@@]45CCCN4C/C(=C\F)C5)nc3N3CCC[C@]4(C3)NC(=O)NC4=O)C2)c2c(CC)c(F)ccc2c1 ; CCc1c(F)ccc2cc(OC(=O)C(C)C)cc(N3CCc4c(nc(OC[C@@]56CCCN5C/C(=C\F)C6)nc4N4CCC[C@]5(C4)NC(=O)NC5=O)C3)c12]
and pharmaceutically acceptable salts thereof.

14. The prodrug compound or salt of claim 1, wherein the compound has the following structure: 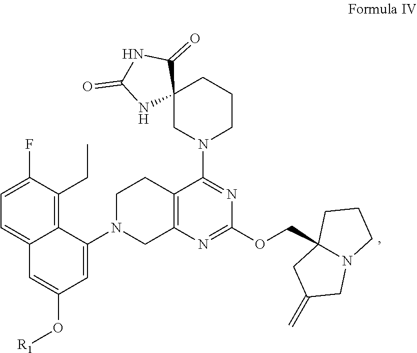
or a pharmaceutically acceptable salt thereof, wherein
R₁ is selected from the group consisting of:
R₃—*, R₃—O—CH₂—*, 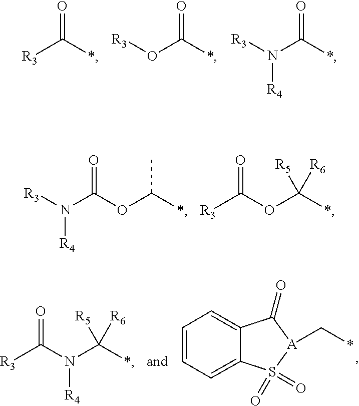
A is CH or N;
R₃ is C₁-C₂₀ alkyl, (C₁-C₄ alkyl)₀₋₁-cycloalkyl, (C₁-C₄ alkyl)₀₋₁-heterocyclyl or (C₁-C₄ alkyl)₀₋₁-aryl, where R₃ is optionally substituted with C₁-C₄ alkyl or —O— C₁-C₄ alkyl;
R₄ is H or R₃; and
R₅ and R₆ are independently selected from the group consisting of H and C₁-C₆ alkyl.

15. The compound or salt of claim 14, wherein A is CH.

16. The compound or salt of claim 14, wherein R₄ is R₃.

17. The compound or salt of claim 14, wherein the compound is selected from the group consisting of: 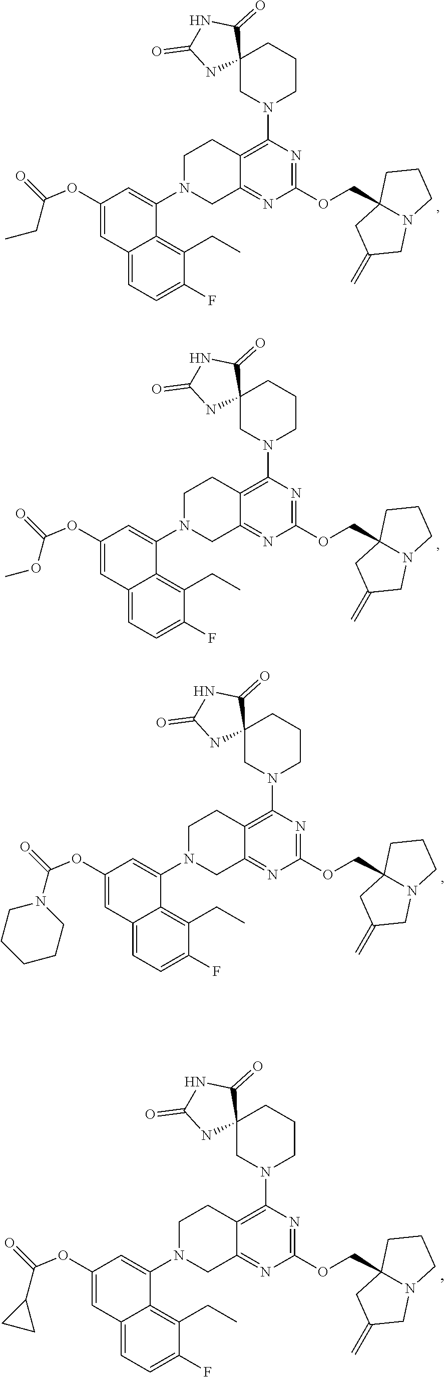 [SMILES: C=C1CN2CCC[C@@]2(COc2nc3c(c(N4CCC[C@]5(C4)NC(=O)NC5=O)n2)CCN(c2cc(OC(=O)CC)cc4ccc(F)c(CC)c24)C3)C1 ; C=C1CN2CCC[C@@]2(COc2nc3c(c(N4CCC[C@]5(C4)NC(=O)NC5=O)n2)CCN(c2cc(OC(=O)OC)cc4ccc(F)c(CC)c24)C3)C1 ; C=C1CN2CCC[C@@]2(COc2nc3c(c(N4CCC[C@]5(C4)NC(=O)NC5=O)n2)CCN(c2cc(OC(=O)N4CCCCC4)cc4ccc(F)c(CC)c24)C3)C1 ; C=C1CN2CCC[C@@]2(COc2nc3c(c(N4CCC[C@]5(C4)NC(=O)NC5=O)n2)CCN(c2cc(OC(=O)C4CC4)cc4ccc(F)c(CC)c24)C3)C1]
and pharmaceutically acceptable salts thereof.

18. A pharmaceutical composition, comprising a therapeutically effective amount of a compound of any one of claims 1-17 or a pharmaceutically acceptable salt thereof, and a pharmaceutically acceptable excipient.

19. A method for inhibiting the wild type KRas, KRas G12A, KRas G12C, KRas G12D, KRas G12R, KRas G12S, KRas G12V, KRas G13D or KRas Q61H activity in a cell, comprising contacting the cell in which inhibition of KRas activity is desired with an effective amount of a compound of according to any one of claims 1-17 or a pharmaceutically acceptable salt thereof, or the pharmaceutical composition of claim 18.

20. A method for treating cancer comprising administering to a patient having cancer a therapeutically effective amount of a compound according to any one of claims 1-17 or a pharmaceutically acceptable salt thereof, or the pharmaceutical composition of claim 18.

21. The method of claim 20, wherein the therapeutically effective amount of the compound is between about 0.01 to 100 mg/kg per day.

22. The method of claim 21, wherein the therapeutically effective amount of the compound is between about 0.1 to 50 mg/kg per day.

23. The method of claim 20, wherein the cancer is selected from the group consisting of Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; and Adrenal glands: neuroblastoma.

24. The method of claim 23, wherein the cancer is a KRas G12A-associated cancer.

25. The method of claim 23, wherein the cancer is a KRas G12C-associated cancer.

26. The method of claim 23, wherein the cancer is a KRas G12D-associated cancer.

27. The method of claim 23, wherein the cancer is a KRas G12R-associated cancer.

28. The method of claim 23, wherein the cancer is a KRas G12S-associated cancer.

29. The method of claim 23, wherein the cancer is a KRas G12V-associated cancer.

30. The method of claim 23, wherein the cancer is a KRas G13D-associated cancer.

31. The method of claim 23, wherein the cancer is a KRas Q61H-associated cancer.

32. The method of claim 23, wherein the cancer is a KRas G12A-associated cancer.

33. The method of claim 23, wherein the cancer is associated with at least one of wild type KRas, KRas G12A, KRas G12C, KRas G12D, KRas G12R, KRas G12S, KRas G12V, KRas G13D or KRas Q61H.

34. The method of any of claims 20-33, wherein the cancer is non-small cell lung cancer, small cell lung cancer, colorectal cancer, rectal cancer or pancreatic cancer.

35. A method for treating cancer in a patient in need thereof, the method comprising (a) determining that the cancer is associated with wild type KRas or a KRas G12A, KRas G12C, KRas G12D, KRas G12R, KRas G12S, KRas G12V, KRas G13D or KRas Q61H mutation; and (b) administering to the patient a therapeutically effective amount of a compound according to any one of claims 1-17 or a pharmaceutically acceptable salt thereof, or the pharmaceutical composition of claim 18.

36. The method of any one of claims 19-35, wherein the administering is done via a route selected from the group consisting of parenteral, intraperitoneal, intradermal, intracardiac, intraventricular, intracranial, intracerebrospinal, intrasynovial, intrathecal administration, intramuscular injection, intravitreous injection, intravenous injection, intra-arterial injection, oral, buccal, sublingual, transdermal, topical, intratracheal, intrarectal, subcutaneous, and topical administration.

37. The method of claim 36, wherein the administration route is oral.

38. The method of claim 36, wherein the administration is intravenous injection.

39. The method of claim 36, wherein the administration route is intramuscular injection.

40. The method of claim 36, wherein the administration route utilizes a delivery device.

41. The method of claim 36, wherein administration is done in a hospital setting.
